SEQUENCE LISTING (67 sequences)

SEQ ID NO: 1 - protein, 73 aa, Homo sapiens
ETCENVDCGPGKKCRMNKKNKPRCVCAPDCSNITWKGPVCGLDGKTYRNECALLKARCKEQPELEVQYQGRCK

SEQ ID NO: 2 - protein, 146 aa, synthetic construct
ETCENVDCGPGKKCRMNKKNKPRCVCAPDCSNITWKGPVCGLDGKTYRNECALLKARCKEQPELEVQYQGRCKETCENVDCGPGKKCRMNKKNKPRCVCAPDCSNITWKGPVCGLDGKTYRNECALLKARCKEQPELEVQYQGRCK

SEQ ID NO: 3 - protein, 147 aa, synthetic construct
METCENVDCGPGKKCRMNKKNKPRCVCAPDCSNITWKGPVCGLDGKTYRNECALLKARCKEQPELEVQYQGRCKETCENVDCGPGKKCRMNKKNKPRCVCAPDCSNITWKGPVCGLDGKTYRNECALLKARCKEQPELEVQYQGRCK

SEQ ID NO: 4 - DNA, 441 bp, synthetic construct
  source 1..441: mol_type = other DNA
atggagacatgcgagaacgtggattgtggaccaggcaagaagtgccggatgaacaagaagaacaagcccagatgcgtgtgcgctcctgactgcagcaacatcacctggaagggacccgtgtgcggcctggatggcaagacataccggaatgagtgcgccctgctgaaggctaggtgtaaggagcagcctgagctggaggtgcagtatcagggccggtgcaaggagacatgcgagaacgtggattgtggaccaggcaagaagtgccggatgaacaagaagaacaagcccagatgcgtgtgcgctcctgactgcagcaacatcacctggaagggacccgtgtgcggcctggatggcaagacataccggaatgagtgcgccctgctgaaggctaggtgtaaggagcagcctgagctggaggtgcagtatcagggccggtgcaag

SEQ ID NO: 5 - protein, 188 aa, synthetic construct
MQVQLVESGGGLVQSGGSLTLTCAASGFTFSAASMRWVRQVPGKGLEWVATIDGTGANSYYSESAKGRFTISRDNARNTLYLQMNNLKPDDTAVYYCANFGRNYWGKGTQVTVSSETCENVDCGPGKKCRMNKKNKPRCVCAPDCSNITWKGPVCGLDGKTYRNECALLKARCKEQPELEVQYQGRCK

SEQ ID NO: 6 - DNA, 564 bp, synthetic construct
  source 1..564: mol_type = other DNA
atgcaagtacagctagttgaatcaggtggagggttggtgcaaagcggtggttctctgaccttgacgtgcgcggcgtctggcttcaccttcagcgctgcctccatgcgttgggtacgtcaggtgccgggtaagggccttgagtgggtcgcaaccatcgacggtactggtgcaaacagctattacagcgagtcggcgaaaggccgttttacgatcagccgtgataatgcgcgtaacaccttatatctgcagatgaataacctgaaaccggatgacaccgcagtttactactgcgcgaactttggtcgcaactattggggtaagggcacgcaagtgaccgtttcctccgaaacctgtgaaaatgtcgattgcggtccgggcaaaaagtgccgtatgaacaagaagaacaagccgcgttgtgtttgcgctccggactgtagcaatattacctggaaaggcccagtgtgcggcctggacggcaaaacctaccgcaacgaatgtgcgctgctgaaagccagatgcaaagagcagccggaactggaggttcaatatcagggtcgctgcaag

SEQ ID NO: 7 - protein, 528 aa, synthetic construct
MKWVTFISLLFLFSSAYSSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKETCENVDCGPGKKCRMNKKNKPRCVCAPDCSNITWKGPVCGLDGKTYRNECALLKARCKEQPELEVQYQGRCK

SEQ ID NO: 8 - DNA, 1584 bp, synthetic construct
  source 1..1584: mol_type = other DNA
atgaagtgggtgaccttcatctctctgctgttcctgttctcttctgcctactctagcgacaccggcaggcccttcgtggagatgtacagcgagatccccgagatcatccacatgaccgagggcagggagctggtgatcccctgcagggtgaccagccccaacatcaccgtgaccctgaagaagttccccctggacaccctgatccccgacggcaagaggatcatctgggacagcaggaagggcttcatcatcagcaacgccacctacaaggagatcggcctgctgacctgcgaggccaccgtgaacggccacctgtacaagaccaactacctgacccacaggcagaccaacaccatcatcgacgtggtgctgagccccagccacggcatcgagctgagcgtgggcgagaagctggtgctgaactgcaccgccaggaccgagctgaacgtgggcatcgacttcaactgggagtaccccagcagcaagcaccagcacaagaagctggtgaacagggacctgaagacccagagcggcagcgagatgaagaagttcctgagcaccctgaccatcgacggcgtgaccaggagcgaccagggcctgtacacctgcgccgccagcagcggcctgatgaccaagaagaacagcaccttcgtgagggtgcacgagaagccacggggaccaaccatcaaaccatgccccccctgtaagtgccccgcacctaacctcctgggtgggccatccgtgtttatcttcccccctaaaattaaggatgtgctgatgatcagcctgagccctatcgtgacatgcgtggtcgtcgacgtgagcgaggatgaccccgacgtccagatctcttggtttgtgaacaacgtggaggttcacaccgcccaaacccagacacacagagaggactataattctactctccgcgttgtgtccgccctgcccatccagcaccaagattggatgtctggtaaggaattcaagtgtaaagtgaacaataaagatctgcccgcaccaatcgaaagaaccatttctaagcctaaaggctccgtgcgggctcctcaggtttacgtcctgccaccccccgaggaagagatgactaagaagcaggtgaccctgacctgtatggtgaccgattttatgcccgaagacatttacgtcgagtggaccaataatggtaagacagaactgaactataaaaatacagagccagtgctcgactctgatggcagctattttatgtactctaagctgagagttgagaagaaaaactgggtggaacggaattcttactcttgctctgtcgtgcacgaaggactgcacaaccaccacactaccaagtccttttctaggacccccggcaaggagacctgcgagaacgtggactgcggccccggcaagaagtgccggatgaacaagaagaacaagccccggtgcgtgtgcgcccccgactgcagcaacatcacctggaagggccccgtgtgcggcctggacggcaagacctaccggaacgagtgcgccctgctgaaggcccggtgcaaggagcagcccgagctggaggtgcagtaccagggccggtgcaag

SEQ ID NO: 9 - protein, 558 aa, synthetic construct
MKWVTFISLLFLFSSAYSLPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKETCENVDCGPGKKCRMNKKNKPRCVCAPDCSNITWKGPVCGLDGKTYRNECALLKARCKEQPELEVQYQGRCK

SEQ ID NO: 10 - DNA, 1674 bp, synthetic construct
  source 1..1674: mol_type = other DNA
atgaagtgggtgaccttcatctctctgctgttcctgttctcttctgcctactctctgcccgcccaggtggccttcaccccctacgcccccgagcccggcagcacctgcaggctgagggagtactacgaccagaccgcccagatgtgctgcagcaagtgcagccccggccagcacgccaaggtgttctgcaccaagaccagcgacaccgtgtgcgacagctgcgaggacagcacctacacccagctgtggaactgggtgcccgagtgcctgagctgcggcagcaggtgcagcagcgaccaggtggagacccaggcctgcaccagggagcagaacaggatctgcacctgcaggcccggctggtactgcgccctgagcaagcaggagggctgcaggctgtgcgcccccctgaggaagtgcaggcccggcttcggcgtggccaggcccggcaccgagaccagcgacgtggtgtgcaagccctgcgcccccggcaccttcagcaacaccaccagcagcaccgacatctgcaggccccaccagatctgcaacgtggtggccatccccggcaacgccagcatggacgccgtgtgcaccagcaccagccccaccaggagcatggcccccggcgccgtgcacctgccccagcccgtgagcaccaggagccagcacacccagcccacccccgagcccagcaccgcccccagcaccagcttcctgctgcccatgggccccagcccccccgccgagggcagcaccggcgacccacggggaccaaccatcaaaccatgccccccctgtaagtgccccgcacctaacctcctgggtgggccatccgtgtttatcttcccccctaaaattaaggatgtgctgatgatcagcctgagccctatcgtgacatgcgtggtcgtcgacgtgagcgaggatgaccccgacgtccagatctcttggtttgtgaacaacgtggaggttcacaccgcccaaacccagacacacagagaggactataattctactctccgcgttgtgtccgccctgcccatccagcaccaagattggatgtctggtaaggaattcaagtgtaaagtgaacaataaagatctgcccgcaccaatcgaaagaaccatttctaagcctaaaggctccgtgcgggctcctcaggtttacgtcctgccaccccccgaggaagagatgactaagaagcaggtgaccctgacctgtatggtgaccgattttatgcccgaagacatttacgtcgagtggaccaataatggtaagacagaactgaactataaaaatacagagccagtgctcgactctgatggcagctattttatgtactctaagctgagagttgagaagaaaaactgggtggaacggaattcttactcttgctctgtcgtgcacgaaggactgcacaaccaccacactaccaagtccttttctaggacccccggcaaggagacctgcgagaacgtggactgcggccccggcaagaagtgccggatgaacaagaagaacaagccccggtgcgtgtgcgcccccgactgcagcaacatcacctggaagggccccgtgtgcggcctggacggcaagacctaccggaacgagtgcgccctgctgaaggcccggtgcaaggagcagcccgagctggaggtgcagtaccagggccggtgcaag

SEQ ID NO: 11 - protein, 541 aa, synthetic construct
MKWVTFISLLFLFSSAYSGNCWLRQAKNGRCQVLYKTELSKEECCSTGRLSTSWTEEDVNDNTLFKWMIFNGGAPNCIPCKETCENVDCGPGKKCRMNKKNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTEKQVTLTCMVTDFMPEDIYVEWTNNGKTELNYKNTEPVLDSDGSYFMYSWLRVEKKNWVERNSYSCSVVHEGLHNHHTTKSFSRTPGK

SEQ ID NO: 12 - DNA, 1623 bp, synthetic construct
  source 1..1623: mol_type = other DNA
atgaagtgggtgaccttcatctctctgctgttcctgttctcttctgcctactctggcaactgctggctgcggcaggccaagaacggccggtgccaggtgctgtacaagaccgagctgagcaaggaggagtgctgcagcaccggccggctgagcaccagctggaccgaggaggacgtgaacgacaacaccctgttcaagtggatgatcttcaacggcggcgcccccaactgcatcccctgcaaggagacctgcgagaacgtggactgcggccccggcaagaagtgccggatgaacaagaagaacaagccccggtgcgtgtgcgcccccgactgcagcaacatcacctggaagggccccgtgtgcggcctggacggcaagacctaccggaacgagtgcgccctgctgaaggcccggtgcaaggagcagcccgagctggaggtgcagtaccagggccggtgcaagaagacctgccgggacgtgttctgccccggcagcagcacctgcgtggtggaccagaccaacaacgcctactgcgtgacctgcaaccggatctgccccgagcccgccagcagcgagcagtacctgtgcggcaacgacggcgtgacctacagcagcgcctgccacctgcggaaggccacctgcctgctgggccggagcatcggcctggcctacgagggcaagtgcatcaaggccaagagctgcgaggacatccagtgcaccggcggcaagaagtgcctgtgggacttcaaggtgggccggggccggtgcagcctgtgcgacgagctgtgccccgacagcaagagcgacgagcccgtgtgcgccagcgacaacgccacctacgccagcgagtgcgccatgaaggaggccgcctgcagcagcggcgtgctgctggaggtgaagcacagcggcagctgcaacagcatcagcccacggggaccaaccatcaaaccatgccccccctgtaagtgccccgcacctaacctcctgggtgggccatccgtgtttatcttcccccctaaaattaaggatgtgctgatgatcagcctgagccctatcgtgacatgcgtggtcgtcgacgtgagcgaggatgaccccgacgtccagatctcttggtttgtgaacaacgtggaggttcacaccgcccaaacccagacacacagagaggactataattctactctccgcgttgtgtccgccctgcccatccagcaccaagattggatgtctggtaaggaattcaagtgtaaagtgaacaataaagatctgcccgcaccaatcgaaagaaccatttctaagcctaaaggctccgtgcgggctcctcaggtttacgtcctgccaccccccgaggaagagatgactgagaagcaggtgaccctgacctgtatggtgaccgattttatgcccgaagacatttacgtcgagtggaccaataatggtaagacagaactgaactataaaaatacagagccagtgctcgactctgatggcagctattttatgtactcttggctgagagttgagaagaaaaactgggtggaacggaattcttactcttgctctgtcgtgcacgaaggactgcacaaccaccacactaccaagtccttttctaggacccccggcaag

SEQ ID NO: 13 - protein, 467 aa, synthetic construct
MKWVTFISLLFLFSSAYSSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPRVYVLPPPEEEMTKKQVTLTCMVTDFMPEDIYVEWTNNGKTELNYKNTEPVLVSDGSYTMYSKLRVEKKNWVERNSYSCSVVHEGLHNHHTTKSFSRTPGKLVPRGSHHHHHH

SEQ ID NO: 14 - DNA, 1401 bp, synthetic construct
  source 1..1401: mol_type = other DNA
atgaagtgggtgaccttcatctctctgctgttcctgttctcttctgcctactctagcgacaccggcaggcccttcgtggagatgtacagcgagatccccgagatcatccacatgaccgagggcagggagctggtgatcccctgcagggtgaccagccccaacatcaccgtgaccctgaagaagttccccctggacaccctgatccccgacggcaagaggatcatctgggacagcaggaagggcttcatcatcagcaacgccacctacaaggagatcggcctgctgacctgcgaggccaccgtgaacggccacctgtacaagaccaactacctgacccacaggcagaccaacaccatcatcgacgtggtgctgagccccagccacggcatcgagctgagcgtgggcgagaagctggtgctgaactgcaccgccaggaccgagctgaacgtgggcatcgacttcaactgggagtaccccagcagcaagcaccagcacaagaagctggtgaacagggacctgaagacccagagcggcagcgagatgaagaagttcctgagcaccctgaccatcgacggcgtgaccaggagcgaccagggcctgtacacctgcgccgccagcagcggcctgatgaccaagaagaacagcaccttcgtgagggtgcacgagaagccacggggaccaaccatcaaaccatgccccccctgtaagtgccccgcacctaacctcctgggtgggccatccgtgtttatcttcccccctaaaattaaggatgtgctgatgatcagcctgagccctatcgtgacatgcgtggtcgtcgacgtgagcgaggatgaccccgacgtccagatctcttggtttgtgaacaacgtggaggttcacaccgcccaaacccagacacacagagaggactataattctactctccgcgttgtgtccgccctgcccatccagcaccaagattggatgtctggtaaggaattcaagtgtaaagtgaacaataaagatctgcccgcaccaatcgaaagaaccatttctaagcctaaaggctccgtgcgggctcctagagtttacgtcctgccaccccccgaggaagagatgactaagaagcaggtgaccctgacctgtatggtgaccgattttatgcccgaagacatttacgtcgagtggaccaataatggtaagacagaactgaactataaaaatacagagccagtgctcgtgtctgatggcagctataccatgtactctaagctgagagttgagaagaaaaactgggtggaacggaattcttactcttgctctgtcgtgcacgaaggactgcacaaccaccacactaccaagtccttttctaggacccccggcaagctggtgcccagaggaagccatcatcatcatcatcac

SEQ ID NO: 15 - protein, 497 aa, synthetic construct
MKWVTFISLLFLFSSAYSLPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPRVYVLPPPEEEMTKKQVTLTCMVTDFMPEDIYVEWTNNGKTELNYKNTEPVLVSDGSYTMYSKLRVEKKNWVERNSYSCSVVHEGLHNHHTTKSFSRTPGKLVPRGSHHHHHH

SEQ ID NO: 16 - DNA, 1491 bp, synthetic construct
  source 1..1491: mol_type = other DNA
atgaagtgggtgaccttcatctctctgctgttcctgttctcttctgcctactctctgcccgcccaggtggccttcaccccctacgcccccgagcccggcagcacctgcaggctgagggagtactacgaccagaccgcccagatgtgctgcagcaagtgcagccccggccagcacgccaaggtgttctgcaccaagaccagcgacaccgtgtgcgacagctgcgaggacagcacctacacccagctgtggaactgggtgcccgagtgcctgagctgcggcagcaggtgcagcagcgaccaggtggagacccaggcctgcaccagggagcagaacaggatctgcacctgcaggcccggctggtactgcgccctgagcaagcaggagggctgcaggctgtgcgcccccctgaggaagtgcaggcccggcttcggcgtggccaggcccggcaccgagaccagcgacgtggtgtgcaagccctgcgcccccggcaccttcagcaacaccaccagcagcaccgacatctgcaggccccaccagatctgcaacgtggtggccatccccggcaacgccagcatggacgccgtgtgcaccagcaccagccccaccaggagcatggcccccggcgccgtgcacctgccccagcccgtgagcaccaggagccagcacacccagcccacccccgagcccagcaccgcccccagcaccagcttcctgctgcccatgggccccagcccccccgccgagggcagcaccggcgacccacggggaccaaccatcaaaccatgccccccctgtaagtgccccgcacctaacctcctgggtgggccatccgtgtttatcttcccccctaaaattaaggatgtgctgatgatcagcctgagccctatcgtgacatgcgtggtcgtcgacgtgagcgaggatgaccccgacgtccagatctcttggtttgtgaacaacgtggaggttcacaccgcccaaacccagacacacagagaggactataattctactctccgcgttgtgtccgccctgcccatccagcaccaagattggatgtctggtaaggaattcaagtgtaaagtgaacaataaagatctgcccgcaccaatcgaaagaaccatttctaagcctaaaggctccgtgcgggctcctagagtttacgtcctgccaccccccgaggaagagatgactaagaagcaggtgaccctgacctgtatggtgaccgattttatgcccgaagacatttacgtcgagtggaccaataatggtaagacagaactgaactataaaaatacagagccagtgctcgtgtctgatggcagctataccatgtactctaagctgagagttgagaagaaaaactgggtggaacggaattcttactcttgctctgtcgtgcacgaaggactgcacaaccaccacactaccaagtccttttctaggacccccggcaagctggtgcccagaggaagccatcatcatcatcatcac

SEQ ID NO: 17 - protein, 645 aa, synthetic construct
MKWVTFISLLFLFSSAYSMDPNAAYVNMSNHHRGLASANVDFAFSLYKHLVALSPKKNIFISPVSISMALAMLSLGTCGHTRAQLLQGLGFNLTERSETEIHQGFQHLHQLFAKSDTSLEMTMGNALFLDGSLELLESFSADIKHYYESEVLAMNFQDWATASRQINSYVKNKTQGKIVDLFSGLDSPAILVLVNYIFFKGTWTQPFDLASTREENFYVDETTVVKVPMMLQSSTISYLHDSELPCQLVQMNYVGNGTVFFILPDKGKMNTVIAALSRDTINRWSAGLTSSQVDLYIPKVTISGVYDLGDVLEEMGIADLFTNQANFSRITQDAQLKSSKVVHKAVLQLNEEGVDTAGSTGVTLNLTSKPIILRFNQPFIIMIFDHFTWSSLFLARVMNPVPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPRVYVLPPPEEEMTKKQVTLTCMVTDFMPEDIYVEWTNNGKTELNYKNTEPVLVSDGSYTMYSKLRVEKKNWVERNSYSCSVVHEGLHNHHTTKSFSRTPGKLVPRGSHHHHHH

SEQ ID NO: 18 - DNA, 1935 bp, synthetic construct
  source 1..1935: mol_type = other DNA
atgaagtgggtgaccttcatctctctgctgttcctgttctcttctgcctactctatggaccccaacgccgcctacgtgaacatgtctaaccaccacaggggcctggcctctgccaacgtggacttcgccttctctctgtacaagcacctggtggccctgtctcccaagaagaacatcttcatctctcccgtgtctatctctatggccctggccatgctgtctctgggcacctgcggccacaccagggcccagctgctgcagggcctgggcttcaacctgaccgagaggtctgagaccgagatccaccagggcttccagcacctgcaccagctgttcgccaagtctgacacctctctggagatgaccatgggcaacgccctgttcctggacggctctctggagctgctggagtctttctctgccgacatcaagcactactacgagtctgaggtgctggccatgaacttccaggactgggccaccgcctctaggcagatcaactcttacgtgaagaacaagacccagggcaagatcgtggacctgttctctggcctggactctcccgccatcctggtgctggtgaactacatcttcttcaagggcacctggacccagcccttcgacctggcctctaccagggaggagaacttctacgtggacgagaccaccgtggtgaaggtgcccatgatgctgcagtcttctaccatctcttacctgcacgactctgagctgccctgccagctggtgcagatgaactacgtgggcaacggcaccgtgttcttcatcctgcccgacaagggcaagatgaacaccgtgatcgccgccctgtctagggacaccatcaacaggtggtctgccggcctgacctcttctcaggtggacctgtacatccccaaggtgaccatctctggcgtgtacgacctgggcgacgtgctggaggagatgggcatcgccgacctgttcaccaaccaggccaacttctctaggatcacccaggacgcccagctgaagtcttctaaggtggtgcacaaggccgtgctgcagctgaacgaggagggcgtggacaccgccggctctaccggcgtgaccctgaacctgacctctaagcccatcatcctgaggttcaaccagcccttcatcatcatgatcttcgaccacttcacctggtcttctctgttcctggccagggtgatgaaccccgtgccacggggaccaaccatcaaaccatgccccccctgtaagtgccccgcacctaacctcctgggtgggccatccgtgtttatcttcccccctaaaattaaggatgtgctgatgatcagcctgagccctatcgtgacatgcgtggtcgtcgacgtgagcgaggatgaccccgacgtccagatctcttggtttgtgaacaacgtggaggttcacaccgcccaaacccagacacacagagaggactataattctactctccgcgttgtgtccgccctgcccatccagcaccaagattggatgtctggtaaggaattcaagtgtaaagtgaacaataaagatctgcccgcaccaatcgaaagaaccatttctaagcctaaaggctccgtgcgggctcctagagtttacgtcctgccaccccccgaggaagagatgactaagaagcaggtgaccctgacctgtatggtgaccgattttatgcccgaagacatttacgtcgagtggaccaataatggtaagacagaactgaactataaaaatacagagccagtgctcgtgtctgatggcagctataccatgtactctaagctgagagttgagaagaaaaactgggtggaacggaattcttactcttgctctgtcgtgcacgaaggactgcacaaccaccacactaccaagtccttttctaggacccccggcaagctggtgcccagaggaagccatcatcatcatcatcac

SEQ ID NO: 19 - protein, 102 aa, Homo sapiens
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTII

SEQ ID NO: 20 - protein, 103 aa, Homo sapiens
DVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEK

SEQ ID NO: 21 - protein, 235 aa, Homo sapiens
LPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGD

SEQ ID NO: 22 - protein, 405 aa, Homo sapiens
MPLLLYTCLLWLPTSGLWTVQAMDPNAAYVNMSNHHRGLASANVDFAFSLYKHLVALSPKKNIFISPVSISMALAMLSLGTCGHTRAQLLQGLGFNLTERSETEIHQGFQHLHQLFAKSDTSLEMTMGNALFLDGSLELLESFSADIKHYYESEVLAMNFQDWATASRQINSYVKNKTQGKIVDLFSGLDSPAILVLVNYIFFKGTWTQPFDLASTREENFYVDETTVVKVPMMLQSSTISYLHDSELPCQLVQMNYVGNGTVFFILPDKGKMNTVIAALSRDTINRWSAGLTSSQVDLYIPKVTISGVYDLGDVLEEMGIADLFTNQANFSRITQDAQLKSSKVVHKAVLQLNEEGVDTAGSTGVTLNLTSKPIILRFNQPFIIMIFDHFTWSSLFLARVMNPV

SEQ ID NO: 23 - protein, 76 aa, synthetic construct
ETCENVDCGPGKKCRMNKKNKPRCVCAPDCSNITWKGPVCGLDGKTYRNECALLKARCKEQPELEVQYQGRCKKTC

SEQ ID NO: 24 - protein, 77 aa, synthetic construct
METCENVDCGPGKKCRMNKKNKPRCVCAPDCSNITWKGPVCGLDGKTYRNECALLKARCKEQPELEVQYQGRCKKTC

SEQ ID NO: 25 - DNA, 231 bp, synthetic construct
  source 1..231: mol_type = other DNA
atggagacatgcgagaacgtggattgtggaccaggcaagaagtgccggatgaacaagaagaacaagcccagatgcgtgtgcgctcctgactgcagcaacatcacctggaagggacccgtgtgcggcctggatggcaagacataccggaatgagtgcgccctgctgaaggctaggtgtaaggagcagcctgagctggaggtgcagtatcagggccggtgcaagaaaacatgt

SEQ ID NO: 26 - protein, 320 aa, Homo sapiens
MVRARHQPGGLCLLLLLLCQFMEDRSAQAGNCWLRQAKNGRCQVLYKTELSKEECCSTGRLSTSWTEEDVNDNTLFKWMIFNGGAPNCIPCKETCENVDCGPGKKCRMNKKNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSIS

SEQ ID NO: 27 - protein, 317 aa, Homo sapiens
MVRARHQPGGLCLLLLLLCQFMEDRSAQAGNCWLRQAKNGRCQVLYKTELSKEECCSTGRLSTSWTEEDVNDNTLFKWMIFNGGAPNCIPCKETCENVDCGPGKKCRMNKKNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCN

SEQ ID NO: 28 - protein, 344 aa, Homo sapiens
MVRARHQPGGLCLLLLLLCQFMEDRSAQAGNCWLRQAKNGRCQVLYKTELSKEECCSTGRLSTSWTEEDVNDNTLFKWMIFNGGAPNCIPCKETCENVDCGPGKKCRMNKKNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEW

SEQ ID NO: 29 - protein, 344 aa, synthetic construct
MVRARHQPGGLCLLLLLLCQFMEDRSAQAGNCWLRQAKNGRCQVLYKTELSKEECCSTGRLSTSWTEEDVNDNTLFKWMIFNGGAPNCIPCKETCENVDCGPGSTCVVDQTNNPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEW

SEQ ID NO: 30 - protein, 576 aa, synthetic construct
MVRARHQPGGLCLLLLLLCQFMEDRSAQAGNCWLRQAKNGRCQVLYKTELSKEECCSTGRLSTSWTEEDVNDNTLFKWMIFNGGAPNCIPCKETCENVDCGPGSTCVVDQTNNPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEWPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 31 - DNA, 1728 bp, synthetic construct
  source 1..1728: mol_type = other DNA
atggttagagccagacatcagcctgggggcctgtgcctgctgctccttcttctgtgtcagttcatggaggaccgcagtgctcaggccggtaactgttggttgagacaggctaagaacggtcggtgccaggtgctctacaaaacagagctctcaaaggaggaatgctgctccactggacgtctaagcacgtcttggactgaggaggatgtcaacgacaatacattgtttaaatggatgatatttaacggcggagcacccaactgcataccttgcaaggagacgtgtgagaatgttgactgtggcccaggttcaacgtgcgtggtggatcagaccaataatcctaggtgcgtgtgcgcccccgactgctccaacattacatggaagggccctgtgtgtgggctggatggtaaaacatatagaaacgaatgtgctttgttgaaagctcgttgtaaagagcagccggagctggaagtccaataccagggccgatgcaaaaagacttgtagagatgtcttctgtccaggtagcagcacctgtgttgtcgaccagactaataacgcttactgtgtgacctgcaataggatctgtccggaacctgcatcaagcgagcaatacctttgtggcaatgacggagtcacttactcaagcgcatgccacctgaggaaggcaacatgcctcctgggcaggagcataggactcgcctatgaaggaaagtgcattaaggcaaaatcttgtgaggacatccaatgcactggcgggaagaagtgtctttgggactttaaagtggggcgcgggagatgctccctctgcgatgaactgtgccccgattccaaaagtgacgaacctgtctgcgcctctgataacgctacctatgcatctgagtgcgcaatgaaggaagccgcatgcagtagcggagtattgcttgaggtgaagcactcaggctcctgtaattccatatcagaggataccgaggaggaggaggaggacgaggatcaggattattcatttccgatttcatcaatcttagagtggccgcgcgggccaacaatcaagccatgcccaccctgtaaatgccccgccccaaacctgctgggaggccctagtgtgtttatcttcccgcccaaaatcaaggatgtgctcatgatctctctaagcccaatcgtgacatgcgtggtcgtcgacgtctccgaggacgaccccgacgtgcaaatttcctggttcgtgaacaatgtcgaggtccacaccgctcagacccaaacccatcgcgaggattacaatagtactctgcgggtggtttcagccctgcctatccagcaccaggattggatgagtggcaaggagtttaagtgtaaggtgaataacaaggacctgccagctcctatcgaaaggacaatatctaagcctaaggggtctgtgcgggcacctcaggtctatgtcctgccacccccagaagaggaaatgacaaagaaacaggtgactctgacatgtatggttaccgacttcatgcctgaggacatatatgtcgagtggaccaataatggaaagactgagctaaactacaaaaataccgaaccagttttagattccgacggcagttacttcatgtactcaaagctgcgggtggagaaaaagaactgggtggagagaaactcatacagttgcagtgtggtgcacgaggggttgcataatcaccacaccaccaaatccttctccagaactccaggaaaa

SEQ ID NO: 32 - protein, 74 aa, Homo sapiens
METCENVDCGPGKKCRMNKKNKPRCVCAPDCSNITWKGPVCGLDGKTYRNECALLKARCKEQPELEVQYQGRCK

SEQ ID NO: 33 - DNA, 222 bp, Homo sapiens
  source 1..222: mol_type = genomic DNA
atggagacatgcgagaacgtggattgtggaccaggcaagaagtgccggatgaacaagaagaacaagcccagatgcgtgtgcgctcctgactgcagcaacatcacctggaagggacccgtgtgcggcctggatggcaagacataccggaatgagtgcgccctgctgaaggctaggtgtaaggagcagcctgagctggaggtgcagtatcagggccggtgcaag

SEQ ID NO: 34 - protein, 127 aa, synthetic construct
MQVQLVESGGGLVQSGGSLTLTCAASGFTFSAASMRWVRQVPGKGLEWVATIDGTGANSYYSESAKGRFTISRDNARNTLYLQMNNLKPDDTAVYYCANFGRNYWGKGTQVTVSSLVPRGSHHHHHH

SEQ ID NO: 35 - DNA, 381 bp, synthetic construct
  source 1..381: mol_type = other DNA
atgcaagtacagctagttgaatcaggtggagggttggtgcaaagcggtggttctctgaccttgacgtgcgcggcgtctggcttcaccttcagcgctgcctccatgcgttgggtacgtcaggtgccgggtaagggccttgagtgggtcgcaaccatcgacggtactggtgcaaacagctattacagcgagtcggcgaaaggccgttttacgatcagccgtgataatgcgcgtaacaccttatatctgcagatgaataacctgaaaccggatgacaccgcagtttactactgcgcgaactttggtcgcaactattggggtaagggcacgcaagtgaccgtttcctccctagtacccaggggtagccaccaccaccaccaccac

SEQ ID NO: 36 - protein, 12 aa, Homo sapiens
KKCRMNKKNKPR

SEQ ID NO: 37 - protein, 232 aa, Mus musculus
PRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 38 - protein, 232 aa, synthetic construct
PRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTEKQVTLTCMVTDFMPEDIYVEWTNNGKTELNYKNTEPVLDSDGSYFMYSWLRVEKKNWVERNSYSCSVVHEGLHNHHTTKSFSRTPGK

SEQ ID NO: 39 - protein, 232 aa, synthetic construct
PRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPRVYVLPPPEEEMTKKQVTLTCMVTDFMPEDIYVEWTNNGKTELNYKNTEPVLVSDGSYTMYSKLRVEKKNWVERNSYSCSVVHEGLHNHHTTKSFSRTPGK

SEQ ID NO: 40 - protein, 205 aa, Homo sapiens
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEK

SEQ ID NO: 41 - protein, 75 aa, Homo sapiens
KTCRDVFCPGSSTCVVDQTNNAYCVTCNRICPEPASSEQYLCGNDGVTYSSACHLRKATCLLGRSIGLAYEGKCI

SEQ ID NO: 42 - protein, 77 aa, Homo sapiens
KAKSCEDIQCTGGKKCLWDFKVGRGRCSLCDELCPDSKSDEPVCASDNATYASECAMKEAACSSGVLLEVKHSGSCN

SEQ ID NO: 43 - protein, 63 aa, Homo sapiens
GNCWLRQAKNGRCQVLYKTELSKEECCSTGRLSTSWTEEDVNDNTLFKWMIFNGGAPNCIPCK

SEQ ID NO: 44 - protein, 426 aa, Homo sapiens
MPLLWLRGFLLASCWIIVRSSPTPGSEGHSAAPDCPSCALAALPKDVPNSQPEMVEAVKKHILNMLHLKKRPDVTQPVPKAALLNAIRKLHVGKVGENGYVEIEDDIGRRAEMNELMEQTSEIITFAESGTARKTLHFEISKEGSDLSVVERAEVWLFLKVPKANRTRTKVTIRLFQQQKHPQGSLDTGEEAEEVGLKGERSELLLSEKVVDARKSTWHVFPVSSSIQRLLDQGKSSLDVRIACEQCQESGASLVLLGKKKKKEEEGEGKKKGGGEGGAGADEEKEQSHRPFLMLQARQSEDHPHRRRRRGLECDGKVNICCKKQFFVSFKDIGWNDWIIAPSGYHANYCEGECPSHIAGTSGSSLSFHSTVINHYRMRGHSPFANLKSCCVPTKLRPMSMLYYDDGQNIIKKDIQNMIVEECGCS

SEQ ID NO: 45 - protein, 375 aa, Homo sapiens
MQKLQLCVYIYLFMLIVAGPVDLNENSEQKENVEKEGLCNACTWRQNTKSSRIEAIKIQILSKLRLETAPNISKDVIRQLLPKAPPLRELIDQYDVQRDDSSDGSLEDDDYHATTETIITMPTESDFLMQVDGKPKCCFFKFSSKIQYNKVVKAQLWIYLRPVETPTTVFVQILRLIKPMKDGTRYTGIRSLKLDMNPGTGIWQSIDVKTVLQNWLKQPESNLGIEIKALDENGHDLAVTFPGPGEDGLNPFLEVKVTDTPKRSRRDFGLDCDEHSTESRCCRYPLTVDFEAFGWDWIIAPKRYKANYCSGECEFVFLQKYPHTHLVHQANPRGSAGPCCTPTKMSPINMLYFNGKEQIIYGKIPAMVVDRCGCS

SEQ ID NO: 46 - protein, 407 aa, Homo sapiens
MVLAAPLLLGFLLLALELRPRGEAAEGPAAAAAAAAAAAAAGVGGERSSRPAPSVAPEPDGCPVCVWRQHSRELRLESIKSQILSKLRLKEAPNISREVVKQLLPKAPPLQQILDLHDFQGDALQPEDFLEEDEYHATTETVISMAQETDPAVQTDGSPLCCHFHFSPKVMFTKVLKAQLWVYLRPVPRPATVYLQILRLKPLTGEGTAGGGGGGRRHIRIRSLKIELHSRSGHWQSIDFKQVLHSWFRQPQSNWGIEINAFDPSGTDLAVTSLGPGAEGLHPFMELRVLENTKRSRRNLGLDCDEHSSESRCCRYPLTVDFEAFGWDWIIAPKRYKANYCSGQCEYMFMQKYPHTHLVQQANPRGSAGPCCTPTKMSPINMLYFNDKQQIIYGKIPGMVVDRCGCS

SEQ ID NO: 47 - protein, 73 aa, Homo sapiens
ETCENVDCGPGKKCRMNKKNKPRCVCAPDCSNITWKGPVCGLDGKTYRNECALLKARCKEAPELEVQYQGRCK

SEQ ID NO: 48 - protein, 74 aa, Homo sapiens
METCENVDCGPGKKCRMNKKNKPRCVCAPDCSNITWKGPVCGLDGKTYRNECALLKARCKEAPELEVQYQGRCK

SEQ ID NO: 49 - DNA, 222 bp, Homo sapiens
  source 1..222: mol_type = genomic DNA
atggagacatgcgagaacgtggattgtggaccaggcaagaagtgccggatgaacaagaagaacaagcccagatgcgtgtgcgctcctgactgcagcaacatcacctggaagggacccgtgtgcggcctggatggcaagacataccggaatgagtgcgccctgctgaaggctaggtgtaaggaggcgcctgagctggaggtgcagtatcagggccggtgcaag

SEQ ID NO: 50 - protein, 73 aa, Homo sapiens
ETCENVDCGPGKKCRMNKKNKPRCVCAPDCSNITWKGPVCGLDGKTYRNECALLKARCKEQPALEVQYQGRCK

SEQ ID NO: 51 - protein, 74 aa, Homo sapiens
METCENVDCGPGKKCRMNKKNKPRCVCAPDCSNITWKGPVCGLDGKTYRNECALLKARCKEQPALEVQYQGRCK

SEQ ID NO: 52 - DNA, 222 bp, Homo sapiens
  source 1..222: mol_type = genomic DNA
atggagacatgcgagaacgtggattgtggaccaggcaagaagtgccggatgaacaagaagaacaagcccagatgcgtgtgcgctcctgactgcagcaacatcacctggaagggacccgtgtgcggcctggatggcaagacataccggaatgagtgcgccctgctgaaggctaggtgtaaggagcagcctgcgctggaggtgcagtatcagggccggtgcaag

SEQ ID NO: 53 - protein, 73 aa, Homo sapiens
ETCENVDCGPGKKCRMNKKNKPRCVCAPDCSNITWKGPVCGLDGKTYRNECALLKARCKEAPALEVQYQGRCK

SEQ ID NO: 54 - protein, 74 aa, Homo sapiens
METCENVDCGPGKKCRMNKKNKPRCVCAPDCSNITWKGPVCGLDGKTYRNECALLKARCKEAPALEVQYQGRCK

SEQ ID NO: 55 - DNA, 222 bp, Homo sapiens
  source 1..222: mol_type = genomic DNA
atggagacatgcgagaacgtggattgtggaccaggcaagaagtgccggatgaacaagaagaacaagcccagatgcgtgtgcgctcctgactgcagcaacatcacctggaagggacccgtgtgcggcctggatggcaagacataccggaatgagtgcgccctgctgaaggctaggtgtaaggaggcgcctgcgctggaggtgcagtatcagggccggtgcaag

SEQ ID NO: 56 - protein, 528 aa, synthetic construct
MKWVTFISLLFLFSSAYSSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKETCENVDCGPGKKCRMNKKNKPRCVCAPDCSNITWKGPVCGLDGKTYRNECALLKARCKEAPELEVQYQGRCK

SEQ ID NO: 57 - DNA, 1584 bp, synthetic construct
  source 1..1584: mol_type = other DNA
atgaagtgggtgaccttcatctctctgctgttcctgttctcttctgcctactctagcgacaccggcaggcccttcgtggagatgtacagcgagatccccgagatcatccacatgaccgagggcagggagctggtgatcccctgcagggtgaccagccccaacatcaccgtgaccctgaagaagttccccctggacaccctgatccccgacggcaagaggatcatctgggacagcaggaagggcttcatcatcagcaacgccacctacaaggagatcggcctgctgacctgcgaggccaccgtgaacggccacctgtacaagaccaactacctgacccacaggcagaccaacaccatcatcgacgtggtgctgagccccagccacggcatcgagctgagcgtgggcgagaagctggtgctgaactgcaccgccaggaccgagctgaacgtgggcatcgacttcaactgggagtaccccagcagcaagcaccagcacaagaagctggtgaacagggacctgaagacccagagcggcagcgagatgaagaagttcctgagcaccctgaccatcgacggcgtgaccaggagcgaccagggcctgtacacctgcgccgccagcagcggcctgatgaccaagaagaacagcaccttcgtgagggtgcacgagaagccacggggaccaaccatcaaaccatgccccccctgtaagtgccccgcacctaacctcctgggtgggccatccgtgtttatcttcccccctaaaattaaggatgtgctgatgatcagcctgagccctatcgtgacatgcgtggtcgtcgacgtgagcgaggatgaccccgacgtccagatctcttggtttgtgaacaacgtggaggttcacaccgcccaaacccagacacacagagaggactataattctactctccgcgttgtgtccgccctgcccatccagcaccaagattggatgtctggtaaggaattcaagtgtaaagtgaacaataaagatctgcccgcaccaatcgaaagaaccatttctaagcctaaaggctccgtgcgggctcctcaggtttacgtcctgccaccccccgaggaagagatgactaagaagcaggtgaccctgacctgtatggtgaccgattttatgcccgaagacatttacgtcgagtggaccaataatggtaagacagaactgaactataaaaatacagagccagtgctcgactctgatggcagctattttatgtactctaagctgagagttgagaagaaaaactgggtggaacggaattcttactcttgctctgtcgtgcacgaaggactgcacaaccaccacactaccaagtccttttctaggacccccggcaaggagacctgcgagaacgtggactgcggccccggcaagaagtgccggatgaacaagaagaacaagccccggtgcgtgtgcgcccccgactgcagcaacatcacctggaagggccccgtgtgcggcctggacggcaagacctaccggaacgagtgcgccctgctgaaggcccggtgcaaggaggcccccgagctggaggtgcagtaccagggccggtgcaag

SEQ ID NO: 58 - protein, 528 aa, synthetic construct
MKWVTFISLLFLFSSAYSSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKETCENVDCGPGKKCRMNKKNKPRCVCAPDCSNITWKGPVCGLDGKTYRNECALLKARCKEQPALEVQYQGRCK

SEQ ID NO: 59 - DNA, 1584 bp, synthetic construct
  source 1..1584: mol_type = other DNA
atgaagtgggtgaccttcatctctctgctgttcctgttctcttctgcctactctagcgacaccggcaggcccttcgtggagatgtacagcgagatccccgagatcatccacatgaccgagggcagggagctggtgatcccctgcagggtgaccagccccaacatcaccgtgaccctgaagaagttccccctggacaccctgatccccgacggcaagaggatcatctgggacagcaggaagggcttcatcatcagcaacgccacctacaaggagatcggcctgctgacctgcgaggccaccgtgaacggccacctgtacaagaccaactacctgacccacaggcagaccaacaccatcatcgacgtggtgctgagccccagccacggcatcgagctgagcgtgggcgagaagctggtgctgaactgcaccgccaggaccgagctgaacgtgggcatcgacttcaactgggagtaccccagcagcaagcaccagcacaagaagctggtgaacagggacctgaagacccagagcggcagcgagatgaagaagttcctgagcaccctgaccatcgacggcgtgaccaggagcgaccagggcctgtacacctgcgccgccagcagcggcctgatgaccaagaagaacagcaccttcgtgagggtgcacgagaagccacggggaccaaccatcaaaccatgccccccctgtaagtgccccgcacctaacctcctgggtgggccatccgtgtttatcttcccccctaaaattaaggatgtgctgatgatcagcctgagccctatcgtgacatgcgtggtcgtcgacgtgagcgaggatgaccccgacgtccagatctcttggtttgtgaacaacgtggaggttcacaccgcccaaacccagacacacagagaggactataattctactctccgcgttgtgtccgccctgcccatccagcaccaagattggatgtctggtaaggaattcaagtgtaaagtgaacaataaagatctgcccgcaccaatcgaaagaaccatttctaagcctaaaggctccgtgcgggctcctcaggtttacgtcctgccaccccccgaggaagagatgactaagaagcaggtgaccctgacctgtatggtgaccgattttatgcccgaagacatttacgtcgagtggaccaataatggtaagacagaactgaactataaaaatacagagccagtgctcgactctgatggcagctattttatgtactctaagctgagagttgagaagaaaaactgggtggaacggaattcttactcttgctctgtcgtgcacgaaggactgcacaaccaccacactaccaagtccttttctaggacccccggcaaggagacctgcgagaacgtggactgcggccccggcaagaagtgccggatgaacaagaagaacaagccccggtgcgtgtgcgcccccgactgcagcaacatcacctggaagggccccgtgtgcggcctggacggcaagacctaccggaacgagtgcgccctgctgaaggcccggtgcaaggagcagcccgccctggaggtgcagtaccagggccggtgcaag

SEQ ID NO: 60 - protein, 528 aa, synthetic construct
MKWVTFISLLFLFSSAYSSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKETCENVDCGPGKKCRMNKKNKPRCVCAPDCSNITWKGPVCGLDGKTYRNECALLKARCKEAPALEVQYQGRCK

SEQ ID NO: 61 - DNA, 1584 bp, synthetic construct
  source 1..1584: mol_type = other DNA
atgaagtgggtgaccttcatctctctgctgttcctgttctcttctgcctactctagcgacaccggcaggcccttcgtggagatgtacagcgagatccccgagatcatccacatgaccgagggcagggagctggtgatcccctgcagggtgaccagccccaacatcaccgtgaccctgaagaagttccccctggacaccctgatccccgacggcaagaggatcatctgggacagcaggaagggcttcatcatcagcaacgccacctacaaggagatcggcctgctgacctgcgaggccaccgtgaacggccacctgtacaagaccaactacctgacccacaggcagaccaacaccatcatcgacgtggtgctgagccccagccacggcatcgagctgagcgtgggcgagaagctggtgctgaactgcaccgccaggaccgagctgaacgtgggcatcgacttcaactgggagtaccccagcagcaagcaccagcacaagaagctggtgaacagggacctgaagacccagagcggcagcgagatgaagaagttcctgagcaccctgaccatcgacggcgtgaccaggagcgaccagggcctgtacacctgcgccgccagcagcggcctgatgaccaagaagaacagcaccttcgtgagggtgcacgagaagccacggggaccaaccatcaaaccatgccccccctgtaagtgccccgcacctaacctcctgggtgggccatccgtgtttatcttcccccctaaaattaaggatgtgctgatgatcagcctgagccctatcgtgacatgcgtggtcgtcgacgtgagcgaggatgaccccgacgtccagatctcttggtttgtgaacaacgtggaggttcacaccgcccaaacccagacacacagagaggactataattctactctccgcgttgtgtccgccctgcccatccagcaccaagattggatgtctggtaaggaattcaagtgtaaagtgaacaataaagatctgcccgcaccaatcgaaagaaccatttctaagcctaaaggctccgtgcgggctcctcaggtttacgtcctgccaccccccgaggaagagatgactaagaagcaggtgaccctgacctgtatggtgaccgattttatgcccgaagacatttacgtcgagtggaccaataatggtaagacagaactgaactataaaaatacagagccagtgctcgactctgatggcagctattttatgtactctaagctgagagttgagaagaaaaactgggtggaacggaattcttactcttgctctgtcgtgcacgaaggactgcacaaccaccacactaccaagtccttttctaggacccccggcaaggagacctgcgagaacgtggactgcggccccggcaagaagtgccggatgaacaagaagaacaagccccggtgcgtgtgcgcccccgactgcagcaacatcacctggaagggccccgtgtgcggcctggacggcaagacctaccggaacgagtgcgccctgctgaaggcccggtgcaaggaggcccccgccctggaggtgcagtaccagggccggtgcaag

SEQ ID NO: 62 - protein, 706 aa, synthetic construct
MKWVTFISLLFLFSSAYSMDPNAAYVNMSNHHRGLASANVDFAFSLYKHLVALSPKKNIFISPVSISMALAMLSLGTCGHTRAQLLQGLGFNLTERSETEIHQGFQHLHQLFAKSDTSLEMTMGNALFLDGSLELLESFSADIKHYYESEVLAMNFQDWATASRQINSYVKNKTQGKIVDLFSGLDSPAILVLVNYIFFKGTWTQPFDLASTREENFYVDETTVVKVPMMLQSSTISYLHDSELPCQLVQMNYVGNGTVFFILPDKGKMNTVIAALSRDTINRWSAGLTSSQVDLYIPKVTISGVYDLGDVLEEMGIADLFTNQANFSRITQDAQLKSSKVVHKAVLQLNEEGVDTAGSTGVTLNLTSKPIILRFNQPFIIMIFDHFTWSSLFLARVMNPVPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKETCENVDCGPGKKCRMNKKNKPRCVCAPDCSNITWKGPVCGLDGKTYRNECALLKARCKEQPELEVQYQGRCK

SEQ ID NO: 63 - DNA, 2118 bp, synthetic construct
  source 1..2118: mol_type = other DNA
atgaagtgggtgaccttcatctctctgctgttcctgttctctagcgcttactctatggaccccaacgccgcctacgtgaacatgtctaaccaccacaggggcctggcctctgccaacgtggacttcgccttctctctgtacaagcacctggtggccctgtctcccaagaagaacatcttcatctctcccgtgtctatctctatggccctggccatgctgtctctgggcacctgcggccacaccagggcccagctgctgcagggcctgggcttcaacctgaccgagaggtctgagaccgagatccaccagggcttccagcacctgcaccagctgttcgccaagtctgacacctctctggagatgaccatgggcaacgccctgttcctggacggctctctggagctgctggagtctttctctgccgacatcaagcactactacgagtctgaggtgctggccatgaacttccaggactgggccaccgcctctaggcagatcaactcttacgtgaagaacaagacccagggcaagatcgtggacctgttctctggcctggactctcccgccatcctggtgctggtgaactacatcttcttcaagggcacctggacccagcccttcgacctggcctctaccagggaggagaacttctacgtggacgagaccaccgtggtgaaggtgcccatgatgctgcagtcttctaccatctcttacctgcacgactctgagctgccctgccagctggtgcagatgaactacgtgggcaacggcaccgtgttcttcatcctgcccgacaagggcaagatgaacaccgtgatcgccgccctgtctagggacaccatcaacaggtggtctgccggcctgacctcttctcaggtggacctgtacatccccaaggtgaccatctctggcgtgtacgacctgggcgacgtgctggaggagatgggcatcgccgacctgttcaccaaccaggccaacttctctaggatcacccaggacgcccagctgaagtcttctaaggtggtgcacaaggccgtgctgcagctgaacgaggagggcgtggacaccgccggctctaccggcgtgaccctgaacctgacctctaagcccatcatcctgaggttcaaccagcccttcatcatcatgatcttcgaccacttcacctggtcttctctgttcctggccagggtgatgaaccccgtgcctcgaggaccaaccatcaaaccatgccccccctgtaagtgccccgcacctaacctcctgggtgggccatccgtgtttatcttcccccctaaaattaaggatgtgctgatgatcagcctgagccctatcgtgacatgcgtggtcgtcgacgtgagcgaggatgaccccgacgtccagatctcttggtttgtgaacaacgtggaggttcacaccgcccaaacccagacacacagagaggactataattctactctccgcgttgtgtccgccctgcccatccagcaccaagattggatgtctggtaaggaattcaagtgtaaagtgaacaataaagatctgcccgcaccaatcgaaagaaccatttctaagcctaaaggctccgtgcgggctcctcaggtttacgtcctgccaccccccgaggaagagatgactaagaagcaggtgaccctgacctgtatggtgaccgattttatgcccgaagacatttacgtcgagtggaccaataatggtaagacagaactgaactataaaaatacagagccagtgctcgactctgatggcagctattttatgtactctaagctgagagttgagaagaaaaactgggtggaacggaattcttactcttgctctgtcgtgcacgaaggactgcacaaccaccacactaccaagtccttttctaggacccccggcaaggagacctgcgagaacgtggactgcggccccggcaagaagtgccggatgaacaagaagaacaagccccggtgcgtgtgcgcccccgactgcagcaacatcacctggaagggccccgtgtgcggcctggacggcaagacctaccggaacgagtgcgccctgctgaaggcccggtgcaaggagcagcccgagctggaggtgcagtaccagggccggtgcaag

SEQ ID NO: 64 - protein, 76 aa, synthetic construct
ETCENVDCGPGKKCRMNKKNKPRCVCAPDCSNITWKGPVCGLDGKTYRNECALLKARCKEAPELEVQYQGRCKKTC

SEQ ID NO: 65 - protein, 76 aa, synthetic construct
ETCENVDCGPGKKCRMNKKNKPRCVCAPDCSNITWKGPVCGLDGKTYRNECALLKARCKEQPALEVQYQGRCKKTC

SEQ ID NO: 66 - protein, 76 aa, synthetic construct
ETCENVDCGPGKKCRMNKKNKPRCVCAPDCSNITWKGPVCGLDGKTYRNECALLKARCKEAPALEVQYQGRCKKTC

SEQ ID NO: 67 - protein, 18 aa, Homo sapiens
MKWVTFISLLFLFSSAYS

(19) United States
(12) Patent Application Publication
(10) Pub. No.: US 2026/0209291 A1
(43) Pub. Date: Jul. 23, 2026

(54) Extracellular Matrix Binding Compounds for the Localized Loading of Therapeutic or Diagnostic Agents

(71) Applicant: Kamino ApS, Tranbjerg J (DK)

(72) Inventors: Andreas Lodberg, Randers NØ (DK); Christian Brix Folsted Andersen, TranbJerg (DK); Marco Eijken, Åbyhøj (DK)

(21) Appl. No.: 19/135,938

(22) PCT Filed: Dec. 7, 2023
(86) PCT No.: PCT/EP2023/084811

§ 371 Date: Jun. 5, 2025

(30) Foreign Application Priority Data
Dec. 7, 2022 (EP) 22211972.9

(51) Int. Cl. C07K 14/47; A61K 38/00; A61P 27/02; C07K 16/18; C07K 16/28; G01N 33/68

(52) U.S. Cl. CPC C07K 14/4703; A61P 27/02; C07K 16/18; C07K 16/2863; G01N 33/6857; G01N 33/6872; A61K 38/00; C07K 2317/569; C07K 2319/22; C07K 2319/30; C07K 2319/31; G01N 2333/71

(57) ABSTRACT

In the field of biological therapy, a major hurdle is the need for a platform technology that enables high bioavailability and long therapeutic exposure of a biological therapy in the setting of localized disease. Classical approaches result in problematic trade-offs such as toxicity and off-target side effects. The present disclosure provides compounds comprising follistatin domain 1 (FSD1) of follistatin (FST) which are able to bind biological structures, such as the extracellular matrix, through heparan sulfates, without bearing the undesirable neutralizing effects on activin A, myostatin, and GDF11 activities of e.g. full-length follistatin. The invention relates to fusion proteins or conjugated proteins, and compositions thereof, constructs or vectors encoding thereof, medical uses thereof and methods for biological therapy.

### TECHNICAL FIELD

The present invention relates to compounds comprising follistatin domain 1 (FSD1) of follistatin (FST) binding to heparan sulfates present on cell surfaces, extracellular matrix and basement membrane of mammalian tissues.

### BACKGROUND

The therapeutic use of proteins (biological therapy) has been accelerated by half-life extending technologies. These technologies include recombinant fusion with proteins with inherently long serum half-life (such as IgG Fc, transferrin, or albumin), fusion with a polypeptide to increase overall size and hydrodynamic radius, or altering the glycosylation profile. Predominantly, half-life extending technologies optimize systemic delivery, i.e. to the entire organism, of the biological therapy (REF. 1). When a disease is generalized this systemic delivery is desirable. However, when a disease is localized to a specific tissue area or organ or physiological compartment, systemic therapy will affect tissues that were not meant to be targeted. Moreover, the bioavailability of a systemic therapy may be low in target tissues as a result of global distribution between physiological compartments in the body (REF. 2).

Attempts to direct biological therapy to a specific tissue includes targeting cell surface proteins, such as cluster of differentiation (CD) proteins. For example, an antibody may be bispecific to CD3 (a T cell co-receptor) and a tumor antigen, bridging interaction between the T cell and the tumor cell (REF. 2). While selectivity for a CD protein may direct a therapy to a subset of cells it does not remove the occurrence of toxicity and off-target side effects (REFs 3-5). In part, this may be because CD proteins or similar marker proteins are never entirely exclusive to the desired cell type and because the biological therapy is still distributed systemically (REFs 3-5).

There is a need for a platform technology that enables high bioavailability and long therapeutic exposure of a biological therapy in the setting of localized disease, such as a solid tumor or an eye disease. Localized and confined fixation of biological therapies to a tissue or organ would likely at the same time keep systemic toxicity and off-target side effects to a minimum. This requirement is not fulfilled by current platform strategies for systemic half-life extension and marker protein targeting.

### SUMMARY

The invention is defined in the attached claims.

The inventors of the present invention have developed compounds, such as fusion proteins or conjugated proteins comprising polypeptides (P1) comprising follistatin domain 1 (FSD1) of follistatin (FST), which are able to bind biological structures, such as the extracellular matrix, through heparan sulfates, without bearing the undesirable neutralizing effects on activin A, myostatin, and GDF11 activities of e.g. full-length follistatin.

The polypeptides (P1) disclosed form a versatile platform which can be used for fusion and conjugation to therapeutic or diagnostic agents, as exemplified with the 4 types of compounds developed by the inventors. The compounds of the present invention can be used for the localized loading, binding of therapeutic or diagnostic agents to biological structures, and improve the local half-life of the therapeutic or diagnostic agents they bear. The inventors have also shown that the developed compounds are taken up intracellularly, such as to the cytosol or nucleus.

A first aspect of the present invention relates to a fusion protein or conjugated protein comprising: (i) one or more polypeptide(s) (P1) consisting of at least one FSD1 domain comprising or consisting of a polypeptide having at least 70% such as at least 80%, for instance at least 90%, such as at least 95%, for instance at least 99% sequence identity to SEQ ID NO: 1, and (ii) a therapeutic or diagnostic agent.

A second aspect of the present invention relates to one or more polynucleotides which upon expression encode the fusion protein or conjugated protein of the present invention.

A third aspect of the present invention relates to one or more constructs or vectors comprising the one or more polynucleotides of the second aspect or encoding the fusion protein of the first aspect.

A fourth aspect of the present invention relates to a host cell comprising the one or more polynucleotides of the second aspect or the one or more constructs or vectors of the third aspect.

A fifth aspect of the present invention relates to a composition comprising the fusion protein or conjugated protein of the first aspect, the one or more polynucleotides of the second aspect, the one or more constructs or vectors of the third aspect, the host cell of the fourth aspect, or mixtures thereof.

A sixth aspect of the present invention relates to the use of the fusion protein or conjugated protein of the first aspect, the one or more polynucleotides of the second aspect, the one or more constructs or vectors of the third aspect, the host cell of the fourth aspect, or the composition of the fifth aspect in medicine.

A seventh aspect of the present invention relates to a method of treatment of a disease or condition, comprising administering to a subject an effective amount of the fusion protein or conjugated protein of the first aspect, the one or more polynucleotides of the second aspect, the one or more constructs or vectors of the third aspect, the host cell of the fourth aspect, or the composition of the fifth aspect.

An eighth aspect of the present invention relates to the use of the fusion protein or conjugated protein of the first aspect, the one or more polynucleotides of the second aspect, the one or more constructs or vectors of the third aspect, the host cell of the fourth aspect, or the composition of the fifth aspect in the manufacture of a medicament for the treatment of a disease or condition, for example an ophthalmologic condition, an oncogenic condition, or an inflammatory condition.

A ninth aspect of the present invention relates to a method of increasing the local half-life of a therapeutic or diagnostic agent at the site of administration, comprising obtaining the fusion protein or conjugated protein of the first aspect, the one or more polynucleotides of the second aspect, the one or more constructs or vectors of the third aspect, the host cell of the fourth aspect, or the composition of the fifth aspect, wherein the therapeutic or diagnostic agent (ii) as described in the present invention, or the therapeutic or diagnostic moiety identical or different as described in the present invention comprises or consists of said therapeutic or diagnostic agent.

A tenth aspect of the present invention relates to a method of increasing the local in-vivo half-life of a therapeutic or diagnostic agent said method comprising the steps of:
a) providing a therapeutic or diagnostic agent;
b) obtaining the fusion protein or conjugated protein of the first aspect, wherein the therapeutic or diagnostic agent of step a) is the therapeutic or diagnostic agent (ii) as described herein,
thereby increasing the local in-vivo half-life of said therapeutic or diagnostic agent.

An eleventh aspect of the present invention relates to a method of increasing the binding of a therapeutic or diagnostic agent to the extracellular matrix comprising administering to a subject the fusion protein or conjugated protein of the first aspect, the one or more polynucleotides of the second aspect, the one or more constructs or vectors of the third aspect, the host cell of the fourth aspect, or the composition of the fifth aspect, wherein the therapeutic or diagnostic agent (ii) as described in the present invention, or the therapeutic or diagnostic moiety identical or different as described in the present invention comprises or consists of said therapeutic or diagnostic agent.

A twelfth aspect of the present invention relates to a method of increasing the binding of a therapeutic or diagnostic agent to a predefined organ comprising administering to a subject the fusion protein or conjugated protein of the first aspect, the one or more polynucleotides of the second aspect, the one or more constructs or vectors of the third aspect, the host cell of the fourth aspect, or the composition of the fifth aspect, wherein the therapeutic or diagnostic agent (ii) as described in the present invention, or the therapeutic or diagnostic moiety identical or different as described in the present invention comprises or consists of said therapeutic or diagnostic agent.

A thirteenth aspect of the present invention relates to a method of increasing the intracellular uptake of a therapeutic or diagnostic agent comprising administering to a subject the fusion protein or conjugated protein of the first aspect, the one or more polynucleotides of the second aspect, the one or more constructs or vectors of the third aspect, the host cell of the fourth aspect, or the composition of the fifth aspect, wherein the therapeutic or diagnostic agent (ii) as described in the present invention, or the therapeutic or diagnostic moiety identical or different as described in the present invention comprises or consists of said therapeutic or diagnostic agent.

A fourteenth aspect of the present invention relates to a method of degrading an intracellular protein, said method comprising the step of coupling the fusion protein or conjugated protein as described herein to a ligand of E3 ubiquitin ligase, wherein the therapeutic or diagnostic agent (ii) of said fusion protein or conjugated protein binds to said intracellular protein.

A fifteenth aspect of the present invention relates to one or more constructs or vectors encoding the one or more polypeptide(s) (P1) (i) of the fusion protein or conjugated protein as described herein.

A sixteenth aspect of the present invention relates to a composition comprising one or more polypeptides selected from the group consisting of: FSD1 (Q124A) of sequence ID NO: 47, FSD1 (E126A) of sequence ID NO: 50, and FSD1 (Q124 E126A) of sequence ID NO: 53, or variants thereof, having at least 70% such as at least 80%, for instance at least 90%, such as at least 95%, for instance at least 96%, such as at least 97%, for instance 98%, such as at least 99% sequence identity thereto.

### DESCRIPTION OF DRAWINGS

FIG. 1: Compound design

The conceptual art for four types of compounds is presented. For all types, FSD1 may be applied with or without multimerization and with or without a linker. A. FSD1 fused as a module to any other protein (Type 1). B. Homodimeric (i) or heterodimeric (ii) Fc-FSD1 fused as a module to any other protein (Type 2). C. FSD1 covalently coupled to any regular antibody or any other therapeutic or diagnostic agent (i) or an anti-Fc nanobody-FSD1 fusion associating with any regular antibody or any other therapeutic or diagnostic agent, which the nanobody has an affinity to (ii) (Type 3). D. Heterodimeric Fc fused to either FST or FSD1 on one entity and any other protein fused to the other entity (Type 4).

FIG. 2: Heparin-affinity chromatography

A. Chromatograms showing elution profiles (A₂₈₀) of FSD1, FSD1- or FST291-linked compounds and therapeutic agents (left axis). Linear gradients from 0%-100% buffer B (2 M NaCl) are shown as straight lines (right axis). Individual peaks in the elution profile are marked by numbers (#1 or #2). B. Table showing the elution concentration ([NaCl]) of the individual peaks of the samples. Nb (nanobody).

FIG. 3: Covalent coupling of FSD1 to an anti-TNFα antibody (Adalimumab)

A and B. Chromatograms showing elution profiles (A₂₈₀, left axis) of Adalimumab (A) or Adalimumab covalently coupled to FSD1 (B). Linear gradients from 0%-100% buffer B (2 M NaCl) are shown as straight lines (right axis). C. SDS-PAGE of fractions 7-12 of the heparin-affinity chromatography of Adalimumab covalently coupled to FSD1.

FIG. 4: Bioassay-based validation of activin A neutralization

A luciferase-based bioassay of pSmad2/3 activation was used to evaluate neutralization of the growth factor, activin A. FSD1 (monomer or dimer) (A), type 2 design and type 4 design compounds (B and C), native full-length follistatin₃₁₅ (FST315, SEQ ID NO: 28) and FST315 with an altered heparin binding site fused to a murine Fc fragment (FST315dHBS-mFc, SEQ ID NO: 30, SEQ ID NO: 31) (A) were evaluated in a concentration range up to 8.2 nM. Commercial counterparts, VGFR(½)-hFc (Aflibercept) or TNFR2-hFc (Etanercept) were also evaluated in a concentration range up to 8.2 nM (B and C). Pos control (positive control), Neg control (negative control). TNFR2 (Tumor Necrosis Factor Receptor 2), VGFR ½ (Ligand binding domains of Vascular Endothelial Growth Factor Receptors 1 and 2). All results presented as mean±SEM.

FIG. 5: Bioassay-based validation of myostatin neutralization

A luciferase-based bioassay of pSmad2/3 activation was used to evaluate neutralization of the growth factor, myostatin. FSD1 (monomer or dimer) (A), type 2 design and type 4 design compounds (B and C), native full-length follistatin₃₁₅ (FST315, SEQ ID NO: 28) and FST315 with an altered heparin binding site fused to a murine Fc fragment (FST315dHBS-mFc, SEQ ID NO: 30, SEQ ID NO: 31) (A) were evaluated in a concentration range up to 8.2 nM. Commercial counterparts, VGFR(½)-hFc (Aflibercept) or TNFR2-hFc (Etanercept) were also evaluated in a concentration range up to 8.2 nM (B and C). Pos control (positive control), Neg control (negative control). All results presented as mean±SEM.

FIG. 6: Bioassay-based validation of GDF11 neutralization

A luciferase-based bioassay of pSmad2/3 activation was used to evaluate neutralization of the growth factor, GDF11 (Growth Differentiation Factor 11). FSD1 (monomer or dimer) (A), type 2 design and type 4 design compounds. (B and C), native full-length follistatin₃₁₅ (FST315, SEQ ID NO: 28) and FST315 with an altered heparin binding site fused to a murine Fc fragment (FST315dHBS-mFc, SEQ ID NO: 30, SEQ ID NO: 31) (A) were evaluated in a concentration range up to 8.2 nM. Commercial counterparts, VGFR(½)-hFc (Aflibercept) or TNFR2-hFc (Etanercept) were also evaluated in a concentration range up to 8.2 nM (B and C). Pos control (positive control), Neg control (negative control). All results presented as mean±SEM.

FIG. 7: Ligand-binding affinity determined by surface plasmon resonance

Left panels: Surface plasmon resonance sensorgrams of VEGF (Vascular Endothelial Growth Factor) binding to immobilized type 2 design or type 4 design compounds comprising VGFR(½), commercial VGFR(½)-hFc (Aflibercept), and a negative control (FST315dHBS-mFc, SEQ ID NO: 30, SEQ ID NO: 31). Right panels: Surface plasmon resonance sensorgrams of TNFα (Tumor Necrosis Factor alpha) binding to immobilized type 2 design or type 4 design compounds comprising TNFR2, commercial TNFR2-hFc, (Etanercept), and a negative control (FST315dHBS-mFc, SEQ ID NO: 30, SEQ ID NO: 31).

FIG. 8: Binding to extracellular matrix

Plates precoated with extracellular matrix (ECM) extract (Matrigel) were used to evaluate the binding profile of type 2 design and type 4 design compounds to ECM. A. ECM binding curves of standalone FSD1 (monomer or dimer), native full-length follistatin₂₈₈ (FST288), and FST315dHBS-mFc (SEQ ID NO: 30, SEQ ID NO: 31), as detected by colorimetry. B. Type 2 and type 4 design compounds comprising VGFR(½), commercial VGFR(½)-hFc (Aflibercept), and a control in the form of standalone FSD1. C. Type 2 and type 4 design compounds comprising TNFR2, commercial TNFR2-hFc (Etanercept), and a control in the form of standalone FSD1. All results presented as mean±SEM.

FIG. 9: In vivo estimation of compound half-life in skeletal muscle in mice using IVIS

Type 2 design or type 4 design compounds with VGFR(½) as the therapeutic moiety were administered intramuscularly in the gastrocnemius muscles of naïve mice and compared to commercial VGFR(½)-hFc (Aflibercept) or PBS (Phosphate Buffered Saline). A. Longitudinal follow-up on fluorescent intensity from the gastrocnemius muscle estimated using an IVIS Spectrum in vivo imaging system. B. IVIS scans of all mice on the final day (96 hours after treatment). C. Fluorescent intensity of excised gastrocnemius muscle. D. IVIS scans of excised gastrocnemius muscles. E. Fluorescent intensity of serum samples on the final day (96 hours after treatment). F. IVIS scans of serum samples in 1.5 mL microcentrifuge tubes. All results presented as mean±SEM. Data analyzed using a one-way ANOVA including all groups. Multiple comparisons corrected using Bonferroni's test. *P<0.05 vs PBS, ^{#}P<0.05 vs VGFR(½)/FST291-mFc, ^{†}P<0.05 vs VGFR(½)-hFc (Aflibercept).

FIG. 10: In vivo estimation of type 4 design compound half-life in eye tissue of mice using IVIS

Type 4 design compound with VGFR(½) as the therapeutic moiety was administered intravitreally in the eyes of naïve mice and compared to commercial VGFR(½)-hFc (Aflibercept) or PBS. A. Longitudinal follow-up on fluorescent intensity from the eye estimated using an IVIS Spectrum in vivo imaging system. B. Fluorescent intensity of enucleated eye on the final day (96 hours after treatment). C. IVIS scans of enucleated eyes. All results presented as mean±SEM. Data analyzed using a one-way ANOVA including all groups. Multiple comparisons corrected using Bonferroni's test. *P<0.05 vs PBS, ^{†}P<0.05 vs VGFR(½)-hFc (Aflibercept).

FIG. 11: Ex vivo estimation of type 4 design compound half-life using IVIS in porcine kidney during normothermic machine perfusion

Type 4 design compound with TNFR2 as the therapeutic moiety was administered via the arterial line in the perfused kidney and compared to commercial TNFR2-hFc (Aflibercept) or PBS. A. Fluorescent intensity of porcine kidney midline section 6 hours after treatment. B. IVIS scans of porcine kidney midline sections. C. Fluorescent intensity of porcine kidney outer cortex section 6 hours after treatment. D. IVIS scans of porcine kidney outer cortex sections.

FIG. 12: Intracellular uptake of the platform after 18 hours of compound incubation

Confocal images of adherent HEK293 cells. Cells were incubated for 18 hours with ATTO 488 fluorescently labelled type 1 design compound with anti-vimentin-nanobody as the therapeutic or diagnostic moiety (anti-vimentin-nanobody-FSD1), anti-vimentin-nanobody, or DMEM alone. After compound incubation, cells were washed and incubated with Hoechst and Lysotracker to visualize the nucleus and lysosomes, respectively. A. Maximum intensity projection of a Z-stack from each sample. B.

Orthogonal projection showing x and y cut planes of the anti-vimentin-nanobody-FSD1 sample alone.

FIG. 13: In vivo demonstration of compound ECM binding in skeletal muscle NIR730 fluorescently labeled type 2 design or type 4 design compounds with VGFR(½) as the therapeutic moiety were administered intramuscularly in the gastrocnemius muscles of naïve mice and compared to VGFR(½)-hFc (Aflibercept) or PBS. A. DAPI stained and Sirius Red stained serial sections of gastrocnemius muscle. DAPI staining allowed visualization of cell nuclei. Fluorescent signal from NIR730 allowed visualization of the administered compounds. Sirius Red staining allowed visualization of connective tissue strokes within the muscle tissue. B. Higher-magnification of two areas of the VGFR(½)-mFc-FSD1 treated gastrocnemius muscle. Area 1 is a magnification of the parenchyma (P) and the connective tissue (CT). Area 2 is a magnification of a blood vessel.

FIG. 14: In vivo evaluation of type 2 and type 4 design compound half-lives in eye tissue of mice using fluorescence fundus imaging

Type 2 and type 4 design compounds with VGFR(½) as the therapeutic moiety were administered intravitreally in the eyes of naïve mice and compared to commercial VGFR(½)-hFc (Aflibercept) or PBS. A+B. Images of in vivo fluorescence funduscopy at baseline (A) and 96 hours after treatment (B). C. Average fluorescent intensity of fundus images 96 hours after treatment. All results presented as mean±SEM. Data analyzed using a one-way ANOVA including all groups. Multiple comparisons corrected using Bonferroni's test. *=P<0.05 vs PBS, #=P<0.05 vs VGFR(½)/FST291-mFc, †=P<0.05 vs VGFR(½)-hFc (Aflibercept).

FIG. 15: In vivo efficacy of type 2 design compound in a disease model of wet age-related macular degeneration

Type 2 design compound with VGFR(½) as the therapeutic moiety was administered intravitreally in the eyes of naïve mice and compared to commercial VGFR(½)-hFc (Aflibercept) or Mouse IgG2a isotype control. A. Representative images of CD31 and Isolectin positive CNV lesions in the RPE/choroidal flat-mount. Images were individually contrast and brightness adjusted for optimal visual representation of the CNV lesion in black and white. Scale bar=150 μm. B. Average CNV lesion area per eye estimated from CD31 and Isolectin immunohistochemical staining of the RPE/choroidal flat-mount. In a QQ plot for normal distribution, one outlier was identified and excluded in the VGFR(½)-mFc-FSD1 group. One eye in the VGFR(½)-hFc (Aflibercept) group was inadvertently destroyed during sample preparation. Two eyes were excluded according to the predetermined CNV lesion exclusion criteria: one eye from the VGFR(½)-mFc-FSD1 group and one eye from the VGFR(½)-hFc (Aflibercept) group. All results presented as mean±SEM. Data analyzed using a one-way ANOVA including all groups. Multiple comparisons corrected using Bonferroni's test. *=P<0.05 vs Mouse IgG2a isotype control.

FIG. 16: Intracellular uptake of the platform after 3 hours of compound incubation

Maximum intensity projection of a Z-stack of confocal images of adherent HEK293 cells. Cells were incubated for 3 hours with ATTO 488 fluorescently labeled type 1 design compound with anti-vimentin-nanobody as the therapeutic or diagnostic moiety (anti-vimentin-nanobody-FSD1) or anti-vimentin-nanobody. After compound incubation, cells were washed and incubated with Hoechst and Lysotracker to visualize the nucleus and lysosomes, respectively.

FIG. 17: Intracellular uptake of the platform after 18 hours of compound incubation imaged using Airyscan superresolution confocal imaging

Maximum intensity projection of a Z-stack of Airyscan superresolution confocal images of adherent HEK293 cells. Cells were incubated for 18 hours with ATTO 488 fluorescently labeled type 1 design compound with anti-vimentin-nanobody as the therapeutic or diagnostic moiety (anti-vimentin-nanobody-FSD1) or anti-vimentin-nanobody. After compound incubation, cells were washed and incubated with Hoechst to visualize the nucleus. Cells treated with anti-vimentin-nanobody did not provide sufficient signal to make a superresolution image.

FIG. 18: Intracellular uptake of the platform in vivo

Maximum intensity projection of a Z-stack of confocal images of gastrocnemius muscle. ATTO 488 fluorescently labeled Type 1 design compound with anti-vimentin-nanobody as the therapeutic or diagnostic moiety (anti-vimentin-nanobody-FSD1) was administered intramuscularly in gastrocnemius of naïve mice and compared to anti-vimentin-nanobody. Mice were euthanized 18 hours after injection and the gastrocnemius was formalin fixed, sectioned, and mounted with DAPI-containing mountant. P, parenchyma. CT, connective tissue.

FIG. 19: Extended bioassay-based validation of activin A, myostatin, and GDF11 neutralization by type 1 design compound

A luciferase-based bioassay of pSmad2/3 activation was used to evaluate neutralization of the growth factors, activin A (A), myostatin (B), or GDF11 (C). FSD1 (monomer or dimer) and type 1 design compound with anti-vimentin-nanobody as the therapeutic or diagnostic moiety (anti-vimentin-nb-FSD1) were evaluated in a concentration range up to 1 M. Pos control (positive control), Neg control (negative control). All results presented as mean±SEM.

FIG. 20: Bioassay-based validation of activin A and myostatin neutralization by FSD1 mutants

A luciferase-based bioassay of pSmad2/3 activation was used to evaluate neutralization of the growth factors, activin A (A) and myostatin (B). Native FSD1 (referred to simply as “FSD1”) and FSD1 mutants: FSD1 (Q124A), FSD1 (E126A), and FSD1 (Q124A, E126A) were evaluated in a concentration range up to 822 nM. Pos control (positive control), Neg control (negative control). All results presented as mean±SEM.

FIG. 21: Bioassay-based validation of activin A neutralization by type 2 design compound utilizing FSD1 mutants

A luciferase-based bioassay of pSmad2/3 activation was used to evaluate neutralization of the growth factors, activin A (A), myostatin (B), and GDF11 (C). Type 2 design compound utilizing native FSD1, VGFR(½)-mFc-FSD1, and the commercial counterpart, VGFR(½)-hFc (Aflibercept), were evaluated in a concentration range up to 1 μM. Type 2 design compounds utilizing mutant FSD1: VGFR(½)-mFc-FSD1 (Q124A), VGFR(½)-mFc-FSD1 (E126A), VGFR(½)-mFc-FSD1 (Q124A, E126A) were evaluated in a concentration range up to 750 nM (Activin A) or 63 nM (myostatin and GDF11). Pos control (positive control), Neg control (negative control). All results presented as mean±SEM.

FIG. 22: Binding to extracellular matrix by multimeric FSD1, type 3 design compound, and type 2 design compound utilizing FSD1 mutants

Plates precoated with extracellular matrix (ECM) extract (Matrigel) were used to evaluate the ECM binding profile of multimeric FSD1, type 3 design compound, and type 2 design compounds utilizing FSD1 mutants as detected by colorimetry. A. ECM binding curves of standalone multimeric FSD1 (FSD1, FSD1-FSD1, and FSD1-FSD1-FSD1). B. ECM binding curves of three different fractions of type 3 design compound, anti-TNFα antibody-FSD1 (Adalimumab-FSD1), with increasing heparin affinity (low, medium, or high). Commercial Adalimumab was included as a control. C. ECM binding curves of type 2 design compounds utilizing either native FSD1, VGFR(½)-mFc-FSD1, or FSD1 mutants: VGFR(½)-mFc-FSD1 (Q124A), VGFR(½)-mFc-FSD1 (E126A), and VGFR(½)-mFc-FSD1 (Q124A, E126A), All results presented as mean±SEM.

FIG. 23: Bioassay-based validation of glucocorticoid neutralization

A luciferase-based bioassay of glucocorticoid response element (GRE) activation was used to evaluate neutralization of the endogenous glucocorticoid, cortisol, or the synthetic glucocorticoid, prednisolone 21-hemisuccinate. Type 2 and type 4 design compounds with CBG as the therapeutic moiety were tested in a concentration range up to 2 μM. Pos control (positive control), Neg control (negative control). All results presented as mean±SEM.

FIG. 24: Using the platform to degrade intracellular protein

Western blot of lysates from HEK293 cells treated for 24 hours with Type 1 design compound with anti-vimentin-nanobody as the therapeutic moiety (anti-vimentin-nb-FSD1), anti-vimentin-nb, or DMEM alone. Both compounds were coupled to an E3 ligase ligand (VHL ligand) to mediate proteasomal degradation of the target protein. A. Western blot of vimentin and GAPDH (loading control). Lane 1: Size marker. Lane 2: DMEM lysate. Lane 3: anti-vimentin-nb coupled to VHL ligand lysate. Lane 4: anti-vimentin-nb-FSD1 coupled to VHL ligand lysate. B. Vimentin band intensity normalized to GAPDH band intensity.

### DETAILED DESCRIPTION

### Definitions

As used herein, the singular forms “a”, “an”, and “the” include plural forms unless the context clearly dictates otherwise.

It will also be appreciated that “one or more” can be interchangeably substituted by “a plurality”, or “at least one”.

The term “compound” as used herein encompasses molecules comprising or consisting of a Follistatin domain 1 (FSD1) and a therapeutic or diagnostic agent. For example, the term compound encompasses the fusion proteins and conjugated proteins of the present invention comprising or consisting of a Follistatin domain 1 (FSD1) and a therapeutic or diagnostic agent.

A “fusion protein” as used herein refers to a hybrid polypeptide which comprises protein domains from at least two distinct polypeptides which are not normally or naturally fused together in a single amino acid sequence. A fusion protein may include a single amino acid sequence that contains two entirely distinct amino acid sequences or two similar or identical polypeptide sequences, provided that these sequences are not normally found together in the same configuration in a single amino acid sequence found in nature. A protein domain of the fusion protein may thus be located at the amino-terminal portion of the fusion protein or at the carboxy-terminal protein thus forming an “amino-terminal fusion protein” or a “carboxy-terminal fusion protein,” respectively. Fusion proteins may also contain a linker polypeptide in between the constituent polypeptides of the fusion protein. The term “fusion construct” or “fusion protein construct” is generally meant to refer to a polynucleotide encoding a fusion protein.

A “conjugated protein” as used herein refers to a hybrid polypeptide which comprises one or more protein domains bound to a non-polypeptide component, e.g. a low molecular compound, a nucleic acid, a sugar chain, or a nanoparticle.

“Follistatin” (FST) as used herein may refer to all forms of follistatin including, by way of example, the protein cores and molecular weight forms which have been identified as arising from the alternatively spliced mRNAs FS-315, FS-303, and FS-288 Accordingly, it should also be understood to include reference to any isoforms which may arise from alternative splicing of follistatin mRNA or mutant or polymorphic forms of follistatin. It should still further be understood to extend to any protein encoded by the follistatin (FST) gene, any subunit polypeptide, such as precursor forms which may be generated, and any follistatin protein, whether existing as a monomer, multimer or fusion protein.

Follistatin domain 1 (FSD1) as used herein refers to the first of the three follistatin domains FSD1 (SEQ ID NO: 1), FSD2 (SEQ ID NO: 41) and FSD3 (SEQ ID NO: 42).

A “module” as used herein may be used interchangeably with “unit” and “part”, e.g. as a (poly) peptide module fused or bound as a fusion protein or a conjugated protein to another polypeptide or non-polypeptide moiety.

As used herein, the term “therapeutic agent” refers to a biological or chemical agent used for treatment, curing, mitigating, or preventing deleterious conditions in a subject. The term “therapeutic agent” also includes substances and agents for combating a disease, condition, or disorder of a subject, and includes drugs, diagnostics, and instrumentation. “Therapeutic agent” also includes anything used in medical diagnosis, or in restoring, correcting, or modifying physiological functions. Therapeutic agents include for instance therapeutic polypeptides, therapeutic polynucleotides, therapeutic small molecules, for example, but not limited to therapeutic antibodies and fragments thereof, and antibiotics.

As used herein a “therapeutic moiety” refers to a portion of a therapeutic agent that renders at least part of the therapeutic effect of the agent. A therapeutic moiety may thus be a polypeptide having therapeutic activity and constructs including such polypeptides, for example the therapeutic moiety can be a ligand receptor providing a therapeutic effect. The therapeutic moiety can be any moiety useful for therapy including, but not limited to, antibiotics, anti-inflammatory agents, anti-tumor drugs, cytotoxins, anti-viral agents, and radioactive moieties. “Therapeutic moiety” includes prodrugs of bioactive moieties, constructs in which more than one therapeutic moiety is bound to a carrier, e.g, multivalent moieties.

A “diagnostic agent” as used herein refers to an agent that may be used to detect, image and/or monitor the presence and/or progression of a condition(s), pathological disorder(s) and/or disease(s).

As used herein a “diagnostic moiety” refers to a portion of a diagnostic agent which attached or separated from the diagnostic agent renders at least part of the diagnostic effect of the agent e.g. an imaging moiety.

As used herein, the term “click chemistry” refers to the use of chemical building blocks to drive a linkage reaction with appropriate complementary sites in other blocks. These chemical reactions (e.g., including, but not limited to, those between azide and alkyne groups) are specific and result in covalent linkage between the two molecules. The compounds of the present invention, such as the fusion proteins and conjugated proteins of the present invention may be obtained by a production step involving click-chemistry.

As used herein IgG Fc refers to the C-terminal region of an IgG heavy chain.

“Full-length antibody” as used herein, refers to an antibody including at least 2 heavy and 2 light chains. The term includes antibodies with heavy chains that contain a Fc region. A full-length antibody can be a native sequence antibody or a recombinant antibody. A full-length antibody can be for example human, humanized, murine, murinized and/or affinity matured. Antibodies can be of any immunoglobulin class including IgG, IgM, IgE, IgA, IgD and any subclass thereof. A full-length antibody may comprise further domains, such as e.g. a scFv or a scFab conjugated to one or more of the chains of the full length antibody. These conjugates are also encompassed by the term “full-length antibody”.

“Activin A” as used herein refers to the active form of the homodimer of the polypeptide inhibin beta A (BA) chains. “Activin A” refers to the activin protein having UniProt Accession No: P08476 (SEQ ID NO: 44), also referred to as Inhibin subunit beta A. Activins A, B, and AB are the homodimers and heterodimer respectively of two polypeptide chains, βA and βB.

“Myostatin” as used herein refers to active forms of the myostatin protein having UniProt Accession No: 014793 (SEQ ID NO: 45), also referred to as “GDF8” or “GDF-8”, Growth differentiation factor 8. Myostatin is a negative regulator of skeletal muscle mass.

“GDF11” or “GDF-11” as used herein refers to active forms of the Growth differentiation factor 11, also referred to as bone morphogenetic protein 11 (BMP-11) having UniProt Accession No: 095390 (SEQ ID NO: 46).

“Half-life” as used herein refers to the pharmacokinetic property of a compound or agent that is a measure of the mean survival time of the compound following their administration. The half-life can be expressed as the time required to eliminate 50 percent of a known quantity of the compound or agent from the patient's body or a specific compartment thereof, for example, as measured in serum, i.e., circulating half-life, or in other tissues. “Local half-life” refers to the half-life of a compound locally, such as at the site of administration of the compound or agent, for example at a specific organ, such as the local half-life of the compound or agent in muscle, or for example the local half-life of the compound or agent in the eye.

As used herein, “rcsb id” refers to the Research Collaboratory for Structural Bioinformatics (RCSB) Protein Data Bank (PDB) ID assigned to a protein. A unique identifier is assigned to each entry in the PDB, a comprehensive resource that provides information about the three-dimensional structures of biological macromolecules.

As used herein “CNV” refers to choroidal neovascularization, such as, but not limited to, choroidal neovascularization in age-related macular degeneration (AMD).

As used herein “RPE” refers to the retinal pigment epithelium.

As used herein “variant” refers to polypeptides or proteins which differ by one or more amino acid from the sequence from which they are derived. Typically, variants will have at least 70%, such as at least 80%, for instance at least 90%, such as at least 95%, for instance at least 96%, such as at least 97%, for instance at least 98%, such as at least 99% sequence identity to the sequence from which they are derived. “variants” as used herein include mutant polypeptides obtained by mutagenesis, such as mutant polypeptides obtained by one or more point mutation(s) in the sequence from which they are derived.

As used herein, “PROTAC” refers to Proteolysis Targeting Chimeras. PROTACs consist of two covalently linked protein-binding molecules: one capable of engaging an E3 ubiquitin ligase, and another that binds to a target protein meant for degradation, such as intracellular degradation by ubiquitination.

### Fusion Protein or Conjugated Proteins Structures

The present invention relates to fusion or conjugated proteins structures comprising one or more polypeptide(s) (P1) comprising or consisting of at least one FSD1 domain. Such polypeptides P1, and fusion or conjugated proteins comprising thereof, are able to bind biological structures, such as the extracellular matrix, through heparan sulfates, without bearing the undesirable neutralizing effects on activin A, myostatin, and GDF11 activities of e.g. full-length follistatin.

The present invention thus provides a platform technology enabling, but not limited to, increased binding and therapeutic exposure of therapeutic or diagnostic agents, with reduced off-target side effects.

### Type 1

In one aspect, the present invention relates to a fusion protein or conjugated protein comprising:
(i) one or more polypeptide(s) (P1) comprising or consisting of at least one FSD1 domain comprising or consisting of a polypeptide having at least 70%, such as at least 80%, for instance at least 90%, such as at least 95%, for instance at least 99% sequence identity to SEQ ID NO: 1, and
(ii) a therapeutic or diagnostic agent.

In some embodiments, the fusion protein or conjugated protein comprises or consists of a polypeptide having at least 70% sequence identity, such as at least 80% sequence identity, for example at least 90% sequence identity, such as at least 95% sequence identity, for example 95% sequence identity with a polypeptide as set forth in the group consisting of: SEQ ID NO: 2, SEQ ID NO: 3 and SEQ ID NO: 5.

In other embodiments, the fusion protein or conjugated protein comprises or consists of a polypeptide encoded by a polynucleotide having at least 70% sequence identity, such as at least 80% sequence identity, for example at least 90% sequence identity, such as at least 95% sequence identity, for example 95% sequence identity with a polypeptide as set forth in the group consisting of: SEQ ID NO: 4 and SEQ ID NO: 6.

### Type 2

The skilled person will appreciate that the evolution of techniques within the field of antibody engineering have enabled the development of novel classes of therapeutic and diagnostic agents based on the engineering of the Fc domain of immunoglobulins (Ig), such as an IgG. Such compounds include for instance therapeutic or diagnostic Fc-Fusion proteins and derivatives thereof, which are composed of one or more therapeutic or diagnostic moieties C-terminally-linked to an Ig Fc domain.

In one embodiment, the present invention relates to a fusion protein or conjugated protein comprising:
(i) one or more polypeptide(s) (P1) comprising or consisting of at least one FSD1 domain comprising or consisting of a polypeptide having at least 70%, such as at least 80%, for instance at least 90%, such as at least 95%, for instance at least 99% sequence identity to SEQ ID NO: 1, and
(ii) a therapeutic or diagnostic agent, wherein the therapeutic or diagnostic agent comprises or consists of an immunoglobulin Fc domain; and wherein the one or more polypeptide(s) (P1) are linked C-terminally to said immunoglobulin Fc domain, preferably wherein said immunoglobulin is an IgG, IgA, IgM, IgE, or IgD.

The immunoglobulin Fc domain of the fusion protein or conjugated protein of the present invention may be composed of two identical protein fragments, each comprising a CH2 and a CH3 domain, such as native IgG Fc regions. In preferred embodiments, the immunoglobulin Fc domain of the fusion protein or conjugated protein of the present invention may be composed of different protein fragments, for example protein fragments comprising one or more different mutation(s) in the CH2 and/or CH3 domains of the Fc region.

Thus, in one embodiment, the immunoglobulin Fc domain of the fusion protein or conjugated protein of the present invention is an IgG Fc domain and comprises or consists of an IgG Fc-homodimer or an IgG Fc-heterodimer.

The Fc-heterodimers and/or fusion protein or conjugated protein of the present invention comprising said Fc heterodimers may be produced by protein engineering techniques known in the art, such as for instance made using an scFV-Fc/Fc expression system, cloned on plasmids, and transfected in cells for expression (such as HEK293 cells or CHO cells), followed by harvesting of the supernatant and purification of the proteins produced. In particular, the skilled person will know that heterodimer formation can be favored using approaches such as the “knobs-into-holes” technique and others, such as DD-KK variants with asymmetric electrostatic interactions, IgG/IgA strand-exchange engineered domains, HA-TF variants with asymmetric hydrophobic interactions.

In preferred embodiments, the fusion protein or conjugated protein of the present invention comprises or consists of an IgG Fc-homodimer, wherein each of the monomer of the IgG Fc-homodimer is C-terminally linked to at least one polypeptide (P1) comprising or consisting of at least one FSD1 domain comprising or consisting of a polypeptide having at least 70%, such as at least 80%, for instance at least 90%, such as at least 95%, for instance at least 99% sequence identity to SEQ ID NO: 1, and wherein each of the monomer of the IgG Fc-homodimer is linked N-terminally to a therapeutic or diagnostic moiety identical or different.

Such fusion protein or conjugated proteins may be noted herein with the general formula:

(therapeutic or diagnostic moiety)−Fc−(P1).

Human and mouse-derived Fc regions may further be noted herein hFc and mFc, respectively.

In other preferred embodiments, the fusion protein or conjugated protein of the present invention comprises or consists of an IgG Fc-heterodimer, wherein each of the monomer of the IgG Fc-heterodimer is linked C-terminally to at least one polypeptide (P1) comprising or consisting of at least one FSD1 domain comprising or consisting of a polypeptide having at least 70%, such as at least 80%, for instance at least 90%, such as at least 95%, for instance at least 99% sequence identity to SEQ ID NO: 1, and wherein each of the monomer of the IgG Fc-heterodimer is N-terminally linked to a therapeutic or diagnostic moiety identical or different.

In some embodiments, the fusion protein or conjugated protein comprises or consists of a polypeptide having at least 70% sequence identity, such as at least 80% sequence identity, for example at least 90% sequence identity, such as at least 95% sequence identity, for example 95% sequence identity with a polypeptide as set forth in the group consisting of: SEQ ID NO: 7, and SEQ ID NO: 9.

In other embodiments, the fusion protein or conjugated protein comprises or consists of a polypeptide encoded by a polynucleotide having at least 70% sequence identity, such as at least 80% sequence identity, for example at least 90% sequence identity, such as at least 95% sequence identity, for example 95% sequence identity with a polypeptide as set forth in the group consisting of: SEQ ID NO: 8 and SEQ ID NO: 10.

### Type 4

The at least one polypeptide (P1) comprising at least one FSD1 domain of the fusion proteins or conjugated proteins of the present invention may be linked N-terminally to an IgG Fc domain.

In one embodiment, the invention relates to a fusion protein or conjugated protein of the present invention comprising:
(i) one or more polypeptide(s) (P1) comprising or consisting of at least one FSD1 domain comprising or consisting of a polypeptide having at least 70%, such as at least 80%, for instance at least 90%, such as at least 95%, for instance at least 99% sequence identity to SEQ ID NO: 1, and
(ii) a therapeutic or diagnostic agent; wherein the therapeutic or diagnostic agent comprises or consists of an IgG Fc-heterodimer, wherein at least one of the monomer of the IgG Fc-heterodimer is linked N-terminally to at least one polypeptide (P1) comprising or consisting of at least one FSD1 domain comprising or consisting of a polypeptide having at least 70%, such as at least 80%, for instance at least 90%, such as at least 95%, for instance at least 99% sequence identity to SEQ ID NO: 1, and wherein at least one of the monomer of the IgG Fc-heterodimer is linked N-terminally to a therapeutic or diagnostic moiety identical or different.

Such fusion protein or conjugated proteins may be noted herein with the general formula:

(therapeutic or diagnostic molecule)/(P1)−Fc.

Human and mouse-derived Fc regions may further be noted herein hFc and mFc, respectively.

The fusion protein or conjugated protein of the present invention may be linked to one or more polypeptide (P1), for instance one or more polypeptides (P1), such as polypeptides (P1) polymers, such as at least three polypeptides (P1), for example at least four polypeptides, such as at least five polypeptides (P1), for example at least ten polypeptides (P1), such as at least twenty-five polypeptides (P1) comprising or consisting of at least one FSD1 domain comprising or consisting of a polypeptide having at least 70%, such as at least 80%, for instance at least 90%, such as at least 95%, for instance at least 99% sequence identity to SEQ ID NO: 1.

Thus, in some preferred embodiments, at least one of the monomers of the IgG Fc-heterodimer of the fusion protein or conjugated protein of the present invention is linked N-terminally to at least one polypeptide (P1), for example at least two polypeptides (P1), such as at least three polypeptides (P1), for example at least four polypeptides, such as at least five polypeptides (P1) comprising at least one FSD1 domain comprising or consisting of a polypeptide having at least 70%, such as at least 80%, for instance at least 90%, such as at least 95%, for instance at least 99% sequence identity to SEQ ID NO: 1.

For some applications, it may be beneficial that the therapeutic or diagnostic agent or therapeutic or diagnostic moiety targets, for instance binds a specific protein, such as a specific protein involved in a disease or a condition. For example, in cases where a receptor is known to bind said specific protein, the therapeutic or diagnostic moiety may for instance be a receptor of said specific protein. The specific protein may be a protein involved for instance with immune, inflammatory, or angiogenic response. The skilled person will appreciate that conditions such as, but not limited to cancer, diabetes, arthritis, asthma, cardiovascular diseases, and chronic inflammatory diseases may benefit from targeting the immune, inflammatory or angiogenic response.

In other embodiments, the therapeutic or diagnostic agent, or the at least one therapeutic or diagnostic moiety identical or different of the fusion protein or conjugated protein of the present invention is one or more cytokine receptor.

The skilled person will appreciate that cytokines are immune-modulating agents and are extensively used in cellular communication. The term cytokines encompasses a wide range of polypeptide regulators, such as interferons, interleukins, chemokines or Tumor Necrosis Factor.

In further embodiments, the therapeutic or diagnostic agent, or the at least one therapeutic or diagnostic moiety identical or different of the fusion protein or conjugated protein of the present invention comprises or consists of the ligand binding domain of Tumor Necrosis Factor Receptor 2 (TNFR2) (SEQ ID NO: 21).

In yet other embodiments, the therapeutic or diagnostic agent, or the at least one therapeutic or diagnostic moiety identical or different of the fusion protein or conjugated protein of the present invention comprises or consists of the ligand binding domain of one or more growth factor receptor.

Examples of growth factor receptors include, but are not limited to: insulin growth factor receptors (IGF-1R, IR and IRR); activin type IIA and IIB receptors (ActRIIA, ActRIIB); epidermal growth factor family receptors (EGFR, ErbB2, and ErbB4); platelet derived growth factor receptors (PDGFRs), vascular endothelial growth factor receptors (VGFRs), tyrosine kinase with immunoglobulin-like and epidermal growth factor homology domains (TIE-2), macrophage colony stimulating factor (c-fms), c-kit, c-met, fibroblast growth factor receptors (FGFRs), hepatocyte growth factor receptors (HGFRs), Trk receptors (TrkA, TrkB, and TrkC), ephrin (Eph) receptors and the RET proto-oncogene.

In certain embodiments, the therapeutic or diagnostic agent, or the at least one therapeutic or diagnostic moiety identical or different of the fusion protein or conjugated protein of the present invention comprises or consists of the ligand binding domain of VGFR1 (SEQ ID NO: 19).

In other embodiments, the therapeutic or diagnostic agent, or the at least one therapeutic or diagnostic moiety identical or different of the fusion protein or conjugated protein of the present invention comprises or consists of the ligand binding domain of VGFR2 (SEQ ID NO: 20).

In preferred embodiments, the one or more polypeptide(s) (P1) (i) comprising or consisting of at least one FSD1 domain comprise or consist of a polypeptide having at least 70% sequence identity, such as at least 80% identity, for instance at least 90% identity, such as at least 95% identity, for instance 99% identity to SEQ. ID. NO: 26.

In some embodiments, the IgG Fc-heterodimer of the fusion protein or the conjugate protein of the present invention comprises or consists of a monomer having at least 70% sequence identity, for example at least 80% identity, such as least 90% identity, for example at least 95% identity, such as at least 99% identity with SEQ ID NO: 11 and a monomer having at least 80% sequence identity, for example at least 85% identity, such as least 90% identity, for example at least 95% identity, such as at least 99% identity with a sequence selected from the group consisting of: SEQ ID NO: 13, SEQ ID NO: 15, and SEQ ID NO: 17.

In other embodiments, the IgG Fc-heterodimer of the fusion protein or the conjugate protein of the present invention comprises or consists of a monomer encoded by a sequence having at least 70% sequence identity, for example at least 80% identity, such as least 90% identity, for example at least 95% identity, such as at least 99% identity with SEQ ID NO: 12 and a monomer encoded by a sequence having at least 80% sequence identity, for example at least 85% identity, such as least 90% identity, for example at least 95% identity, such as at least 99% identity with a sequence selected from the group consisting of: SEQ ID NO: 14, SEQ ID NO: 16, SEQ ID NO: 18.

### Type 3

The at least one polypeptide (P1) comprising at least one FSD1 domain of the fusion proteins or conjugated proteins of the present invention may be linked to a full-length antibody.

Thus another embodiment of the present invention relates to a fusion protein or conjugated protein comprising:
(i) one or more polypeptide(s) (P1) comprising or consisting of at least one FSD1 domain comprising or consisting of a polypeptide having at least 70%, such as at least 80%, for instance at least 90%, such as at least 95%, for instance at least 99% sequence identity to SEQ ID NO: 1, and
(ii) a therapeutic or diagnostic agent;
wherein the one or more polypeptide(s) (P1) (i) and the therapeutic or diagnostic agent are linked covalently or by non-covalent interaction.

In further embodiments, the one or more polypeptide(s) (P1) (i) and the therapeutic or diagnostic agent are linked covalently using click chemistry,

In other embodiments, the one or more polypeptide(s) (P1) (i) and the therapeutic or diagnostic agent are linked by a non-covalent streptavidin-biotin interaction.

In yet other embodiments, the one or more polypeptide(s) (P1) (i) and the therapeutic or diagnostic agent are linked by a nanobody with affinity for the therapeutic or diagnostic agent, such as an anti-Fc nanobody.

In some embodiments, the therapeutic or diagnostic agent comprises or consists of a full-length antibody.

The full-length antibody may be multi-specific antibody such as for instance a bi-specific antibody.

In preferred embodiments, the full-length antibody is a bi-specific therapeutic antibody.

For some applications of the fusion proteins or conjugated proteins as described herein, it may be preferable that the FSD1 domains of the one or more polypeptides (P1) further comprise residues allowing chemical conjugation of the one or more polypeptides (P1) to the therapeutic or diagnostic agent (ii). The additional residues may be for example, but not limited to, lysine-threonine-cysteine (also referred to herein as lys-thr-cys or KTC), or other residues providing a free C-terminal cysteine.

Therefore, in some embodiments, the one or more polypeptide(s) (P1) (i) comprise or consist of a sequence having at least 70% similarity, such as at least 80% similarity, for instance at least 90% similarity, such as at least 95% similarity, for instance at least 99% similarity with a sequence selected from the group consisting of: SEQ ID NO: 23, SEQ ID NO: 24, SEQ ID NO: 64, SEQ ID NO: 65, and SEQ ID NO: 66.

In some embodiments the one or more polypeptide(s) (P1) (i) comprise or consist of a sequence having at least 70% similarity, such as at least 80% similarity, for instance at least 90% similarity, such as at least 95% similarity, for instance at least 99% similarity with a sequence selected from the group consisting of: SEQ ID NO: 23 and SEQ ID NO: 24.

In some embodiments the one or more polypeptide(s) (P1) (i) are encoded by a sequence comprising or consisting of a sequence having at least 70% similarity, such as at least 80% similarity, for instance at least 90% similarity, such as at least 95% similarity, for instance at least 99% similarity with SEQ ID NO: 25.

### Variant FSD1 Domains

In some embodiments of the fusion protein or conjugated protein of the present invention, the at least one FSD1 domain comprising or consisting of a polypeptide having at least 70%, such as at least 80%, for instance at least 90%, such as at least 95%, for instance at least 99% sequence identity to SEQ ID NO: 1 is a variant of said FSD1 domain defined by a further polypeptide sequence as described herein, having at least 70%, such as at least 80%, for instance at least 90%, such as at least 95%, for instance at least 99% sequence identity to SEQ ID NO: 1.

Therefore in preferred embodiments, the at least one FSD1 domain of the (i) one or more polypeptides (P1) comprises or consists of a variant FSD1 domain encoded by a sequence selected from the group consisting of: SEQ ID NO: 47, SEQ ID NO: 50, and SEQ ID NO: 53, or a polypeptide having at least 70% such as at least 80%, for instance at least 90%, such as at least 95%, for instance at least 96%, such as at least 97%, for instance 98%, such as at least 99% sequence identity thereto.

Thus in some embodiments, the fusion protein or conjugated protein of the present invention comprises:
(i) one or more polypeptide(s) (P1) comprising or consisting of at least one FSD1 domain comprising or consisting of a variant FSD1 domain encoded by a sequence selected from the group consisting of: SEQ ID NO: 47, SEQ ID NO: 50, and SEQ ID NO: 53, or a polypeptide having at least 70% such as at least 80%, for instance at least 90%, such as at least 95%, for instance at least 96%, such as at least 97%, for instance 98%, such as at least 99% sequence identity thereto,
and
(ii) a therapeutic or diagnostic agent.

In some embodiments, the variant FSD1 domain is selected from the group consisting of: FSD1 (Q124A) of sequence ID NO: 47, FSD1 (E126A) of sequence ID NO: 50, and FSD1 (Q124 E126A) of sequence ID NO: 53.

### Functional Features of the Fusion Protein or Conjugated Proteins

### Absence of Activin A, Myostatin and GDF-11 Neutralization

The one or more polypeptide(s) (P1) comprising at least one FSD1 domain of the present invention do not neutralize the activity of the growth factors, such as the activin A (SEQ ID NO: 44), myostatin (SEQ ID NO: 45), and GDF11 (SEQ ID NO: 46). This neutralization is present in contrary with full-length follistatins, such as follistatins comprising the N-terminal domain of follistatin (SEQ. ID NO: 43) and domains 1, 2 and 3 (FSD1 (SEQ ID NO: 1), FSD2 (SEQ ID NO: 41) and FSD3 (SEQ ID NO: 42). Importantly, the neutralization of these growth factors is associated with biological effects on muscle growth (such as skeletal muscle anabolism) which are undesirable for the versatile use of (P1) polypeptides as a platform for developing fusion proteins or conjugated proteins. The lack of neutralization of activin A, myostatin, and/or GDF11 may be measured through any assay known in the art, for example measuring the absence of downstream signalling activation by these growth factors, for instance the absence of intracellular pSmad2/3 signalling activation, such as measured using a reporter gene bioassay of phosphorylated Smad2/3 signaling as in the Examples herein.

In preferred embodiments of the present invention, the one or more polypeptide(s) (P1) of the fusion protein or conjugated protein do not neutralize the activity of activin A (SEQ ID NO: 44), myostatin (SEQ ID NO: 45), and/or GDF11 (SEQ ID NO: 46).

In some other embodiments, the fusion protein or conjugated protein do not neutralize the activity of myostatin (SEQ ID NO: 45), GDF11 (SEQ ID NO: 46) and/or activin A (SEQ ID NO: 44) in a concentration range up to at least 5 nM, such as at least 7.5 nM, for example at least 8 nM, such as at least 8.2 nM, for example at least 8.4 nM, such as at least 8.6 nM, for example at least 8.8 nM, such as at least 9 nM, for example at least 10 nM, such as at least 12 nM, for example at least 15 nM, such as at least 20 nM, for example at least 30 nM, such as at least 40 nM, for example at least 50 nM, such as at least 60 nM, for example at least 61 nM, such as at least 62 nM, for example at least 63 nM, such as at least 64 nM, for example at least 64 nM, such as at least 65 nM, for example at least 70 nM, such as at least 80 nM, for example at least 100 nM, such as at least 150 nM, for example at least 200 nM, such as at least 500 nM, for example at least 600 nM, such as at least 700 nM, for example at least 750 nM, such as at least 800 nM, for example at least 820 nM, such as at least 821 nM, for example at least 822 nM, such as at least 830 nM, for example at least 1 μM, such as at least 2 μM, for example at least 3 μM, such as at least 4 μM, for example at least 5 μM, such as at least 6 μM, for example at least 7 μM, such as at least 8 μM, for example at least 9UM, such as at least 10UM, wherein the neutralization, or absence thereof, is determined by reporter gene bioassay of phosphorylated Smad2/3 signaling

In some embodiments, the fusion protein or conjugated protein do not neutralize the activity of myostatin, GDF11 and/or activin A in a concentration range up to at least 5 nM, such as at least 7.5 nM, for example at least 8 nM, such as at least 8.2 nM, for example at least 8.4 nM, such as at least 8.6 nM, for example at least 8.8 nM, such as at least 9 nM, for example at least 10 nM, such as at least 12 nM, for example at least 15 nM, such as at least 20 nM, for example at least 30 nM, such as at least 40 nM, for example at least 50 nM, such as at least 80 nM, for example at least 100 nM, such as at least 150 nM, for example at least 200 nM.

In further embodiments, the one or more polypeptide(s) (P1) (i) do not neutralize the activity of myostatin (SEQ ID NO: 45), GDF11 (SEQ ID NO: 46) and/or activin A (SEQ ID NO: 44) in a concentration range up to at least 5 nM, such as at least 7.5 nM, for example at least 8 nM, such as at least 8.2 nM, for example at least 8.4 nM, such as at least 8.6 nM, for example at least 8.8 nM, such as at least 9 nM, for example at least 10 nM, such as at least 12 nM, for example at least 15 nM, such as at least 20 nM, for example at least 30 nM, such as at least 40 nM, for example at least 50 nM, such as at least 60 nM, for example at least 61 nM, such as at least 62 nM, for example at least 63 nM, such as at least 64 nM, for example at least 64 nM, such as at least 65 nM, for example at least 70 nM, such as at least 80 nM, for example at least 100 nM, such as at least 150 nM, for example at least 200 nM, such as at least 500 nM, for example at least 600 nM, such as at least 700 nM, for example at least 750 nM, such as at least 800 nM, for example at least 820 nM, such as at least 821 nM, for example at least 822 nM, such as at least 830 nM, for example at least 1 μM, such as at least 2 μM, for example at least 3 μM, such as at least 4 μM, for example at least 5 μM, such as at least 6 μM, for example at least 7 μM, such as at least 8UM, for example at least 9UM, such as at least 10 μM, wherein the neutralization, or absence thereof, is determined by reporter gene bioassay of phosphorylated Smad2/3 signaling.

In further embodiments, the one or more polypeptide(s) (P1) do not neutralize the activity of myostatin, GDF11 and/or activin A in a concentration range up to at least 5 nM, such as at least 7.5 nM, for example at least 8 nM, such as at least 8.2 nM, for example at least 8.4 nM, such as at least 8.6 nM, for example at least 8.8 nM, such as at least 9 nM, for example at least 10 nM, such as at least 12 nM, for example at least 15 nM, such as at least 20 nM, for example at least 30 nM, such as at least 40 nM, for example at least 50 nM, such as at least 80 nM, for example at least 100 nM, such as at least 150 nM, for example at least 200 nM.

In some embodiments, wherein the fusion protein or conjugated protein as described herein is FSD1-FSD1, Type 2 compounds as described herein, VGFR(½)-mFc-FSD1 and/or TNFR2-mFc-FSD1, the fusion protein or conjugated protein do not neutralize the activity of activin A, myostatin, or GDF11 signaling at a concentration range up to 8.2 nM.

In some embodiments, wherein the fusion protein or conjugated protein as described herein is FSD1-FSD1, the fusion protein or conjugated protein do not neutralize the activity of activin A, myostatin, or GDF11 signaling at a concentration range up to 1 μM.

In other embodiments, FSD1 domain variants selected from the group consisting of: FSD1 (Q124A), FSD1 (E126A), and FSD1 (Q124A, E126A) do not neutralize activin A or myostatin at a concentration range up to 822 nM.

### Heparan Sulfate Binding

The one or more polypeptide(s) (P1) comprising at least one FSD1 domain of the present invention bind heparan sulfate (HS). The skilled person will appreciate that HS is a major component of the extracellular matrix (ECM) and can be found as both free HS chains and as the HS chains of HS proteoglycans, for example cell-surface proteoglycans. HS comprises a domain structure of segregated blocks of repeating β-D-glucuronic acid-(1→4)-N-acetylated β-D-glucosamine disaccharides (NA domains) and blocks of highly sulfated, heparin-like α-L-iduronic acid-(1→4)-N-sulfated β-D-glucosamine disaccharides (NS domains), with a small proportion of mixed sequences of N-acetylated and N-sulfated disaccharides separating the two domains.

As a modular protein platform, such as for the loading of biological agents to the ECM and/or to specific organs, and/or the increase of the half-life of therapeutic or diagnostic agents, it is thus beneficial that the one or more polypeptides (P1) (i) bind heparin sulfate.

In some embodiments of the present invention, the one or more polypeptide(s) (P1) (i) of the fusion protein or conjugated protein of the present invention bind heparan sulfate.

In preferred embodiments, the one or more polypeptide(s) (P1) (i) comprising at least one FSD1 domain of the present invention are encoded by a sequence comprising or consisting of a sequence having at least 70% identity, such as at least 80% identity, for instance at least 90% identity, such as at least 95% identity, for example at least 99% identity with SEQ. ID. NO: 33.

In preferred embodiments, the one or more polypeptide(s) (P1) (i) comprising at least one FSD1 domain of the present invention comprise or consist of a sequence having at least 100% identity with SEQ. ID. NO: 1.

### Linkers

In some embodiments, the fusion protein or conjugated protein of the present invention further comprises a linker between the one or more polypeptide(s) (P1) (i) comprising at least one FSD1 domain and the therapeutic or diagnostic agent (ii).

In further embodiments, the linker is a chemical linker.

### Therapeutic or Diagnostic Agents

In preferred embodiments, the therapeutic or diagnostic agent, or the therapeutic or diagnostic moiety identical or different of the fusion proteins or conjugated proteins of the present invention is selected from the group consisting of: a peptide, a protein, such as an antibody or fragment thereof, a nanobody, a streptavidin, a glycoprotein, or an interleukin, a nucleic acid, and a small molecule.

In other embodiments, the fusion protein or conjugated protein further comprises a detectable moiety. The detectable moiety may for instance be selected from the group consisting of: a fluorescent protein, a gold nanoparticle, a radioactive isotope, biotin or a derivative thereof, and an enzyme.

In preferred embodiments, the therapeutic or diagnostic agent (ii), or the therapeutic or diagnostic moiety identical or different of the fusion proteins or conjugated proteins of the present invention binds a target selected from the group consisting of: cluster of differentiation (CD) proteins, cytokines, such as interleukins, growth factors, such as colony-stimulating factors, immune checkpoint proteins, angiogenic factors, hemostatic factors, chemotaxic factors, neurotrophic factors, inflammatory proteins, tumor antigens, bacterial proteins, and viral proteins.

In some embodiments, the therapeutic or diagnostic agent (ii), or the therapeutic or diagnostic moiety identical or different of the fusion proteins or conjugated proteins of the present invention comprises or consists of the ligand binding domain of a protein selected from the group consisting of: TNFR2, VGFR1, VGFR2, and CBG (Corticosteroid Binding Globulin).

In further embodiments, the therapeutic or diagnostic agent (ii), or the therapeutic or diagnostic moiety identical or different of the fusion proteins or conjugated proteins of the present invention comprises or consists of the ligand binding domain of a protein having at least 70% similarity, such as at least 80% similarity, for instance at least 90% similarity, such as at least 95% similarity, for example at least 99% similarity with a sequence selected form the group consisting of: SEQ ID NO: 19, SEQ ID NO: 20, SEQ ID NO: 21 and SEQ ID NO: 22.

In another embodiments, the therapeutic or diagnostic agent, or the therapeutic or diagnostic moiety identical or different of the fusion proteins or conjugated proteins of the present invention comprises or consists of an anti-vimentin nanobody.

In some embodiments, the anti-vimentin nanobody has at least 70% similarity, such as at least 80% similarity, for instance at least 90% similarity, such as at least 95% similarity, for example at least 99% similarity with SEQ ID NO: 34.

In yet further embodiments, the anti-vimentin nanobody is encoded by a sequence having at least 70% similarity, such as at least 80% similarity, for instance at least 90% similarity, such as at least 95% similarity, for example at least 99% similarity with SEQ ID NO: 35.

In some embodiments, the fusion protein or conjugated protein is as described in the Type 2 section herein i.e. comprising:
(i) one or more polypeptide(s) (P1) consisting of at least one FSD1 domain comprising or consisting of a polypeptide having at least 70%, such as at least 80%, for instance at least 90%, such as at least 95%, for instance at least 99% sequence identity to SEQ ID NO: 1, and
(ii) a therapeutic or diagnostic agent, wherein the therapeutic or diagnostic agent comprises or consists of an immunoglobulin Fc domain; and wherein the one or more polypeptide(s) (P1) are linked C-terminally to said immunoglobulin Fc domain, preferably wherein said immunoglobulin is an IgG, IgA, IgM, IgE, or IgD,
   wherein the therapeutic or diagnostic agent is selected from the list consisting of: an anti-VEGF agent, an anti-TNFα agent and a mutated CBG.

In preferred embodiments, the fusion protein or conjugated protein comprises:
(i) two polypeptide(s) (P1) consisting of one FSD1 domain comprising or consisting of a polypeptide having at least 70%, such as at least 80%, for instance at least 90%, such as at least 95%, for instance at least 99% sequence identity to SEQ ID NO: 1, and
(ii) a therapeutic or diagnostic agent, wherein the therapeutic or diagnostic agent comprises or consists of an immunoglobulin Fc domain; and wherein the two polypeptide(s) (P1) are linked C-terminally to said immunoglobulin Fc domain, preferably wherein said immunoglobulin is an IgG, IgA, IgM, IgE, or IgD,
   wherein the therapeutic or diagnostic agent is selected from the list consisting of: an anti-VEGF agent, an anti-TNFα agent and a mutated CBG.

In other embodiments, the fusion protein or conjugated protein is ubiquitinylated.

In some embodiments, the therapeutic or diagnostic agent, or the therapeutic or diagnostic moiety identical or different of the fusion proteins or conjugated proteins of the present invention bind to a target and the fusion protein or conjugated protein is able to bind heparan sulfate and said target at the same time.

### Platform Strategy

### Local Half-Life Increase

The functional properties of the one or more (P1) (i) polypeptides described herein (e.g. heparin sulfate binding property) enable the use of said polypeptides as modules as a platform technology to increase the half-life of the therapeutic or diagnostic agent, or the therapeutic or diagnostic moiety identical or different as described herein.

Thus, in some embodiments, the fusion protein or conjugated protein has a longer local half-life at the site of administration than the therapeutic or diagnostic agent, or the therapeutic or diagnostic moiety identical or different alone.

### ECM Binding

The functional properties of the (P1) polypeptides described herein (e.g. heparin sulfate binding) enable the use of said polypeptides as modules as a platform technology to increase the binding of the therapeutic or diagnostic agent, or the therapeutic or diagnostic moiety identical or different as described herein to the ECM.

Binding to the ECM may be measured by any method known in the art, for instance using in vitro ECM binding assays, such as colorimetry or fluorescence-based assays. The half maximal effective concentration (EC50) may for example be used to assess the concentration of the compound to be tested to achieve 50% of the total binding capacity, for instance 50% of the binding capacity of the ECM surface tested.

Thus, in some embodiments, the fusion protein or conjugated protein increases the binding of the therapeutic or diagnostic agent, or of the therapeutic or diagnostic moiety identical or different to the extracellular matrix compartment compared to the therapeutic or diagnostic agent, or the therapeutic or diagnostic moiety identical or different alone.

In some embodiments, such as wherein the fusion protein or conjugated protein of the present invention is linked to one or more polypeptide (P1), for instance one or more polypeptides (P1), such as polypeptides (P1) polymers, such as at least three polypeptides (P1), for example at least four polypeptides, such as at least five polypeptides (P1), for example at least ten polypeptides (P1), such as at least twenty-five polypeptides (P1) comprising or consisting of at least one FSD1 domain comprising or consisting of a polypeptide having at least 70%, such as at least 80%, for instance at least 90%, such as at least 95%, for instance at least 99% sequence identity to SEQ ID NO: 1, the ECM binding increases with increasing number of polypeptides (P1).

For example, in some embodiments, FSD1-FSD1-FSD1 compound has a higher ECM binding affinity, such as higher ECM binding affinity than FSD1-FSD1 as measured by the ECM binding assays described herein. In other embodiments, FSD1-FSD1 compound has a higher ECM binding affinity, such as higher ECM binding affinity than FSD1 as measured by the ECM binding assays described herein.

### Organ Loading

In some applications, it may be beneficial that the increased binding of the fusion proteins or conjugated proteins of the present invention takes place at a specific predefined structure, such as a predefined organ or a tumor.

The person skilled in the art will appreciate that the increased binding and delivery for instance of a therapeutic or diagnostic agent to a disease located in a specific compartment, such as an organ, is likely to improve the efficacy of the therapeutic and/or diagnostic effect of said compound.

Thus, in some embodiments, the fusion protein or conjugated protein increases the binding of the therapeutic or diagnostic agent (ii), or of the therapeutic or diagnostic moiety identical or different, to a predefined organ or a tumor compared to the therapeutic or diagnostic agent, or the therapeutic or diagnostic moiety identical or different, alone.

The increased binding may be measured by a decrease in the EC50 i.e. the concentration of the compound to be tested, such as the therapeutic or diagnostic agent (ii), or the therapeutic or diagnostic moiety identical or different, to achieve 50% of the total binding capacity, for instance 50% of the binding capacity of the predefined organ or a tumor tested.

Thus in preferred embodiments, the EC50 of the therapeutic or diagnostic agent (ii) or the therapeutic or diagnostic moiety identical or different, to a predefined organ or a tumor, compared to the therapeutic or diagnostic agent (ii) or the therapeutic or diagnostic moiety identical or different alone is decreased by at least a factor 2, such at least a factor 5, for example at least a factor 10, such as at least a factor 20, for example at least a factor 50, such as at least a factor 80, for example at least a factor 100, such as at least a factor 250, for example at least a factor 500, such at least a factor 1000, for example at least a factor 5000, such as at least a factor 10000, for example at least a factor 100000.

In further embodiments, the predefined organ is selected from the group consisting of: a musculoskeletal system organ, a digestive system organ, a respiratory system organ. a urinary system organ, a reproductive organ, and endocrine system organ, a circulatory system organ, a nervous system organ, an hematopoietic organ and an integumentary system organ.

The organ may be selected from the group consisting of: kidney, eye, liver, heart, lungs, bladder, pancreas, gallbladder, intestine, prostate, brain, skin, muscle, bone, hematopoietic tissue and subcutaneous tissue, such as a joint or synovial tissue.

### Intracellular Uptake

In some embodiments, the fusion protein or the conjugated protein increases the intracellular uptake of the therapeutic or diagnostic agent (ii), or of the therapeutic or diagnostic moiety identical or different compared to the therapeutic or diagnostic agent (ii), or the therapeutic or diagnostic moiety identical or different alone.

### Multimers of (P1) Polypeptides Comprising FSD1

The fusion proteins or conjugated proteins of the present invention may be linked to one or more polypeptides (P1) as described herein.

In preferred embodiments, the therapeutic or diagnostic agent, or the therapeutic or diagnostic moiety identical or different of the fusion protein or conjugated protein of the present invention is linked to at least one polypeptide (P1) (i), for example at least two polypeptides (P1) (i), such as at least three polypeptides (P1) (i), for example at least four polypeptides, such as at least five polypeptides (P1) (i) comprising or consisting of at least one FSD1 domain comprising or consisting of a polypeptide having at least 70% such as at least 80%, for instance at least 90%, such as at least 95%, for instance at least 99% sequence identity to SEQ ID NO: 1.

In other embodiments, the therapeutic or diagnostic agent comprises or consists of a Fc-dimer, each monomer of the Fc-homodimer or Fc-heterodimer is linked to at least one polypeptide (P1) (i), for example at least two polypeptides (P1) (i), such as at least three polypeptides (P1) (i), for example at least four polypeptides, such as at least five polypeptides (P1) (i) comprising or consisting of at least one FSD1 domain comprising or consisting of a polypeptide having at least 70%, such as at least 80%, for instance at least 90%, such as at least 95%, for instance at least 99% sequence identity to SEQ ID NO: 1.

### Multimers of FSD1 Domains and Linkage

It may be beneficial, for instance to solve steric issues, that the at least one polypeptide (P1) (i) comprise or consist of several FSD1 domains as described herein arranged as multimers. For instance, at least one of the at least one polypeptide (P1) (i) may comprise or consist of at least two FSD1 domains (dimer).

In further embodiments, at least one of the at least one polypeptide (P1) (i) of the fusion protein or conjugated protein comprises or consists of a multimer of FSD1 domains, such as at least two FSD1 domains, for example at least three FSD1 domains, for example at least four FSD1 domains, such as at least five FSD1 domains comprising or consisting of a polypeptide having at least 70%, such as at least 80%, for instance at least 90%, such as at least 95%, for instance at least 99% sequence identity to SEQ ID NO: 1.

In yet other embodiments, at least 2 monomers of the multimers of FSD1 domains of the fusion protein or conjugated protein of the present invention are joined by a linker.

In some embodiments, the fusion protein or conjugated protein comprises or consists of a polypeptide having at least 70% sequence identity, such as at least 80% sequence identity, for example at least 90% sequence identity, such as at least 95% identity, for example at least 99% identity with a sequence selected from the group consisting of: SEQ ID NO: 2, SEQ ID NO: 3, SEQ ID NO: 5, SEQ ID NO: 7, and SEQ ID NO: 9.

In other embodiments, the fusion protein or conjugated protein comprises or consists of a polypeptide having at least 70% sequence identity, such as at least 80% sequence identity, for example at least 90% sequence identity, such as at least 95% identity, for example at least 99% identity with SEQ ID NO: 11 and a polypeptide having at least 70% sequence identity, such as at least 80% sequence identity, for example at least 90% sequence identity, such as at least 95% identity, for example at least 99% identity with a sequence selected from the group consisting of: SEQ ID NO: 13, SEQ ID NO: 15, and SEQ ID NO: 17.

### Polynucleotides

Another aspect of the present invention relates to one or more polynucleotides which upon expression encode the fusion protein or conjugated protein as described herein. In some embodiments, the one or more polynucleotides have at least 70% sequence identity, such as at least 80% sequence identity, for example at least 90% sequence identity, such as at least 95% identity, for example at least 99% identity with a polynucleotide selected from the group consisting of: SEQ ID NO: 4, SEQ ID NO: 6, SEQ ID NO: 8, and SEQ ID NO: 10.

In other embodiments, the one or more polynucleotides have at least 70% sequence identity, such as at least 80% sequence identity, for example at least 90% sequence identity, such as at least 95% identity, for example at least 99% identity with a combination of SEQ ID NO: 12 and at least one polynucleotide selected from the group consisting of: SEQ ID NO: 14, SEQ ID NO: 16, and SEQ ID NO: 18.

### Constructs or Vectors

A further aspect of the present invention relates to one or more constructs or vectors comprising the one or more polynucleotides as described herein and/or encoding the fusion protein or conjugated protein as described herein.

The skilled person will appreciate that polynucleotides and construct or vectors for the expression of the compounds of the present invention may further comprise additional sequences known in the art, such as sequences used to facilitate for example the production, expression or purification of the compounds. The additional sequence may be, but not limited to, purification tags known in the art, such as polyhistidine tag(s).

### Host Cells

The skilled person will appreciate that the expression of recombinant proteins, such as the fusion protein or conjugated proteins as described herein requires an expression system, such as a mammalian, insect, yeast, bacterial, algal or cell-free expression system.

An aspect of the present invention relates to a host cell comprising the one or more polynucleotides as described herein or the one or more constructs or vectors as described herein.

In preferred embodiments, the host cell as described herein is a mammalian cell.

In even preferred embodiments, the host cell is a human cell, such as a human embryonic kidney 293 cell (HEK293).

In other embodiments, the host cell is a Chinese hamster ovary (CHO) cell.

### Compositions

Another aspect of the present invention relates to a composition comprising the fusion protein or conjugated protein as described herein, the one or more polynucleotides as described herein, the one or more constructs or vectors as described herein, the host cell as described herein, or mixtures thereof.

In preferred embodiments the composition as described herein is a pharmaceutical composition.

Another aspect of the present invention relates to a composition comprising one or more polypeptides selected from the group consisting of: FSD1 (Q124A) of sequence ID NO: 47, FSD1 (E126A) of sequence ID NO: 50, and FSD1 (Q124 E126A) of sequence ID NO: 53, or variants thereof, having at least 70% such as at least 80%, for instance at least 90%, such as at least 95%, for instance at least 96%, such as at least 97%, for instance 98%, such as at least 99% sequence identity thereto.

### Medical Uses and Methods of Treatment.

In further embodiments, the fusion protein or conjugated protein as described herein, the one or more polynucleotides as described herein, the one or more constructs or vectors as described herein, the host cell as described herein, or the composition as described herein are for use in medicine.

In preferred embodiments, the fusion protein or conjugated protein as described herein, the one or more polynucleotides as described herein, the one or more constructs or vectors as described herein, the host cell as described herein, or the composition as described herein are for use in the treatment of an ophthalmologic condition, an oncogenic condition, an inflammatory condition, a neurological condition, a cardiovascular condition, a metabolic condition, a respiratory condition, a musculoskeletal condition, an ageing-related condition.

In more preferred embodiments, the fusion protein or conjugated protein as described herein, the one or more polynucleotides as described herein, the one or more constructs or vectors as described herein, the host cell as described herein, or the composition as described herein are for use in the treatment of an ophthalmologic condition, an oncogenic condition, or an inflammatory condition.

In some embodiments, the ophthalmologic condition is neovascular age-related macular degeneration (wet AMD). In further embodiments, the ophthalmologic condition is neovascular age-related macular degeneration (wet AMD) and the therapeutic or diagnostic agent (ii) of the fusion protein or conjugated protein as described herein is an anti-VEGF agent, preferably wherein the fusion protein or conjugated protein is as described in the Type 2 section herein.

In some embodiments wherein the fusion protein or conjugated protein as described herein, the one or more polynucleotides as described herein, the one or more constructs or vectors as described herein, the host cell as described herein, or the composition as described herein are for use in the treatment of an inflammatory condition, the therapeutic or diagnostic agent (ii) of the fusion protein or conjugated protein as described herein is an anti-TNFα agent, preferably wherein the fusion protein or conjugated protein is as described in the Type 2 section herein.

In some embodiments wherein the fusion protein or conjugated protein as described herein, the one or more polynucleotides as described herein, the one or more constructs or vectors as described herein, the host cell as described herein, or the composition as described herein are for use in the treatment of a musculoskeletal condition, such as steroid-induced myopathy, the therapeutic or diagnostic agent (ii) of the fusion protein or conjugated protein as described herein is a mutated CBG, preferably wherein the fusion protein or conjugated protein is as described in the Type 2 section herein.

In some embodiments wherein the fusion protein or conjugated protein as described herein, the one or more polynucleotides as described herein, the one or more constructs or vectors as described herein, the host cell as described herein, or the composition as described herein are for use in the treatment of an oncogenic condition, or an autoimmune condition, the therapeutic or diagnostic agent (ii) of the fusion protein or conjugated protein as described herein is Interleukin 2 (IL-2), preferably wherein the fusion protein or conjugated protein is as described in the Type 1 section herein.

In other embodiments, the inflammatory condition is caused by organ transplantation.

In yet other embodiments, the medical use is in a subject donating or receiving an organ, and the donated or transplanted organ is selected from the group consisting of: kidney, heart, lung, bone marrow, and liver.

Another aspect of the present invention relates to a method of treatment of a disease or condition, comprising administering to a subject an effective amount of the fusion protein or conjugated protein as described herein, the one or more polynucleotides as described herein, the one or more constructs or vectors as described herein, the host cell as described herein, or the composition as described herein.

A further aspect of the present invention relates to a method of treating an ophthalmologic condition, an inflammatory condition, or an oncogenic condition, comprising administering to a subject an effective amount of the fusion protein or conjugated protein as described herein, the one or more polynucleotides as described herein, the one or more constructs or vectors as described herein, the host cell as described herein, or the composition as described herein.

Yet another aspect of the present invention relates to the use of the fusion protein or conjugated protein as described herein, the one or more polynucleotides as described herein, the one or more constructs or vectors as described herein, the host cell as described herein, or the composition as described herein in the manufacture of a medicament for the treatment of a disease or condition, for example an ophthalmologic condition, an oncogenic condition, or an inflammatory condition.

In some embodiments of the use of the fusion protein or conjugated protein as described herein, or of the method as described herein the fusion protein or conjugated protein, the one or more polynucleotide, the one or more constructs or vectors, the host cell, or the composition as described herein is administered systematically.

In some embodiments of the use of the fusion protein or conjugated protein as described herein, or of the method as described herein the fusion protein or conjugated protein, the one or more polynucleotide, the one or more constructs or vectors, the host cell, or the composition as described herein is administered locally.

In some embodiments of the use of the fusion protein or conjugated protein as described herein, or of the method as described herein the fusion protein or conjugated protein, the one or more polynucleotide, the one or more constructs or vectors, the host cell, or the composition as described herein is administered intraadiposally, intraarterially, intraarticularly, intracranially, intradermally, intralesionally, intramuscularly, intranasally, intraocularly, intraosseously, intrapericardially, intraperitoneally, intrapleurally, intraprostatically, intrarectally, intrathecally, intratracheally, intratumorally, intraumbilically, intravaginally, intravenously, intravesicularlly, intravitreally, or liposomally.

### Method of Increasing the Local Half-Life of a Therapeutic or Diagnostic Agent

An aspect of the present invention relates to a method of increasing the local half-life of a therapeutic or diagnostic agent at the site of administration, comprising obtaining the fusion protein or conjugated protein as described herein, the one or more polynucleotides as described herein, the one or more constructs or vectors as described herein, the host cell as described herein, or the composition as described herein, wherein the therapeutic or diagnostic agent, or the therapeutic or diagnostic moiety identical or different of the fusion protein or conjugated protein as described herein comprises or consists of said therapeutic or diagnostic agent.

A further aspect of the present invention relates to a method of increasing the in vivo local half-life of a therapeutic or diagnostic agent in a subject at the site of administration, comprising administering to said subject the fusion protein or conjugated protein as described herein, the one or more polynucleotides as described herein, the one or more constructs or vectors as described herein, the host cell as described herein, or the composition as described herein, wherein the therapeutic or diagnostic agent or the therapeutic or diagnostic moiety identical or different of the fusion protein or conjugated protein as described herein comprises or consists of said therapeutic or diagnostic agent.

In some embodiments, the fusion protein or conjugated protein as described herein, the one or more polynucleotides as described herein, the one or more constructs or vectors as described herein, the host cell as described herein, or the composition as described herein is administered to a subject suffering from an ophthalmologic condition, an oncogenic condition, an inflammatory condition, a neurological condition, a cardiovascular condition, a metabolic condition, a respiratory condition, a musculoskeletal condition, an ageing-related condition.

In a preferred embodiment, the fusion protein or conjugated protein, or the composition is administered to a subject suffering from an ophthalmologic condition and the fusion protein or conjugated protein, or the composition is administered intravitreally, subretinally or suprachoroidally.

In further embodiments, the ophthalmologic condition is wet AMD.

In other embodiments, the fusion protein or conjugated protein, or the composition is administered to a subject donating or receiving an organ, preferably a kidney, heart, lung, bone marrow, or liver, even more preferably a kidney, or administered to said organ during ex vivo perfusion, and the therapeutic or diagnostic agent comprises or consists of a compound reducing inflammation.

In preferred embodiments, the local half-life of the therapeutic or diagnostic agent is increased by at least 6h, such as at least 12 h, for instance at least 24 h, such as at least 48h, for instance at least 72h, such as at least 96h, for instance at least 120h, such as at least one week, for instance at least 2 weeks, such as at least 4 weeks, for instance at least 8 weeks, such as at least 3 months, for instance at least 6 months, such as at least 12 months at the site of administration by the methods of the present invention.

### Method of Increasing ECM Binding of a Therapeutic or Diagnostic Agent

Another aspect of the present invention relates to a method of increasing the binding of a therapeutic or diagnostic agent to the extracellular matrix, comprising administering to a subject the fusion protein or conjugated protein as described herein, the one or more polynucleotides as described herein, the one or more constructs or vectors as described herein, the host cell as described herein, or the composition according as described herein, wherein the therapeutic or diagnostic agent, or the therapeutic or diagnostic moiety identical or different of the fusion protein or conjugated protein as described herein comprises or consists of said therapeutic or diagnostic agent.

### Method of Increasing the Binding of a Therapeutic or Diagnostic Agent to a Predefined Organ.

An aspect of the present invention relates to a method of increasing the binding of a therapeutic or diagnostic agent to a predefined organ, comprising administering to a subject the fusion protein or conjugated protein as described herein, the one or more polynucleotides as described herein, the one or more constructs or vectors as described herein, the host cell as described herein, or the composition according as described herein, wherein the therapeutic or diagnostic agent, or the therapeutic or diagnostic moiety identical or different of the fusion protein or conjugated protein as described herein comprises or consists of said therapeutic or diagnostic agent.

### Method of Increasing the Intracellular Uptake of a Therapeutic or Diagnostic Agent.

An aspect of the present invention relates to a method of increasing the intracellular uptake of a therapeutic or diagnostic agent, comprising administering to a subject the fusion protein or conjugated protein as described herein, the one or more polynucleotides as described herein, the one or more constructs or vectors as described herein, the host cell as described herein, or the composition according as described herein, wherein the therapeutic or diagnostic agent, or the therapeutic or diagnostic moiety identical or different of the fusion protein or conjugated protein as described herein comprises or consists of said therapeutic or diagnostic agent.

In further embodiments, said therapeutic or diagnostic agent is transported to the cytosol, such as to cytoskeleton filaments, or to the nucleus.

### Method for Degrading Intracellular Protein Using a Proteolysis Targeting Chimera (PROTAC) Strategy.

The fusion protein or conjugated proteins of the present invention may be used for degrading intracellular protein using a proteolysis targeting chimera (PROTAC) strategy.

In some embodiments, the fusion protein or conjugated protein of the present invention further comprises a PROTAC linker, preferably wherein the PROTAC linker is selected from the group consisting of: flexible aliphatic linkers, flexible PEGylated linkers, flexible heterochains, rigid linkers, triazole-based linkers, Bio-orthogonal clickable linkers and photo-switchable linkers.

An aspect of the invention therefore relates to a method of degrading an intracellular protein, said method comprising the step of coupling the fusion protein or conjugated protein according to any one of the preceding items to a ligand of E3 ubiquitin ligase, wherein the therapeutic or diagnostic agent (ii) of said fusion protein or conjugated protein binds to said intracellular protein.

In some embodiments, the ligand of E3 ubiquitin ligase is (S, R, S)-AHPC, preferably the ligand of E3 ubiquitin ligase is (S, R, S)-AHPC-PEG8-NHS.

In other embodiments, the E3 ubiquitin ligase is the Von-Hippel Lindau (VHL) tumor suppressor protein.

In some embodiments, the therapeutic or diagnostic agent (ii) is an anti-vimentin nanobody.

In other embodiments, the fusion protein or conjugated protein is a Type 1 protein as described herein, preferably the fusion protein or conjugated protein is anti-vimentin-nanobody-FSD1.

### Subjects of Medical Uses and Methods.

In preferred embodiments of the present invention, the subject of the use of the fusion protein or conjugated protein described herein or the subject in the methods as described herein is a human or a non-human animal.

### Administration Routes of the Uses and Methods of the Invention.

In some embodiments of the uses and methods of the present invention, the fusion protein or conjugated protein as described herein, the one or more polynucleotides as described herein, the one or more constructs or vectors as described herein, the host cell according as described herein, or the composition as described herein is administered systematically.

In other embodiments of the uses and methods of the present invention, the fusion protein or conjugated protein as described herein, the one or more polynucleotides as described herein, the one or more constructs or vectors as described herein, the host cell according as described herein, or the composition as described herein is administered locally.

In further embodiments of the uses and methods of the present invention, the fusion protein or conjugated protein as described herein, the one or more polynucleotides as described herein, the one or more constructs or vectors as described herein, the host cell according as described herein, or the composition as described herein is administered intraadiposally, intraarterially, intraarticularly, intracranially, intradermally, intralesionally, intramuscularly, intranasally, intraocularally, intraosseously, intrapericardially, intraperitoneally, intrapleurally, intraprostatically, intrarectally, intrathecally, intratracheally, intratumorally, intraumbilically, intravaginally, intravenously, intravesicularlly, intravitreally, or liposomally.

### EXAMPLES

### Example 1: Construct Design and Sequence Optimization

### Aim:

The aim of this example was to develop four types of protein designs, which combined therapeutic activity and strong affinity to heparan sulfates that are present in both the ECM and in the glycocalyx of cells.

### Material and Methods:

In constructs designed for mammalian expression systems, the serum albumin preproprotein signal peptide (MKWVTFISLLFLFSSAYS) was used as a leader sequence to facilitate extracellular expression (REF. 1). A vector containing an IRES2 sequence was used for bicistronic expression of the protein of interest and an intracellular fluorescent marker. All sequences were optimized for expression in mammalian cells. For bacterial expression, the gene encoding the protein of interest was optimized for bacterial expression and cloned into the pET22B(+) vector.

### Results:

Common to all four designs was the presence of FSD1 (either as a monomer or multimer) or a full-length FST variant as a platform that provided affinity to heparan sulfate. A linker may optionally be applied in all designs between FSD1 and the fused moiety.

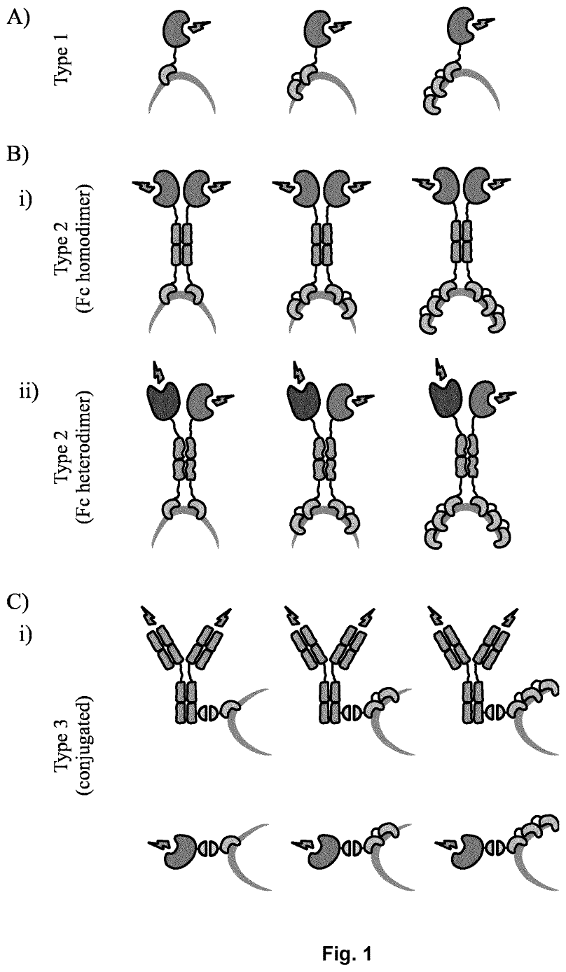

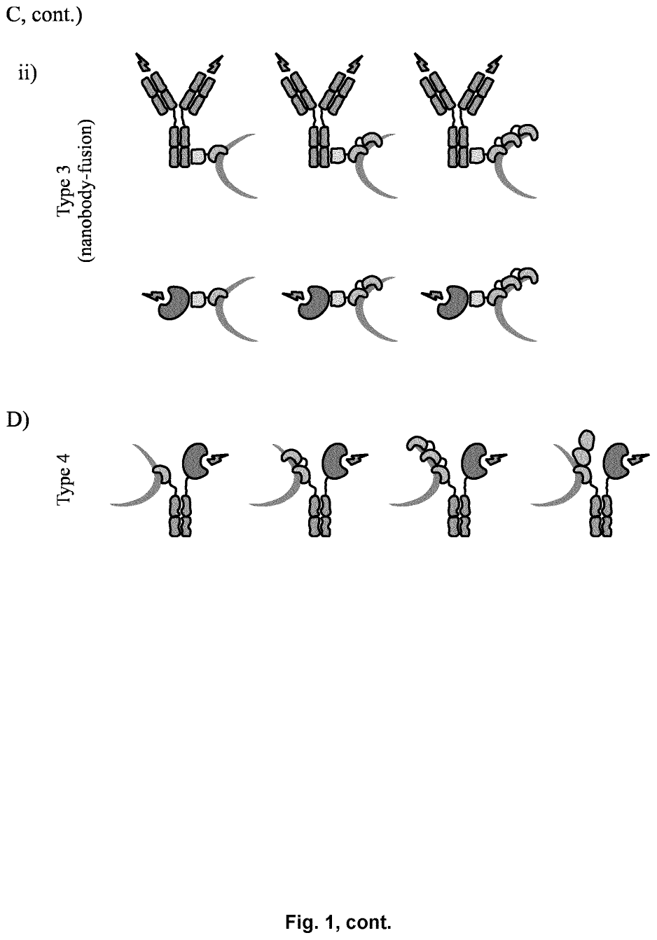

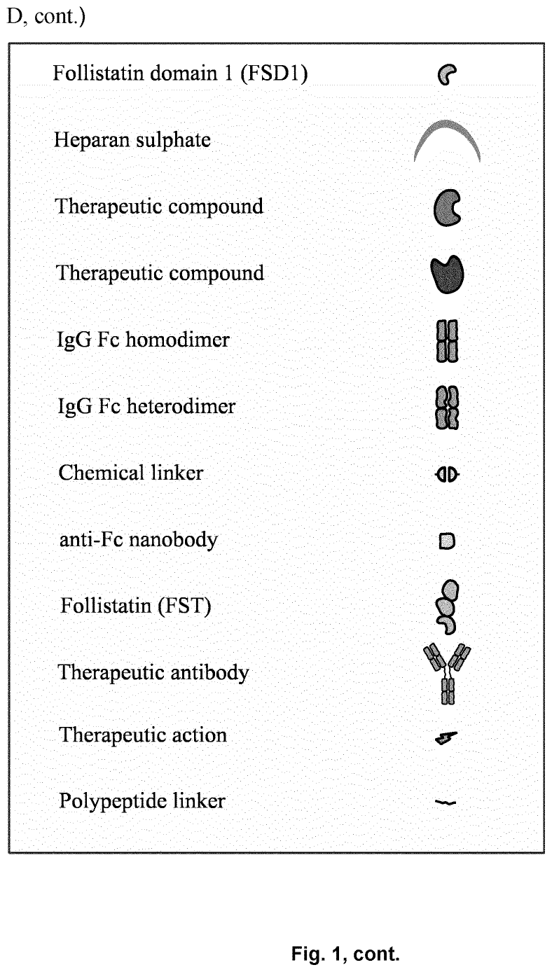

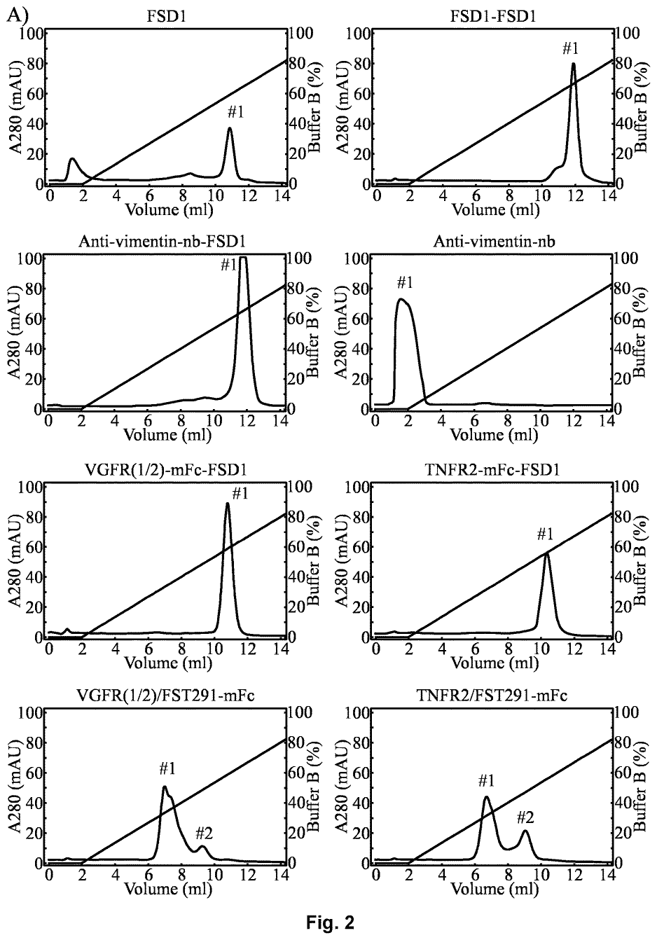

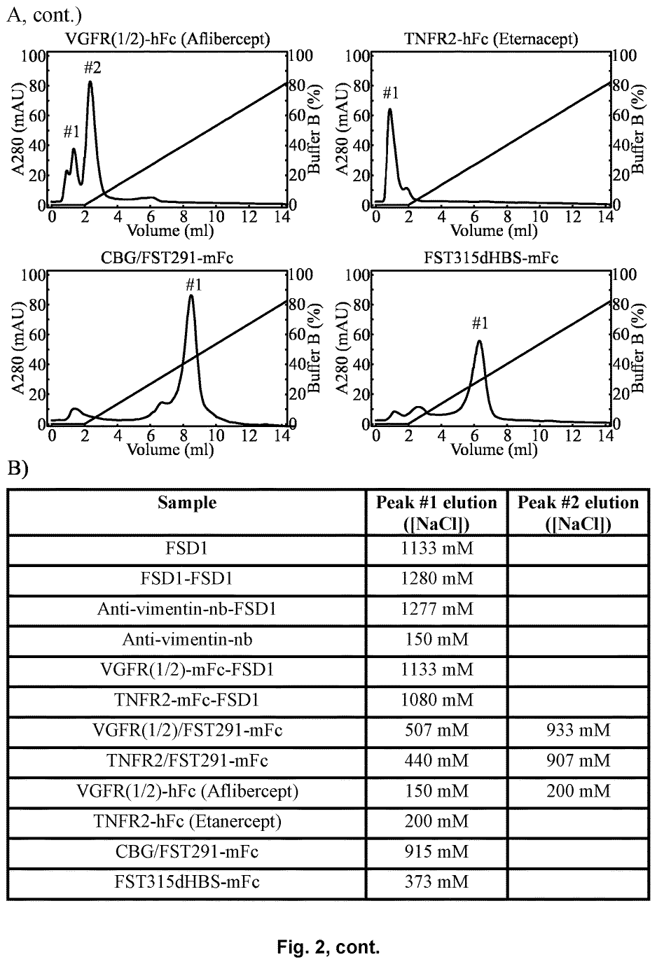

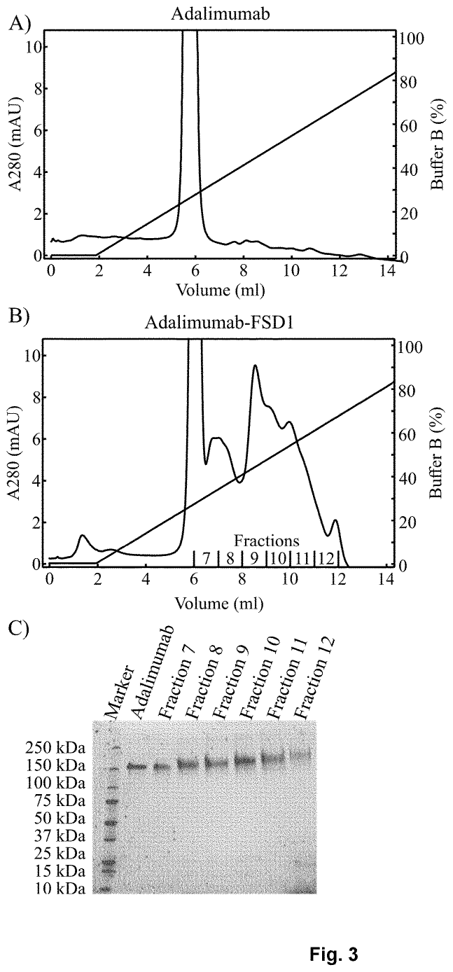

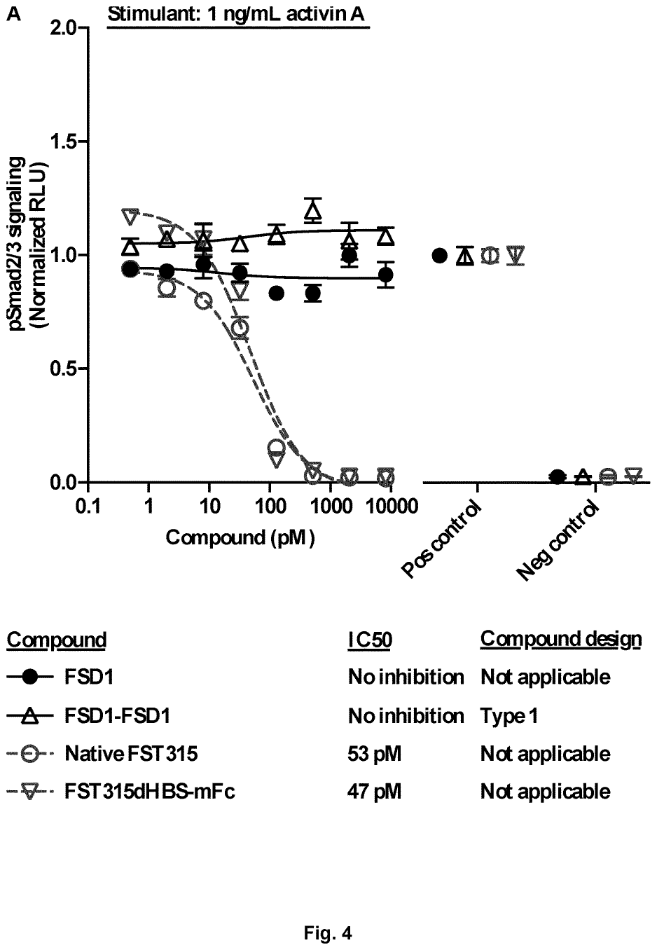

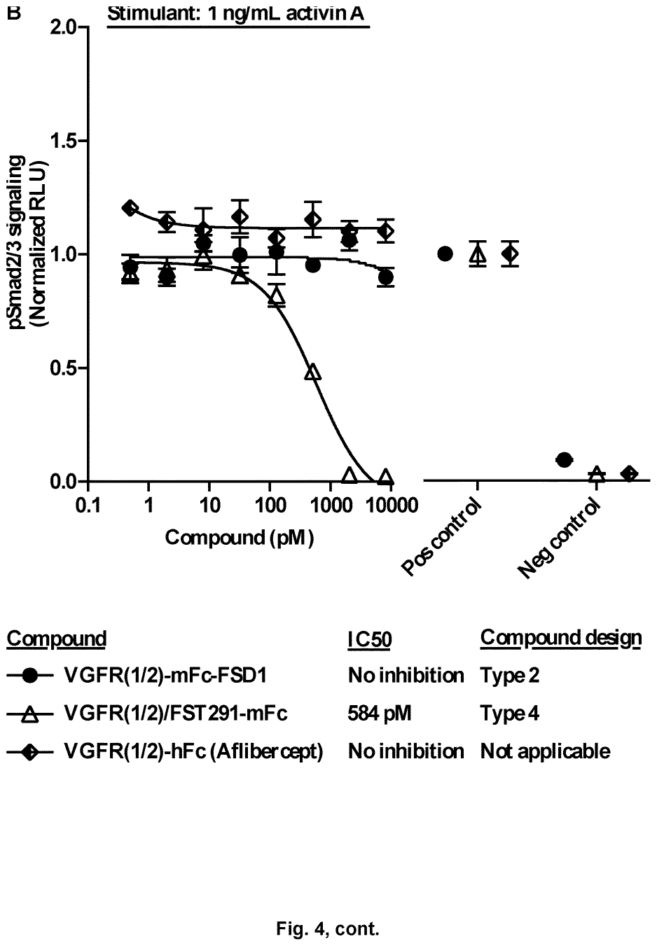

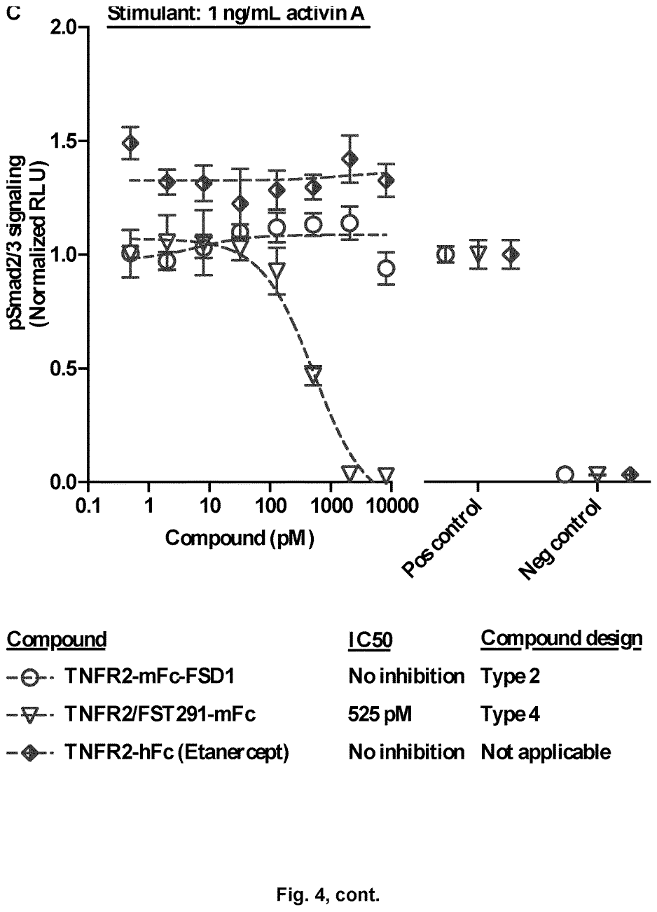

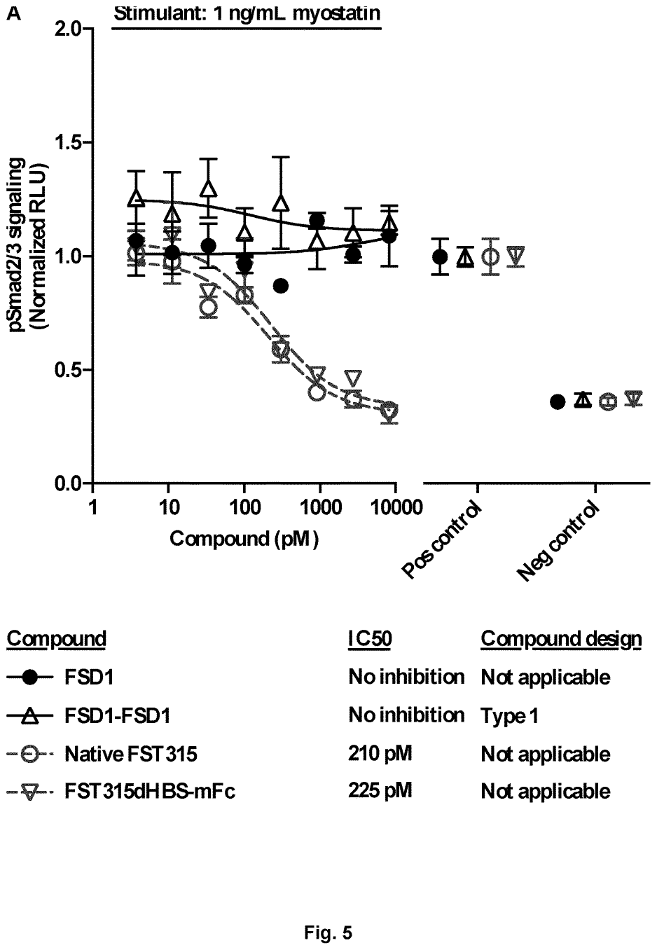

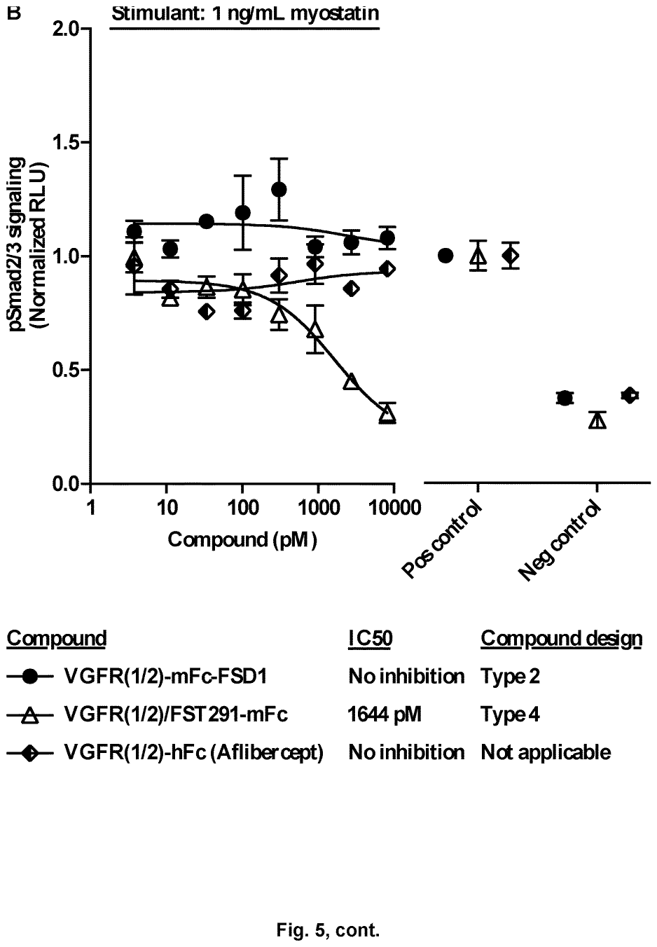

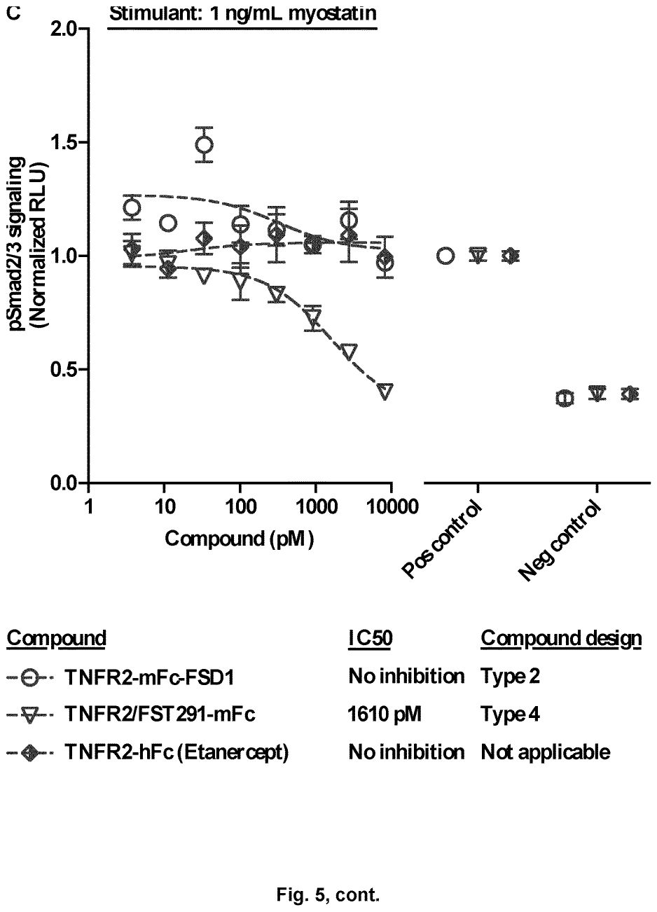

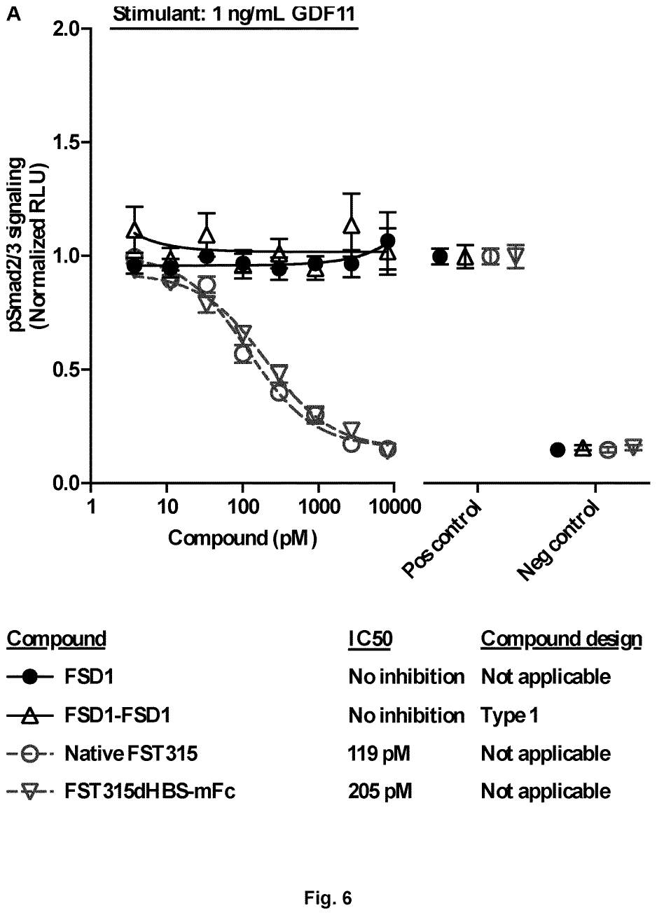

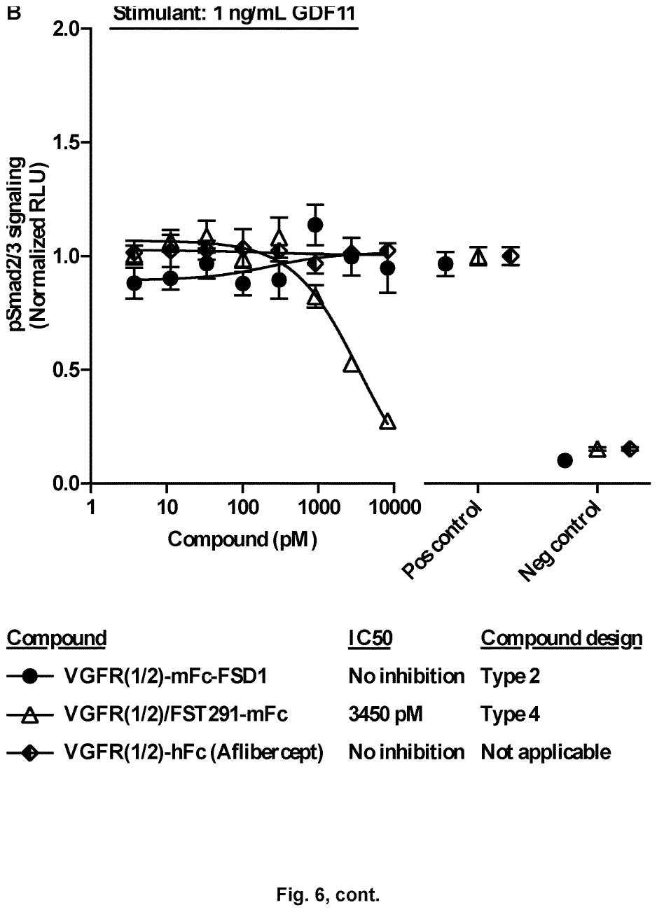

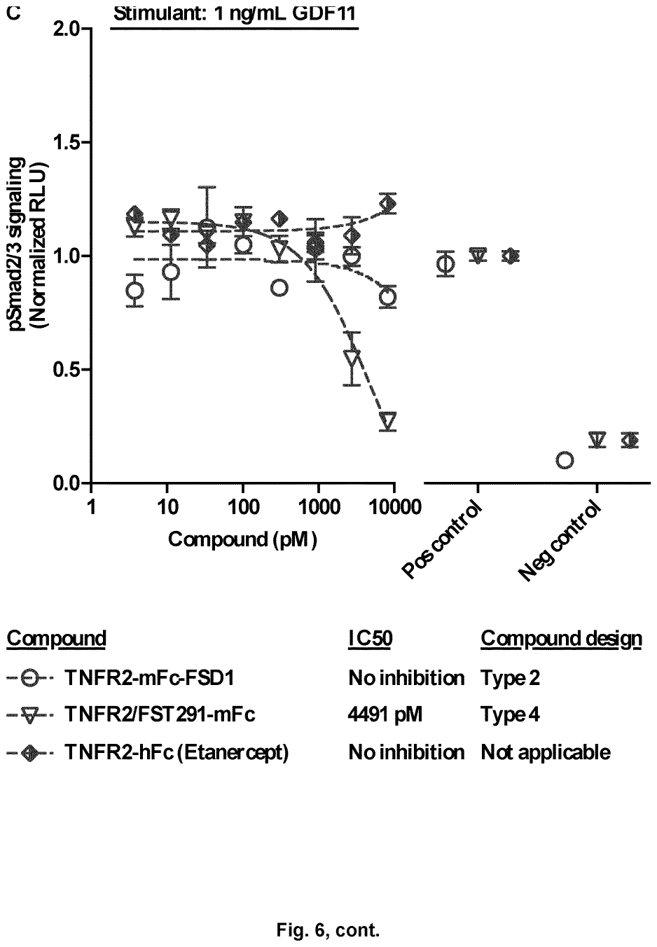

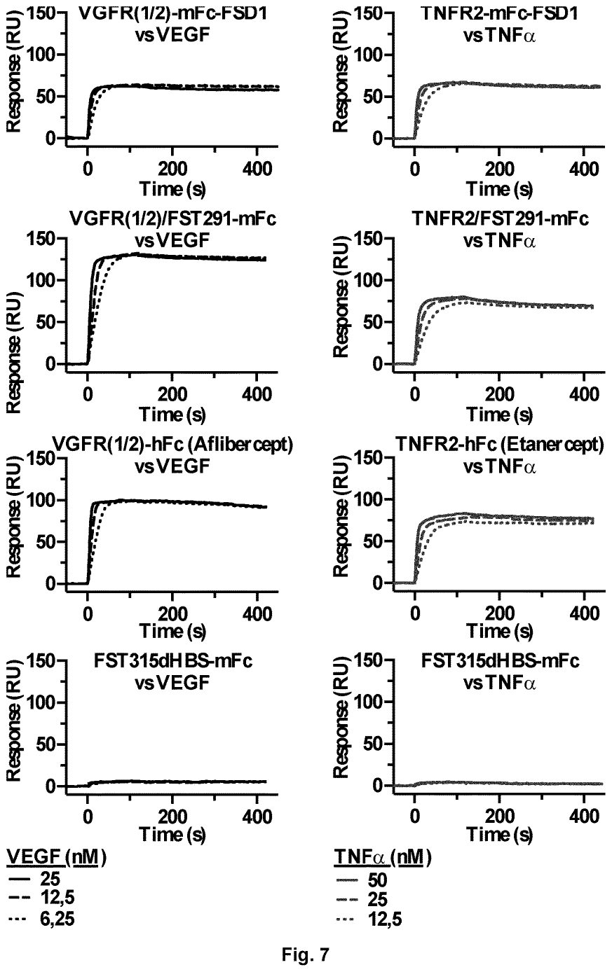

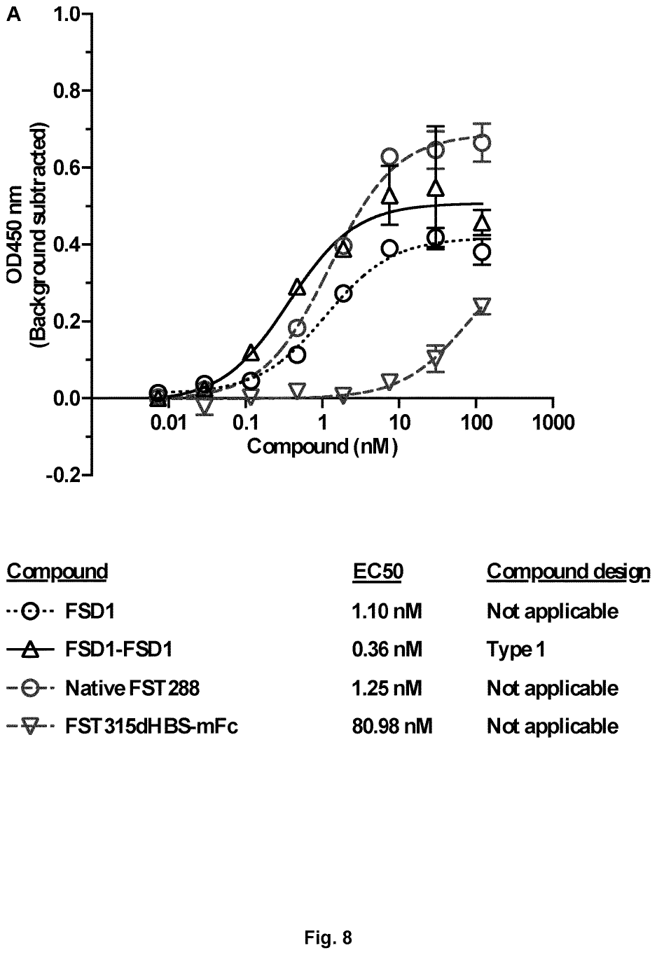

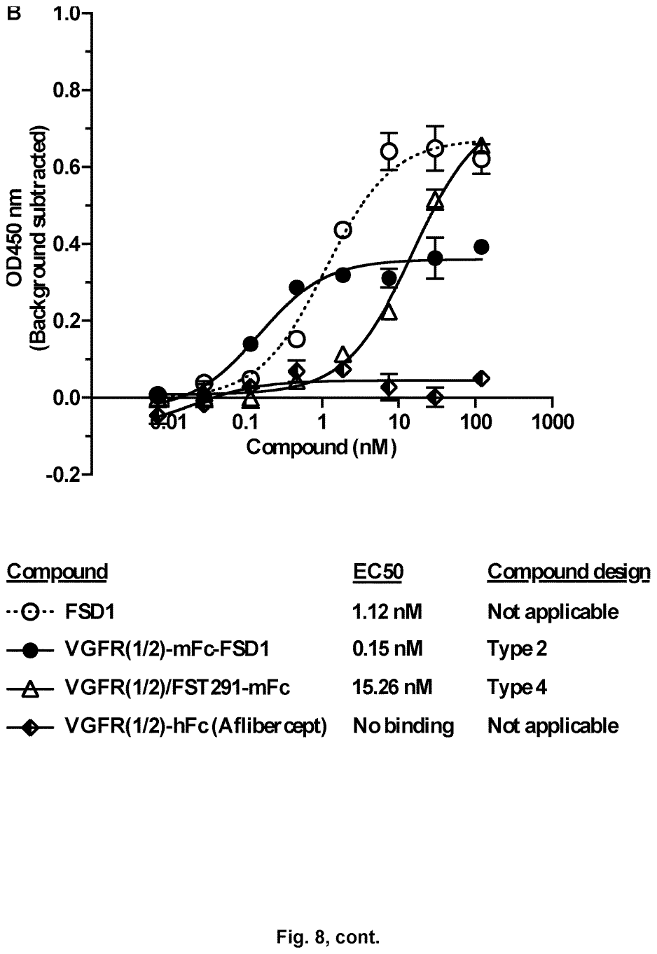

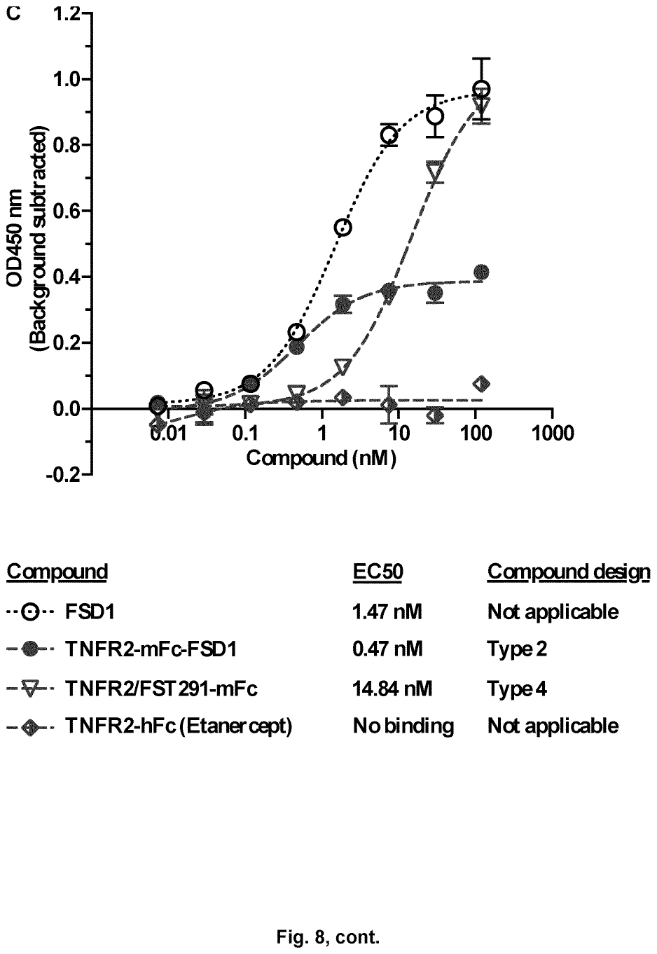

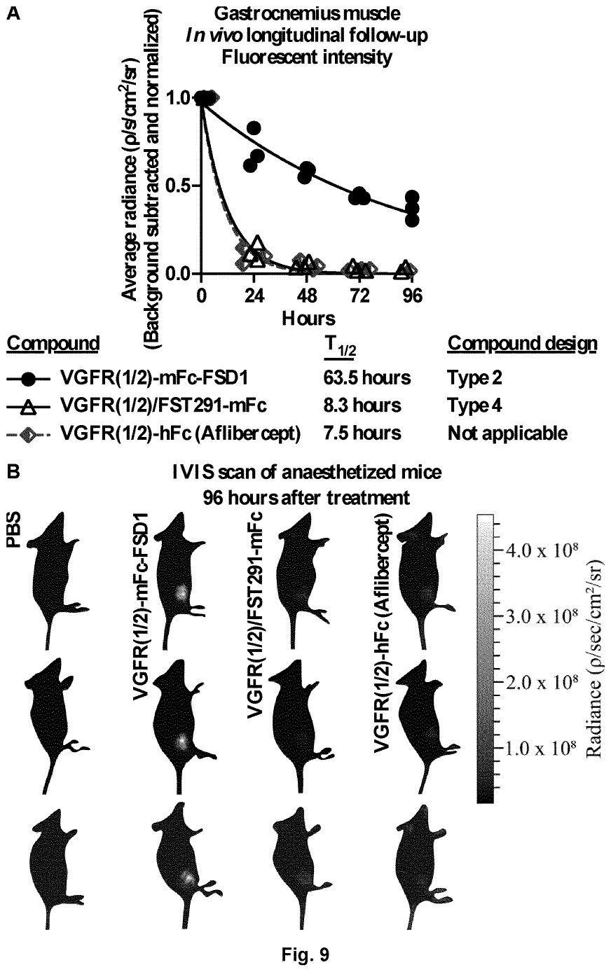

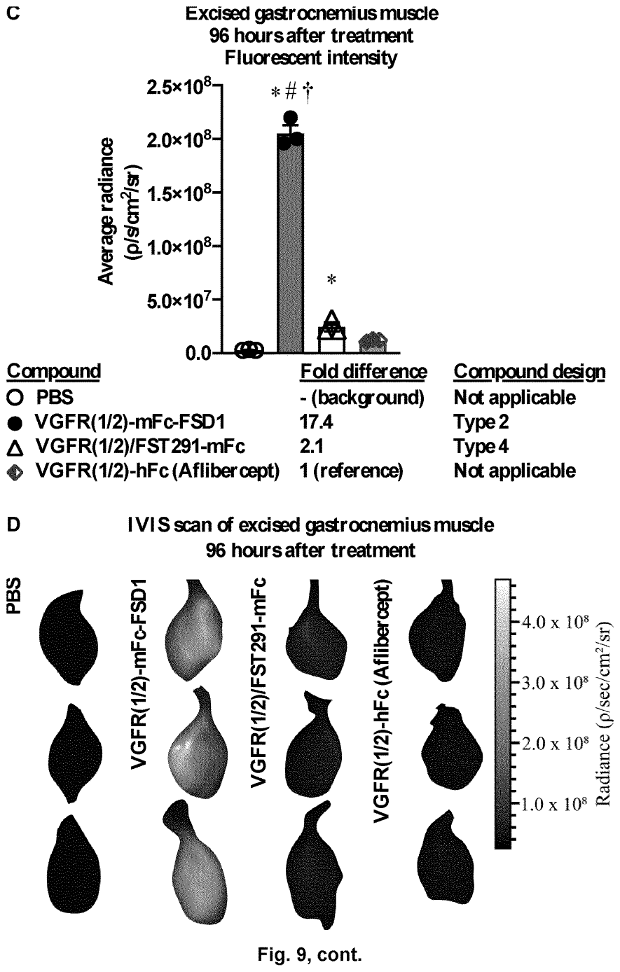

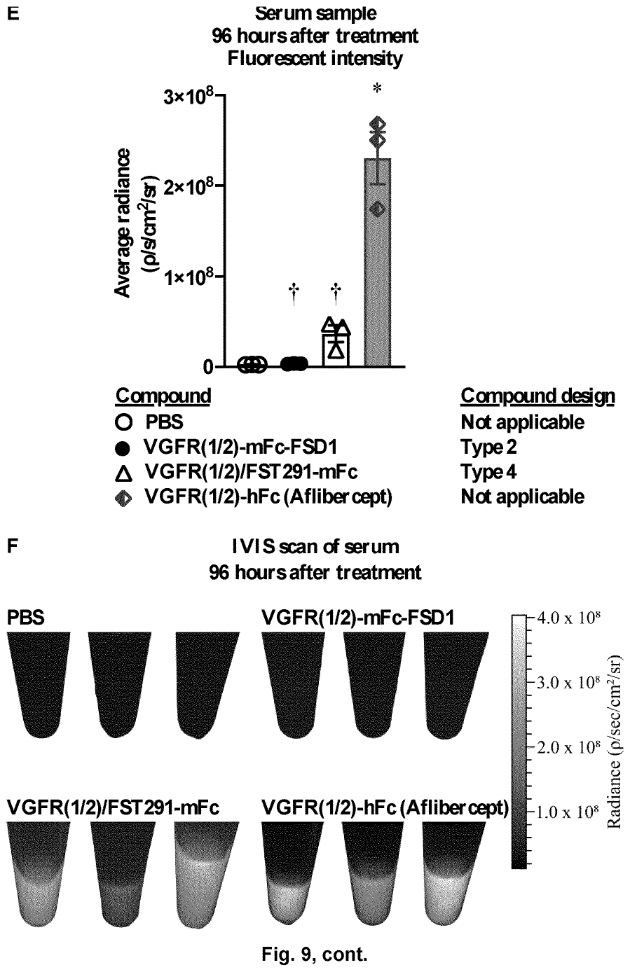

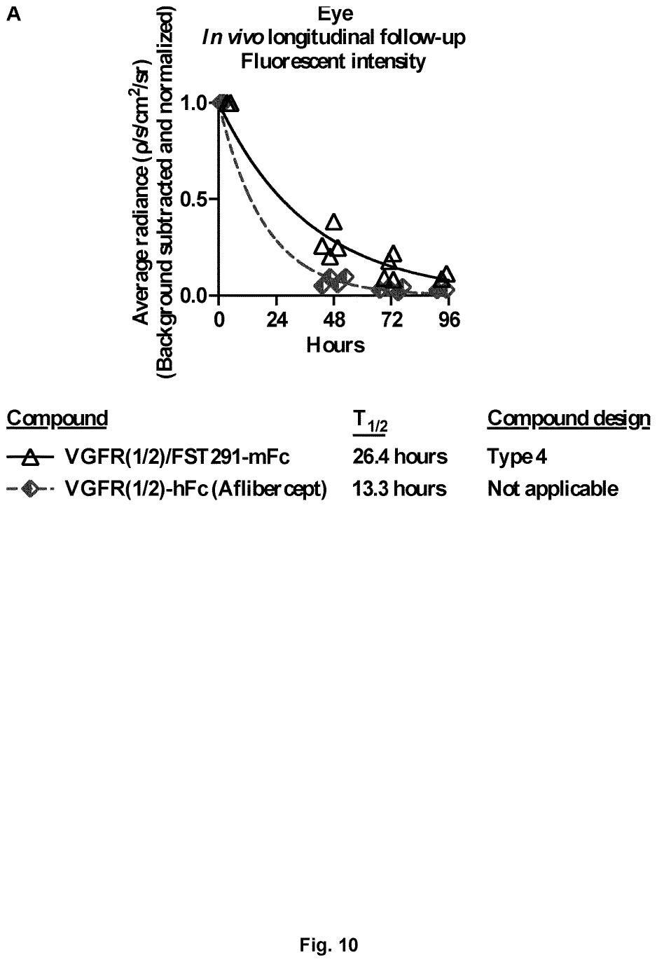

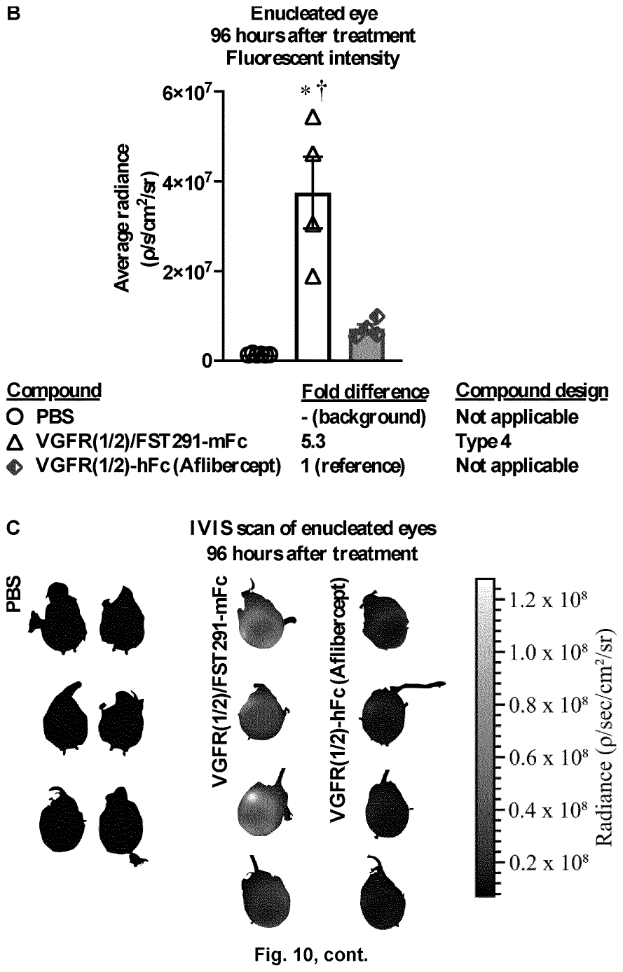

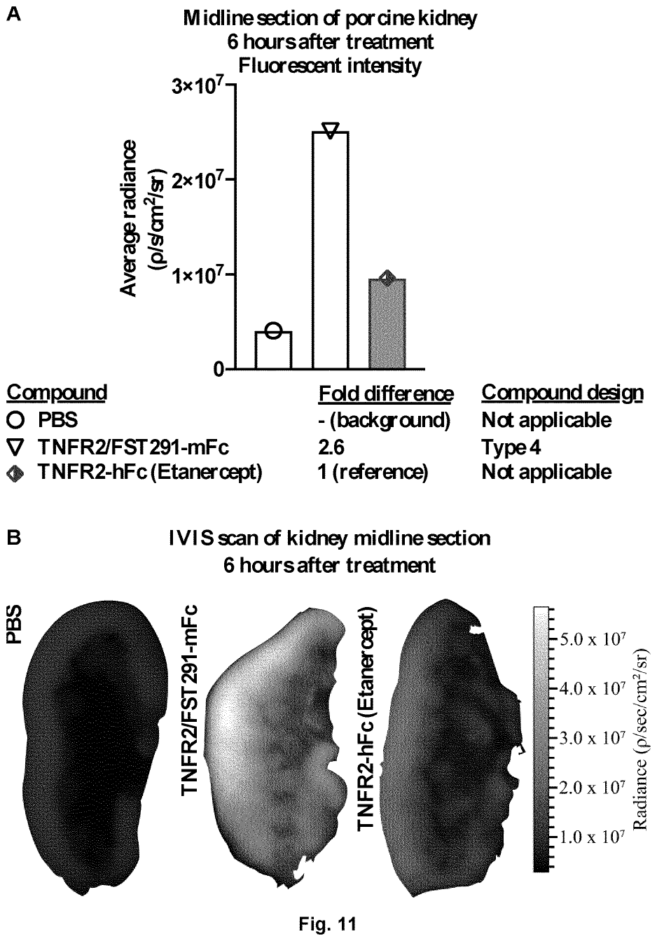

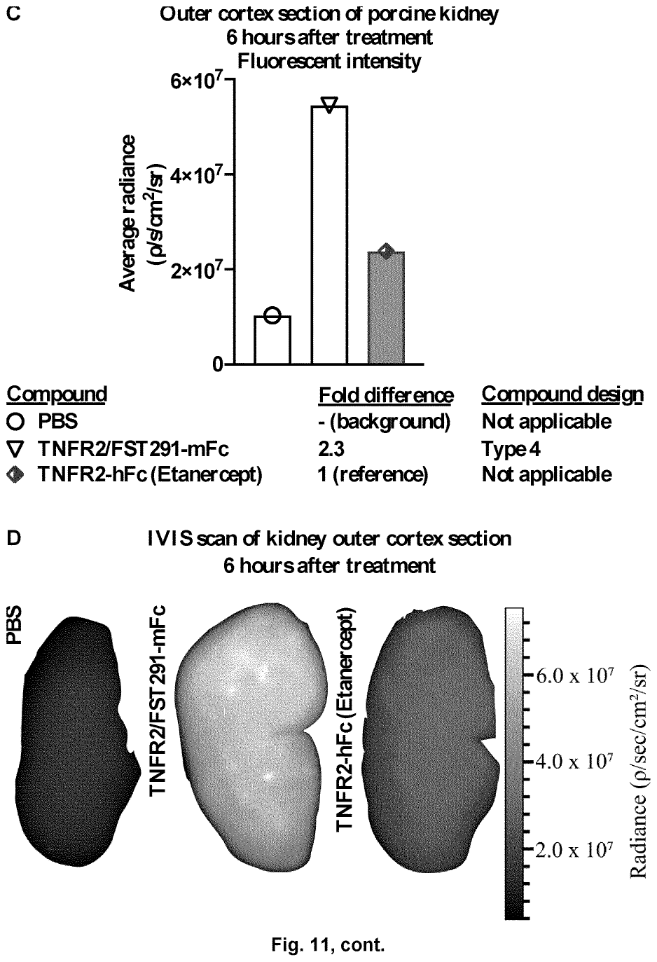

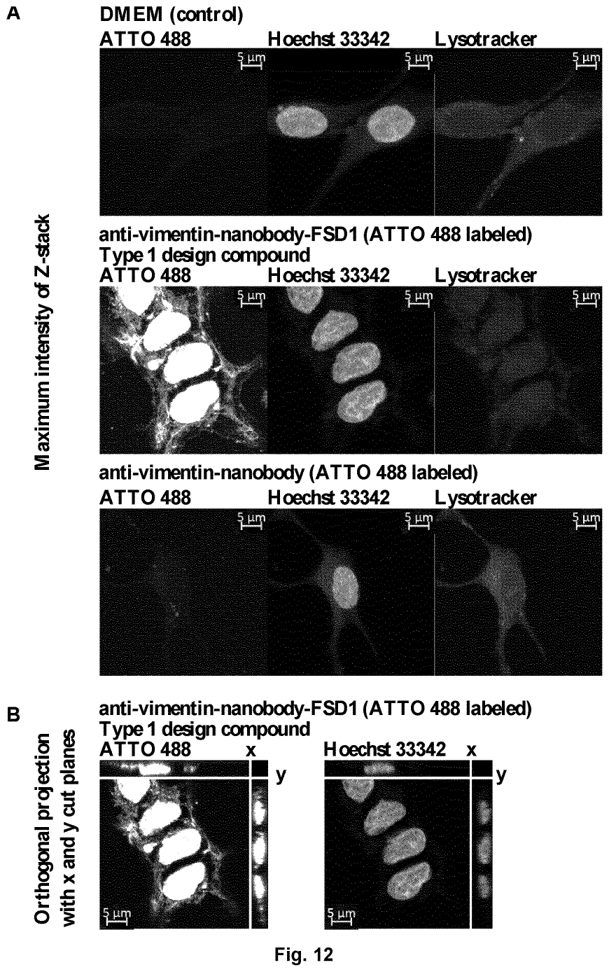

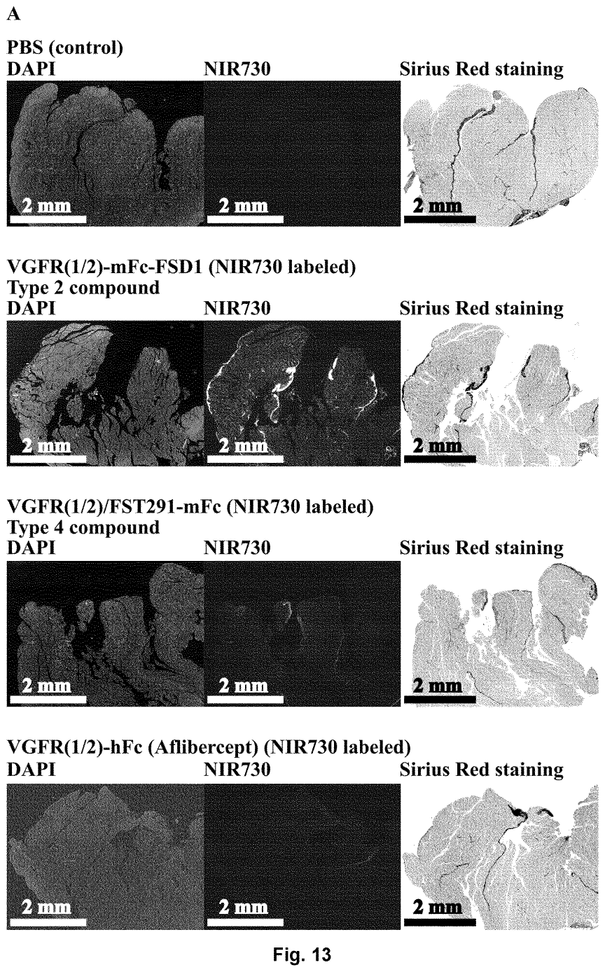

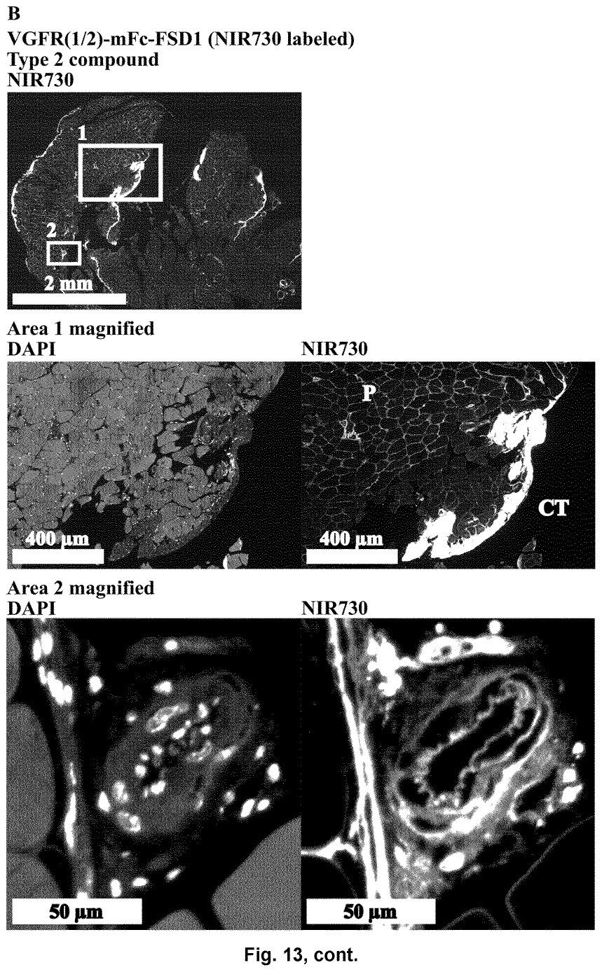

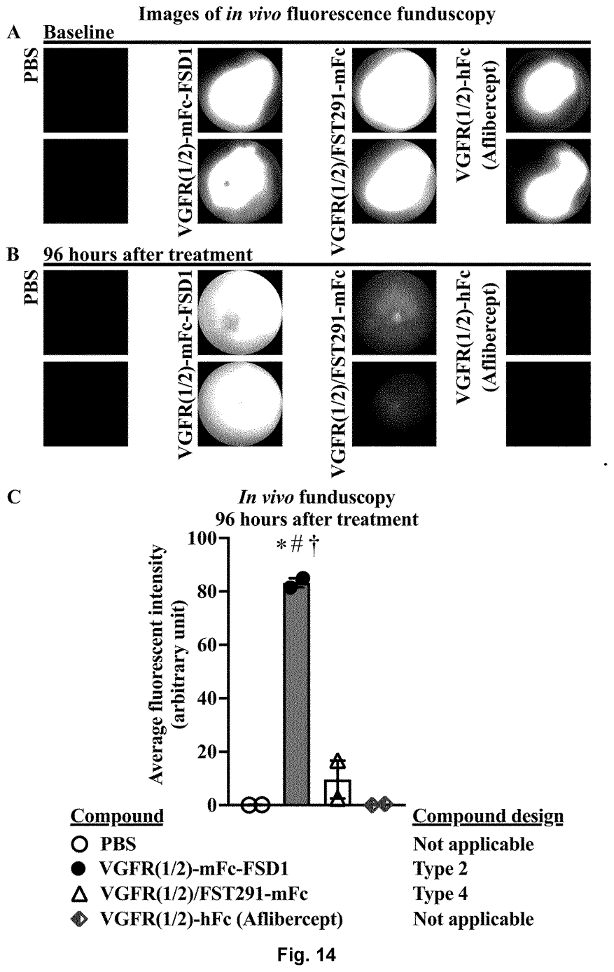

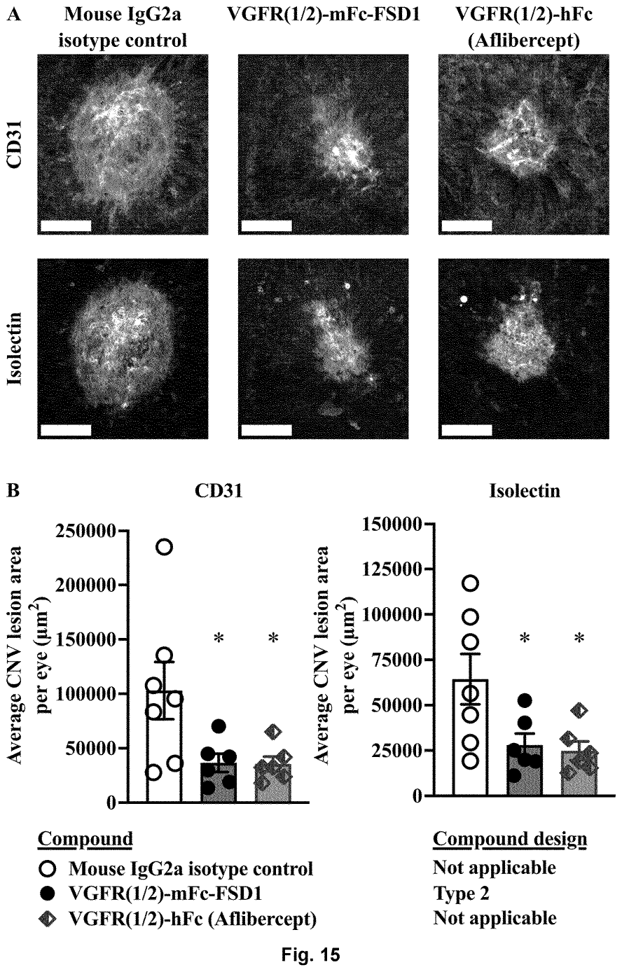

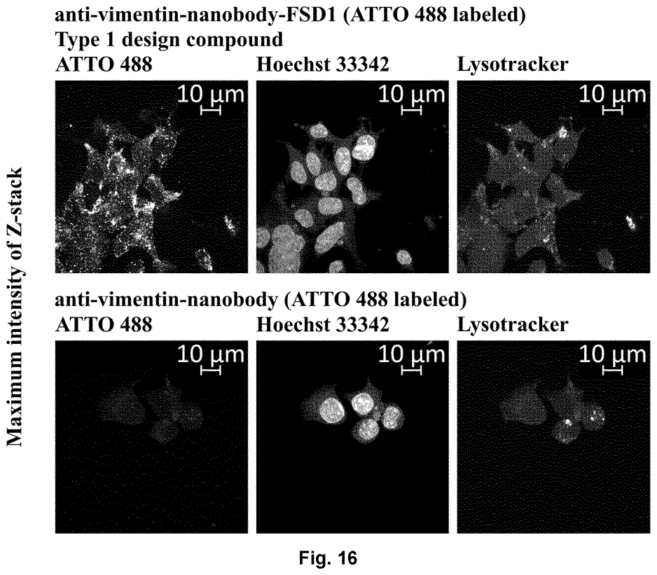

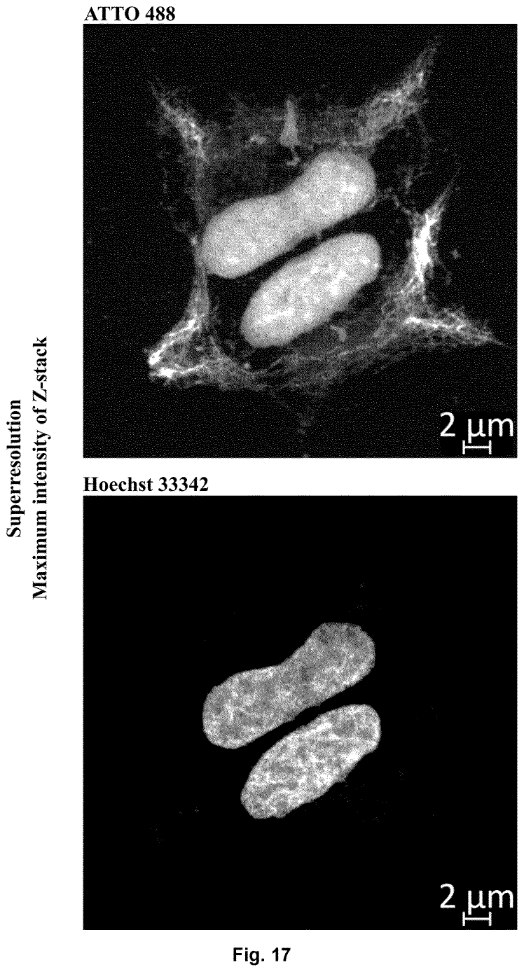

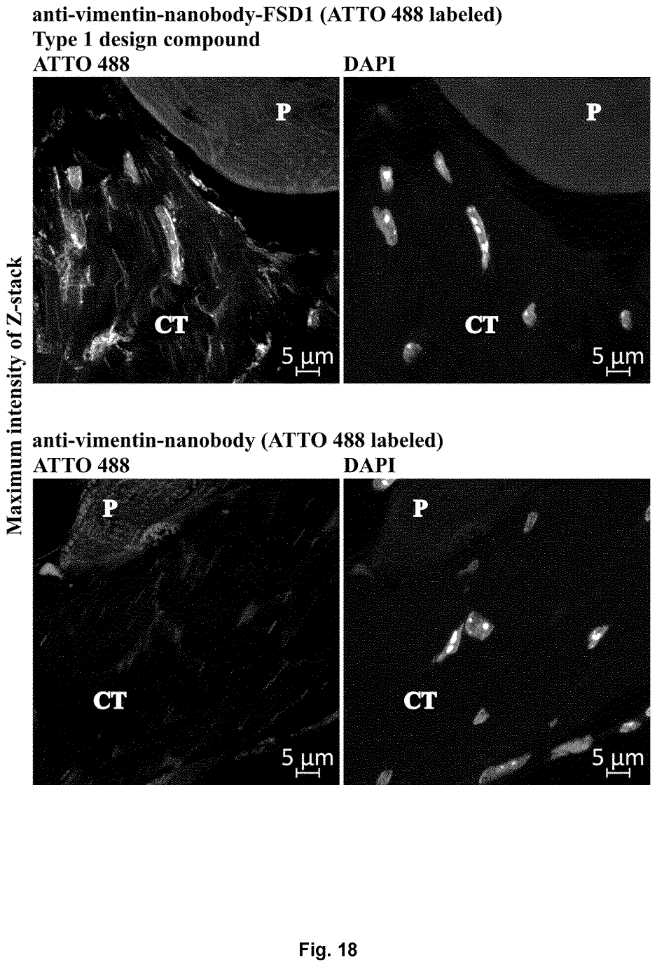

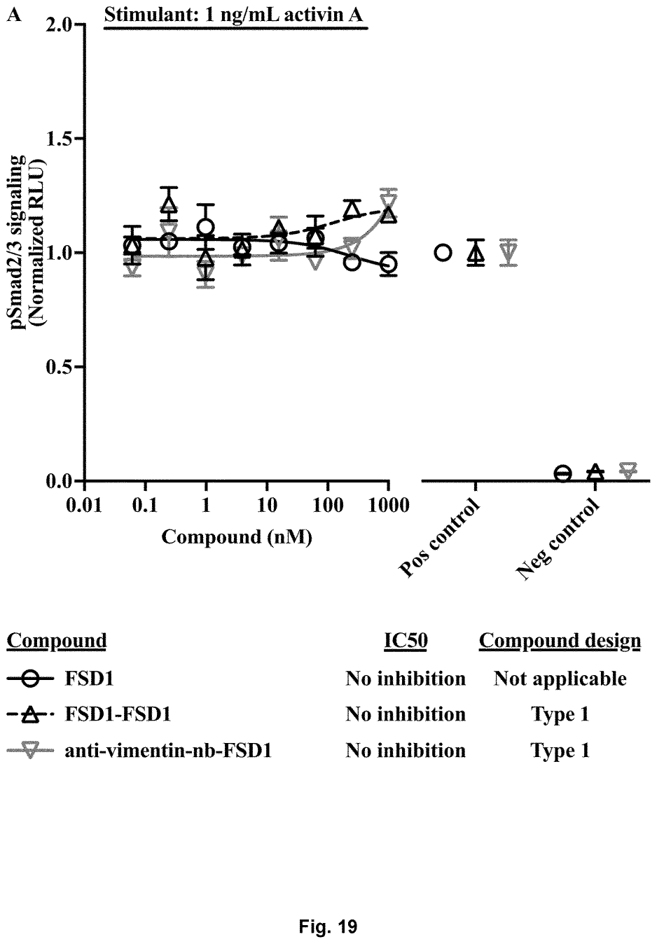

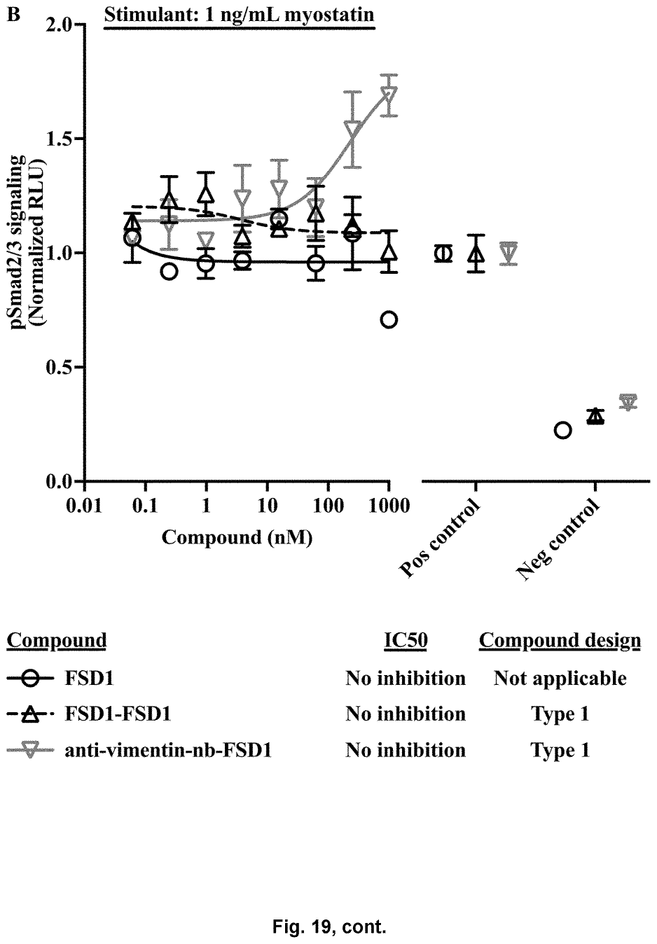

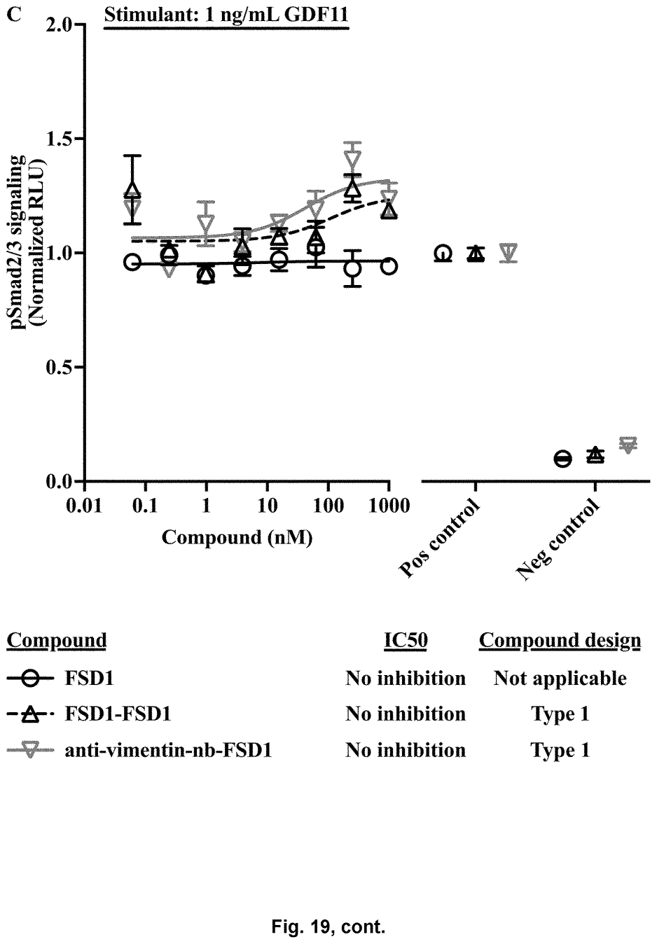

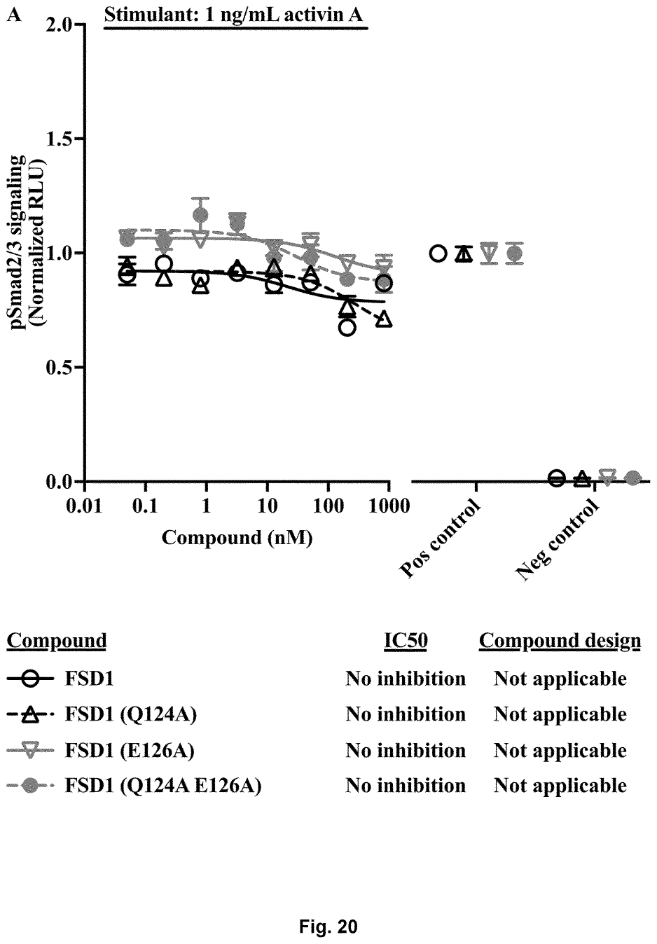

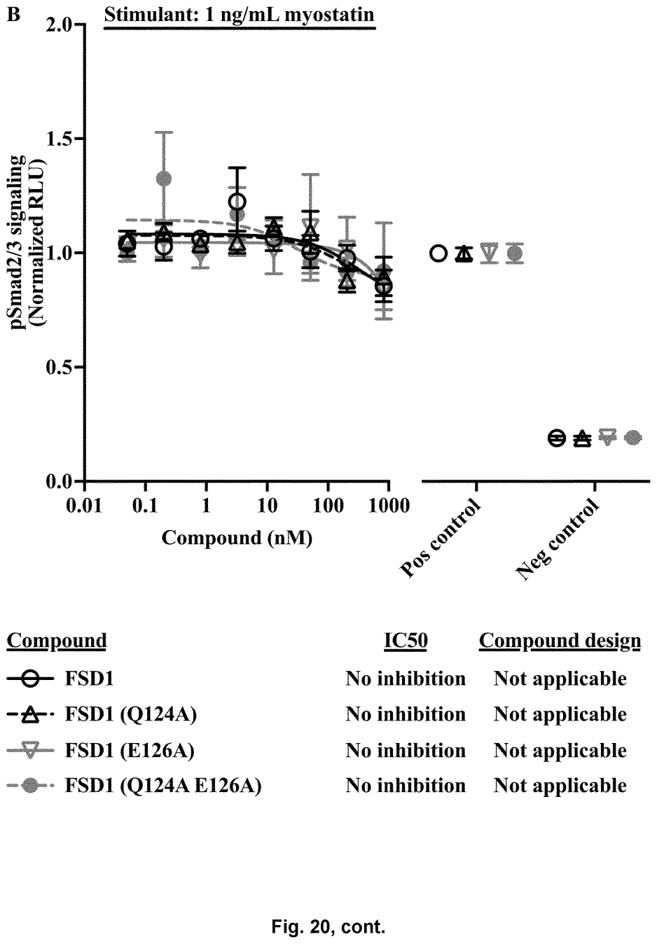

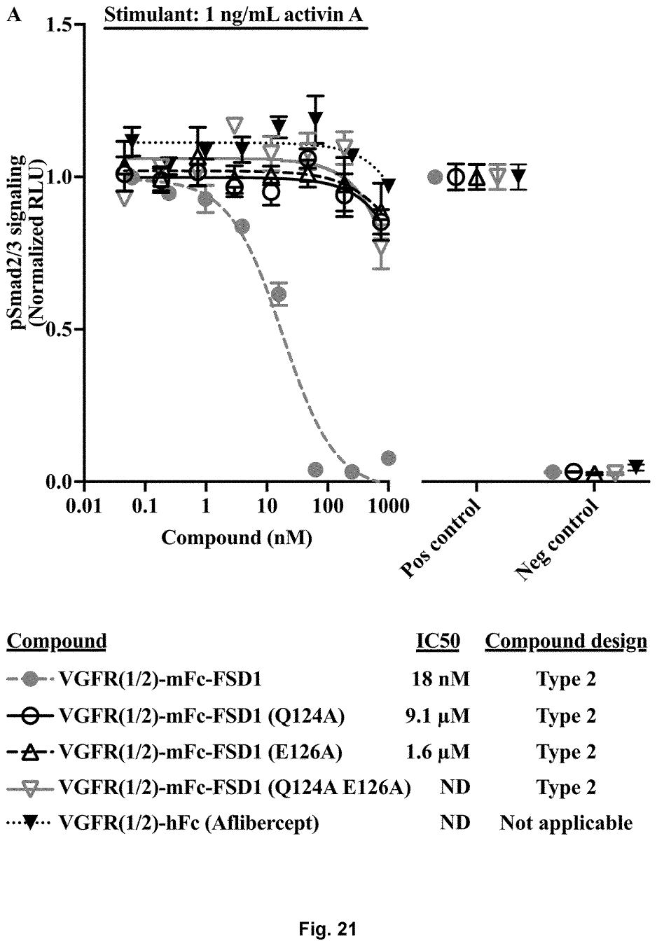

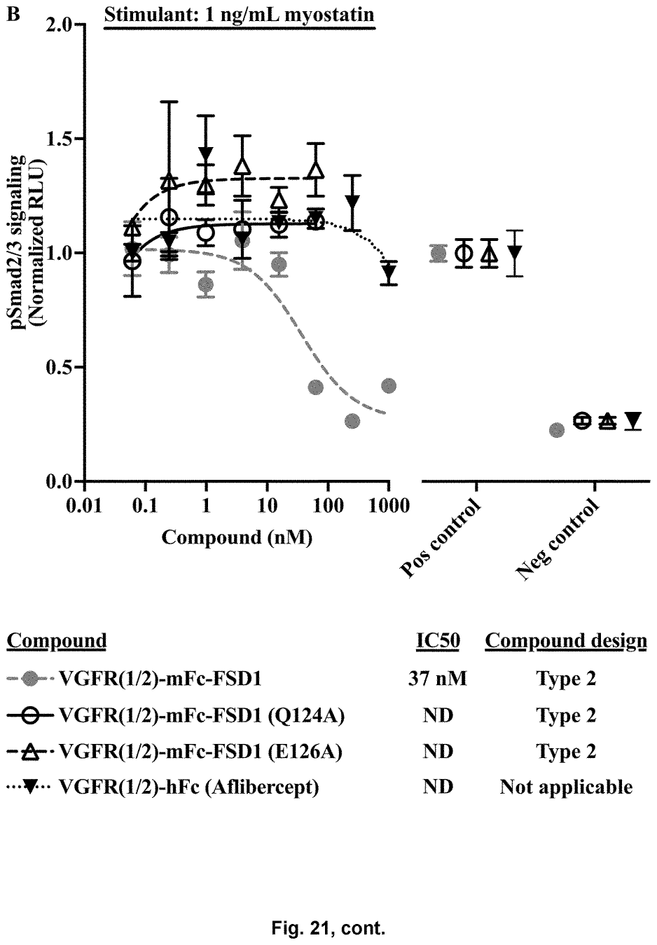

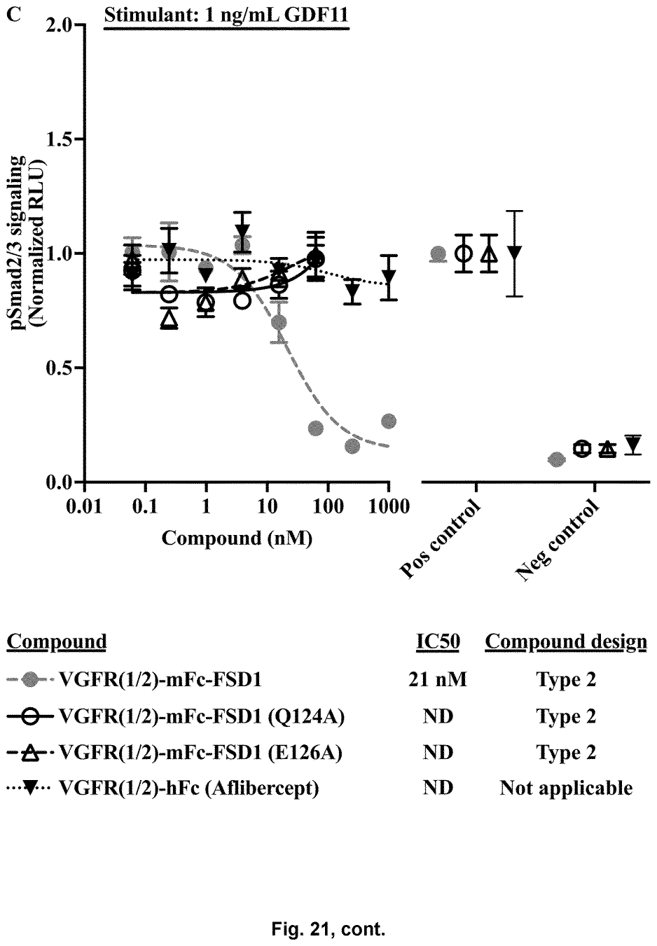

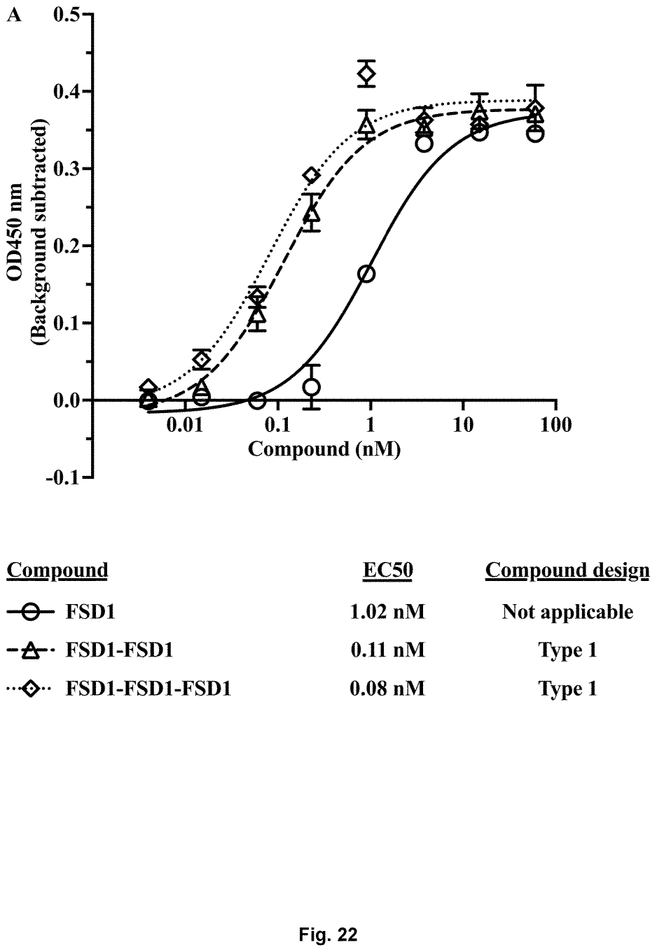

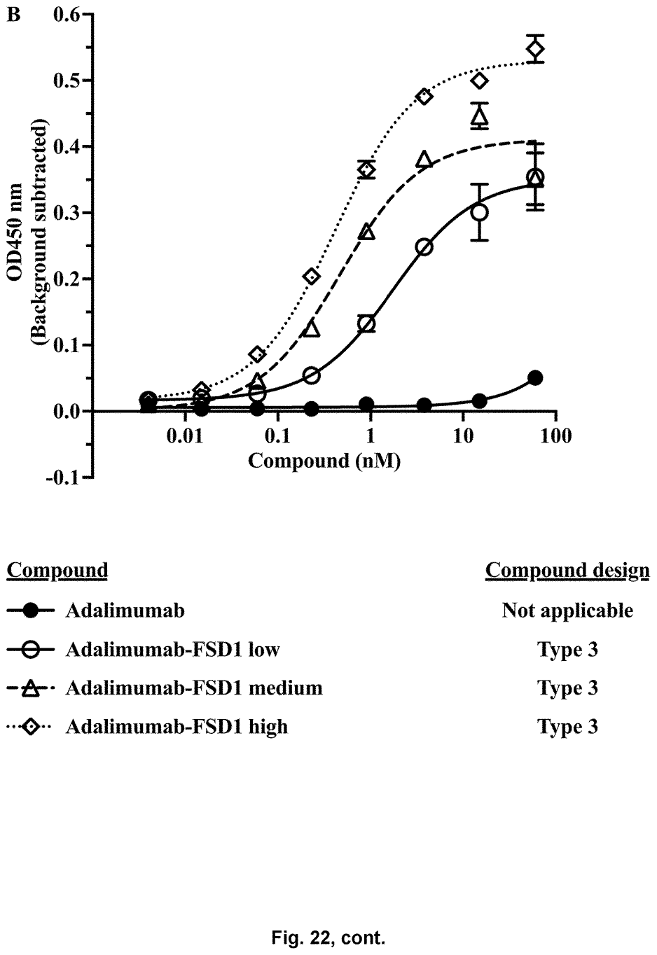

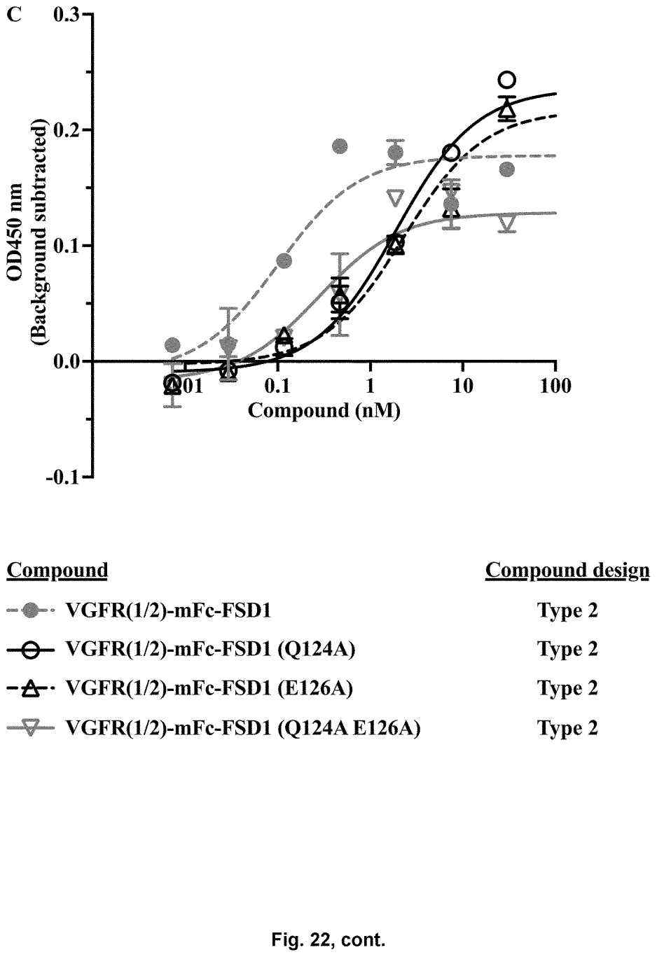

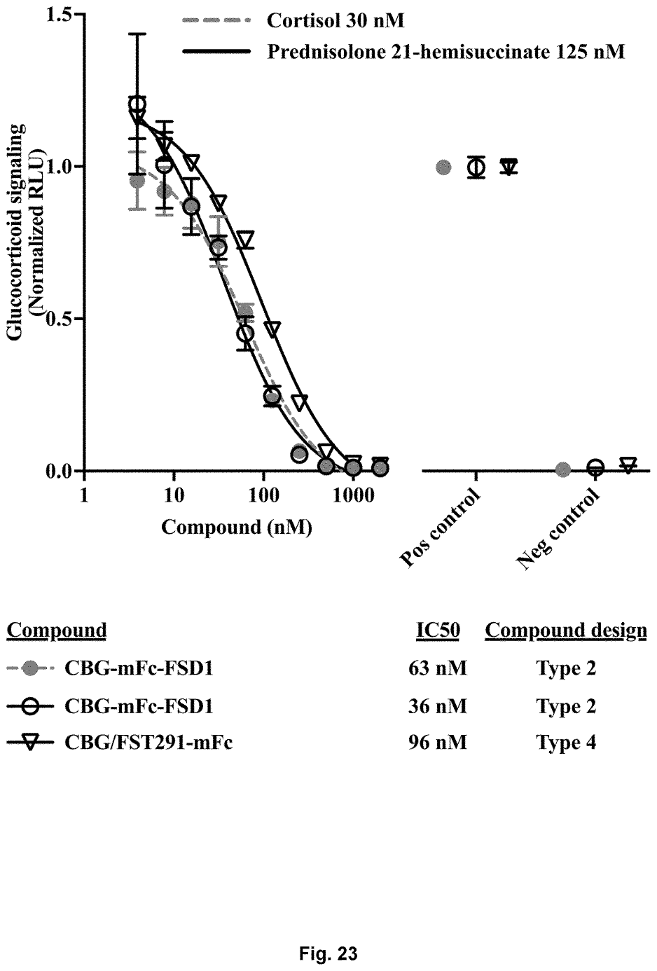

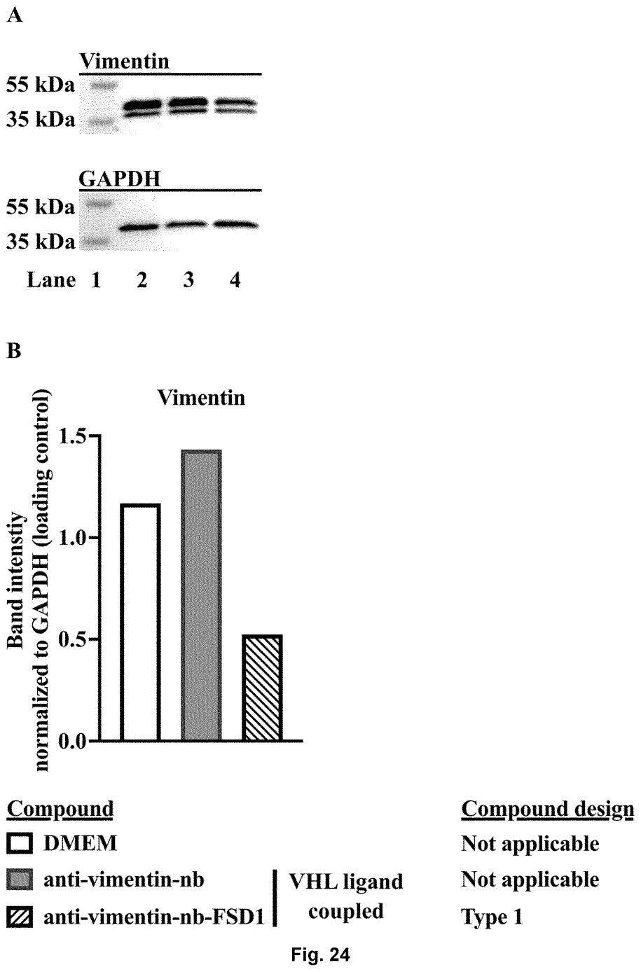

In type 1 design, the gene encoding the therapeutic or diagnostic protein was fused to FSD1 (monomer or multimer) for expression of a single polypeptide chain containing both therapeutic or diagnostic moiety and FSD1 (FIG. 1A). Llama anti-vimentin heavy-chain variable domain (VHH or nanobody (nb)) fused to FSD1 (anti-vimentin-nb-FSD1) was an example molecule of a type 1 design.

Type 2 designs were based on the immunoglobulin fragment crystallizable (Fc) platform (homodimeric or heterodimeric) (FIG. 1B). Here, the therapeutic/diagnostic moiety was fused to the N-terminal end of the Fc and FSD1 (monomer or multimer) was fused to the C-terminal end of the Fc. The homodimeric Fc platform facilitated the design of dimeric therapeutic or dimeric diagnostic proteins (FIG. 1Bi), whereas the heterodimeric Fc platform made it possible to design compounds containing two different therapeutic or diagnostic moieties (FIG. 1Bii). An example of a type 2 design was VGFR(½)-mFc-FSD1 or TNFR2-mFc-FSD1. A VGRF(½)-mFc-FSD1 construct with FSD1 residues Glutamine 124 or Glutamate 126, or both, mutated to alanine was also included in order to validate the lack of activin A, myostatin, and/or GDF11 neutralization.

Type 3 designs allowed FSD1 (monomer or multimer) to be used as a platform with any regular antibody that was obtained commercially or expressed as a singular entity (FIG. 1C). The type 3 designs included FSD1 covalently coupled (using click chemistry) to any regular antibody (FIG. 1Ci) or FSD1 expressed together with an anti-Fc single-chain VHH nanobody that will allow “mix and use” with any regular antibody at point-of-care (FIG. 1Cii). Type 3 designs also include FSD1 fused to streptavidin for conjugation with a biotinylated therapeutic or diagnostic protein or biotinylated FSD1 for conjugation with a streptavidin-fused protein. An example of a type 3 design was anti-tumor necrosis factor alpha (TNFα) antibody coupled to FSD1 (anti-TNFα antibody-FSD1).

Type 4 designs were based on the Fc heterodimeric platform, where either full-length FST or FSD1 (monomer or multimer) replaced one of the Fab regions of the Fc heterodimer and the other Fab region was replaced by the therapeutic or diagnostic moiety (FIG. 1D). Type 4 design included a C-terminal histidine-tag on the Fc chain that did not contain full-length FST or FSD1 to aid in the purification procedure. An example of a type 4 design was VGFR(½)/FST291-mFc, TNFR2/FST291-mFc, or CBG/FST291-mFc.

For chemical conjugation of FSD1 to an existing therapeutic antibody, a FSD1 version (FSD1 (KTC)) was designed that contained three additional, native C-terminal residues (lys-thr-cys). The free C-terminal cysteine of FSD1 (KTC) allowed conjugation with different types of click-chemistry compounds containing maleimide.

The homodimeric Fc platform used in type two designs was based on mouse IgG2A. The heterodimeric Fc platform used in both type two designs and type four designs was based on the “knobs-in-holes” methodology (REF 2). The “knobs-in-holes” method employed two Fc chains (F_{C}_{_{A}} and F_{C}_{_{B}}) with points mutations introduced to promote heterodimer formation rather than homodimer formation.

Additional standalone proteins, FSD1, FSD1-FSD1, and FSD1-FSD1-FSD1 were designed for validation of affinity for heparan sulfate and lack of activin A, myostatin, and/or GDF11 neutralization.

### Conclusion:

The four designs each represented different strategies to construct therapeutic or diagnostic moieties fused to FSD1 or FST as a platform for heparan sulfate affinity.

### Example 2: Protein Expression and Purification

### Aim:

The aim of this example was to generate cell lines that expressed the desired recombinant proteins and purify them using various chromatographic methods.

### Material and Methods:

### Mammalian Expression System:

Cloning vectors were transformed in DH5alpha competent E. coli cells (ThermoFisher Scientific #18265017). A Maxiprep kit was used for high-yield plasmid purification (Qiagen #12162). Plasmids were transfected using lipofection in a mammalian expression platform based on Chinese hamster ovary cells (CHO). (REF. 6) Monoclonal cell lines were seeded based on fluorescent intensity using a cell sorter. The clones expressing the highest protein of interest titre were then selected based on dot blots and propagated to suspension. Proteins were purified from filtered conditioned media using either standalone or a combination of protein A (Cytiva, #GE17-0403-01), IMAC (Roche, #6781535001, complete His-Tag Purification Column), or heparin chromatography (Cytiva, #GE17-0407-01). Protein purity was verified using SDS-PAGE.

### Bacterial Expression:

Vectors for bacterial expression were transformed into the E. coli strain BL21 (DE3) for expression. The cells were grown in LB media (Sigma-Aldrich, #L3022) containing 100 μg/ml ampicillin (Sigma-Aldrich, #A9518) at 37° C. in a shaking incubator. Expression was induced using 0.1 mM IPTG (Sigma-Aldrich, #16758) and the temperature was subsequently decreased to 20° C. After expression overnight, the cells were harvested by centrifugation and stored at −20° C. Cells were lysed by sonication and cell debris removed by centrifugation. The recombinant proteins were purified using cation exchange chromatography or heparin chromatography and purity verified using SDS-PAGE.

### Results:

Type 2 compounds utilizing native FSD1, VGFR(½)-mFc-FSD1 and TNFR2-mFc-FSD1, and type 4 compounds, VGFR(½)/FST291-mFc and TNFR2/FST291-mFc and CBG/FST291-mFc, were successfully expressed in the mammalian expression system and purified using affinity chromatography. Type 2 compounds utilizing mutant FSD1: VGFR(½)-mFc-FSD1 (Q124A), VGFR(½)-mFc-FSD1 (E126A), and VGFR(½)-mFc-FSD1 (Q124A, E126A) were successfully expressed in the mammalian expression system and purified using affinity chromatography. Type 1 compound (anti-vimentin-nb-FSD1), FSD1 (KTC) (used in type 3 compound design), and standalone proteins, FSD1, FSD1-FSD1, and FSD1-FSD1-FSD1 were successfully expressed in E. coli and purified using cation-exchange or heparin chromatography. Standalone mutant FSD1 proteins: FSD1 (Q124A), FSD1 (E126A), and FSD1 (Q124A, E126A) were successfully expressed in E. coli and purified using cation-exchange or heparin chromatography. Anti-vimentin-nb (with a His tag) was expressed successfully in E. coli and purified using IMAC (the protein was used as a control in studies involving the type 1 compound (anti-vimentin-nb-FSD1)).

### Conclusion:

Recombinant fusion with native or mutant FSD1 or full-length FST as a platform technology yielded proteins that could be expressed, purified, and folded correctly in accordance with the designs described.

### Example 3: Heparin-Affinity Chromatography

### Aim:

The aim of this example was to estimate the heparin affinity of the produced compounds using heparin-affinity chromatography and compare them to commercial counterparts (if such existed), such as VGFR(½)-hFc (Aflibercept) and TNFR2-hFc (Etanercept)

### Material and Methods:

Approximately 200 μg sample was loaded on a 1 ml HiTrap Heparin HP affinity column (Cytiva, #GE17-0407-01) equilibrated in buffer A (200 mM NaCl, Tris-HCl pH 7.6) and eluted by running a 15 ml gradient from 0-100% buffer B (2000 mM NaCl, Tris-Hcl pH 7.6). The eluted protein was monitored by UV absorbance (A₂₈₀).

### Results:

The standalone compounds FSD1 and FSD1-FSD1 both showed high heparin affinity and eluted from the column at 1133 mM and 1280 mM NaCl, respectively (FIG. 2A+B). This indicated that fusion of multiple FSD1 domains leads to an increased heparin affinity.

Fusion of FSD1 to the anti-vimentin nanobody (type 1) lead to a strong binding to heparin eluting af 1277 mM NaCl, whereas the nanobody itself did not bind the column (FIG. 2A-B).

The type 2 compounds VGFR(½)-mFc-FSD1 and TNFR2-mFc-FSD1 both showed high affinity to heparin and eluting at 1133 mM and 1080 mM NaCl, respectively (FIG. 2A-B).

The type 4 compounds VGFR(½)/FST291-mFc and TNFR2/FST291-mFc both showed an elution profile with two peaks indicating two distinct conformations with different heparin affinities (FIG. 2A-B)—a low affinity conformation denoted peak 1 (eluting at 507 mM NaCl for VGFR(½)/FST291-mFc and 440 mM NaCl for TNFR2/FST291-mFc) and a high affinity conformation (eluting at 933 mM NaCl for VGFR(½)/FST291-mFc and 907 mM NaCl for TNFR2/FST291-mFc). The commercial VGFR(½)-hFc (Aflibercept) and TNFR2-hFc (Etanercept) showed no affinity to heparin. Also, the FST315dHBS-mFc (SEQ ID NO: 30, SEQ ID NO: 31) showed very low heparin affinity in agreement with it containing an altered heparin-binding site (REF. 3).

The type 4 compound, CBG/FST291-mFc, showed high affinity to heparin and eluting at 915 mM (FIG. 2A-B).

### Conclusion:

The example showed that the designed Type 1, Type 2 and Type 4 compounds have a high heparin affinity.

### Example 4: Conjugation of Protein Using Click-Chemistry

### Aim:

The aim of this example was to conjugate FSD1 to an anti-TNFα antibody (Adalimumab) using click-chemistry.

### Material and Methods:

The C-terminal cysteine of FSD1 (KTC) was conjugated with DBCO-maleimide. Adalimumab was oxidized using sodium periodate and subsequently conjugated with aminooxy-PEG3-azide. Conjugated FSD1 and conjugated Adalimumab were mixed in a 2:1 ratio in order to covalently link the two compounds.

### Results:

Oxidation of antibody N-linked glycosylations with sodium periodate resulted in multiple aldehyde groups that was conjugated by aminooxy-groups. Equally, the FSD1 (KTC) was conjugated by maleimide-groups. Using these groups together with the click-chemistry compounds azide and DBCO, FSD1 (KTC) was covalently coupled to Adalimumab. Although the conjugation was far from complete, we observed multiple fractions containing Adalimumab with increased affinity to heparin (FIG. 3A+B). SDS-PAGE showed that these fractions increased in size proportional to their heparin affinity (FIG. 3C), indicating that multiple conjugations of FSD1 (KTC) to Adalimumab leads to increasing heparin affinity.

### Conclusion:

Using click-chemistry, it was possible to conjugate FSD1 (KTC) to an existing antibody in order to apply heparin affinity to the antibody. The example confirms that it is possible to create Type 3 (i) compounds with high heparin affinity.

### Example 5: Bioassays

### Aim:

The aim of this example was to quantify neutralization of activin A, myostatin, or GDF11 and to confirm the absence of said neutralization in the compounds claimed in the present invention.

### Material and Methods:

Growth factor neutralization of activin A (R&D Systems, #338-AC), myostatin (R&D Systems, #788-G8-010), or GDF11 (R&D Systems, #1958-GD-010) was quantified using a reporter gene bioassay of phosphorylated Smad signaling. Phosphorylated Smad2 or Smad3 forms a complex with Smad4 that translocates to the nucleus and recognizes the repetitive base sequence known as the Smad-binding element (SBE), 5′-CAGAC-3′. HEK293 cells were transfected with a construct containing a twelve times repeated SBE motif upstream of a minimal promoter controlling luciferase expression (REF. 4). The HEK293 cell line was propagated to a stable line with a strong luciferase responsiveness to phosphorylated Smad2 or Smad3 signaling induced by growth factor stimulation. Briefly, cells were stimulated with activin A, myostatin, or GDF11 and cotreated with a concentration range of recombinant proteins starting at 8.2 nM, 63 nM, 750 nM, 822 nM, or 1 μM, as previously described (REF. 3). After sixteen to twenty hours, cells were lysed (Promega, Glo Lysis Buffer, #E2661), luciferase substrate was added (Promega, Steady-Glo Luciferase Assay System, #E2520), and the luminescent signal was analyzed using a plate reader (PerkinElmer, EnSpire 2300). Three to six positive controls (growth factor stimulation without inhibitor) and negative controls (no growth factor and no inhibitor) were included. Data was analyzed and half maximal inhibitory concentration (IC50) was calculated using nonlinear regression with three parameters and no weighting and no constraints (Graphpad, Prism version 9.4.1).

### Results:

FSD1, FSD1-FSD1, or type 2 design compounds, VGFR(½)-mFc-FSD1 or TNFR2-mFc-FSD1, had no neutralizing effect on activin A (FIG. 4), myostatin (FIG. 5), or GDF11 signaling (FIG. 6) in the tested concentration range up to 8.2 nM. Similarly, commercial therapeutic agents, TNFR2-hFc (Etanercept) or VGFR(½)-hFc (Aflibercept), did not neutralize growth factor signaling in the tested concentration range up to 8.2 nM (FIGS. 4B-C, 5B-C, and 6B-C). In contrast, activin A, myostatin, and GDF11 were neutralized with an IC50 in the pM range by native FST315 (SEQ ID NO: 28) and FST315dHBS-mFc (a recombinant FST315 protein fused to a murine Fc fragment wherein the heparin binding sequence [HBS] of FSD1 has been replaced by a structurally related sequence, SEQ ID NO: 30, SEQ ID NO: 31) (REF. 3) (FIGS. 4A, 5A, and 6A). Type 4 design compounds containing a single moiety of full-length FST, VGFR(½)/FST291-mFc or TNFR2/FST291-mFc, neutralized growth factors with an IC50 in the mid pM range to low nM range (FIGS. 4B-C, 5B-C, and 6B-C). When tested in a concentration range up to 1 μM, FSD1 and FSD1-FSD1 had no neutralizing effect on activin A, myostatin, or GDF11 signaling (FIG. 19). The type 1 design compound, anti-vimentin-nanobody-FSD1, increased pSmad2/3 signaling substantially when myostatin was used as stimulant and to a lesser extent when activin A or GDF11 were used as stimulants, in all cases starting from around 100 nM in the tested concentration range up to 1 μM (FIG. 19). When tested in a concentration range up to 822 nM, single entity FSD1 mutants: FSD1 (Q124A), FSD1 (E126A), or FSD1 (Q124A, E126A) had no neutralizing effect on activin A or myostatin (FIG. 20). When tested in a concentration range up to 1 μM, type 2 design compound with native FSD1, VGFR(½)-mFc-FSD1, neutralized activin A, myostatin, and GDF11 signaling with an IC50 in the low nM range, demonstrating an inhibitory effect on growth factor signaling when moving beyond a compound concentration of 8.2 nM (compare FIGS. 4B, 5B, and 6B to FIG. 21). However, when the FSD1 mutants (Q124A or E126A) were applied in the type 2 design instead of native FSD1, the neutralizing effect on activin A was reduced (to the low UM range when extrapolated) or abolished (myostatin and GDF11) in the tested concentration ranges (FIG. 21). The commercial counterpart, VGFR(½)-hFc (Aflibercept), did not neutralize activin A signaling in the tested concentration range up to 1 μM.

### Conclusion:

FSD1 as a monomer or dimer (FSD1-FSD1) had no neutralizing effect on the signaling activity of activin A, myostatin, or GDF11 in a concentration range up to at least 1 μM. Consistent herewith, type 2 design compounds based on the native FSD1 platform did not show any neutralizing activity against activin A, myostatin, or GDF11, when tested in a concentration range up to 8.2 nM. However, when moving beyond 8.2 nM, type 2 design compound based on native FSD1 neutralized activin A, myostatin, and GDF11 signaling with an IC50 in the low nM range. This was likely because of the avidity effect of the Fc dimer on native FSD1. By replacing native FSD1 with selected FSD1 mutants in the type 2 design: FSD1 (Q124A), FSD1 (E126A), or FSD1 (Q124A, E126A), neutralization of activin A signaling was further reduced (IC50 low UM range) and neutralization of myostatin and GDF11 signaling was abolished in the tested concentration ranges. The mutations were selected based on a structural analysis of the crystal structure of Follistatin in complex with myostatin. Here, Gln124 and Glu126 from FSD1 were identified as the primary residues engaging in direct interaction with myostatin. The type 1 design compound, anti-vimentin-nanobody-FSD1 increased pSmad2/3 signaling starting at around 100 nM. An interaction between vimentin and Smad2/3 was reported in which phosphorylated vimentin augmented pSmad2/3 signaling (REF 13). In the circumstance that FSD1 bestows its fusion partner (anti-vimentin-nanobody) with an ability to enter the cytosol, this could impact on the interaction between vimentin and Smad2/3 and give rise to the observed increase in pSmad2/3 signaling. Type 4 design compounds containing a full-length FST moiety retained a growth factor neutralizing activity.

### Example 6: Surface Plasmon Resonance

### Aim:

The aim of this example was to quantify the affinity of the produced compounds for TNFα and VEGF.

### Material and Methods

Surface plasmon resonance (SPR) experiments were carried out on a Biacore 3000 (Cytiva). A CM5 chip for capture of the compounds was prepared according to the instructions of the mouse antibody capture kit (Cytiva, #BR100838). VGFR(½)-mFc-FSD1, TNFR2-mFc-FSD1, VGFR(½)/FST291-mFc, TNFR2/FST291-mFc, VGFR(½)-hFc (Aflibercept), TNFR2-hFc (Etanercept), and FST315dHBS-mFc (SEQ ID NO: 30, SEQ ID NO: 31) were captured to approximately 1,000 RU (Resonance Units). TNFα (R&D Systems, #210-TA-005,) or VEGF (R&D Systems, #293-VE-010) were then injected for 120 s followed by a 300 s dissociation phase. At the end of each binding cycle, non-covalently attached molecules were removed from both surfaces by a 180 s regeneration with 10 mM glycine, pH 1.7. All proteins were diluted in running buffer (10 mm HEPES, pH 7.5, 150 mm NaCl, 2 mM CaCl₂) and 0.05% Tween 20). A flow rate of 30 μl/min was used in all steps of the experiments. Binding analysis was performed at 25° C., and data were collected at a rate of 1 Hz. Using the BIAevaluation 4.1.1 software (Cytiva), recorded signals were referenced; the signal from the in-line reference flow cell was subtracted as was the signal from a blank run (0 nM analyte).

### Results:

Both VGFR(½)-mFc-FSD1 and VGFR(½)/FST291-mFc displayed very strong and almost irreversible binding to VEGF (FIG. 7). Likewise, TNFR2-mFc-FSD1 and TNFR2/FST291-mFc displayed very strong and almost irreversible binding to TNFα. These affinities matched the VEGF/TNFα affinities of the commercial therapeutic agents, VGFR(½)-hFc (Aflibercept) and TNFR2-hFc (Etanercept). Full-length FST315 with mutated heparin-binding site (SEQ ID NO: 29) showed no affinity to either VEGF or TNFα.

### Conclusion:

VGFR(½)-mFc-FSD1, VGFR(½)/FST291-mFc, TNFR2-mFc-FSD1 and TNFR2/FST291-mFc have similar ligand affinities as their commercial counterparts.

### Example 7: ECM Extract Binding Experiments

### Aim:

The aim of this example was to estimate the binding affinity to extracellular matrix (ECM) extract of the platform claimed in the present invention and to demonstrate the lack of binding for commercial therapeutic agents.

### Material and Methods

Binding to ECM was quantified using a modified version of a previously described colorimetric in vitro assay based on ECM extract coated plates (Corning, BioCoat Matrigel #354607) (REF. 5). ECM contains various heparan sulfates that will fixate tested compounds throughout wash cycles depending on the strength of the interaction between the compound and heparan sulfate. Briefly, plates were blocked with 100 UL per well 100% StartingBlock (Thermo Scientific, #37543) overnight. Tested compound was serially diluted in blocking buffer in a concentration range starting at 60 nM or 120 nM and added to the plate in triplicate (50 μL). Plates were incubated for two hours at room temperature and then washed four times with 200 μL tris-buffered saline containing 0.1% Tween20 and 10% StartingBlock. A two-hour primary incubation step was performed with a mixture of biotinylated anti-FST antibody (0.1 μg/mL, R&D Systems, #BAF669) and biotinylated anti-human IgG antibody (0.065 μg/mL, Invitrogen #31774) (50 μL per well). When relevant, incubation with a single primary antibody instead of a mixture was used. Binding of the biotinylated anti-human IgG antibody to commercial therapeutic agents, VGFR(½)-hFc (Aflibercept) or TNFR2-hFc (Etanercept), was validated at the same concentrations in a direct ELISA. A one-hour secondary incubation step was performed with streptavidin conjugated to horseradish peroxidase (streptavidin-HRP, 1:400 dilution, R&D Systems, #DY998) (50 μL per well). Primary antibodies and streptavidin-HRP was diluted in 100% StartingBlock and each incubation step was followed by four washes with 200 μL washing solution. TMB substrate solution (Thermo Scientific, #N301) was added (100 μL per well) and color development was followed by eye and stopped with an equal volume of Stop Solution (Thermo Scientific, #N600). The color reaction was evaluated at 450 nm wavelength using a plate reader (PerkinElmer, EnSpire 2300). Background was determined by color development in blocking buffer. Data was analyzed and half maximal effective concentration (EC50) was calculated using nonlinear regression with three parameters and no weighting and no constraints (Graphpad, Prism version 9.4.1). The calculated EC50 was a relative measure of how strongly compound bound to ECM. The highest plateau for each curve did not represent affinity to ECM but was a function of how well the primary antibodies recognized the tested compound and for how long the color reaction was allowed to continue.

### Results:

The OD450 curves of FSD1, FSD1-FSD1, and FSD1-FSD1-FSD1 demonstrated a left-shift as an indication of increased ECM affinity for each module of FSD1 present, with EC50 values ranging from 0.08 nM to 1 nM (FIG. 22A). The greatest shift of the curve was observed between one and two FSD1 modules. In accordance, type 2 design compound, VGFR(½)-mFc-FSD1 or TNFR2-mFc-FSD1, demonstrated EC50 values less than 0.5 nM (FIG. 8B-C). Native FST288 (SEQ ID NO: 27) displayed an EC50 in the 1 nM range (FIG. 8A), while type 4 design compound,

VGFR(½)/FST291-mFc or TNFR2/FST291-mFc, with one full-length FST moiety displayed EC50s around 15 nM (FIG. 8B-C). FST315dHBS-mFc (which lacked the heparin binding site, SEQ ID NO: 30, SEQ ID NO: 31) had a low-but traceable-ECM affinity with an EC50 around 80 nM (FIG. 8A). There was no indication of ECM binding by commercial therapeutic agents, VGFR(½)-hFc (Aflibercept, FIG. 8B) or TNFR2-hFc (Etanercept, FIG. 8C). To demonstrate the utility of the type 3 design, FSD1 (KTC) was covalently linked to a commercially available anti-TNFα antibody, Adalimumab (described in Example 4). Fractions with different heparin affinity were collected during heparin-affinity chromatography, indicating that a varying number of FSD1 modules were conjugated to Adalimumab. The fractions were marked Adalimumab-FSD1 low, medium, or high based on increasing heparin affinity. The exact number of FSD1 (KTC) modules coupled to Adalimumab in each fraction was not known and likely represented a continuum. Therefore, the exact molecular weights could not be determined and as such, EC50 values were not determinable. However, in the same manner as was observed with multimeric FSD1, the OD450 curves of the Adalimumab-FSD1 fractions demonstrated a left-shift from low to high as an indication of increased ECM affinity (FIG. 22B). Adalimumab without the present platform invention displayed limited, if any, binding to the ECM (FIG. 22B). Similar to type 2 design protein utilizing native FSD1, VGFR(½)-mFc-FSD1, type 2 design protein utilizing FSD1 mutants: VGFR(½)-mFc-FSD1 (Q124A), VGFR(½)-mFc-FSD1

(E126A), and VGFR(½)-mFc-FSD1 (Q124A, E126A) also displayed binding to ECM (FIG. 22C).

### Conclusion:

When used as a platform, FSD1 either as a standalone monomer, multimer, or as part of full-length FST bestowed compounds with an ability to bind ECM extract and reside in it throughout twelve wash cycles. The ECM affinity increased with the number of FSD1 moieties in the compound. Type 2 design protein utilizing FSD1 mutants also displayed binding to ECM extract. FSD1-FSD1 or type 2 design compounds demonstrated threefold to sevenfold lower EC50 compared to FSD1 and Native FST288 and thirtyfold to a hundredfold lower EC50 compared to type 4 design compounds.

### Example 8: In Vivo Estimation of Type 2 and Type 4 Design Compound Half-Lives in Skeletal Muscle in Mice Using IVIS

### Aim:

The aim of this example was to estimate the half-lives of type 2 and type 4 design compounds in skeletal muscle in mice using fluorescence.

### Material and Methods

### Compound Labelling:

The compound was fluorescently labeled according to the manufacturer's protocol with NIR730 (Sigma-Aldrich, #92315), a label for the near infrared area. After labeling, compound concentration was determined using the Pierce Coomassie (Bradford) Protein Assay Kit (Thermo Scientific, #23200).

### Animal Experiment:

Twelve 16-week-old female C57BL/6 mice were divided into four groups: PBS (vehicle), VGFR(½)-mFc-FSD1 (type 2 compound), VGFR(½)/FST291 (type 4 compound), and VGFR(½)-hFc (Aflibercept). During anesthesia, the right hind limb was shaved to expose the calf muscles. A single dose of 25 μg, fluorescently labeled compound was administered intramuscularly in a volume of 5 μL in the gastrocnemius muscle. Mice received a subcutaneous injection of pain relief (Carprofen 5 mg/kg) to counter any pain resulting from tension in the muscle. Immediately after injection and every 24 hours, mice were scanned for fluorescence using an IVIS Spectrum (PerkinElmer) in vivo imaging system. Exposure time was set at 1 second for all scans (whole-animal, excised muscle, or serum) and excitation/emission was set at 675/760 nm wavelength. Mice were euthanized after 4 days and the right gastrocnemius was dissected and blood was collected and centrifuged for serum. Gastrocnemius muscle and serum samples were scanned immediately to evaluate the fluorescent intensity. Data was analyzed using Living Image version 4.3.1 (Caliper Life Sciences, Inc.)

### Results:

In skeletal muscle, type 2 design compound, VGFR(½)-mFc-FSD1, displayed a half-life of 63.5 hours (FIG. 9A-B) and a statistically significant, 17.4 fold higher average radiance in the excised gastrocnemius muscle (FIG. 9C-D) compared to commercial VGFR(½)-hFc (Aflibercept). Type 4 design compound, VGFR(½)/FST291-mFc, displayed a half-life of 8.3 hours (FIG. 9A-B) and had a significantly higher radiance in the gastrocnemius muscle compared to PBS but not VGFR(½)-hFc (Aflibercept) (FIG. 9C-D). Commercial VGFR(½)-hFc (Aflibercept) had a half-life of 7.5 hours and did not display a significantly different average radiance in excised skeletal muscle compared to PBS, indicating a very low local presence. Notably, the presence of type 2 compound was not detectable in serum after 96 hours (FIG. 9E-F). Although some radiance could be observed in serum from mice treated with type 4 design compound, the level was not significantly different from PBS treated mice. Finally, serum from mice treated with commercial VGFR(½)-hFc (Aflibercept) exhibited profuse radiance, indicating high systemic presence.

### Conclusion:

In skeletal muscle, type 2 design compound, VGFR(½)-mFc-FSD1, displayed an almost tenfold longer half-life than its commercial counterpart, VGFR(½)-hFc (Aflibercept). As a result, type 2 design compound still displayed prominent radiance 96 hours after injection in the gastrocnemius muscle. Type 4 design compound was also detectable in the skeletal muscle after 96 hours, while radiance from VGFR(½)-hFc (Aflibercept) was not different from PBS treated samples. In serum, VGFR(½)-hFc (Aflibercept) exhibited profuse radiance, while radiance from type 2 and type 4 design compound was not different from PBS treated mice.

### Example 9: In Vivo Estimation of Type 4 Design Compound Half-Life in Eye Tissue of Mice Using IVIS

### Aim:

The aim of this example was to estimate the half-life of type 4 design compound in eye tissue in mice using fluorescence.

### Material and Methods

### Compound Labelling:

Compound was fluorescently labeled according to the manufacturer's protocol with NIR730 (Sigma-Aldrich, #92315), a label for the near infrared area. After labeling, compound concentration was determined using the Pierce Coomassie (Bradford) Protein Assay Kit (Thermo Scientific, #23200).

### Animal Experiment:

Seven 12-week-old female BALB/c mice were divided into two groups: PBS (n=3, vehicle) or active treatment (n=4, treatment depending on eye). In the PBS group, mice received PBS in both left and right eyes. In the active treatment group, mice received VGFR(½)-hFc (Aflibercept) in the left eye and VGFR(½)/FST291 (type 4 compound) in the right eye. During anesthesia, pupils were dilated using Tropicamid eye drops and a single dose of 10 μg, fluorescently labeled compound was administered intravitreally in a volume of 1 μL in the eye. Eyes were treated with Chloramphenicol post injection and mice received a subcutaneous injection of pain relief (Carprofen 5 mg/kg) to counter pain resulting from tension in the eye. Mice were scanned for fluorescence immediately after injection, at 48 hours, at 72 hours, and at 96 hours using an IVIS Spectrum (PerkinElmer) in vivo imaging system. Exposure time was set at 0.5 seconds for in vivo scans and 20 seconds for ex vivo scans. Excitation/emission was set at 675/760 nm wavelength. Mice were euthanized after 4 days and both eyes were enucleated. Data was analyzed using Living Image version 4.3.1 (Caliper Life Sciences, Inc.)

### Results:

In eye tissue, type 4 design compound, VGFR(½)/FST291-mFc, displayed a half-life of 26.4 hours (FIG. 10A) and a 5.3 fold higher average radiance in the enucleated eye (FIG. 10B-C) compared to commercial VGFR(½)-hFc (Aflibercept). VGFR(½)-hFc (Aflibercept) had a half-life of 13.3 hours and did not display a significantly different radiance in the enucleated eye compared to PBS.

### Conclusion:

Type 4 design compound, VGFR(½)/FST291-mFc, displayed a twofold longer half-life than its commercial counterpart, VGFR(½)-hFc (Aflibercept). The presence of type 4 design compound was still traceable in the enucleated eye 96 hours after injection.

### Example 10: Ex Vivo Estimation of Type 4 Design Compound Half-Life Using IVIS in Porcine Kidney During Normothermic Machine Perfusion

### Aim:

The aim of this example was to estimate the half-life of type 4 design compound in ex vivo perfused porcine kidney using fluorescence.

### Material and Methods

### Compound Labelling:

Compound was fluorescently labeled according to the manufacturer's protocol with NIR730 (Sigma-Aldrich, #92315), a label for the near infrared area. After labeling, compound concentration was determined using the Pierce Coomassie (Bradford) Protein Assay Kit (Thermo Scientific, #23200).

Ex vivo delivery to the kidney during normothermic machine perfusion (NMP): Female, Danish Landrace pigs weighing approximately 60 kg were used for nephrectomy and red blood cell isolation as described earlier (REF. 6). After nephrectomy, kidneys were flushed with isotonic saline (Fresenius Kabi) followed by a flush with Belzer UW cold storage solution (Bridge to life, UK) and stored in Belzer UW cold storage solution at 4° C. for about 16 hours. Prior to perfusion, kidneys were flushed with isotonic saline and connected to a basic perfusion setup. Two kidneys from the same pig were perfused using red blood cell-based perfusate (as previously described) (6) under oxygenation for 6 hours at 37° C. with a flow around 400-500 mL/min. In total, one mg labeled type 2 design compound, TNFR2/FST291-mFc, or TNFR2-hFc (Etanercept) was diluted in 10 mL perfusate and administered 5 minutes after the start of perfusion by slow infusion (60 mL/h) via the arterial line. At the end of perfusion, the kidney was flushed by 1.5 L isotonic saline to remove perfusate solution. Kidneys were sliced at the outer cortex and the midline and analyzed using an IVIS

Spectrum (PerkinElmer) in vivo imaging system. Exposure time was set at 8 seconds for the midline section and 6 seconds for the outer cortex section. Excitation/emission was set at 675/760 nm wavelength. Data was analyzed using Living Image version 4.3.1 (Caliper Life Sciences, Inc.). To demonstrate background fluorescence an additional pig kidney was scanned from an independent experiment in which 10 ml of PBS was infused into the arterial line during NMP.

### Results:

In ex vivo perfused porcine kidney, type 4 design compound, TNFR2/FST291-mFc, displayed a twofold higher average radiance in the both midline sections (FIG. 11A-B) and cortical sections (FIG. 11C-D) compared to commercial TNFR2-hFc (Etanercept).

### Conclusion:

When delivering a therapeutic agent during ex vivo normothermic machine perfusion, type 4 design compound, TNFR2/FST291-mFc, displayed a twofold higher radiance than its commercial counterpart, TNFR2-hFc (Etanercept), indicating higher exposure with type 4 design compound.

### Example 11: Intracellular Uptake Study In Vitro

### Aim:

The aim of this example was to evaluate the intracellular uptake of the claimed platform invention in vitro.

### Material and Methods

### Compound Labelling:

The compound was fluorescently labeled according to the manufacturer's protocol with ATTO 488 (ATTO-TEC GmbH, #AD 488), a label for the green fluorescent protein spectrum. After labeling, compound concentration was determined using the Pierce Coomassie (Bradford) Protein Assay Kit (Thermo Scientific, #23200).

### Intracellular Uptake Study:

Briefly, HEK293 cells were seeded on a cover slip glass (Thorlabs, #CG15NH) treated with poly-D-lysine (ThermoFisher, #A3890401) to promote adhesion. Cells were seeded in DMEM (ThermoFisher, #11995073) supplemented with 10% fetal bovine serum (FBS). After 24 hours, cells were washed with PBS and compounds (1 μM) were added in 100% DMEM without FBS. A control sample using 100% DMEM without added compound was also included. After 3 hours and 18 hours of incubation, cells were washed 3 times with PBS. Then, cells were incubated for 30 minutes with a mixture of Hoechst 33342 (3 μg/mL) (ThermoFisher, #H3570) and Lysotracker Red DND-99 (75 nM) (ThermoFisher, #L7528) to visualize cell nucleus and lysosomes, respectively. Cells were then washed 3 times with PBS and fixed using 4% formalin for 30 minutes. Cover slips were mounted using ProLong Glass Antifade Mountant (ThermoFisher, #P36980). Confocal imaging was conducted using a Zeiss LSM800 laser scanning confocal microscope (Carl Zeiss Microscopy GmbH, Oberkochen, Germany) equipped with 2 GaAsP and one Airyscan detector and Zen Blue Edition software (Carl Zeiss Microscopy GmbH, version 2.5). Confocal images were taken with the PlanApo x63 Oil NA 1.4 objective. For regular confocal imaging, ATTO 488 was excited using a 488 nm diode laser at 2.20% (3-hour samples) or 0.45% (18-hour samples) and detected at wavelengths 510-575 nm. Hoechst 33342 was excited using a 405 nm diode laser at 0.50% (both 3- and 18-hour samples) and detected at wavelengths 400-510 nm. Lysotracker Red DND-99 was excited using a 561 nm diode laser at 2.60% (both 3-hour and 18-hour samples) and detected at wavelengths 575-700 nm. For each sample, a Z-stack was recorded with an interval of 0.270 μm (3-hour samples) or 0.170 μm (18-hour samples). For all scans, pixel time was 1.84 μs and, averaging was set at 4× in the repeat per line mode and mean intensity method. For Airyscan superresolution imaging, ATTO 488 was excited using the 488 nm diode laser at 0.03% and detected at wavelength 490-580 nm and Hoechst 33342 was excited using the 405 nm diode laser at 0.10% and detected at wavelength 400-469. Images were processed using Fiji (v 2.14.0/1.54f). All images were equally contrast and brightness adjusted for ATTO 488 and Lysotracker Red DND-99 signal within each timepoint. Hoechst 33342 signal was individually adjusted for contrast and brightness in 3-hour samples and equally adjusted in 18-hour samples.

### Results:

In HEK293 cells, intense fluorescent signal from ATTO 488 indicated that type 1 design compound, anti-vimentin-nanobody-FSD1, was taken up by the cells after 3 hours of incubation (FIG. 16). In contrast, very little ATTO 488 signal was found in anti-vimentin-nanobody treated cells after 3 hours of incubation (FIG. 16). The ATTO 488 signal of anti-vimentin-nanobody-FSD1 at 3 hours appeared in a punctuate pattern characteristic of endocytosis. After 18 hours of incubation, the punctuate ATTO 488 signal of anti-vimentin-nanobody-FSD1 treated cells had been replaced by a fluorescent signal resembling interwoven threads (or filaments) that curved and stretched throughout the cells (FIG. 12A and FIG. 17). In contrast, very little ATTO 488 signal or no signal at all was found in anti-vimentin-nanobody treated cells or cells treated with DMEM alone (FIG. 12A). In cells treated with anti-vimentin-nanobody-FSD1 there was no apparent overlap between lysosomes (Lysotracker Red DND-99) and ATTO 488 signal, indicating that the type 1 compound was not located within a lysosomal unit (FIG. 12A and FIG. 16). The filament like structures apparent in cells treated with anti-vimentin-nanobody-FSD1 indicated that the type 1 compound escaped endosomal uptake and moved to the cytosol, where it attached to vimentin—an intermediate filament protein that forms part of the cytoskeleton. Moreover, the nuclei of cells treated with anti-vimentin-nanobody-FSD1 displayed very high ATTO 488 fluorescent intensity, indicating that the type 1 compound did not only move to the cytosol but also moved inside the cell nucleus. In an orthogonal projection it is possible to visualize the x and y cut planes simultaneously (FIG. 12B). Here, it was apparent that the ATTO 488 fluorescence was not only located at the membrane surface but was also located between the nucleus and the surface in both the x and y cut planes. This further corroborated the notion that anti-vimentin-nanobody-FSD1 was present in the cytosol itself and not just in relation to the cell membrane. There was no nucleus or filament like structures visualized with ATTO 488 fluorescence in anti-vimentin-nanobody or DMEM treated cells. This indicated that without the present platform Type 1 design, the anti-vimentin-nanobody was not taken up by cells to any great extent.

### Conclusion:

The present platform invention, here exemplified by the type 1 design compound, anti-vimentin-nanobody-FSD1, was taken up by cells and allowed visualization of vimentin filaments in the cytosol. The type 1 design compound also appeared to enter the nucleus of the cell. Taken together, these data indicated that the present platform invention can bestow therapeutic or diagnostic agents with an ability to enter the cytosol and the cell nucleus.

### Example 12: Preparation of Variants of FSD1

### Aim:

The aim of this example was to apply structural analysis to generate FSD1 mutants lacking neutralizing activity on growth factors, activin A, myostatin, and GDF11.

### Material and Methods

The crystal structure of the Follistatin-Myostatin complex (rcsb id: 3HH2) was applied for structural analysis of interactions between FSD1 and Myostatin. The inventors found that Glu126 (glutamic acid at amino acid residue position 126) from FSD1 forms a salt bridge with Lys39 (lysine at amino acid residue position 39) from Myostatin. In addition, it was found that Gln124 (glutamine at amino acid residue position 124) from FSD1 forms a hydrogen bond with Asn83 (asparagine at amino acid residue position 83) from Myostatin. Apart from these specific interactions, no salt bridges or hydrogen bonds were observed between FSD1 and Myostatin. The inventors mutated FSD1 Glu126 and/or Gln124 to alanine at these positions, to obtain the variant FSD1 domains E126A, Q124A and Q124A E126A, respectively, used herein, to disrupt the interaction, and prevent FSD1 from binding to and neutralizing the effects of Myostatin. The mutations were introduced by site-directed mutagenesis using the InFusion HD cloning kit (Clontech).

The GDF11 residues corresponding to Myostatin Lys39 and Asn83 are conserved and a similar interaction of GDF11 with FSD1 was hypothesized. In Activin A the residues corresponding to Myostatin Lys39 and Asn83 are His36 and Ser90. Hence, the interaction of FSD1 with Activin A is expected to be weaker.

### Conclusion

In conclusion, by mutating FSD1 Glu126 and Gln124 to alanine, the inventors tested their hypothesis to disrupt any interaction of FSD1 with the growth factors Activin A, Myostatin and GDF11. The variant FSD1 domains E126A, Q124A and Q124A E126A, used herein, were obtained.

### Example 13: In Vivo Demonstration of ECM Binding by Type 2 and Type 4 Design Compounds

### Aim:

The aim of this example was to demonstrate in vivo ECM binding and to visualize increased local presence of Type 2 or Type 4 design compounds in a tissue compared to a non-FSD1 fused compound.

### Materials and Methods

The example was based on material harvested from the animals as treated in Example 8. Briefly, PBS (vehicle), VGFR(½)-mFc-FSD1 (Type 2 compound), VGFR(½)/FST291 (Type 4 compound), or VGFR(½)-hFc (Aflibercept, Bayer AG, Leverkusen, Germany) was injected intramuscularly in the right gastrocnemius muscle of C57BL/6 mice (n=3 per group). Compounds were fluorescently labeled with NIR730. Four days after injection, the gastrocnemius muscle was dissected and immersion-fixed in 4% formaldehyde for 48 hours. The gastrocnemius muscle was ethanol dehydrated, embedded in paraffin, and serial sections were cut using a microtome at 2 μm thickness. Sections in-series were either stained with Sirius Red (a common stain to visualize connective tissue) or left unstained and mounted with DAPI containing mountant (#P36962, ProLong Diamond Antifade Mountant with DAPI) on microscope slides. Slides were imaged with an Olympus VS120 slide scanner equipped with Spectra X light engine and Semrock pentafilter (DAPI/FITC/Cy3/Cy5/Cy7 Penta LED HC Filter Set #F68-050) with Hamamatsu ORCA-FLASH4.0 V2 (QE82%) camera. Images were taken with the x20 Air NA 0.75 objective. DAPI was excited with 395/25 bandpass filter and emission bandpass 425/50, exposure 5 ms. NIR730 was excited with 730/40 bandpass filter and emission bandpass 800/100, exposure 150 ms. Images were processed using Fiji (v 2.14.0/1.54f). All images were equally contrast and brightness adjusted for DAPI, except VGFR(½)-mFc-FSD1 that received less adjustment because of a stronger signal. All images were equally adjusted for NIR730 signal.

### Results:

In skeletal muscle, the presence of type 2 design compound, VGFR(½)-mFc-FSD1, was readily visible by its NIR730 fluorescent signal (FIG. 13A). In contrast, no NIR730 signal was observed in PBS treated muscle. A weak NIR730 signal was detected in both VGFR(½)/FST291-mFc (type 4 design compound) and VGFR(½)-hFc (Aflibercept) treated muscle. The intense signal from VGFR(½)-mFc-FSD1 was present in two distinct compartments. The first compartment, which exhibited the strongest signal, was identical to the distribution of the connective tissue strokes within the muscle, visualized by Sirius Red staining. The second compartment formed a network of fluorescent signal throughout the muscle parenchyma (FIG. 13B, Area 1 magnified) and the vascular tissue penetrating the muscle (FIG. 13B, Area 2 magnified). The NIR730 signal from the parenchyma was either emitted from inside the muscle fibers beneath the sarcolemma (subsarcolemmal region) or in close proximity to the sarcolemma on the extracellular surface. The NIR730 signal from the vascular tissue enabled visualization of all three layers of the vasculature (intima, media, and adventitia). Similarly to the parenchyma, VGFR(½)-mFc-FSD1 signal either emitted from inside cells of the vasculature or from the extracellular compartment, or both.

### Conclusion:

ECM is a major constituent of connective tissue and likely served as a reservoir for the claimed platform invention. In this way, the local presence of Type 2 design compound, VGFR(½)-mFc-FSD1, in skeletal muscle was substantially increased, indicated by the intense NIR730 fluorescent signal of the compound. In contrast, both VGFR(½)/FST291-mFc (Type 4 design compound) and VGFR(½)-hFc (Aflibercept) demonstrated limited NIR730 signal, indicating little to no presence of these compounds four days after administration.

### Example 14: In Vivo Evaluation of Type 2 and Type 4 Design Compound Half-Lives in Eye Tissue of Mice Using Fluorescence Fundus Imaging

### Aim:

The aim of this example was to evaluate the half-life of Type 2 and Type 4 design compounds in eye tissue of mice using fluorescence funduscopy.

### Materials and Methods

### Compound Labelling:

The compound was fluorescently labelled according to the manufacturer's protocol with ATTO 488 (ATTO-TEC GmbH, #AD 488), a label for the green fluorescent protein spectrum. After labelling, compound concentration was determined using the Pierce Coomassie (Bradford) Protein Assay Kit (Thermo Scientific, #23200).

### Animal Experiment:

Eight 12-weeks-old female BALB/c mice were distributed into four groups with two mice in each group: PBS (vehicle), VGFR(½)-mFc-FSD1 (type 2 compound), VGFR(½)/FST291 (Type 4 compound), and VGFR(½)-hFc (Aflibercept, Bayer AG, Leverkusen, Germany). During anaesthesia, Tropicamide (Mydriacyl 0.5%) eye drops were applied to dilate the pupil. A single dose of 10 μg, fluorescently labelled compound was administered intravitreally in a volume of 1 μL by an experienced operator. Injected eyes were treated with Chloramphenicol gel (Kloramfenikol, 1%) to prevent infection and mice received a subcutaneous injection of pain relief (Carprofen 5 mg/kg) after the procedure. Immediately after intravitreal administration, baseline fluorescence fundus imaging (Micron IV, Phoenix Research Laboratories, OR, USA) was carried out with an exposure of 200 ms to verify correct delivery and fluorescent signal of the compounds. Four days after treatment, mice were anesthetized and fluorescence fundus imaging was performed again with an exposure of 500 ms. Fluorescent intensity of the images was quantified using Fiji (v 2.14.0/1.54f).

### Results:

Fluorescence funduscopy of the eye at baseline showed no statistically significant difference in average fluorescent intensity between groups (FIG. 14A). However, four days after treatment, eyes treated with VGFR(½)-mFc-FSD1 (type 2 compound) showed significantly higher average fluorescent intensity than eyes treated with

VGFR(½)/FST291-mFc (Type 4 compound) or VGFR(½)-hFc (Aflibercept) (FIG. 14 B-C). The fluorescent signal from VGFR(½)/FST291-mFc or VGFR(½)-hFc (Aflibercept) treated eyes did not differ from the signal of PBS treated eyes or each other four days after treatment (FIG. 14 C).

### Conclusion:

In eye tissue, Type 2 design compound, VGFR(½)-mFc-FSD1, displayed a superior in-eye half-life profile than Type 4 design compound, VGFR(½)/FST291-mFc, and VGFR(½)-hFc (Aflibercept). This was evidenced by significantly greater fluorescent signal of VGFR(½)-mFc-FSD1 treated eyes four days after treatment compared to the other compounds.

### Example 15: In Vivo Efficacy of Type 2 Design Compound in a Disease Model of Wet Age-Related Macular Degeneration

### Aim:

The aim of this example was to investigate the efficacy of Type 2 design compound in the choroidal neovascularization (CNV) disease model of wet age-related macular degeneration (wet AMD) in mice.

### Materials and Methods

### Animal Experiment:

Twenty-three 8-weeks-old male C57BL6/J mice were distributed into three groups: Mouse IgG2a isotype control (Invitrogen, #02-6200; n=7), VGFR(½)-mFc-FSD1 (type 2 compound, n=8), and VGFR(½)-hFc (Aflibercept, Bayer AG, Leverkusen, Germany; n=8). During anesthesia, Tropicamide (Mydriacyl 0.5%) eye drops were applied to dilate the pupil. Laser induction of CNV was performed using an image-guided laser system (Micron IV, Phoenix Research Laboratories, OR, USA) in accordance with the method described by Gong et al. (REF. 7) Laser settings were: wavelength 532 nm and 240 mW; duration: 70 ms; and size: 50 μm. In the eye, four laser burns were applied in a clockwise rotation (12, 3, 6 and 9 o'clock). The distance between two laser burns and the distance between a laser burn and the optic nerve was approximately two disc-diameters of the optic nerve. After CNV induction, a single dose of 10 μg compound was administered intravitreally in a volume of 1 μL by an experienced and compound-blinded operator. The compound vehicle used was PBS. Injected eyes were treated with Chloramphenicol gel (Kloramfenikol, 1%) to prevent infection and mice received a subcutaneous injection of pain relief (Carprofen 5 mg/kg) after the procedure. One week after CNV induction and compound injection, mice were euthanized and eyes were enucleated and fixed in 4% paraformaldehyde for 2 hours at room temperature and then washed with PBS.

Retinal pigment epithelium/choroidal flat-mounting and immunohistochemistry: Retinal pigment epithelium (RPE)/choroidal flat-mounting was performed as described by Askou et al. (REF. 8) In a 96-wells plate, flat-mounts were permeabilized and blocked in PBB buffer (1×PBS, 4% BSA [Millipore, #81-068-3], and 0.5% Triton X-100 [Millipore, #1.08603]) for 2 hours at 4° C. To visualize the vasculature, samples were incubated with a rat primary anti-mouse CD31 antibody (BD Pharmingen, #557355) in PBB at a dilution of 1:100 and biotin conjugated Isolectin GS-IB4 (Invitrogen, #121414) in PBB at a dilution of 1:100 overnight at 4° C. Samples were washed 4 times in PBS-X washing solution (1×PBS and 0.5% Triton X-100) and incubated with a goat secondary anti-rat IgG antibody conjugated with Alexa Fluor 568 (Invitrogen, #A-11077) in PBB at a dilution of 1:500 and streptavidin conjugated Alexa Fluor 405 (Invitrogen, #S32351) in PBB at a dilution of 1:100 for 2 hours at room temperature followed by 6 washes in PBS-X. RPE/choroidal flat-mounts were mounted on microscope slides and imaged with an Olympus VS120 slide scanner equipped with Spectra X light engine and Semrock pentafilter (DAPI/FITC/Cy3/Cy5/Cy7 Penta LED HC Filter Set #F68-050) with Hamamatsu ORCA-FLASH4.0 V2 (QE82%) camera. Images were taken with the x20 Air NA 0.75 objective. Alexa Fluor 405 was excited with 395/25 bandpass filter and emission bandpass 425/50, exposure 10 ms. Alexa Fluor 568 was excited with 575/25 bandpass filter and emission bandpass 600/60, exposure 10 ms. Images were processed using Fiji (v 2.14.0/1.54f). For quantification of average CNV lesion area per eye, samples were equally contrast and brightness adjusted and global threshold filtered. The threshold was eye balled in 5 samples to determine optimal low and high cut off values. Samples were blinded to the investigator during quantification. Exclusion of CNV lesions were based on the recommendations of Gong et al (REF. 7) and took place before unblinding.

### Results:

In the CNV disease model of wet AMD in mice, immunohistochemical staining of the RPE/choroidal flat-mount for CD31 and Isolectin provided two complementary approaches for visualization of CNV lesions (FIG. 15A). Based on both CD31 and Isolectin staining, eyes treated with either VGFR(½)-mFc-FSD1 (Type 2 compound) or VGFR(½)-hFc (Aflibercept) had statistically significant reductions in CNV lesion area compared to eyes treated with Mouse IgG2a isotype control (FIG. 15 B). No significant difference was observed in CNV lesion area estimated by both CD31 and Isolectin staining between VGFR(½)-mFc-FSD1 and VGFR(½)-hFc (Aflibercept) treated eyes.

### Conclusion:

In the CNV disease model of wet AMD in mice, Type 2 design compound, VGFR(½)-mFc-FSD1, was equally effective in reducing CNV lesion area as the commercial counterpart, VGFR(½)-hFc (Aflibercept). The results indicated that the present platform invention, here exemplified by a Type 2 design compound, did not interfere with the ligand neutralizing effect of the therapeutic moiety (i.e. VGFR(½)). In this experimental setup, the therapeutic effect of VGFR(½)-mFc-FSD1 was not expected to exceed that of VGFR(½)-hFc (Aflibercept) because both compounds were administered on the same day as CNV was induced.

### Example 16: Intracellular Uptake Study In Vivo

### Aim:

The aim of this example was to evaluate the intracellular uptake of the claimed platform invention in vivo.

### Materials and Methods

### Compound Labelling:

The compound was fluorescently labeled according to the manufacturer's protocol with ATTO 488 (ATTO-TEC GmbH, #AD 488), a label for the green fluorescent protein spectrum. After labeling, compound concentration was determined using the Pierce Coomassie (Bradford) Protein Assay Kit (Thermo Scientific, #23200).

### Animal Experiment:

Two 12-week-old female BALB/c mice were allocated for injection with either anti-vimentin-nanobody-FSD1 (type 1 compound, n=1) or anti-vimentin-nanobody (n=1). During anaesthesia, the right hind limb was shaved to expose the calf muscles. A single dose of 8 μg, fluorescently labeled compound was administered intramuscularly in a volume of 5 μl in the gastrocnemius muscle. Mice received a subcutaneous injection of pain relief (Carprofen 5 mg/kg) to counter any pain resulting from tension in the muscle. Eighteen hours after injection, the gastrocnemius muscle was dissected and immersion-fixed in 4% formaldehyde. The gastrocnemius muscle was ethanol dehydrated, embedded in paraffin, and sectioned at 4 μm thickness using a microtome. Sections were left unstained and mounted with DAPI containing mountant (#P36962, ProLong Diamond Antifade Mountant with DAPI) on microscope slides. Confocal imaging was conducted using a Zeiss LSM800 laser scanning confocal microscope (Carl Zeiss Microscopy GmbH, Oberkochen, Germany) equipped with 2 GaAsP and one Airyscan detector and Zen Blue Edition software (Carl Zeiss Microscopy GmbH, version 2.5). Confocal images were taken with the PlanApo x63 Oil NA 1.4 objective. ATTO 488 was excited using a 488 nm diode laser at 1.30% and detected at wavelengths 510-575 nm. DAPI was excited using a 405 nm diode laser at 1.00% and detected at wavelengths 400-510 nm. For each sample, a Z-stack was recorded with an interval of 0.190 μm. For all scans, pixel time was 1.10 μs and averaging was set at 4x in the repeat per line mode and mean intensity method. Images were processed using Fiji (v 2.14.0/1.54f). All images were equally contrast and brightness adjusted for ATTO 488 and DAPI.

### Results:

In skeletal muscle, the presence of type 1 design compound, anti-vimentin-nanobody-FSD1, was readily visible by its ATTO 488 fluorescent signal (FIG. 18). In contrast, no ATTO 488 signal was detected from anti-vimentin-nanobody injected muscle. The signal from anti-vimentin-nanobody-FSD1 was most prominent in cells residing in the connective tissue, some of which appeared with a spindle-shaped morphology characteristic of fibroblasts. The fluorescent ATTO 488 signal resembled interwoven threads (or filaments) that curved and stretched throughout the cells and in relation to the cell nuclei (DAPI stain). No clear fluorescent features were detected in the muscle parenchyma.

### Conclusion:

The present platform invention, here exemplified by a type 1 design compound, anti-vimentin-nanobody-FSD1, was taken up by cells and allowed visualization of vimentin filaments. This would only be possible if the compound had entered the cytosol subsequent to cellular uptake. In contrast, no ATTO 488 signal was detected from the specimen injected with anti-vimentin-nanobody, indicating that the nanobody alone was not able to enter the cytosol of cells without the present platform invention. The intermediate filament protein, vimentin, is not expressed in all cell types, but it is a hallmark of cells of mesenchymal origin, including fibroblasts (REFs 9, 10). No distinct ATTO 488 signal was detected in the parenchyma of anti-vimentin-nanobody-FSD1 injected muscle, which is consistent with the fact that vimentin is not expressed in mature and intact skeletal muscle fibers (REF. 11). Thus, the anti-vimentin-nanobody-FSD1 taken up by the parenchyma would likely be dispersed throughout the cytosol of skeletal muscle fibers and not concentrated at the target protein, vimentin. The volume of a skeletal muscle fiber is substantially greater than most other cell types and any fluorescent ATTO 488 signal from the parenchyma was likely diluted beyond the point of detection in this study. Taken together, these data indicated that the present platform invention can bestow therapeutic or diagnostic agents with an ability to enter the cytosol and possibly the cell nucleus.

### Example 17: Neutralization of Glucocorticoid Signaling

### Aim:

The aim of this example was to quantify neutralization of glucocorticoid signaling in compounds designed to capture and neutralize glucocorticoids based on type 2 design or type 4 design of the present platform invention.

### Material and Methods

Glucocorticoid neutralization of cortisol (Supelco, #C-106-1ML) or prednisolone 21-hemisuccinate (a water-soluble format of prednisolone, Sigma-Aldrich, #P4153-1G) was quantified using a reporter gene bioassay of glucocorticoid response element (GRE) activation (REF. 12). HEK293 cells were transfected with a construct containing a three times repeated GRE motif upstream of a minimal promoter controlling luciferase expression. The HEK293 cell line was propagated to a stable line with a strong luciferase responsiveness to glucocorticoid signaling induced by endogenous or synthetic glucocorticoids. Briefly, cells were stimulated with cortisol or prednisolone 21-hemisuccinate and cotreated with a concentration range of recombinant proteins starting at 2 μM. After sixteen to twenty hours, cells were lysed (Promega, Glo Lysis Buffer, #E2661), luciferase substrate was added (Promega, Steady-Glo Luciferase Assay System, #E2520), and the luminescent signal was analyzed using a plate reader (PerkinElmer, EnSpire 2300). Six or more positive controls (glucocorticoid without inhibitor) and negative controls (no glucocorticoid and no inhibitor) were included. Data was analyzed and half maximal inhibitory concentration (IC50) was calculated using nonlinear regression with three parameters and no weighting and no constraints (Graphpad, Prism version 9.4.1).

### Results:

Compounds containing CBG fused to type 2 (CBG-mFc-FSD1) or type 4 design (CBG/FST291-mFc) of the present platform invention neutralized both endogenous glucocorticoid signaling (cortisol) or synthetic glucocorticoid signaling (prednisolone 21-hemisuccinate) with IC50 values in the low to mid nM range, when tested in a concentration range up to 2 μM. The type 2 design compound had two CBG moieties and, accordingly, approximately double the ligand affinity of the type 4 design compound with one CBG moiety.

### Conclusion:

CBG, which normally acts as a serum glucocorticoid transport or buffer protein, can be harnessed for glucocorticoid neutralization, which was exemplified here using both the type 2 and type 4 design of the present platform invention. By fusing CBG with the present platform invention, a localized glucocorticoid neutralizing compound was developed. Since the present platform invention remained relatively localized to the tissue to which it was injected (depending on the number of FSD1 modules, as shown in Examples 7 and 8), no systemic effect of the glucocorticoid neutralizing compound is expected.

### Example 18: Degradation of Intracellular Protein Using the Present Platform Invention

### Aim:

The aim of this example was to demonstrate the utility of the present platform invention to degrade intracellular protein using a proteolysis targeting chimera (PROTAC) strategy.

### Materials and Methods

### Generation of a PROTAC Nanobody:

Anti-vimentin-nanobody and anti-vimentin-nanobody-FSD1 (Type 1 compound) were coupled to (S, R, S)-AHPC-PEG8-NHS, a ligand for the Von Hippel-Lindau tumor suppressor protein (VHL) linked to an NHS ester via a PEG8 linker (Broadpharm, #BP-25703). Briefly, each compound was incubated with the VHL ligand in a buffer of 500 mM KCl, 20 mM Hepes, and pH 8.3 for 1 hour. The buffer was then swapped to PBS and protein concentration was determined using NanoDrop 2000c (Thermo Scientific, #ND-2000C).

### Degradation of Vimentin In Vitro:

HEK293 cells were seeded in DMEM (ThermoFisher, #11995073) supplemented with 2% FBS at 600.000 cells per well in a Nunclon Delta treated 6 well plate (ThermoFisher, #140685). At the time of seeding, PROTAC nanobody compounds were added (500 nM) and a control condition without compound was also included (DMEM control). After 24 hours, cells were trypsinized and transferred to a microcentrifuge tube and centrifuged at 200×g for 5 minutes at room temperature. Medium was removed and the pellet was washed with PBS and centrifuged again at 200×g for 5 minutes. The PBS was removed and the pellet was resuspended in 100 UL RIPA buffer (ThermoFisher, #89901) with 1 mM phenylmethanesulfonyl fluoride (PMSF, Sigma-Aldrich, #93482). The resulting homogenate was incubated at 4° C. while shaking at 750 RPM for 30 minutes and then centrifuged at 16,000×g at 4° C. for 20 minutes. The supernatant (lysate) was stored at −20° C. and the protein concentration was determined using the Pierce Coomassie (Bradford) Protein Assay Kit (Thermo Scientific, #23200) with lysate diluted 1/10 in PBS.

### Western Blot:

For western blotting, 16 μg total protein was run on a stain-free SDS-PAGE and analyzed for total protein using Gel Doc EZ System (Bio-Rad, #1708270). The gel was transferred to a PVDF membrane and blocked in 5% skimmed milk for 1 hour. A one-hour incubation step was performed with a primary rabbit polyclonal anti-vimentin antibody (Invitrogen, #PA5-27231) at a dilution of 1:10,000 in 5% skimmed milk. After 5 washes in tris-buffered saline with 0.1% Tween20 (TBST), a one-hour incubation step was performed with a secondary goat anti-rabbit IgG-peroxidase antibody (Sigma-Aldrich, #A₀₅₄₅) at a dilution of 1:5,000 in 5% skimmed milk. After 5 washes in TBST, the blot was imaged using the Invitrogen iBright FL1500 Imaging System (Invitrogen, #A44241) with an exposure of 14 minutes. The iBright Analysis Software (version 1.8.1) was used to quantitate the background-adjusted vimentin band intensity. Next, a western blot for glyceraldehyde 3-phosphate dehydrogenase (GAPDH) as a loading control was performed on the same membrane. The same procedure as described above was followed with a primary mouse monoclonal anti-GAPDH antibody (Invitrogen, #MA5-15738) at a dilution of 1:1,000 and a secondary goat anti-mouse IgG-peroxidase antibody (Sigma-Aldrich, #A2554) at a dilution of 1:5,000. The blot was imaged with an exposure of 35 seconds and the background-adjusted GAPDH band intensity was quantitated.

### Results:

In HEK293 cells, 24 hours of treatment with VHL ligand coupled anti-vimentin-nanobody-FSD1 (Type 1 compound) reduced the vimentin level substantially (GAPDH normalized) compared to lysate from cells treated with DMEM control or VHL coupled anti-vimentin-nanobody (FIG. 24A-B). Anti-vimentin-nanobody (without FSD1) did not reduce the vimentin level compared to DMEM control. To ensure data consistency, the vimentin band intensity was also quantified using Fiji and normalized with the stain-free total protein estimation (instead of GAPDH intensity). In this analysis, similar results were obtained (data not shown).

### Conclusion:

The present platform invention, here exemplified by the Type 1 design compound, facilitated degradation of vimentin (a constitutively expressed, intracellular protein), when used in a PROTAC strategy. In contrast, anti-vimentin-nanobody coupled to VHL ligand but without the platform invention did not degrade vimentin. The results indicated that the present platform invention can bestow therapeutic or diagnostic agents with an ability to enter the cytosol. In this way, intracellular proteins may be degraded by recombinantly fusing or chemically coupling the platform invention with, for example, a nanobody against an intracellular target protein and then fusing or coupling this compound (nanobody-FSD1) to an E3 ligase ligand.

### REFERENCES

1. Kober, L. et al. (2013) Optimized signal peptides for the development of high expressing CHO cell lines. Biotechnol. Bioeng. 110, 1164-1173
2. Choi, H.-J. et al. (2013) A Heterodimeric Fc-Based Bispecific Antibody Simultaneously Targeting VEGFR-2 and Met Exhibits Potent Antitumor Activity.
3. Dennler, S. et al. (1998) Direct binding of Smad3 and Smad4 to critical TGF beta-inducible elements in the promoter of human plasminogen activator inhibitor-type 1 gene. EMBO J. 17, 3091-3100
4. Lodberg, A. et al. (2019) A follistatin-based molecule increases muscle and bone mass without affecting the red blood cell count in mice. FASEB J. 33, 6001-6010
5. Pearsall, R. S. et al. (2019) Follistatin-based ligand trap ACE-083 induces localized hypertrophy of skeletal muscle with functional improvement in models of neuromuscular disease. Sci. Rep. 9, 11392
6. Lohmann, S. et al. (2021) Mesenchymal stromal cell treatment of donor kidneys during ex vivo normothermic machine perfusion: A porcine renal autotransplantation study. Am. J. Transplant 21, 2348-2359
7. Gong, Y. et al. (2015) Optimization of an Image-Guided Laser-Induced Choroidal Neovascularization Model in Mice. PLOS One 10
8. Askou, A. L. et al. (2017) Suppression of Choroidal Neovascularization in Mice by Subretinal Delivery of Multigenic Lentiviral Vectors Encoding Anti-Angiogenic MicroRNAs. Hum. Gene Ther. Methods 28, 222-233
9. Ostrowska-Podhorodecka, Z. et al. (2022) Impact of Vimentin on Regulation of Cell Signaling and Matrix Remodeling. Front Cell Dev Biol 10, 869069.
10. Kidd, M. E., et al. (2014) The role of Vimentin intermediate filaments in the progression of lung cancer. Am J Respir Cell Mol Biol 50, 1-6 (2014).
11. Vater, R., et al. (1994) The expression of vimentin in satellite cells of regenerating skeletal muscle in vivo. The Histochemical Journal 26:12 26, 916-928
12. Novotna, A., Pavek, P. & Dvorak, Z. (2012) Construction and characterization of a reporter gene cell line for assessment of human glucocorticoid receptor activation. European Journal of Pharmaceutical Sciences 47, 842-847
13. Jang H R, Shin S B, Kim C H, et al. (2021) PLK1/vimentin signaling facilitates immune escape by recruiting Smad2/3 to PD-L1 promoter in metastatic lung adenocarcinoma [published correction appears in Cell Death Differ. 2021 Aug. 17;:]. Cell Death Differ. 2021; 28 (9): 2745-2764. doi:10.1038/s41418-021-00781-4

Sequence listing
SEQ ID NO: 1
Human FSD1
ETCENVDCGPGKKCRMNKKNKPRCVCAPDCSNITWKGPVCGLDGKTYRNECALLKA
RCKEQPELEVQYQGRCK
SEQ ID NO: 2
Human FSD1-FSD1
ETCENVDCGPGKKCRMNKKNKPRCVCAPDCSNITWKGPVCGLDGKTYRNECALLKA
RCKEQPELEVQYQGRCKETCENVDCGPGKKCRMNKKNKPRCVCAPDCSNITWKGP
VCGLDGKTYRNECALLKARCKEQPELEVQYQGRCK
SEQ ID NO: 3
Human FSD1-FSD1 as a single entity produced in E. coli contains an
initiator methionine (M) amino acid.
METCENVDCGPGKKCRMNKKNKPRCVCAPDCSNITWKGPVCGLDGKTYRNECALL
KARCKEQPELEVQYQGRCKETCENVDCGPGKKCRMNKKNKPRCVCAPDCSNITWK
GPVCGLDGKTYRNECALLKARCKEQPELEVQYQGRCK
SEQ ID NO: 4
Polynucleotide sequence encoding Human FSD1-FSD1 as a single
entity produced in E. coli contains an initiator
methionine (M) amino acid.
ATGGAGACATGCGAGAACGTGGATTGTGGACCAGGCAAGAAGTGCCGGATGAAC
AAGAAGAACAAGCCCAGATGCGTGTGCGCTCCTGACTGCAGCAACATCACCTGG
AAGGGACCCGTGTGCGGCCTGGATGGCAAGACATACCGGAATGAGTGCGCCCTG
CTGAAGGCTAGGTGTAAGGAGCAGCCTGAGCTGGAGGTGCAGTATCAGGGCCGG
TGCAAGGAGACATGCGAGAACGTGGATTGTGGACCAGGCAAGAAGTGCCGGATG
AACAAGAAGAACAAGCCCAGATGCGTGTGCGCTCCTGACTGCAGCAACATCACCT
GGAAGGGACCCGTGTGCGGCCTGGATGGCAAGACATACCGGAATGAGTGCGCC
CTGCTGAAGGCTAGGTGTAAGGAGCAGCCTGAGCTGGAGGTGCAGTATCAGGGC
CGGTGCAAG
SEQ ID NO: 5
anti-vimentin-nanobody-FSD1. Expressed in E. coli and therefore
contains an initiator methionine (M) amino acid.
MQVQLVESGGGLVQSGGSLTLTCAASGFTFSAASMRWVRQVPGKGLEWVATIDGT
GANSYYSESAKGRFTISRDNARNTLYLQMNNLKPDDTAVYYCANFGRNYWGKGTQV
TVSSETCENVDCGPGKKCRMNKKNKPRCVCAPDCSNITWKGPVCGLDGKTYRNEC
ALLKARCKEQPELEVQYQGRCK
SEQ ID NO: 6
Polynucleotide encoding anti-vimentin-nanobody-FSD1.
Expressed in E. coli and therefore contains an
initiator methionine (M) amino acid.
ATGCAAGTACAGCTAGTTGAATCAGGTGGAGGGTTGGTGCAAAGCGGTGGTTCTC
TGACCTTGACGTGCGCGGCGTCTGGCTTCACCTTCAGCGCTGCCTCCATGCGTT
GGGTACGTCAGGTGCCGGGTAAGGGCCTTGAGTGGGTCGCAACCATCGACGGTA
CTGGTGCAAACAGCTATTACAGCGAGTCGGCGAAAGGCCGTTTTACGATCAGCCG
TGATAATGCGCGTAACACCTTATATCTGCAGATGAATAACCTGAAACCGGATGACA
CCGCAGTTTACTACTGCGCGAACTTTGGTCGCAACTATTGGGGTAAGGGCACGCA
AGTGACCGTTTCCTCCGAAACCTGTGAAAATGTCGATTGCGGTCCGGGCAAAAAG
TGCCGTATGAACAAGAAGAACAAGCCGCGTTGTGTTTGCGCTCCGGACTGTAGCA
ATATTACCTGGAAAGGCCCAGTGTGCGGCCTGGACGGCAAAACCTACCGCAACG
AATGTGCGCTGCTGAAAGCCAGATGCAAAGAGCAGCCGGAACTGGAGGTTCAAT
ATCAGGGTCGCTGCAAG
SEQ ID NO: 7
VGFR(1/2)-mFc-FSD1. Serum albumin preproprotein signal peptide in
bold, murine IgG2A underlined, FSD1 double underlined.
MKWVTFISLLFLFSSAYSSDTGRPFVEMYSEIPEIIHMTEGRELVIPCRVTSPNITVTLK
KFPLDTLIPDGKRIIWDSRKGFIISNATYKEIGLLTCEATVNGHLYKTNYLTHRQTNTIIDV
VLSPSHGIELSVGEKLVLNCTARTELNVGIDFNWEYPSSKHQHKKLVNRDLKTQSGSE
MKKFLSTLTIDGVTRSDQGLYTCAASSGLMTKKNSTFVRVHEKPRGPTIKPCPPCKCP
APNLLGGPSVFIFPPKIKDVLMISLSPIVTCVVVDVSEDDPDVQISWFVNNVEVHTAQT
QTHREDYNSTLRVVSALPIQHQDWMSGKEFKCKVNNKDLPAPIERTISKPKGSVRAP
QVYVLPPPEEEMTKKQVTLTCMVTDFMPEDIYVEWTNNGKTELNYKNTEPVLDSDGS
YFMYSKLRVEKKNWVERNSYSCSVVHEGLHNHHTTKSFSRTPGKETCENVDCGPGK
KCRMNKKNKPRCVCAPDCSNITWKGPVCGLDGKTYRNECALLKARCKEQPELEVQY
QGRCK
SEQ ID NO: 8
Polynucleotide sequence encoding VGFR(1/2)-mFc-FSD1.
ATGAAGTGGGTGACCTTCATCTCTCTGCTGTTCCTGTTCTCTTCTGCCTACTCTAG
CGACACCGGCAGGCCCTTCGTGGAGATGTACAGCGAGATCCCCGAGATCATCCA
CATGACCGAGGGCAGGGAGCTGGTGATCCCCTGCAGGGTGACCAGCCCCAACAT
CACCGTGACCCTGAAGAAGTTCCCCCTGGACACCCTGATCCCCGACGGCAAGAG
GATCATCTGGGACAGCAGGAAGGGCTTCATCATCAGCAACGCCACCTACAAGGA
GATCGGCCTGCTGACCTGCGAGGCCACCGTGAACGGCCACCTGTACAAGACCAA
CTACCTGACCCACAGGCAGACCAACACCATCATCGACGTGGTGCTGAGCCCCAG
CCACGGCATCGAGCTGAGCGTGGGCGAGAAGCTGGTGCTGAACTGCACCGCCA
GGACCGAGCTGAACGTGGGCATCGACTTCAACTGGGAGTACCCCAGCAGCAAGC
ACCAGCACAAGAAGCTGGTGAACAGGGACCTGAAGACCCAGAGCGGCAGCGAGA
TGAAGAAGTTCCTGAGCACCCTGACCATCGACGGCGTGACCAGGAGCGACCAGG
GCCTGTACACCTGCGCCGCCAGCAGCGGCCTGATGACCAAGAAGAACAGCACCT
TCGTGAGGGTGCACGAGAAGCCACGGGGACCAACCATCAAACCATGCCCCCCCT
GTAAGTGCCCCGCACCTAACCTCCTGGGTGGGCCATCCGTGTTTATCTTCCCCCC
TAAAATTAAGGATGTGCTGATGATCAGCCTGAGCCCTATCGTGACATGCGTGGTC
GTCGACGTGAGCGAGGATGACCCCGACGTCCAGATCTCTTGGTTTGTGAACAAC
GTGGAGGTTCACACCGCCCAAACCCAGACACACAGAGAGGACTATAATTCTACTC
TCCGCGTTGTGTCCGCCCTGCCCATCCAGCACCAAGATTGGATGTCTGGTAAGGA
ATTCAAGTGTAAAGTGAACAATAAAGATCTGCCCGCACCAATCGAAAGAACCATTT
CTAAGCCTAAAGGCTCCGTGCGGGCTCCTCAGGTTTACGTCCTGCCACCCCCCG
AGGAAGAGATGACTAAGAAGCAGGTGACCCTGACCTGTATGGTGACCGATTTTAT
GCCCGAAGACATTTACGTCGAGTGGACCAATAATGGTAAGACAGAACTGAACTAT
AAAAATACAGAGCCAGTGCTCGACTCTGATGGCAGCTATTTTATGTACTCTAAGCT
GAGAGTTGAGAAGAAAAACTGGGTGGAACGGAATTCTTACTCTTGCTCTGTCGTG
CACGAAGGACTGCACAACCACCACACTACCAAGTCCTTTTCTAGGACCCCCGGCA
AGGAGACCTGCGAGAACGTGGACTGCGGCCCCGGCAAGAAGTGCCGGATGAAC
AAGAAGAACAAGCCCCGGTGCGTGTGCGCCCCCGACTGCAGCAACATCACCTGG
AAGGGCCCCGTGTGCGGCCTGGACGGCAAGACCTACCGGAACGAGTGCGCCCT
GCTGAAGGCCCGGTGCAAGGAGCAGCCCGAGCTGGAGGTGCAGTACCAGGGCC
GGTGCAAG
SEQ ID NO: 9
TNFR2-mFc-FSD1. Serum albumin preproprotein signal peptide in bold,
murine IgG2A underlined, FSD1 double underlined.
MKWVTFISLLFLFSSAYSLPAQVAFTPYAPEPGSTCRLREYYDQTAQMCCSKCSPGQ
HAKVFCTKTSDTVCDSCEDSTYTQLWNWVPECLSCGSRCSSDQVETQACTREQNRI
CTCRPGWYCALSKQEGCRLCAPLRKCRPGFGVARPGTETSDVVCKPCAPGTFSNTT
SSTDICRPHQICNVVAIPGNASMDAVCTSTSPTRSMAPGAVHLPQPVSTRSQHTQPT
PEPSTAPSTSFLLPMGPSPPAEGSTGDPRGPTIKPCPPCKCPAPNLLGGPSVFIFPPKI
KDVLMISLSPIVTCVVVDVSEDDPDVQISWFVNNVEVHTAQTQTHREDYNSTLRVVSA
LPIQHQDWMSGKEFKCKVNNKDLPAPIERTISKPKGSVRAPQVYVLPPPEEEMTKKQ
VTLTCMVTDFMPEDIYVEWTNNGKTELNYKNTEPVLDSDGSYFMYSKLRVEKKNWVE
RNSYSCSVVHEGLHNHHTTKSFSRTPGKETCENVDCGPGKKCRMNKKNKPRCVCA
PDCSNITWKGPVCGLDGKTYRNECALLKARCKEQPELEVQYQGRCK
SEQ ID NO: 10
Polynucleotide encoding TNFR2-mFc-FSD1
ATGAAGTGGGTGACCTTCATCTCTCTGCTGTTCCTGTTCTCTTCTGCCTACTCTCT
GCCCGCCCAGGTGGCCTTCACCCCCTACGCCCCCGAGCCCGGCAGCACCTGCA
GGCTGAGGGAGTACTACGACCAGACCGCCCAGATGTGCTGCAGCAAGTGCAGCC
CCGGCCAGCACGCCAAGGTGTTCTGCACCAAGACCAGCGACACCGTGTGCGACA
GCTGCGAGGACAGCACCTACACCCAGCTGTGGAACTGGGTGCCCGAGTGCCTGA
GCTGCGGCAGCAGGTGCAGCAGCGACCAGGTGGAGACCCAGGCCTGCACCAGG
GAGCAGAACAGGATCTGCACCTGCAGGCCCGGCTGGTACTGCGCCCTGAGCAAG
CAGGAGGGCTGCAGGCTGTGCGCCCCCCTGAGGAAGTGCAGGCCCGGCTTCGG
CGTGGCCAGGCCCGGCACCGAGACCAGCGACGTGGTGTGCAAGCCCTGCGCCC
CCGGCACCTTCAGCAACACCACCAGCAGCACCGACATCTGCAGGCCCCACCAGA
TCTGCAACGTGGTGGCCATCCCCGGCAACGCCAGCATGGACGCCGTGTGCACCA
GCACCAGCCCCACCAGGAGCATGGCCCCCGGCGCCGTGCACCTGCCCCAGCCC
GTGAGCACCAGGAGCCAGCACACCCAGCCCACCCCCGAGCCCAGCACCGCCCC
CAGCACCAGCTTCCTGCTGCCCATGGGCCCCAGCCCCCCCGCCGAGGGCAGCA
CCGGCGACCCACGGGGACCAACCATCAAACCATGCCCCCCCTGTAAGTGCCCCG
CACCTAACCTCCTGGGTGGGCCATCCGTGTTTATCTTCCCCCCTAAAATTAAGGAT
GTGCTGATGATCAGCCTGAGCCCTATCGTGACATGCGTGGTCGTCGACGTGAGC
GAGGATGACCCCGACGTCCAGATCTCTTGGTTTGTGAACAACGTGGAGGTTCACA
CCGCCCAAACCCAGACACACAGAGAGGACTATAATTCTACTCTCCGCGTTGTGTC
CGCCCTGCCCATCCAGCACCAAGATTGGATGTCTGGTAAGGAATTCAAGTGTAAA
GTGAACAATAAAGATCTGCCCGCACCAATCGAAAGAACCATTTCTAAGCCTAAAG
GCTCCGTGCGGGCTCCTCAGGTTTACGTCCTGCCACCCCCCGAGGAAGAGATGA
CTAAGAAGCAGGTGACCCTGACCTGTATGGTGACCGATTTTATGCCCGAAGACAT
TTACGTCGAGTGGACCAATAATGGTAAGACAGAACTGAACTATAAAAATACAGAGC
CAGTGCTCGACTCTGATGGCAGCTATTTTATGTACTCTAAGCTGAGAGTTGAGAAG
AAAAACTGGGTGGAACGGAATTCTTACTCTTGCTCTGTCGTGCACGAAGGACTGC
ACAACCACCACACTACCAAGTCCTTTTCTAGGACCCCCGGCAAGGAGACCTGCGA
GAACGTGGACTGCGGCCCCGGCAAGAAGTGCCGGATGAACAAGAAGAACAAGCC
CCGGTGCGTGTGCGCCCCCGACTGCAGCAACATCACCTGGAAGGGCCCCGTGT
GCGGCCTGGACGGCAAGACCTACCGGAACGAGTGCGCCCTGCTGAAGGCCCGG
TGCAAGGAGCAGCCCGAGCTGGAGGTGCAGTACCAGGGCCGGTGCAAG
SEQ ID NO: 11
FST291-mFc knobs-in-holes A chain (used together with either
VGFR(1/2) B chain, TNFR2 B chain, or CBG B chain).
Serum albumin preproprotein signal peptide in bold, murine
IgG2A A chain underlined.
MKWVTFISLLFLFSSAYSGNCWLRQAKNGRCQVLYKTELSKEECCSTGRLSTSWTE
EDVNDNTLFKWMIFNGGAPNCIPCKETCENVDCGPGKKCRMNKKNKPRCVCAPDCS
NITWKGPVCGLDGKTYRNECALLKARCKEQPELEVQYQGRCKKTCRDVFCPGSSTC
VVDQTNNAYCVTCNRICPEPASSEQYLCGNDGVTYSSACHLRKATCLLGRSIGLAYE
GKCIKAKSCEDIQCTGGKKCLWDFKVGRGRCSLCDELCPDSKSDEPVCASDNATYAS
ECAMKEAACSSGVLLEVKHSGSCNSISPRGPTIKPCPPCKCPAPNLLGGPSVFIFPPKI
KDVLMISLSPIVTCVVVDVSEDDPDVQISWFVNNVEVHTAQTQTHREDYNSTLRVVSA
LPIQHQDWMSGKEFKCKVNNKDLPAPIERTISKPKGSVRAPQVYVLPPPEEEMTEKQ
VTLTCMVTDFMPEDIYVEWTNNGKTELNYKNTEPVLDSDGSYFMYSWLRVEKKNWV
ERNSYSCSVVHEGLHNHHTTKSFSRTPGK
SEQ ID NO: 12
Polynucleotide encoding FST291-mFc knobs-in-holes A chain
(used together with either VGFR(1/2) B chain,
TNFR2 B chain, or CBG B chain).
ATGAAGTGGGTGACCTTCATCTCTCTGCTGTTCCTGTTCTCTTCTGCCTACTCTGG
CAACTGCTGGCTGCGGCAGGCCAAGAACGGCCGGTGCCAGGTGCTGTACAAGAC
CGAGCTGAGCAAGGAGGAGTGCTGCAGCACCGGCCGGCTGAGCACCAGCTGGA
CCGAGGAGGACGTGAACGACAACACCCTGTTCAAGTGGATGATCTTCAACGGCG
GCGCCCCCAACTGCATCCCCTGCAAGGAGACCTGCGAGAACGTGGACTGCGGCC
CCGGCAAGAAGTGCCGGATGAACAAGAAGAACAAGCCCCGGTGCGTGTGCGCCC
CCGACTGCAGCAACATCACCTGGAAGGGCCCCGTGTGCGGCCTGGACGGCAAG
ACCTACCGGAACGAGTGCGCCCTGCTGAAGGCCCGGTGCAAGGAGCAGCCCGA
GCTGGAGGTGCAGTACCAGGGCCGGTGCAAGAAGACCTGCCGGGACGTGTTCT
GCCCCGGCAGCAGCACCTGCGTGGTGGACCAGACCAACAACGCCTACTGCGTGA
CCTGCAACCGGATCTGCCCCGAGCCCGCCAGCAGCGAGCAGTACCTGTGCGGC
AACGACGGCGTGACCTACAGCAGCGCCTGCCACCTGCGGAAGGCCACCTGCCTG
CTGGGCCGGAGCATCGGCCTGGCCTACGAGGGCAAGTGCATCAAGGCCAAGAG
CTGCGAGGACATCCAGTGCACCGGCGGCAAGAAGTGCCTGTGGGACTTCAAGGT
GGGCCGGGGCCGGTGCAGCCTGTGCGACGAGCTGTGCCCCGACAGCAAGAGCG
ACGAGCCCGTGTGCGCCAGCGACAACGCCACCTACGCCAGCGAGTGCGCCATG
AAGGAGGCCGCCTGCAGCAGCGGCGTGCTGCTGGAGGTGAAGCACAGCGGCAG
CTGCAACAGCATCAGCCCACGGGGACCAACCATCAAACCATGCCCCCCCTGTAA
GTGCCCCGCACCTAACCTCCTGGGTGGGCCATCCGTGTTTATCTTCCCCCCTAAA
ATTAAGGATGTGCTGATGATCAGCCTGAGCCCTATCGTGACATGCGTGGTCGTCG
ACGTGAGCGAGGATGACCCCGACGTCCAGATCTCTTGGTTTGTGAACAACGTGGA
GGTTCACACCGCCCAAACCCAGACACACAGAGAGGACTATAATTCTACTCTCCGC
GTTGTGTCCGCCCTGCCCATCCAGCACCAAGATTGGATGTCTGGTAAGGAATTCA
AGTGTAAAGTGAACAATAAAGATCTGCCCGCACCAATCGAAAGAACCATTTCTAAG
CCTAAAGGCTCCGTGCGGGCTCCTCAGGTTTACGTCCTGCCACCCCCCGAGGAA
GAGATGACTGAGAAGCAGGTGACCCTGACCTGTATGGTGACCGATTTTATGCCCG
AAGACATTTACGTCGAGTGGACCAATAATGGTAAGACAGAACTGAACTATAAAAAT
ACAGAGCCAGTGCTCGACTCTGATGGCAGCTATTTTATGTACTCTTGGCTGAGAG
TTGAGAAGAAAAACTGGGTGGAACGGAATTCTTACTCTTGCTCTGTCGTGCACGA
AGGACTGCACAACCACCACACTACCAAGTCCTTTTCTAGGACCCCCGGCAAG
SEQ ID NO: 13
VGFR(1/2)-mFc knobs-in-holes B chain. Serum albumin
preproprotein signal peptide in bold, murine IgG2A B
chain underlined. C-terminal thrombin site and
his tag double underlined.
MKWVTFISLLFLFSSAYSSDTGRPFVEMYSEIPEIIHMTEGRELVIPCRVTSPNITVTLK
KFPLDTLIPDGKRIIWDSRKGFIISNATYKEIGLLTCEATVNGHLYKTNYLTHRQTNTIIDV
VLSPSHGIELSVGEKLVLNCTARTELNVGIDFNWEYPSSKHQHKKLVNRDLKTQSGSE
MKKFLSTLTIDGVTRSDQGLYTCAASSGLMTKKNSTFVRVHEKPRGPTIKPCPPCKCP
APNLLGGPSVFIFPPKIKDVLMISLSPIVTCVVVDVSEDDPDVQISWFVNNVEVHTAQT
QTHREDYNSTLRVVSALPIQHQDWMSGKEFKCKVNNKDLPAPIERTISKPKGSVRAP
RVYVLPPPEEEMTKKQVTLTCMVTDFMPEDIYVEWTNNGKTELNYKNTEPVLVSDGS
YTMYSKLRVEKKNWVERNSYSCSVVHEGLHNHHTTKSFSRTPGKLVPRGSHHHHHH
SEQ ID NO: 14
Polynucleotide encoding VGFR(1/2)-mFc knobs-in-holes B chain.
ATGAAGTGGGTGACCTTCATCTCTCTGCTGTTCCTGTTCTCTTCTGCCTACTCTAG
CGACACCGGCAGGCCCTTCGTGGAGATGTACAGCGAGATCCCCGAGATCATCCA
CATGACCGAGGGCAGGGAGCTGGTGATCCCCTGCAGGGTGACCAGCCCCAACAT
CACCGTGACCCTGAAGAAGTTCCCCCTGGACACCCTGATCCCCGACGGCAAGAG
GATCATCTGGGACAGCAGGAAGGGCTTCATCATCAGCAACGCCACCTACAAGGA
GATCGGCCTGCTGACCTGCGAGGCCACCGTGAACGGCCACCTGTACAAGACCAA
CTACCTGACCCACAGGCAGACCAACACCATCATCGACGTGGTGCTGAGCCCCAG
CCACGGCATCGAGCTGAGCGTGGGCGAGAAGCTGGTGCTGAACTGCACCGCCA
GGACCGAGCTGAACGTGGGCATCGACTTCAACTGGGAGTACCCCAGCAGCAAGC
ACCAGCACAAGAAGCTGGTGAACAGGGACCTGAAGACCCAGAGCGGCAGCGAGA
TGAAGAAGTTCCTGAGCACCCTGACCATCGACGGCGTGACCAGGAGCGACCAGG
GCCTGTACACCTGCGCCGCCAGCAGCGGCCTGATGACCAAGAAGAACAGCACCT
TCGTGAGGGTGCACGAGAAGCCACGGGGACCAACCATCAAACCATGCCCCCCCT
GTAAGTGCCCCGCACCTAACCTCCTGGGTGGGCCATCCGTGTTTATCTTCCCCCC
TAAAATTAAGGATGTGCTGATGATCAGCCTGAGCCCTATCGTGACATGCGTGGTC
GTCGACGTGAGCGAGGATGACCCCGACGTCCAGATCTCTTGGTTTGTGAACAAC
GTGGAGGTTCACACCGCCCAAACCCAGACACACAGAGAGGACTATAATTCTACTC
TCCGCGTTGTGTCCGCCCTGCCCATCCAGCACCAAGATTGGATGTCTGGTAAGGA
ATTCAAGTGTAAAGTGAACAATAAAGATCTGCCCGCACCAATCGAAAGAACCATTT
CTAAGCCTAAAGGCTCCGTGCGGGCTCCTAGAGTTTACGTCCTGCCACCCCCCG
AGGAAGAGATGACTAAGAAGCAGGTGACCCTGACCTGTATGGTGACCGATTTTAT
GCCCGAAGACATTTACGTCGAGTGGACCAATAATGGTAAGACAGAACTGAACTAT
AAAAATACAGAGCCAGTGCTCGTGTCTGATGGCAGCTATACCATGTACTCTAAGCT
GAGAGTTGAGAAGAAAAACTGGGTGGAACGGAATTCTTACTCTTGCTCTGTCGTG
CACGAAGGACTGCACAACCACCACACTACCAAGTCCTTTTCTAGGACCCCCGGCA
AGCTGGTGCCCAGAGGAAGCCATCATCATCATCATCAC
SEQ ID NO: 15
TNFR2-mFc knobs-in-holes B chain. Serum albumin preproprotein
signal peptide in bold, murine IgG2A B chain underlined.
C-terminal thrombin site and his tag double underlined.
MKWVTFISLLFLFSSAYSLPAQVAFTPYAPEPGSTCRLREYYDQTAQMCCSKCSPGQ
HAKVFCTKTSDTVCDSCEDSTYTQLWNWVPECLSCGSRCSSDQVETQACTREQNRI
CTCRPGWYCALSKQEGCRLCAPLRKCRPGFGVARPGTETSDVVCKPCAPGTFSNTT
SSTDICRPHQICNVVAIPGNASMDAVCTSTSPTRSMAPGAVHLPQPVSTRSQHTQPT
PEPSTAPSTSFLLPMGPSPPAEGSTGDPRGPTIKPCPPCKCPAPNLLGGPSVFIFPPKI
KDVLMISLSPIVTCVVVDVSEDDPDVQISWFVNNVEVHTAQTQTHREDYNSTLRVVSA
LPIQHQDWMSGKEFKCKVNNKDLPAPIERTISKPKGSVRAPRVYVLPPPEEEMTKKQ
VTLTCMVTDFMPEDIYVEWTNNGKTELNYKNTEPVLVSDGSYTMYSKLRVEKKNWVE
RNSYSCSVVHEGLHNHHTTKSFSRTPGKLVPRGSHHHHHH
SEQ ID NO: 16
Polynucleotide encoding TNFR2-mFc knobs-in-holes B chain.
ATGAAGTGGGTGACCTTCATCTCTCTGCTGTTCCTGTTCTCTTCTGCCTACTCTCT
GCCCGCCCAGGTGGCCTTCACCCCCTACGCCCCCGAGCCCGGCAGCACCTGCA
GGCTGAGGGAGTACTACGACCAGACCGCCCAGATGTGCTGCAGCAAGTGCAGCC
CCGGCCAGCACGCCAAGGTGTTCTGCACCAAGACCAGCGACACCGTGTGCGACA
GCTGCGAGGACAGCACCTACACCCAGCTGTGGAACTGGGTGCCCGAGTGCCTGA
GCTGCGGCAGCAGGTGCAGCAGCGACCAGGTGGAGACCCAGGCCTGCACCAGG
GAGCAGAACAGGATCTGCACCTGCAGGCCCGGCTGGTACTGCGCCCTGAGCAAG
CAGGAGGGCTGCAGGCTGTGCGCCCCCCTGAGGAAGTGCAGGCCCGGCTTCGG
CGTGGCCAGGCCCGGCACCGAGACCAGCGACGTGGTGTGCAAGCCCTGCGCCC
CCGGCACCTTCAGCAACACCACCAGCAGCACCGACATCTGCAGGCCCCACCAGA
TCTGCAACGTGGTGGCCATCCCCGGCAACGCCAGCATGGACGCCGTGTGCACCA
GCACCAGCCCCACCAGGAGCATGGCCCCCGGCGCCGTGCACCTGCCCCAGCCC
GTGAGCACCAGGAGCCAGCACACCCAGCCCACCCCCGAGCCCAGCACCGCCCC
CAGCACCAGCTTCCTGCTGCCCATGGGCCCCAGCCCCCCCGCCGAGGGCAGCA
CCGGCGACCCACGGGGACCAACCATCAAACCATGCCCCCCCTGTAAGTGCCCCG
CACCTAACCTCCTGGGTGGGCCATCCGTGTTTATCTTCCCCCCTAAAATTAAGGAT
GTGCTGATGATCAGCCTGAGCCCTATCGTGACATGCGTGGTCGTCGACGTGAGC
GAGGATGACCCCGACGTCCAGATCTCTTGGTTTGTGAACAACGTGGAGGTTCACA
CCGCCCAAACCCAGACACACAGAGAGGACTATAATTCTACTCTCCGCGTTGTGTC
CGCCCTGCCCATCCAGCACCAAGATTGGATGTCTGGTAAGGAATTCAAGTGTAAA
GTGAACAATAAAGATCTGCCCGCACCAATCGAAAGAACCATTTCTAAGCCTAAAG
GCTCCGTGCGGGCTCCTAGAGTTTACGTCCTGCCACCCCCCGAGGAAGAGATGA
CTAAGAAGCAGGTGACCCTGACCTGTATGGTGACCGATTTTATGCCCGAAGACAT
TTACGTCGAGTGGACCAATAATGGTAAGACAGAACTGAACTATAAAAATACAGAGC
CAGTGCTCGTGTCTGATGGCAGCTATACCATGTACTCTAAGCTGAGAGTTGAGAA
GAAAAACTGGGTGGAACGGAATTCTTACTCTTGCTCTGTCGTGCACGAAGGACTG
CACAACCACCACACTACCAAGTCCTTTTCTAGGACCCCCGGCAAGCTGGTGCCCA
GAGGAAGCCATCATCATCATCATCAC
SEQ ID NO: 17
CBG-mFc knobs-in-holes B chain. Serum albumin preproprotein signal
peptide in bold, murine IgG2A B chain underlined.
C-terminal thrombin site and his tag double underlined.
MKWVTFISLLFLFSSAYSMDPNAAYVNMSNHHRGLASANVDFAFSLYKHLVALSPKK
NIFISPVSISMALAMLSLGTCGHTRAQLLQGLGFNLTERSETEIHQGFQHLHQLFAKSD
TSLEMTMGNALFLDGSLELLESFSADIKHYYESEVLAMNFQDWATASRQINSYVKNKT
QGKIVDLFSGLDSPAILVLVNYIFFKGTWTQPFDLASTREENFYVDETTVVKVPMMLQS
STISYLHDSELPCQLVQMNYVGNGTVFFILPDKGKMNTVIAALSRDTINRWSAGLTSS
QVDLYIPKVTISGVYDLGDVLEEMGIADLFTNQANFSRITQDAQLKSSKVVHKAVLQLN
EEGVDTAGSTGVTLNLTSKPIILRFNQPFIIMIFDHFTWSSLFLARVMNPVPRGPTIKPC
PPCKCPAPNLLGGPSVFIFPPKIKDVLMISLSPIVTCVVVDVSEDDPDVQISWFVNNVE
VHTAQTQTHREDYNSTLRVVSALPIQHQDWMSGKEFKCKVNNKDLPAPIERTISKPK
GSVRAPRVYVLPPPEEEMTKKQVTLTCMVTDFMPEDIYVEWTNNGKTELNYKNTEPV
LVSDGSYTMYSKLRVEKKNWVERNSYSCSVVHEGLHNHHTTKSFSRTPGK
LVPRGSHHHHHH
SEQ ID NO: 18
Polynucleotide encoding CBG-mFc knobs-in-holes B chain.
ATGAAGTGGGTGACCTTCATCTCTCTGCTGTTCCTGTTCTCTTCTGCCTACTCTAT
GGACCCCAACGCCGCCTACGTGAACATGTCTAACCACCACAGGGGCCTGGCCTC
TGCCAACGTGGACTTCGCCTTCTCTCTGTACAAGCACCTGGTGGCCCTGTCTCCC
AAGAAGAACATCTTCATCTCTCCCGTGTCTATCTCTATGGCCCTGGCCATGCTGTC
TCTGGGCACCTGCGGCCACACCAGGGCCCAGCTGCTGCAGGGCCTGGGCTTCA
ACCTGACCGAGAGGTCTGAGACCGAGATCCACCAGGGCTTCCAGCACCTGCACC
AGCTGTTCGCCAAGTCTGACACCTCTCTGGAGATGACCATGGGCAACGCCCTGTT
CCTGGACGGCTCTCTGGAGCTGCTGGAGTCTTTCTCTGCCGACATCAAGCACTAC
TACGAGTCTGAGGTGCTGGCCATGAACTTCCAGGACTGGGCCACCGCCTCTAGG
CAGATCAACTCTTACGTGAAGAACAAGACCCAGGGCAAGATCGTGGACCTGTTCT
CTGGCCTGGACTCTCCCGCCATCCTGGTGCTGGTGAACTACATCTTCTTCAAGGG
CACCTGGACCCAGCCCTTCGACCTGGCCTCTACCAGGGAGGAGAACTTCTACGT
GGACGAGACCACCGTGGTGAAGGTGCCCATGATGCTGCAGTCTTCTACCATCTCT
TACCTGCACGACTCTGAGCTGCCCTGCCAGCTGGTGCAGATGAACTACGTGGGC
AACGGCACCGTGTTCTTCATCCTGCCCGACAAGGGCAAGATGAACACCGTGATCG
CCGCCCTGTCTAGGGACACCATCAACAGGTGGTCTGCCGGCCTGACCTCTTCTCA
GGTGGACCTGTACATCCCCAAGGTGACCATCTCTGGCGTGTACGACCTGGGCGA
CGTGCTGGAGGAGATGGGCATCGCCGACCTGTTCACCAACCAGGCCAACTTCTC
TAGGATCACCCAGGACGCCCAGCTGAAGTCTTCTAAGGTGGTGCACAAGGCCGT
GCTGCAGCTGAACGAGGAGGGCGTGGACACCGCCGGCTCTACCGGCGTGACCC
TGAACCTGACCTCTAAGCCCATCATCCTGAGGTTCAACCAGCCCTTCATCATCATG
ATCTTCGACCACTTCACCTGGTCTTCTCTGTTCCTGGCCAGGGTGATGAACCCCG
TGCCACGGGGACCAACCATCAAACCATGCCCCCCCTGTAAGTGCCCCGCACCTA
ACCTCCTGGGTGGGCCATCCGTGTTTATCTTCCCCCCTAAAATTAAGGATGTGCT
GATGATCAGCCTGAGCCCTATCGTGACATGCGTGGTCGTCGACGTGAGCGAGGA
TGACCCCGACGTCCAGATCTCTTGGTTTGTGAACAACGTGGAGGTTCACACCGCC
CAAACCCAGACACACAGAGAGGACTATAATTCTACTCTCCGCGTTGTGTCCGCCC
TGCCCATCCAGCACCAAGATTGGATGTCTGGTAAGGAATTCAAGTGTAAAGTGAA
CAATAAAGATCTGCCCGCACCAATCGAAAGAACCATTTCTAAGCCTAAAGGCTCC
GTGCGGGCTCCTAGAGTTTACGTCCTGCCACCCCCCGAGGAAGAGATGACTAAG
AAGCAGGTGACCCTGACCTGTATGGTGACCGATTTTATGCCCGAAGACATTTACG
TCGAGTGGACCAATAATGGTAAGACAGAACTGAACTATAAAAATACAGAGCCAGT
GCTCGTGTCTGATGGCAGCTATACCATGTACTCTAAGCTGAGAGTTGAGAAGAAA
AACTGGGTGGAACGGAATTCTTACTCTTGCTCTGTCGTGCACGAAGGACTGCACA
ACCACCACACTACCAAGTCCTTTTCTAGGACCCCCGGCAAGCTGGTGCCCAGAG
GAAGCCATCATCATCATCATCAC
SEQ ID NO: 19:
Part of the extracellular domain of native human
VGFR1 that binds VEGF (immunoglobulin like type 2)
SDTGRPFVEMYSEIPEIIHMTEGRELVIPCRVTSPNITVTLKKFPLDTLIPDGKRIIWDSR
KGFIISNATYKEIGLLTCEATVNGHLYKTNYLTHRQTNTII
SEQ ID NO: 20: Part of the extracellular domain of
native human VGFR2 that binds
VEGF (immunoglobulin like type 3)
DVVLSPSHGIELSVGEKLVLNCTARTELNVGIDFNWEYPSSKHQHKKLVNRDLKTQSG
SEMKKFLSTLTIDGVTRSDQGLYTCAASSGLMTKKNSTFVRVHEK
SEQ ID NO: 21: Native human TNFR2 ectodomain (TNFa binding domain)
LPAQVAFTPYAPEPGSTCRLREYYDQTAQMCCSKCSPGQHAKVFCTKTSDTVCDSC
EDSTYTQLWNWVPECLSCGSRCSSDQVETQACTREQNRICTCRPGWYCALSKQEG
CRLCAPLRKCRPGFGVARPGTETSDVVCKPCAPGTFSNTTSSTDICRPHQICNVVAIP
GNASMDAVCTSTSPTRSMAPGAVHLPQPVSTRSQHTQPTPEPSTAPSTSFLLPMGP
SPPAEGSTGD
SEQ ID NO: 22: Native human CBG. Native signal peptide in bold.
MPLLLYTCLLWLPTSGLWTVQAMDPNAAYVNMSNHHRGLASANVDFAFSLYKHLVA
LSPKKNIFISPVSISMALAMLSLGTCGHTRAQLLQGLGFNLTERSETEIHQGFQHLHQL
FAKSDTSLEMTMGNALFLDGSLELLESFSADIKHYYESEVLAMNFQDWATASRQINSY
VKNKTQGKIVDLFSGLDSPAILVLVNYIFFKGTWTQPFDLASTREENFYVDETTVVKVP
MMLQSSTISYLHDSELPCQLVQMNYVGNGTVFFILPDKGKMNTVIAALSRDTINRWSA
GLTSSQVDLYIPKVTISGVYDLGDVLEEMGIADLFTNQANFSRITQDAQLKSSKVVHKA
VLQLNEEGVDTAGSTGVTLNLTSKPIILRFNQPFIIMIFDHFTWSSLFLARVMNPV
SEQ ID NO: 23: Human FSD1(KTC)
ETCENVDCGPGKKCRMNKKNKPRCVCAPDCSNITWKGPVCGLDGKTYRNECALLKA
RCKEQPELEVQYQGRCKKTC
SEQ ID NO: 24: Human FSD1(KTC) as a single entity
produced in E coli contains an
initiator methionine (M) amino acid.
METCENVDCGPGKKCRMNKKNKPRCVCAPDCSNITWKGPVCGLDGKTYRNECALL
KARCKEQPELEVQYQGRCKKTC
SEQ ID NO: 25
Polynucleotide encoding Human FSD1(KTC) as a single entity
produced in E coli containing an initiator methionine
(M) amino acid:
ATGGAGACATGCGAGAACGTGGATTGTGGACCAGGCAAGAAGTGCCGGATGAAC
AAGAAGAACAAGCCCAGATGCGTGTGCGCTCCTGACTGCAGCAACATCACCTGG
AAGGGACCCGTGTGCGGCCTGGATGGCAAGACATACCGGAATGAGTGCGCCCTG
CTGAAGGCTAGGTGTAAGGAGCAGCCTGAGCTGGAGGTGCAGTATCAGGGCCGG
TGCAAGAAAACATGT
SEQ ID NO: 26
Native human follistatin 291. Native signal peptide in bold.
MVRARHQPGGLCLLLLLLCQFMEDRSAQAGNCWLRQAKNGRCQVLYKTELSKEEC
CSTGRLSTSWTEEDVNDNTLFKWMIFNGGAPNCIPCKETCENVDCGPGKKCRMNKK
NKPRCVCAPDCSNITWKGPVCGLDGKTYRNECALLKARCKEQPELEVQYQGRCKKT
CRDVFCPGSSTCVVDQTNNAYCVTCNRICPEPASSEQYLCGNDGVTYSSACHLRKA
TCLLGRSIGLAYEGKCIKAKSCEDIQCTGGKKCLWDFKVGRGRCSLCDELCPDSKSD
EPVCASDNATYASECAMKEAACSSGVLLEVKHSGSCNSIS
SEQ ID NO: 27
Native human follistatin 288. Native signal peptide in bold.
MVRARHQPGGLCLLLLLLCQFMEDRSAQAGNCWLRQAKNGRCQVLYKTELSKEEC
CSTGRLSTSWTEEDVNDNTLFKWMIFNGGAPNCIPCKETCENVDCGPGKKCRMNKK
NKPRCVCAPDCSNITWKGPVCGLDGKTYRNECALLKARCKEQPELEVQYQGRCKKT
CRDVFCPGSSTCVVDQTNNAYCVTCNRICPEPASSEQYLCGNDGVTYSSACHLRKA
TCLLGRSIGLAYEGKCIKAKSCEDIQCTGGKKCLWDFKVGRGRCSLCDELCPDSKSD
EPVCASDNATYASECAMKEAACSSGVLLEVKHSGSCN
SEQ ID NO: 28
Native human follistatin 315. Native signal peptide in bold.
MVRARHQPGGLCLLLLLLCQFMEDRSAQAGNCWLRQAKNGRCQVLYKTELSKEEC
CSTGRLSTSWTEEDVNDNTLFKWMIFNGGAPNCIPCKETCENVDCGPGKKCRMNKK
NKPRCVCAPDCSNITWKGPVCGLDGKTYRNECALLKARCKEQPELEVQYQGRCKKT
CRDVFCPGSSTCVVDQTNNAYCVTCNRICPEPASSEQYLCGNDGVTYSSACHLRKA
TCLLGRSIGLAYEGKCIKAKSCEDIQCTGGKKCLWDFKVGRGRCSLCDELCPDSKSD
EPVCASDNATYASECAMKEAACSSGVLLEVKHSGSCNSISEDTEEEEEDEDQDYSFP
ISSILEW
SEQ ID NO: 29
Human Follistatin 315 wherein the heparin binding
sequence (HBS) of FSD1 has been replaced by a
tructurally related sequence. Native signal peptide
in bold.
MVRARHQPGGLCLLLLLLCQFMEDRSAQAGNCWLRQAKNGRCQVLYKTELSKEEC
CSTGRLSTSWTEEDVNDNTLFKWMIFNGGAPNCIPCKETCENVDCGPGSTCVVDQT
NNPRCVCAPDCSNITWKGPVCGLDGKTYRNECALLKARCKEQPELEVQYQGRCKKT
CRDVFCPGSSTCVVDQTNNAYCVTCNRICPEPASSEQYLCGNDGVTYSSACHLRKA
TCLLGRSIGLAYEGKCIKAKSCEDIQCTGGKKCLWDFKVGRGRCSLCDELCPDSKSD
EPVCASDNATYASECAMKEAACSSGVLLEVKHSGSCNSISEDTEEEEEDEDQDYSFP
ISSILEW
SEQ ID NO: 30
Human Follistatin 315 fused to a murine IgG2A Fc fragment
(underlined), wherein the heparin binding sequence (HBS)
of FSD1 has been replaced by a structurally related sequence.
The protein encoded by this sequence is referred to
as FST315dHBS-mFc. Native signal peptide in bold.
MVRARHQPGGLCLLLLLLCQFMEDRSAQAGNCWLRQAKNGRCQVLYKTELSKEEC
CSTGRLSTSWTEEDVNDNTLFKWMIFNGGAPNCIPCKETCENVDCGPGSTCVVDQT
NNPRCVCAPDCSNITWKGPVCGLDGKTYRNECALLKARCKEQPELEVQYQGRCKKT
CRDVFCPGSSTCVVDQTNNAYCVTCNRICPEPASSEQYLCGNDGVTYSSACHLRKA
TCLLGRSIGLAYEGKCIKAKSCEDIQCTGGKKCLWDFKVGRGRCSLCDELCPDSKSD
EPVCASDNATYASECAMKEAACSSGVLLEVKHSGSCNSISEDTEEEEEDEDQDYSFP
ISSILEWPRGPTIKPCPPCKCPAPNLLGGPSVFIFPPKIKDVLMISLSPIVTCVVVDVSED
DPDVQISWFVNNVEVHTAQTQTHREDYNSTLRVVSALPIQHQDWMSGKEFKCKVNN
KDLPAPIERTISKPKGSVRAPQVYVLPPPEEEMTKKQVTLTCMVTDFMPEDIYVEWTN
NGKTELNYKNTEPVLDSDGSYFMYSKLRVEKKNWVERNSYSCSVVHEGLHNHHTTK
SFSRTPGK
SEQ ID NO: 31
Polynucleotide sequence encoding Human Follistatin 315
fused to a murine IgG2A Fc fragment, wherein the
heparin binding sequence (HBS) of FSD1 has
been replaced by a structurally related sequence.
ATGGTTAGAGCCAGACATCAGCCTGGGGGCCTGTGCCTGCTGCTCCTTCTTCTGT
GTCAGTTCATGGAGGACCGCAGTGCTCAGGCCGGTAACTGTTGGTTGAGACAGG
CTAAGAACGGTCGGTGCCAGGTGCTCTACAAAACAGAGCTCTCAAAGGAGGAATG
CTGCTCCACTGGACGTCTAAGCACGTCTTGGACTGAGGAGGATGTCAACGACAAT
ACATTGTTTAAATGGATGATATTTAACGGCGGAGCACCCAACTGCATACCTTGCAA
GGAGACGTGTGAGAATGTTGACTGTGGCCCAGGTTCAACGTGCGTGGTGGATCA
GACCAATAATCCTAGGTGCGTGTGCGCCCCCGACTGCTCCAACATTACATGGAAG
GGCCCTGTGTGTGGGCTGGATGGTAAAACATATAGAAACGAATGTGCTTTGTTGA
AAGCTCGTTGTAAAGAGCAGCCGGAGCTGGAAGTCCAATACCAGGGCCGATGCA
AAAAGACTTGTAGAGATGTCTTCTGTCCAGGTAGCAGCACCTGTGTTGTCGACCA
GACTAATAACGCTTACTGTGTGACCTGCAATAGGATCTGTCCGGAACCTGCATCA
AGCGAGCAATACCTTTGTGGCAATGACGGAGTCACTTACTCAAGCGCATGCCACC
TGAGGAAGGCAACATGCCTCCTGGGCAGGAGCATAGGACTCGCCTATGAAGGAA
AGTGCATTAAGGCAAAATCTTGTGAGGACATCCAATGCACTGGCGGGAAGAAGTG
TCTTTGGGACTTTAAAGTGGGGCGCGGGAGATGCTCCCTCTGCGATGAACTGTGC
CCCGATTCCAAAAGTGACGAACCTGTCTGCGCCTCTGATAACGCTACCTATGCAT
CTGAGTGCGCAATGAAGGAAGCCGCATGCAGTAGCGGAGTATTGCTTGAGGTGA
AGCACTCAGGCTCCTGTAATTCCATATCAGAGGATACCGAGGAGGAGGAGGAGG
ACGAGGATCAGGATTATTCATTTCCGATTTCATCAATCTTAGAGTGGCCGCGCGG
GCCAACAATCAAGCCATGCCCACCCTGTAAATGCCCCGCCCCAAACCTGCTGGG
AGGCCCTAGTGTGTTTATCTTCCCGCCCAAAATCAAGGATGTGCTCATGATCTCTC
TAAGCCCAATCGTGACATGCGTGGTCGTCGACGTCTCCGAGGACGACCCCGACG
TGCAAATTTCCTGGTTCGTGAACAATGTCGAGGTCCACACCGCTCAGACCCAAAC
CCATCGCGAGGATTACAATAGTACTCTGCGGGTGGTTTCAGCCCTGCCTATCCAG
CACCAGGATTGGATGAGTGGCAAGGAGTTTAAGTGTAAGGTGAATAACAAGGACC
TGCCAGCTCCTATCGAAAGGACAATATCTAAGCCTAAGGGGTCTGTGCGGGCACC
TCAGGTCTATGTCCTGCCACCCCCAGAAGAGGAAATGACAAAGAAACAGGTGACT
CTGACATGTATGGTTACCGACTTCATGCCTGAGGACATATATGTCGAGTGGACCA
ATAATGGAAAGACTGAGCTAAACTACAAAAATACCGAACCAGTTTTAGATTCCGAC
GGCAGTTACTTCATGTACTCAAAGCTGCGGGTGGAGAAAAAGAACTGGGTGGAGA
GAAACTCATACAGTTGCAGTGTGGTGCACGAGGGGTTGCATAATCACCACACCAC
CAAATCCTTCTCCAGAACTCCAGGAAAA
SEQ ID NO 32: Human FSD1 as a single entity produced
in E coli contains an initiator methionine (M) amino acid.
METCENVDCGPGKKCRMNKKNKPRCVCAPDCSNITWKGPVCGLDGKTYRNECALL
KARCKEQPELEVQYQGRCK
SEQ ID NO: 33
Polynucleotide encoding Human FSD1 as a single
entity produced in E coli containing an initiator
methionine (M) amino acid
ATGGAGACATGCGAGAACGTGGATTGTGGACCAGGCAAGAAGTGCCGGATGAAC
AAGAAGAACAAGCCCAGATGCGTGTGCGCTCCTGACTGCAGCAACATCACCTGG
AAGGGACCCGTGTGCGGCCTGGATGGCAAGACATACCGGAATGAGTGCGCCCTG
CTGAAGGCTAGGTGTAAGGAGCAGCCTGAGCTGGAGGTGCAGTATCAGGGCCGG
TGCAAG
SEQ ID NO: 34
anti-vimentin-nanobody. Expressed in E. coli and
therefore contains an initiator methionine (M)
amino acid. C-terminal thrombin site and his tag
double underlined
MQVQLVESGGGLVQSGGSLTLTCAASGFTFSAASMRWVRQVPGKGLEWVATIDGT
GANSYYSESAKGRFTISRDNARNTLYLQMNNLKPDDTAVYYCANFGRNYWGKGTQV
TVSSLVPRGSHHHHHH
SEQ ID NO: 35
Polynucleotide encoding anti-vimentin-nanobody. Expressed in E. coli
and therefore containing an initiator methionine (M) amino acid
ATGCAAGTACAGCTAGTTGAATCAGGTGGAGGGTTGGTGCAAAGCGGTGGTTCTC
TGACCTTGACGTGCGCGGCGTCTGGCTTCACCTTCAGCGCTGCCTCCATGCGTT
GGGTACGTCAGGTGCCGGGTAAGGGCCTTGAGTGGGTCGCAACCATCGACGGTA
CTGGTGCAAACAGCTATTACAGCGAGTCGGCGAAAGGCCGTTTTACGATCAGCCG
TGATAATGCGCGTAACACCTTATATCTGCAGATGAATAACCTGAAACCGGATGACA
CCGCAGTTTACTACTGCGCGAACTTTGGTCGCAACTATTGGGGTAAGGGCACGCA
AGTGACCGTTTCCTCCCTAGTACCCAGGGGTAGCCACCACCACCACCACCAC
SEQ ID NO: 36: Heparin binding domain of human FSD1
KKCRMNKKNKPR
SEQ ID NO: 37: Murine IgG2A Fc
PRGPTIKPCPPCKCPAPNLLGGPSVFIFPPKIKDVLMISLSPIVTCVVVDVSEDDPDVQI
SWFVNNVEVHTAQTQTHREDYNSTLRVVSALPIQHQDWMSGKEFKCKVNNKDLPAPI
ERTISKPKGSVRAPQVYVLPPPEEEMTKKQVTLTCMVTDFMPEDIYVEWTNNGKTEL
NYKNTEPVLDSDGSYFMYSKLRVEKKNWVERNSYSCSVVHEGLHNHHTTKSFSRTP
GK
SEQ ID NO: 38
Murine IgG2A Fc knobs-in-holes A chain
PRGPTIKPCPPCKCPAPNLLGGPSVFIFPPKIKDVLMISLSPIVTCVVVDVSEDDPDVQI
SWFVNNVEVHTAQTQTHREDYNSTLRVVSALPIQHQDWMSGKEFKCKVNNKDLPAPI
ERTISKPKGSVRAPQVYVLPPPEEEMTEKQVTLTCMVTDFMPEDIYVEWTNNGKTEL
NYKNTEPVLDSDGSYFMYSWLRVEKKNWVERNSYSCSVVHEGLHNHHTTKSFSRTP
GK
SEQ ID NO: 39
Murine IgG2A Fc knobs-in-holes B chain
PRGPTIKPCPPCKCPAPNLLGGPSVFIFPPKIKDVLMISLSPIVTCVVVDVSEDDPDVQI
SWFVNNVEVHTAQTQTHREDYNSTLRVVSALPIQHQDWMSGKEFKCKVNNKDLPAPI
ERTISKPKGSVRAPRVYVLPPPEEEMTKKQVTLTCMVTDFMPEDIYVEWTNNGKTEL
NYKNTEPVLVSDGSYTMYSKLRVEKKNWVERNSYSCSVVHEGLHNHHTTKSFSRTP
GK
SEQ ID NO: 40
Human VGFR(1/2)
SDTGRPFVEMYSEIPEIIHMTEGRELVIPCRVTSPNITVTLKKFPLDTLIPDGKRIIWDSR
KGFIISNATYKEIGLLTCEATVNGHLYKTNYLTHRQTNTIIDVVLSPSHGIELSVGEKLVL
NCTARTELNVGIDFNWEYPSSKHQHKKLVNRDLKTQSGSEMKKFLSTLTIDGVTRSD
QGLYTCAASSGLMTKKNSTFVRVHEK
SEQ ID NO: 41
Human FSD2
KTCRDVFCPGSSTCVVDQTNNAYCVTCNRICPEPASSEQYLCGNDGVTYSSACHLR
KATCLLGRSIGLAYEGKCI
SEQ ID NO: 42
Human FSD3
KAKSCEDIQCTGGKKCLWDFKVGRGRCSLCDELCPDSKSDEPVCASDNATYASECA
MKEAACSSGVLLEVKHSGSCN
SEQ ID NO: 43: Human follistatin N-terminal domain
GNCWLRQAKNGRCQVLYKTELSKEECCSTGRLSTSWTEEDVNDNTLFKWMIFNGGA
PNCIPCK
SEQ ID NO: 44
Human activin A (inhibin beta A chain)
(signal peptide in bold)
MPLLWLRGFLLASCWIIVRSSPTPGSEGHSAAPDCPSCALAALPKDVPNSQPEMVEA
VKKHILNMLHLKKRPDVTQPVPKAALLNAIRKLHVGKVGENGYVEIEDDIGRRAEMNEL
MEQTSEIITFAESGTARKTLHFEISKEGSDLSVVERAEVWLFLKVPKANRTRTKVTIRLF
QQQKHPQGSLDTGEEAEEVGLKGERSELLLSEKVVDARKSTWHVFPVSSSIQRLLDQ
GKSSLDVRIACEQCQESGASLVLLGKKKKKEEEGEGKKKGGGEGGAGADEEKEQSH
RPFLMLQARQSEDHPHRRRRRGLECDGKVNICCKKQFFVSFKDIGWNDWIIAPSGYH
ANYCEGECPSHIAGTSGSSLSFHSTVINHYRMRGHSPFANLKSCCVPTKLRPMSMLY
YDDGQNIIKKDIQNMIVEECGCS
SEQ ID NO: 45
Myostatin (also known as growth differentiation factor 8)
(signal peptide in bold)
MQKLQLCVYIYLFMLIVAGPVDLNENSEQKENVEKEGLCNACTWRQNTKSSRIEAIKI
QILSKLRLETAPNISKDVIRQLLPKAPPLRELIDQYDVQRDDSSDGSLEDDDYHATTETII
TMPTESDFLMQVDGKPKCCFFKFSSKIQYNKVVKAQLWIYLRPVETPTTVFVQILRLIK
PMKDGTRYTGIRSLKLDMNPGTGIWQSIDVKTVLQNWLKQPESNLGIEIKALDENGHD
LAVTFPGPGEDGLNPFLEVKVTDTPKRSRRDFGLDCDEHSTESRCCRYPLTVDFEAF
GWDWIIAPKRYKANYCSGECEFVFLQKYPHTHLVHQANPRGSAGPCCTPTKMSPINM
LYFNGKEQIIYGKIPAMVVDRCGCS
SEQ ID NO: 46
Growth differentiation factor 11 (GDF11)
MVLAAPLLLGFLLLALELRPRGEAAEGPAAAAAAAAAAAAAGVGGERSSRPAPSVA
PEPDGCPVCVWRQHSRELRLESIKSQILSKLRLKEAPNISREVVKQLLPKAPPLQQILD
LHDFQGDALQPEDFLEEDEYHATTETVISMAQETDPAVQTDGSPLCCHFHFSPKVMF
TKVLKAQLWVYLRPVPRPATVYLQILRLKPLTGEGTAGGGGGGRRHIRIRSLKIELHSR
SGHWQSIDFKQVLHSWFRQPQSNWGIEINAFDPSGTDLAVTSLGPGAEGLHPFMELR
VLENTKRSRRNLGLDCDEHSSESRCCRYPLTVDFEAFGWDWIIAPKRYKANYCSGQC
EYMFMQKYPHTHLVQQANPRGSAGPCCTPTKMSPINMLYFNDKQQIIYGKIPGMVVD
RCGCS
SEQ ID NO: 47
Human FSD1 (Q124A)
ETCENVDCGPGKKCRMNKKNKPRCVCAPDCSNITWKGPVCGLDGKTYRNECALLKA
RCKEAPELEVQYQGRCK
SEQ ID NO: 48
Human FSD1 (Q124A) as a single entity produced
in E. coli contains an initiator methionine (M)
amino acid.
METCENVDCGPGKKCRMNKKNKPRCVCAPDCSNITWKGPVCGLDGKTYRNECALL
KARCKEAPELEVQYQGRCK
SEQ ID NO: 49
Polynucleotide sequence encoding Human FSD1
(Q124A) as a single entity produced in E. coli
containing an initiator methionine (M) amino acid.
ATGGAGACATGCGAGAACGTGGATTGTGGACCAGGCAAGAAGTGCCGGATGAAC
AAGAAGAACAAGCCCAGATGCGTGTGCGCTCCTGACTGCAGCAACATCACCTGG
AAGGGACCCGTGTGCGGCCTGGATGGCAAGACATACCGGAATGAGTGCGCCCTG
CTGAAGGCTAGGTGTAAGGAGGCGCCTGAGCTGGAGGTGCAGTATCAGGGCCG
GTGCAAG
SEQ ID NO: 50
Human FSD1 (E126A)
ETCENVDCGPGKKCRMNKKNKPRCVCAPDCSNITWKGPVCGLDGKTYRNECALLKA
RCKEQPALEVQYQGRCK
SEQ ID NO: 51
Human FSD1 (E126A) as a single entity produced in E. coli
contains an initiator methionine (M) amino acid.
METCENVDCGPGKKCRMNKKNKPRCVCAPDCSNITWKGPVCGLDGKTYRNECALL
KARCKEQPALEVQYQGRCK
SEQ ID NO: 52
Polynucleotide sequence encoding Human FSD1 (E126A) as a
single entity produced in E. coli containing an
initiator methionine (M) amino acid.
ATGGAGACATGCGAGAACGTGGATTGTGGACCAGGCAAGAAGTGCCGGATGAAC
AAGAAGAACAAGCCCAGATGCGTGTGCGCTCCTGACTGCAGCAACATCACCTGG
AAGGGACCCGTGTGCGGCCTGGATGGCAAGACATACCGGAATGAGTGCGCCCTG
CTGAAGGCTAGGTGTAAGGAGCAGCCTGCGCTGGAGGTGCAGTATCAGGGCCG
GTGCAAG
SEQ ID NO: 53
Human FSD1 (Q124A, E126A)
ETCENVDCGPGKKCRMNKKNKPRCVCAPDCSNITWKGPVCGLDGKTYRNECALLKA
RCKEAPALEVQYQGRCK
SEQ ID NO: 54
Human FSD1 (Q124A, E126A) as a single entity produced in
E. coli contains an initiator methionine (M) amino acid.
METCENVDCGPGKKCRMNKKNKPRCVCAPDCSNITWKGPVCGLDGKTYRNECALL
KARCKEAPALEVQYQGRCK
SEQ ID NO: 55
Polynucleotide sequence encoding Human FSD1 (Q124A, E126A)
as a single entity produced in E. coli containing an
initiator methionine (M) amino acid.
ATGGAGACATGCGAGAACGTGGATTGTGGACCAGGCAAGAAGTGCCGGATGAAC
AAGAAGAACAAGCCCAGATGCGTGTGCGCTCCTGACTGCAGCAACATCACCTGG
AAGGGACCCGTGTGCGGCCTGGATGGCAAGACATACCGGAATGAGTGCGCCCTG
CTGAAGGCTAGGTGTAAGGAGGCGCCTGCGCTGGAGGTGCAGTATCAGGGCCG
GTGCAAG
SEQ ID NO: 56
VGFR(1/2)-mFc-FSD1 (Q124A). Serum albumin preproprotein signal
peptide in bold, murine IgG2A underlined, FSD1 (Q124A)
double underlined.
MKWVTFISLLFLFSSAYSSDTGRPFVEMYSEIPEIIHMTEGRELVIPCRVTSPNITVTLK
KFPLDTLIPDGKRIIWDSRKGFIISNATYKEIGLLTCEATVNGHLYKTNYLTHRQTNTIIDV
VLSPSHGIELSVGEKLVLNCTARTELNVGIDFNWEYPSSKHQHKKLVNRDLKTQSGSE
MKKFLSTLTIDGVTRSDQGLYTCAASSGLMTKKNSTFVRVHEKPRGPTIKPCPPCKCP
APNLLGGPSVFIFPPKIKDVLMISLSPIVTCVVVDVSEDDPDVQISWFVNNVEVHTAQT
QTHREDYNSTLRVVSALPIQHQDWMSGKEFKCKVNNKDLPAPIERTISKPKGSVRAP
QVYVLPPPEEEMTKKQVTLTCMVTDFMPEDIYVEWTNNGKTELNYKNTEPVLDSDGS
YFMYSKLRVEKKNWVERNSYSCSVVHEGLHNHHTTKSFSRTPGKETCENVDCGPGK
KCRMNKKNKPRCVCAPDCSNITWKGPVCGLDGKTYRNECALLKARCKEAPELEVQY
QGRCK
SEQ ID NO: 57
Polynucleotide sequence encoding VGFR(1/2)-mFc-FSD1 (Q124A).
ATGAAGTGGGTGACCTTCATCTCTCTGCTGTTCCTGTTCTCTTCTGCCTACTCTAG
CGACACCGGCAGGCCCTTCGTGGAGATGTACAGCGAGATCCCCGAGATCATCCA
CATGACCGAGGGCAGGGAGCTGGTGATCCCCTGCAGGGTGACCAGCCCCAACAT
CACCGTGACCCTGAAGAAGTTCCCCCTGGACACCCTGATCCCCGACGGCAAGAG
GATCATCTGGGACAGCAGGAAGGGCTTCATCATCAGCAACGCCACCTACAAGGA
GATCGGCCTGCTGACCTGCGAGGCCACCGTGAACGGCCACCTGTACAAGACCAA
CTACCTGACCCACAGGCAGACCAACACCATCATCGACGTGGTGCTGAGCCCCAG
CCACGGCATCGAGCTGAGCGTGGGCGAGAAGCTGGTGCTGAACTGCACCGCCA
GGACCGAGCTGAACGTGGGCATCGACTTCAACTGGGAGTACCCCAGCAGCAAGC
ACCAGCACAAGAAGCTGGTGAACAGGGACCTGAAGACCCAGAGCGGCAGCGAGA
TGAAGAAGTTCCTGAGCACCCTGACCATCGACGGCGTGACCAGGAGCGACCAGG
GCCTGTACACCTGCGCCGCCAGCAGCGGCCTGATGACCAAGAAGAACAGCACCT
TCGTGAGGGTGCACGAGAAGCCACGGGGACCAACCATCAAACCATGCCCCCCCT
GTAAGTGCCCCGCACCTAACCTCCTGGGGGGCCATCCGTGTTTATCTTCCCCCC
TAAAATTAAGGATGTGCTGATGATCAGCCTGAGCCCTATCGTGACATGCGTGGTC
GTCGACGTGAGCGAGGATGACCCCGACGTCCAGATCTCTTGGTTTGTGAACAAC
GTGGAGGTTCACACCGCCCAAACCCAGACACACAGAGAGGACTATAATTCTACTC
TCCGCGTTGTGTCCGCCCTGCCCATCCAGCACCAAGATTGGATGTCTGGTAAGGA
ATTCAAGTGTAAAGTGAACAATAAAGATCTGCCCGCACCAATCGAAAGAACCATTT
CTAAGCCTAAAGGCTCCGTGCGGGCTCCTCAGGTTTACGTCCTGCCACCCCCCG
AGGAAGAGATGACTAAGAAGCAGGTGACCCTGACCTGTATGGTGACCGATTTTAT
GCCCGAAGACATTTACGTCGAGTGGACCAATAATGGTAAGACAGAACTGAACTAT
AAAAATACAGAGCCAGTGCTCGACTCTGATGGCAGCTATTTTATGTACTCTAAGCT
GAGAGTTGAGAAGAAAAACTGGGTGGAACGGAATTCTTACTCTTGCTCTGTCGTG
CACGAAGGACTGCACAACCACCACACTACCAAGTCCTTTTCTAGGACCCCCGGCA
AGGAGACCTGCGAGAACGTGGACTGCGGCCCCGGCAAGAAGTGCCGGATGAAC
AAGAAGAACAAGCCCCGGTGCGTGTGCGCCCCCGACTGCAGCAACATCACCTGG
AAGGGCCCCGTGTGCGGCCTGGACGGCAAGACCTACCGGAACGAGTGCGCCCT
GCTGAAGGCCCGGTGCAAGGAGGCCCCCGAGCTGGAGGTGCAGTACCAGGGCC
GGTGCAAG
SEQ ID NO: 58
VGFR(1/2)-mFc-FSD1 (E126A). Serum albumin preproprotein signal
peptide in bold, murine IgG2A underlined, FSD1 (E126A)
double underlined.
MKWVTFISLLFLFSSAYSSDTGRPFVEMYSEIPEIIHMTEGRELVIPCRVTSPNITVTLK
KFPLDTLIPDGKRIIWDSRKGFIISNATYKEIGLLTCEATVNGHLYKTNYLTHRQTNTIIDV
VLSPSHGIELSVGEKLVLNCTARTELNVGIDFNWEYPSSKHQHKKLVNRDLKTQSGSE
MKKFLSTLTIDGVTRSDQGLYTCAASSGLMTKKNSTFVRVHEKPRGPTIKPCPPCKCP
APNLLGGPSVFIFPPKIKDVLMISLSPIVTCVVVDVSEDDPDVQISWFVNNVEVHTAQT
QTHREDYNSTLRVVSALPIQHQDWMSGKEFKCKVNNKDLPAPIERTISKPKGSVRAP
QVYVLPPPEEEMTKKQVTLTCMVTDFMPEDIYVEWTNNGKTELNYKNTEPVLDSDGS
YFMYSKLRVEKKNWVERNSYSCSVVHEGLHNHHTTKSFSRTPGKETCENVDCGPGK
KCRMNKKNKPRCVCAPDCSNITWKGPVCGLDGKTYRNECALLKARCKEQPALEVQY
QGRCK
SEQ ID NO: 59
Polynucleotide sequence encoding VGFR(1/2)-mFc-FSD1 (E126A).
ATGAAGTGGGTGACCTTCATCTCTCTGCTGTTCCTGTTCTCTTCTGCCTACTCTAG
CGACACCGGCAGGCCCTTCGTGGAGATGTACAGCGAGATCCCCGAGATCATCCA
CATGACCGAGGGCAGGGAGCTGGTGATCCCCTGCAGGGTGACCAGCCCCAACAT
CACCGTGACCCTGAAGAAGTTCCCCCTGGACACCCTGATCCCCGACGGCAAGAG
GATCATCTGGGACAGCAGGAAGGGCTTCATCATCAGCAACGCCACCTACAAGGA
GATCGGCCTGCTGACCTGCGAGGCCACCGTGAACGGCCACCTGTACAAGACCAA
CTACCTGACCCACAGGCAGACCAACACCATCATCGACGTGGTGCTGAGCCCCAG
CCACGGCATCGAGCTGAGCGTGGGCGAGAAGCTGGTGCTGAACTGCACCGCCA
GGACCGAGCTGAACGTGGGCATCGACTTCAACTGGGAGTACCCCAGCAGCAAGC
ACCAGCACAAGAAGCTGGTGAACAGGGACCTGAAGACCCAGAGCGGCAGCGAGA
TGAAGAAGTTCCTGAGCACCCTGACCATCGACGGCGTGACCAGGAGCGACCAGG
GCCTGTACACCTGCGCCGCCAGCAGCGGCCTGATGACCAAGAAGAACAGCACCT
TCGTGAGGGTGCACGAGAAGCCACGGGGACCAACCATCAAACCATGCCCCCCCT
GTAAGTGCCCCGCACCTAACCTCCTGGGTGGGCCATCCGTGTTTATCTTCCCCCC
TAAAATTAAGGATGTGCTGATGATCAGCCTGAGCCCTATCGTGACATGCGTGGTC
GTCGACGTGAGCGAGGATGACCCCGACGTCCAGATCTCTTGGTTTGTGAACAAC
GTGGAGGTTCACACCGCCCAAACCCAGACACACAGAGAGGACTATAATTCTACTC
TCCGCGTTGTGTCCGCCCTGCCCATCCAGCACCAAGATTGGATGTCTGGTAAGGA
ATTCAAGTGTAAAGTGAACAATAAAGATCTGCCCGCACCAATCGAAAGAACCATTT
CTAAGCCTAAAGGCTCCGTGCGGGCTCCTCAGGTTTACGTCCTGCCACCCCCCG
AGGAAGAGATGACTAAGAAGCAGGTGACCCTGACCTGTATGGTGACCGATTTTAT
GCCCGAAGACATTTACGTCGAGTGGACCAATAATGGTAAGACAGAACTGAACTAT
AAAAATACAGAGCCAGTGCTCGACTCTGATGGCAGCTATTTTATGTACTCTAAGCT
GAGAGTTGAGAAGAAAAACTGGGTGGAACGGAATTCTTACTCTTGCTCTGTCGTG
CACGAAGGACTGCACAACCACCACACTACCAAGTCCTTTTCTAGGACCCCCGGCA
AGGAGACCTGCGAGAACGTGGACTGCGGCCCCGGCAAGAAGTGCCGGATGAAC
AAGAAGAACAAGCCCCGGTGCGTGTGCGCCCCCGACTGCAGCAACATCACCTGG
AAGGGCCCCGTGTGCGGCCTGGACGGCAAGACCTACCGGAACGAGTGCGCCCT
GCTGAAGGCCCGGTGCAAGGAGCAGCCCGCCCTGGAGGTGCAGTACCAGGGCC
GGTGCAAG
SEQ ID NO: 60
VGFR(1/2)-mFc-FSD1 (Q124A, E126A). Serum albumin preproprotein
signal peptide in bold, murine IgG2A underlined,
FSD1 (Q124A, E126A) double underlined.
MKWVTFISLLFLFSSAYSSDTGRPFVEMYSEIPEIIHMTEGRELVIPCRVTSPNITVTLK
KFPLDTLIPDGKRIIWDSRKGFIISNATYKEIGLLTCEATVNGHLYKTNYLTHRQTNTIIDV
VLSPSHGIELSVGEKLVLNCTARTELNVGIDFNWEYPSSKHQHKKLVNRDLKTQSGSE
MKKFLSTLTIDGVTRSDQGLYTCAASSGLMTKKNSTFVRVHEKPRGPTIKPCPPCKCP
APNLLGGPSVFIFPPKIKDVLMISLSPIVTCVVVDVSEDDPDVQISWFVNNVEVHTAQT
QTHREDYNSTLRVVSALPIQHQDWMSGKEFKCKVNNKDLPAPIERTISKPKGSVRAP
QVYVLPPPEEEMTKKQVTLTCMVTDFMPEDIYVEWTNNGKTELNYKNTEPVLDSDGS
YFMYSKLRVEKKNWVERNSYSCSVVHEGLHNHHTTKSFSRTPGKETCENVDCGPGK
KCRMNKKNKPRCVCAPDCSNITWKGPVCGLDGKTYRNECALLKARCKEAPALEVQY
QGRCK
SEQ ID NO: 61
Polynucleotide sequence encoding VGFR(1/2)-mFc-FSD1 (Q124A,
E126A).
ATGAAGTGGGTGACCTTCATCTCTCTGCTGTTCCTGTTCTCTTCTGCCTACTCTAG
CGACACCGGCAGGCCCTTCGTGGAGATGTACAGCGAGATCCCCGAGATCATCCA
CATGACCGAGGGCAGGGAGCTGGTGATCCCCTGCAGGGTGACCAGCCCCAACAT
CACCGTGACCCTGAAGAAGTTCCCCCTGGACACCCTGATCCCCGACGGCAAGAG
GATCATCTGGGACAGCAGGAAGGGCTTCATCATCAGCAACGCCACCTACAAGGA
GATCGGCCTGCTGACCTGCGAGGCCACCGTGAACGGCCACCTGTACAAGACCAA
CTACCTGACCCACAGGCAGACCAACACCATCATCGACGTGGTGCTGAGCCCCAG
CCACGGCATCGAGCTGAGCGTGGGCGAGAAGCTGGTGCTGAACTGCACCGCCA
GGACCGAGCTGAACGTGGGCATCGACTTCAACTGGGAGTACCCCAGCAGCAAGC
ACCAGCACAAGAAGCTGGTGAACAGGGACCTGAAGACCCAGAGCGGCAGCGAGA
TGAAGAAGTTCCTGAGCACCCTGACCATCGACGGCGTGACCAGGAGCGACCAGG
GCCTGTACACCTGCGCCGCCAGCAGCGGCCTGATGACCAAGAAGAACAGCACCT
TCGTGAGGGTGCACGAGAAGCCACGGGGACCAACCATCAAACCATGCCCCCCCT
GTAAGTGCCCCGCACCTAACCTCCTGGGTGGGCCATCCGTGTTTATCTTCCCCCC
TAAAATTAAGGATGTGCTGATGATCAGCCTGAGCCCTATCGTGACATGCGTGGTC
GTCGACGTGAGCGAGGATGACCCCGACGTCCAGATCTCTTGGTTTGTGAACAAC
GTGGAGGTTCACACCGCCCAAACCCAGACACACAGAGAGGACTATAATTCTACTC
TCCGCGTTGTGTCCGCCCTGCCCATCCAGCACCAAGATTGGATGTCTGGTAAGGA
ATTCAAGTGTAAAGTGAACAATAAAGATCTGCCCGCACCAATCGAAAGAACCATTT
CTAAGCCTAAAGGCTCCGTGCGGGCTCCTCAGGTTTACGTCCTGCCACCCCCCG
AGGAAGAGATGACTAAGAAGCAGGTGACCCTGACCTGTATGGTGACCGATTTTAT
GCCCGAAGACATTTACGTCGAGTGGACCAATAATGGTAAGACAGAACTGAACTAT
AAAAATACAGAGCCAGTGCTCGACTCTGATGGCAGCTATTTTATGTACTCTAAGCT
GAGAGTTGAGAAGAAAAACTGGGTGGAACGGAATTCTTACTCTTGCTCTGTCGTG
CACGAAGGACTGCACAACCACCACACTACCAAGTCCTTTTCTAGGACCCCCGGCA
AGGAGACCTGCGAGAACGTGGACTGCGGCCCCGGCAAGAAGTGCCGGATGAAC
AAGAAGAACAAGCCCCGGTGCGTGTGCGCCCCCGACTGCAGCAACATCACCTGG
AAGGGCCCCGTGTGCGGCCTGGACGGCAAGACCTACCGGAACGAGTGCGCCCT
GCTGAAGGCCCGGTGCAAGGAGGCCCCCGCCCTGGAGGTGCAGTACCAGGGCC
GGTGCAAG
SEQ ID NO: 62
CBG-mFc-FSD1. Serum albumin preproprotein signal peptide in bold,
murine IgG2A underlined, FSD1 double underlined.
MKWVTFISLLFLFSSAYSMDPNAAYVNMSNHHRGLASANVDFAFSLYKHLVALSPKK
NIFISPVSISMALAMLSLGTCGHTRAQLLQGLGFNLTERSETEIHQGFQHLHQLFAKSD
TSLEMTMGNALFLDGSLELLESFSADIKHYYESEVLAMNFQDWATASRQINSYVKNKT
QGKIVDLFSGLDSPAILVLVNYIFFKGTWTQPFDLASTREENFYVDETTVVKVPMMLQS
STISYLHDSELPCQLVQMNYVGNGTVFFILPDKGKMNTVIAALSRDTINRWSAGLTSS
QVDLYIPKVTISGVYDLGDVLEEMGIADLFTNQANFSRITQDAQLKSSKVVHKAVLQLN
EEGVDTAGSTGVTLNLTSKPIILRFNQPFIIMIFDHFTWSSLFLARVMNPVPRGPTIKPC
PPCKCPAPNLLGGPSVFIFPPKIKDVLMISLSPIVTCVVVDVSEDDPDVQISWFVNNVE
VHTAQTQTHREDYNSTLRVVSALPIQHQDWMSGKEFKCKVNNKDLPAPIERTISKPK
GSVRAPQVYVLPPPEEEMTKKQVTLTCMVTDFMPEDIYVEWTNNGKTELNYKNTEPV
LDSDGSYFMYSKLRVEKKNWVERNSYSCSVVHEGLHNHHTTKSFSRTPGKETCENV
DCGPGKKCRMNKKNKPRCVCAPDCSNITWKGPVCGLDGKTYRNECALLKARCKEQ
PELEVQYQGRCK
SEQ ID NO: 63
Polynucleotide sequence encoding CBG-mFc-FSD1.
ATGAAGTGGGTGACCTTCATCTCTCTGCTGTTCCTGTTCTCTAGCGCTTACTCTAT
GGACCCCAACGCCGCCTACGTGAACATGTCTAACCACCACAGGGGCCTGGCCTC
TGCCAACGTGGACTTCGCCTTCTCTCTGTACAAGCACCTGGTGGCCCTGTCTCCC
AAGAAGAACATCTTCATCTCTCCCGTGTCTATCTCTATGGCCCTGGCCATGCTGTC
TCTGGGCACCTGCGGCCACACCAGGGCCCAGCTGCTGCAGGGCCTGGGCTTCA
ACCTGACCGAGAGGTCTGAGACCGAGATCCACCAGGGCTTCCAGCACCTGCACC
AGCTGTTCGCCAAGTCTGACACCTCTCTGGAGATGACCATGGGCAACGCCCTGTT
CCTGGACGGCTCTCTGGAGCTGCTGGAGTCTTTCTCTGCCGACATCAAGCACTAC
TACGAGTCTGAGGTGCTGGCCATGAACTTCCAGGACTGGGCCACCGCCTCTAGG
CAGATCAACTCTTACGTGAAGAACAAGACCCAGGGCAAGATCGTGGACCTGTTCT
CTGGCCTGGACTCTCCCGCCATCCTGGTGCTGGTGAACTACATCTTCTTCAAGGG
CACCTGGACCCAGCCCTTCGACCTGGCCTCTACCAGGGAGGAGAACTTCTACGT
GGACGAGACCACCGTGGTGAAGGTGCCCATGATGCTGCAGTCTTCTACCATCTCT
TACCTGCACGACTCTGAGCTGCCCTGCCAGCTGGTGCAGATGAACTACGTGGGC
AACGGCACCGTGTTCTTCATCCTGCCCGACAAGGGCAAGATGAACACCGTGATCG
CCGCCCTGTCTAGGGACACCATCAACAGGTGGTCTGCCGGCCTGACCTCTTCTCA
GGTGGACCTGTACATCCCCAAGGTGACCATCTCTGGCGTGTACGACCTGGGCGA
CGTGCTGGAGGAGATGGGCATCGCCGACCTGTTCACCAACCAGGCCAACTTCTC
TAGGATCACCCAGGACGCCCAGCTGAAGTCTTCTAAGGTGGTGCACAAGGCCGT
GCTGCAGCTGAACGAGGAGGGCGTGGACACCGCCGGCTCTACCGGCGTGACCC
TGAACCTGACCTCTAAGCCCATCATCCTGAGGTTCAACCAGCCCTTCATCATCATG
ATCTTCGACCACTTCACCTGGTCTTCTCTGTTCCTGGCCAGGGTGATGAACCCCG
TGCCTCGAGGACCAACCATCAAACCATGCCCCCCCTGTAAGTGCCCCGCACCTAA
CCTCCTGGGTGGGCCATCCGTGTTTATCTTCCCCCCTAAAATTAAGGATGTGCTG
ATGATCAGCCTGAGCCCTATCGTGACATGCGTGGTCGTCGACGTGAGCGAGGAT
GACCCCGACGTCCAGATCTCTTGGTTTGTGAACAACGTGGAGGTTCACACCGCCC
AAACCCAGACACACAGAGAGGACTATAATTCTACTCTCCGCGTTGTGTCCGCCCT
GCCCATCCAGCACCAAGATTGGATGTCTGGTAAGGAATTCAAGTGTAAAGTGAAC
AATAAAGATCTGCCCGCACCAATCGAAAGAACCATTTCTAAGCCTAAAGGCTCCGT
GCGGGCTCCTCAGGTTTACGTCCTGCCACCCCCCGAGGAAGAGATGACTAAGAA
GCAGGTGACCCTGACCTGTATGGTGACCGATTTTATGCCCGAAGACATTTACGTC
GAGTGGACCAATAATGGTAAGACAGAACTGAACTATAAAAATACAGAGCCAGTGC
TCGACTCTGATGGCAGCTATTTTATGTACTCTAAGCTGAGAGTTGAGAAGAAAAAC
TGGGTGGAACGGAATTCTTACTCTTGCTCTGTCGTGCACGAAGGACTGCACAACC
ACCACACTACCAAGTCCTTTTCTAGGACCCCCGGCAAGGAGACCTGCGAGAACGT
GGACTGCGGCCCCGGCAAGAAGTGCCGGATGAACAAGAAGAACAAGCCCCGGT
GCGTGTGCGCCCCCGACTGCAGCAACATCACCTGGAAGGGCCCCGTGTGCGGC
CTGGACGGCAAGACCTACCGGAACGAGTGCGCCCTGCTGAAGGCCCGGTGCAA
GGAGCAGCCCGAGCTGGAGGTGCAGTACCAGGGCCGGTGCAAG
SEQ. ID NO: 64
FSD1(KTC) (Q124A)
ETCENVDCGPGKKCRMNKKNKPRCVCAPDCSNITWKGPVCGLDGKTYRNECALLKA
RCKEAPELEVQYQGRCKKTC
SEQ. ID NO: 65
FSD1(KTC) (E126A)
ETCENVDCGPGKKCRMNKKNKPRCVCAPDCSNITWKGPVCGLDGKTYRNECALLKA
RCKEQPALEVQYQGRCKKTC
SEQ. ID NO: 66
FSD1(KTC) (Q124A, E126A)
ETCENVDCGPGKKCRMNKKNKPRCVCAPDCSNITWKGPVCGLDGKTYRNECALLKA
RCKEAPALEVQYQGRCKKTC
SEQ. ID NO: 67
serum albumin preproprotein signal peptide
MKWVTFISLLFLFSSAYS

### Items

1. A fusion protein or conjugated protein comprising:
(i) one or more polypeptide(s) (P1) comprising or consisting of at least one FSD1 domain comprising or consisting of a polypeptide having at least 70%, such as at least 80%, for instance at least 90%, such as at least 95%, for instance at least 99% sequence identity to SEQ ID NO: 1, and
(ii) a therapeutic or diagnostic agent.

2. The fusion protein or conjugated protein according to item 1 comprising:
(i) one or more polypeptide(s) (P1) consisting of at least one FSD1 domain comprising or consisting of a polypeptide having at least 70% such as at least 80%, for instance at least 90%, such as at least 95%, for instance at least 99% sequence identity to SEQ ID NO: 1, and
(ii) a therapeutic or diagnostic agent.

3. The fusion protein or conjugated protein according to item 1 comprising:
(i) one or more polypeptide(s) (P1) comprising or consisting of at least one FSD1 domain comprising or consisting of a polypeptide having at least 70%, such as at least 80%, for instance at least 90%, such as at least 95%, for instance at least 99% sequence identity to SEQ ID NO: 1, and
(ii) a therapeutic or diagnostic agent comprising or consisting of an immunoglobulin domain;
and wherein the one or more polypeptide(s) (P1) (i) are linked C-terminally to said immunoglobulin Fc domain.

4. The fusion protein or conjugated protein according to item 3, wherein said immunoglobulin is selected from the group consisting of: an IgG, an IgA, and IgM, and IgE, and an IgD.

5. The fusion protein or conjugated protein according to any one of the preceding items comprising:
(i) one or more polypeptide(s) (P1) consisting of at least one FSD1 domain comprising or consisting of a polypeptide having at least 70% such as at least 80%, for instance at least 90%, such as at least 95%, for instance at least 99% sequence identity to SEQ ID NO: 1, and
(ii) a therapeutic or diagnostic agent comprising or consisting of an immunoglobulin domain;
and wherein the one or more polypeptide(s) (P1) (i) are linked C-terminally to said immunoglobulin Fc domain, preferably wherein the immunoglobulin is an IgG.

6. The fusion protein or conjugated protein according to any one of items 3 to 5, wherein said immunoglobulin is and IgG, and wherein the IgG Fc domain comprises or consists of an IgG Fc-homodimer or an IgG Fc-heterodimer.

7. The fusion protein or conjugated protein according to item 6, wherein the fusion protein or conjugated protein comprises or consists of an IgG Fc-homodimer, wherein each of the monomer of the IgG Fc-homodimer is C-terminally linked to at least one polypeptide (P1) comprising or consisting of at least one FSD1 domain comprising or consisting of a polypeptide having at least 70% such as at least 80%, for instance at least 90%, such as at least 95%, for instance at least 99% sequence identity to SEQ ID NO: 1, and wherein each of the monomer of the IgG Fc-homodimer is linked N-terminally to a therapeutic or diagnostic moiety identical or different.

8. The fusion protein or conjugated protein according to any one of the preceding items, wherein the fusion protein or conjugated protein comprises or consists of an IgG Fc-homodimer, wherein each of the monomer of the IgG Fc-homodimer is C-terminally linked to at least one polypeptide (P1) (i) consisting of at least one FSD1 domain comprising or consisting of a polypeptide having at least 70% such as at least 80%, for instance at least 90%, such as at least 95%, for instance at least 99% sequence identity to SEQ ID NO: 1,
and wherein each of the monomer of the IgG Fc-homodimer is linked N-terminally to a therapeutic or diagnostic moiety identical or different.

9. The fusion protein or conjugated protein according to item 6, wherein the fusion protein or conjugated protein comprises or consists of an IgG Fc-heterodimer, wherein each of the monomer of the IgG Fc-heterodimer is linked C-terminally to at least one polypeptide (P1) comprising or consisting of at least one FSD1 domain comprising or consisting of a polypeptide having at least 70% such as at least 80%, for instance at least 90%, such as at least 95%, for instance at least 99% sequence identity to SEQ ID NO: 1, and wherein each of the monomer of the IgG Fc-heterodimer is N-terminally linked to a therapeutic or diagnostic moiety identical or different.

10. The fusion protein or conjugated protein according to any one of the preceding items, wherein the fusion protein or conjugated protein comprises or consists of an IgG Fc-heterodimer, wherein each of the monomer of the IgG Fc-heterodimer is linked C-terminally to at least one polypeptide (P1) (i) consisting of at least one FSD1 domain comprising or consisting of a polypeptide having at least 70% such as at least 80%, for instance at least 90%, such as at least 95%, for instance at least 99% sequence identity to SEQ ID NO: 1,
and wherein each of the monomer of the IgG Fc-heterodimer is N-terminally linked to a therapeutic or diagnostic moiety identical or different.

11. The fusion protein or conjugated protein according to item 1 comprising:
(i) one or more polypeptide(s) (P1) (i) comprising or consisting of at least one FSD1 domain comprising or consisting of a polypeptide having at least 70%, such as at least 80%, for instance at least 90%, such as at least 95%, for instance at least 99% sequence identity to SEQ ID NO: 1, and
(ii) a therapeutic or diagnostic agent; wherein the therapeutic or diagnostic agent comprises or consists of an IgG Fc-heterodimer, wherein at least one of the monomer of the IgG Fc-heterodimer is linked N-terminally to at least one of the one or more polypeptide(s) (P1) (i) and wherein at least one of the monomer of the IgG Fc-heterodimer is linked N-terminally to a therapeutic or diagnostic moiety identical or different.

12. The fusion protein or conjugated protein according to any one of the preceding items, wherein at least one of the monomers of the IgG Fc-heterodimer is linked N-terminally to at least one of the one or more polypeptide(s) (P1) (i), for example at least two polypeptides (P1), such as at least three polypeptides (P1), for example at least four polypeptides, such as at least five polypeptides (P1) comprising or consisting of at least one FSD1 domain comprising or consisting of a polypeptide having at least 70% such as at least 80%, for instance at least 90%, such as at least 95%, for instance at least 99% sequence identity to SEQ ID NO: 1.

13. The fusion protein or conjugated protein according to any one of any one of the preceding items wherein the therapeutic or diagnostic agent of any of items 1 to 3, or the at least one the therapeutic or diagnostic moiety identical or different of any one of the preceding items is one or more cytokine receptor.

14. The fusion protein or conjugated protein according to any one of the preceding items wherein the therapeutic or diagnostic agent of any of the preceding items, or the at least one the therapeutic or diagnostic moiety identical or different of any of the preceding items comprises or consists of the ligand binding domain of TNFR2.

15. The fusion protein or conjugated protein according to any of the preceding items wherein the therapeutic or diagnostic agent of any one of the preceding items, or the at least one the therapeutic or diagnostic moiety identical or different of any one of the preceding items comprises or consists of the ligand binding domain of one or more growth factor receptor.

16. The fusion protein or conjugated protein according to any one of the preceding items wherein the therapeutic or diagnostic agent of any one of the preceding items, or the at least one the therapeutic or diagnostic moiety identical or different of any one of the preceding items comprises or consists of the ligand binding domain of VGFR1.

17. The fusion protein or conjugated protein according to any one of the preceding items wherein the therapeutic or diagnostic agent of any one of the preceding items, or the at least one the therapeutic or diagnostic moiety identical or different of any one of the preceding items comprises or consists of the ligand binding domain of VGFR2.

18. The fusion protein or conjugated protein according to any one of the preceding items wherein the one or more polypeptide(s) (P1) comprising or consisting of at least one FSD1 domain comprise or consist of a polypeptide having at least 70% sequence identity, such as at least 80% identity, for instance at least 90% identity, such as at least 95% identity, for instance 99% identity to SEQ. ID. NO: 26.

19. The fusion protein or conjugated protein according to item 1 comprising:
(i) one or more polypeptide(s) (P1) comprising or consisting of at least one FSD1 domain comprising or consisting of a polypeptide having at least 70% such as at least 80%, for instance at least 90%, such as at least 95%, for instance at least 99% sequence identity to SEQ ID NO: 1, and
(ii) a therapeutic or diagnostic agent;
wherein the one or more polypeptide(s) (P1) (i) and the therapeutic or diagnostic agent (ii) are linked covalently or by non-covalent interaction.

20. The fusion protein or conjugated protein according to item 19 wherein the one or more polypeptide(s) (P1) (i) and the therapeutic or diagnostic agent (ii) are linked covalently using click chemistry,

21. The fusion protein or conjugated protein according to item 19 wherein the one or more polypeptide(s) (P1) (i) and the therapeutic or diagnostic agent (ii) are linked by a non-covalent streptavidin-biotin interaction.

22. The fusion protein or conjugated protein according to item 19 wherein the one or more polypeptide(s) (P1) (i) and the therapeutic or diagnostic agent (ii) are linked by a nanobody with affinity for the therapeutic or diagnostic agent, such as an anti-Fc nanobody.

23. The fusion protein or conjugated protein according to any one of the preceding items comprising:
(i) one or more polypeptide(s) (P1) consisting of at least one FSD1 domain comprising or consisting of a polypeptide having at least 70% such as at least 80%, for instance at least 90%, such as at least 95%, for instance at least 99% sequence identity to SEQ ID NO: 1,
   and
(ii) a therapeutic or diagnostic agent;
wherein the one or more polypeptide(s) (P1) (i) and the therapeutic or diagnostic agent (ii) are linked covalently, such as by click chemistry or by non-covalent interaction, such as streptavidin-biotin interaction or by a nanobody with affinity for the therapeutic or diagnostic agent (ii).

24. The fusion protein or conjugated protein according to any one of items 19 to 23, wherein the therapeutic or diagnostic agent (ii) comprises or consists of a full-length antibody.

25. The fusion protein or conjugated protein according to item 24, wherein the full-length antibody a bi-specific therapeutic antibody.

26. The fusion protein or conjugated protein according to any one of the preceding items, wherein the one or more polypeptide(s) (P1) (i) comprise or consist of a sequence having at least 70% similarity, such as at least 80% similarity, for instance at least 90% similarity, such as at least 95% similarity, for instance at least 99% similarity with a sequence selected from the group consisting of: SEQ ID NO: 23, SEQ ID NO: 24, SEQ ID NO: 64, SEQ ID NO: 65, and SEQ ID NO: 66.

27. The fusion protein or conjugated protein according to any one of the preceding items, wherein the one or more polypeptide(s) (P1) (i) comprise or consist of a sequence having at least 70% similarity, such as at least 80% similarity, for instance at least 90% similarity, such as at least 95% similarity, for instance at least 99% similarity with a sequence selected from the group consisting of: SEQ ID NO: 23 and SEQ ID NO: 24.

28 The fusion protein or conjugated protein according to any one of the preceding items, wherein the one or more polypeptide(s) (P1) are encoded by a sequence comprising or consisting of a sequence having at least 70% similarity, such as at least 80% similarity, for instance at least 90% similarity, such as at least 95% similarity, for instance at least 99% similarity with SEQ ID NO: 25.

29 The fusion protein or conjugated protein according to any one of the preceding items, wherein the one or more polypeptide(s) (P1) (i), do not neutralize the activity of myostatin (SEQ ID NO: 45), GDF11 (SEQ ID NO: 46) and/or activin A (SEQ ID NO: 44).

30. The fusion protein or conjugated protein according to any one of the preceding items, wherein said fusion protein or conjugated protein do not neutralize the activity of myostatin (SEQ ID NO: 45), GDF11 (SEQ ID NO: 46) and/or activin A (SEQ ID NO: 44) in a concentration range up to at least 5 nM, such as at least 7.5 nM, for example at least 8 nM, such as at least 8.2 nM, for example at least 8.4 nM, such as at least 8.6 nM, for example at least 8.8 nM, such as at least 9 nM, for example at least 10 nM, such as at least 12 nM, for example at least 15 nM, such as at least 20 nM, for example at least 30 nM, such as at least 40 nM, for example at least 50 nM, such as at least 60 nM, for example at least 61 nM, such as at least 62 nM, for example at least 63 nM, such as at least 64 nM, for example at least 64 nM, such as at least 65 nM, for example at least 70 nM, such as at least 80 nM, for example at least 100 nM, such as at least 150 nM, for example at least 200 nM, such as at least 500 nM, for example at least 600 nM, such as at least 700 nM, for example at least 750 nM, such as at least 800 nM, for example at least 820 nM, such as at least 821 nM, for example at least 822 nM, such as at least 830 nM, for example at least 1 μM, such as at least 2 μM, for example at least 3 μM, such as at least 4 μM, for example at least 5 μM, such as at least 6 μM, for example at least 7UM, such as at least 8UM, for example at least 9 μM, such as at least 10UM, wherein the neutralization, or absence thereof, is determined by reporter gene bioassay of phosphorylated Smad2/3 signaling

31. The fusion protein or conjugated protein according to any one of the preceding items where said fusion protein or conjugated protein do not neutralize the activity of myostatin, GDF11 and/or activin A in a concentration range up to at least 5 nM, such as at least 7.5 nM, for example at least 8 nM, such as at least 8.2 nM, for example at least 8.4 nM, such as at least 8.6 nM, for example at least 8.8 nM, such as at least 9 nM, for example at least 10 nM, such as at least 12 nM, for example at least 15 nM, such as at least 20 nM, for example at least 30 nM, such as at least 40 nM, for example at least 50 nM, such as at least 80 nM, for example at least 100 nM, such as at least 150 nM, for example at least 200 nM.

32. The fusion protein or conjugated protein according to any one of the preceding items, wherein the one or more polypeptide(s) (P1) (i) do not neutralize the activity of myostatin (SEQ ID NO: 45), GDF11 (SEQ ID NO: 46) and/or activin A (SEQ ID NO: 44) in a concentration range up to at least 5 nM, such as at least 7.5 nM, for example at least 8 nM, such as at least 8.2 nM, for example at least 8.4 nM, such as at least 8.6 nM, for example at least 8.8 nM, such as at least 9 nM, for example at least 10 nM, such as at least 12 nM, for example at least 15 nM, such as at least 20 nM, for example at least 30 nM, such as at least 40 nM, for example at least 50 nM, such as at least 60 nM, for example at least 61 nM, such as at least 62 nM, for example at least 63 nM, such as at least 64 nM, for example at least 64 nM, such as at least 65 nM, for example at least 70 nM, such as at least 80 nM, for example at least 100 nM, such as at least 150 nM, for example at least 200 nM, such as at least 500 nM, for example at least 600 nM, such as at least 700 nM, for example at least 750 nM, such as at least 800 nM, for example at least 820 nM, such as at least 821 nM, for example at least 822 nM, such as at least 830 nM, for example at least 1 μM, such as at least 2 μM, for example at least 3 μM, such as at least 4 μM, for example at least 5 μM, such as at least 6 μM, for example at least 7 μM, such as at least 8UM, for example at least 9 μM, such as at least 10UM, wherein the neutralization, or absence thereof, is determined by reporter gene bioassay of phosphorylated Smad2/3 signaling

33. The fusion protein or conjugated protein according to any one of the preceding items where in the one or more polypeptide(s) (P1) (i) do not neutralize the activity of myostatin, GDF11 and/or activin A in a concentration range up to at least 5 nM, such as at least 7.5 nM, for example at least 8 nM, such as at least 8.2 nM, for example at least 8.4 nM, such as at least 8.6 nM, for example at least 8.8 nM, such as at least 9 nM, for example at least 10 nM, such as at least 12 nM, for example at least 15 nM, such as at least 20 nM, for example at least 30 nM, such as at least 40 nM, for example at least 50 nM, such as at least 80 nM, for example at least 100 nM, such as at least 150 nM, for example at least 200 nM.

34. The fusion protein or conjugated protein according to any one of the preceding items, wherein the one or more polypeptide(s) (P1) (i) bind heparan sulfate.

35. The fusion protein or conjugated protein according to any one of the preceding items, wherein the one or more polypeptide(s) (P1) (i) comprising at least one FSD1 domain are encoded by a sequence comprising or consisting of a sequence having at least 70% identity, such as at least 80% identity, for instance at least 90% identity, such as at least 95% identity, for example at least 99% identity with SEQ. ID. NO: 33.

36 The fusion protein or conjugated protein according to any one of the preceding items, wherein the one or more polypeptide(s) (P1) (i) comprising at least one FSD1 domain comprise or consist of a sequence having 100% identity with SEQ. ID. NO: 1.

37. The fusion protein or conjugated protein according to any one of the preceding items, further comprising a linker between the one or more polypeptide(s) (P1) (i) comprising or consisting of at least one FSD1 domain and the therapeutic or diagnostic agent (ii).

38. The fusion protein or conjugated protein according to item 37, wherein the linker is a chemical linker.

39 The fusion protein or conjugated protein according to any one of the preceding items, wherein the therapeutic or diagnostic agent (ii) according to any one of the preceding items, or the therapeutic or diagnostic moiety identical or different of any one of the preceding items, is selected from the group consisting of: a peptide, a protein, such as an antibody or fragment thereof, a nanobody, a glycoprotein, a streptavidin or an interleukin, a nucleic acid, and a small molecule.

40. The fusion protein or conjugated protein according to any one of the preceding items, wherein the fusion protein or conjugated protein further comprises a detectable moiety.

41. The fusion protein or conjugated protein according to item 40 wherein the detectable moiety is selected from the group consisting of: a fluorescent protein, a gold nanoparticle, a radioactive isotope, biotin or a derivative thereof, and an enzyme.

42. The fusion protein or conjugated protein according to any one of the preceding items, wherein the therapeutic or diagnostic agent (ii) of any one of the preceding items, or the therapeutic or diagnostic moiety identical or different of any one of the preceding items binds a target selected from the group consisting of: cluster of differentiation (CD) proteins, cytokines, such as interleukins, growth factors, such as colony-stimulating factors, immune checkpoint proteins, angiogenic factors, hemostatic factors, chemotaxic factors, neurotrophic factors, inflammatory proteins, tumor antigens, bacterial proteins, and viral proteins.

43. The fusion protein or conjugated protein according to item 42, wherein the therapeutic or diagnostic agent (ii) of any one of the preceding items, or the therapeutic or diagnostic moiety identical or different of any one of the preceding items comprises or consists of the ligand binding domain of a protein selected from the group consisting of: TNFR2, VGFR1, VGFR2, and CBG.

44. The fusion protein or conjugated protein according to any one of the preceding items, wherein the therapeutic or diagnostic agent (ii) of any one of the preceding items, or the therapeutic or diagnostic moiety identical or different of any one of the preceding items comprises or consists of the ligand binding domain of a protein having at least 70% similarity, such as at least 80% similarity, for instance at least 90% similarity, such as at least 95% similarity, for example at least 99% similarity with a sequence selected form the group consisting of: SEQ ID NO: 19, SEQ ID NO: 20, SEQ ID NO: 21, and SEQ ID NO: 22.

45. The fusion protein or conjugated protein according to any one of the preceding items, wherein the therapeutic or diagnostic agent (ii) of any the preceding items or the therapeutic or diagnostic moiety identical or different of any one of the preceding items comprises or consists of an anti-vimentin nanobody.

46. The fusion protein according to item 45, wherein the anti-vimentin nanobody has at least 70% similarity, such as at least 80% similarity, for instance at least 90% similarity, such as at least 95% similarity, for example at least 99% similarity with SEQ ID NO: 34.

47. The fusion protein according to any one of items 45 to 46, wherein the anti-vimentin nanobody is encoded by a sequence having at least 70% similarity, such as at least 80% similarity, for instance at least 90% similarity, such as at least 95% similarity, for example at least 99% similarity with SEQ ID NO: 35.

48. The fusion protein or conjugated protein according to any one of the preceding items, wherein the therapeutic or diagnostic agent (ii) of any one of the preceding items, or the therapeutic or diagnostic moiety identical or different of any one of the preceding items comprises or consists of:
(a) the ligand binding domain of a protein selected from the group consisting of: TNFR2 (SEQ ID NO: 21), VGFR1 (SEQ ID NO: 19), VGFR2 (SEQ ID NO: 20), and CBG (SEQ ID NO: 22); and/or
(b) VGFR(½) (SEQ ID NO:40)
(b) an anti-vimentin nanobody having at least 70% similarity, such as at least 80% similarity, for instance at least 90% similarity, such as at least 95% similarity, for example at least 99% similarity with SEQ ID NO: 34; and/or
(c) a full-length antibody, such as an anti-TNFα full-length antibody.

49. The fusion protein or conjugated protein according to any one of the preceding items wherein the fusion protein or conjugated protein is ubiquitinylated.

50. The fusion protein or conjugated protein according to any one of the preceding items, wherein the therapeutic or diagnostic agent (ii) of any one of the preceding items, or the therapeutic or diagnostic moiety identical or different of any one of the preceding items bind to a target and wherein the fusion protein or conjugated protein according to any one of the preceding items is able to bind heparan sulfate and said target at the same time.

51. The fusion protein or conjugated protein according to any one of the preceding items, wherein the fusion protein or conjugated protein has a longer local half-life at the site of administration than the therapeutic or diagnostic agent (ii) of any one of the preceding items, or the therapeutic or diagnostic moiety identical or different of any one of the preceding items, alone.

52. The fusion protein or conjugated protein according to any one of the preceding items, wherein the fusion protein or conjugated protein increases the binding of the therapeutic or diagnostic agent (ii) of any one of the preceding items, or of the therapeutic or diagnostic moiety identical or different of any one of the preceding items to the extracellular matrix compartment compared to the therapeutic or diagnostic agent (ii) of any one of the preceding items, or the therapeutic or diagnostic moiety identical or different of any one of the preceding items alone.

53. The fusion protein or conjugated protein according to any one of the preceding items, wherein the fusion protein or conjugated protein increases the binding of the therapeutic or diagnostic agent (ii) of any of one of the preceding items, or of the therapeutic or diagnostic moiety identical or different of any one of the preceding items to a predefined organ or a tumor compared to the therapeutic or diagnostic agent (ii) of any one of the preceding items, or the therapeutic or diagnostic moiety identical or different of any one of the preceding items alone.

54. The fusion protein or conjugated protein according to any one of the preceding items, wherein the EC50 of the therapeutic or diagnostic agent (ii) or the therapeutic or diagnostic moiety identical or different, to a predefined organ or a tumor, compared to the therapeutic or diagnostic agent (ii) or the therapeutic or diagnostic moiety identical or different alone is decreased by at least a factor 2, such at least a factor 5, for example at least a factor 10, such as at least a factor 20, for example at least a factor 50, such as at least a factor 80, for example at least a factor 100, such as at least a factor 250, for example at least a factor 500, such at least a factor 1000, for example at least a factor 5000, such as at least a factor 10000, for example at least a factor 100000.

55. The fusion protein or conjugated protein according to item 54 wherein the predefined organ is selected from the group consisting of: a musculoskeletal system organ, a digestive system organ, a respiratory system organ. a urinary system organ, a reproductive organ, and endocrine system organ, a circulatory system organ, a nervous system organ, an hematopoietic organ and an integumentary system organ.

56. The fusion protein or conjugated protein according to item 55, wherein the organ is selected from the group consisting of: kidney, eye, liver, heart, lungs, bladder, pancreas, gallbladder, intestine, prostate, brain, skin, muscle, bone, hematopoietic tissue and subcutaneous tissue, such as a joint or synovial tissue.

57. The fusion protein or conjugated protein according to any of the preceding items, wherein the fusion protein or the conjugated protein increases the intracellular uptake of the therapeutic or diagnostic agent (ii) of any of the preceding items, or of the therapeutic or diagnostic moiety identical or different of any one of the preceding items compared to the therapeutic or diagnostic agent (ii) of any one of the preceding items, or the therapeutic or diagnostic moiety identical or different of any one of the preceding items alone.

58. The fusion protein or conjugated protein according to any one of the preceding items, wherein the therapeutic or diagnostic agent (ii) of any of the preceding items, or the therapeutic or diagnostic moiety identical or different of any one of the preceding items is linked to at least one polypeptide (P1) (i), for example at least two polypeptides (P1) (i), such as at least three polypeptides (P1) (i), for example at least four polypeptides, such as at least five polypeptides (P1) (i) comprising or consisting of at least one FSD1 domain comprising or consisting of a polypeptide having at least 70% such as at least 80%, for instance at least 90%, such as at least 95%, for instance at least 99% sequence identity to SEQ ID NO: 1.

59. The fusion protein or conjugated protein according to any one of the preceding items wherein each monomer of the Fc-homodimer or Fc-heterodimer is linked to at least one polypeptide (P1) (i), for example at least two polypeptides (P1) (i), such as at least three polypeptides (P1) (i), for example at least four polypeptides, such as at least five polypeptides (P1) (i) comprising or consisting of at least one FSD1 domain comprising or consisting of a polypeptide having at least 70% such as at least 80%, for instance at least 90%, such as at least 95%, for instance at least 99% sequence identity to SEQ ID NO: 1.

60. The fusion protein or conjugated protein according to any one of the preceding items, wherein at least one of the at least one polypeptide (P1) (i) comprises or consists of a multimer of FSD1 domains, such as at least two FSD1 domains, for example at least three FSD1 domains, for example at least four FSD1 domains, such as at least five FSD1 domains comprising or consisting of a polypeptide having at least 70% such as at least 80%, for instance at least 90%, such as at least 95%, for instance at least 99% sequence identity to SEQ ID NO: 1.

61. The fusion protein or conjugated protein according to item 60, wherein at least 2 monomers of the multimers of FSD1 domain are joined by a linker.

62. The fusion protein or conjugated protein according to any one of the preceding items, wherein the fusion protein or conjugated protein comprises or consists of a polypeptide having at least 70% sequence identity, such as at least 80% sequence identity, for example at least 90% sequence identity, such as at least 95% identity, for example at least 99% identity with a sequence selected from the group consisting of: SEQ ID NO: 2, SEQ ID NO: 3, SEQ ID NO: 5, SEQ ID NO: 7, and SEQ ID NO: 9.

63. The fusion protein or conjugated protein according to any one of the preceding items, wherein the fusion protein or conjugated protein comprises or consists of a polypeptide having at least 70% sequence identity, such as at least 80% sequence identity, for example at least 90% sequence identity, such as at least 95% identity, for example at least 99% identity with SEQ ID NO: 11 and a polypeptide having at least 70% sequence identity, such as at least 80% sequence identity, for example at least 90% sequence identity, such as at least 95% identity, for example at least 99% identity with a sequence selected from the group consisting of: SEQ ID NO: 13, SEQ ID NO: 15, and SEQ ID NO: 17.

64. The fusion protein or conjugated protein according to any one of the preceding items, wherein the fusion protein or conjugated protein comprises or consists of a polypeptide having at least 70% sequence identity, such as at least 80% sequence identity, for example at least 90% sequence identity, such as at least 95% identity, for example at least 99% identity with:
(a) a sequence selected from the group consisting of: SEQ ID NO: 2, SEQ ID NO: 3, SEQ ID NO: 5, SEQ ID NO: 7, SEQ ID NO: 9, SEQ ID NO: 56, SEQ ID NO: 58, SEQ ID NO: 60, and SEQ ID NO: 62; and/or
(b) SEQ ID NO: 11 and a polypeptide having at least 70% sequence identity, such as at least 80% sequence identity, for example at least 90% sequence identity, such as at least 95% identity, for example at least 99% identity with a sequence selected from the group consisting of: SEQ ID NO: 13, SEQ ID NO: 15, and SEQ ID NO: 17.

65. The fusion protein or conjugated protein according to any one of the preceding items, wherein the fusion protein or conjugated protein does not comprise any other of the follistatin domains FSD2, FSD3, and/or a N-terminal follistatin domain, preferably wherein the fusion protein or conjugated protein does not comprise any of the human FSD2 of SEQ ID NO: 41, human FSD3 of SEQ ID NO: 42 and human follistatin N-terminal domain of SEQ ID NO: 43.

66. The fusion protein or conjugated protein according to any one of the preceding items, wherein the one or more polypeptides (P1) (i) do not comprise any other of the follistatin domains FSD2, FSD3, and/or a N-terminal follistatin domain, preferably wherein the fusion protein or conjugated protein does not comprise any of the human FSD2 of SEQ ID NO: 41, human FSD3 of SEQ ID NO: 42 and human follistatin N-terminal domain of SEQ ID NO: 43.

67. The fusion protein or conjugated protein according to any one of the preceding items, wherein the at least one FSD1 domain of the (i) one or more polypeptides (P1) comprises or consists of a variant FSD1 domain encoded by a sequence selected from the group consisting of: SEQ ID NO: 47, SEQ ID NO: 50, and SEQ ID NO: 53, or a polypeptide having at least 70% such as at least 80%, for instance at least 90%, such as at least 95%, for instance at least 96%, such as at least 97%, for instance 98%, such as at least 99% sequence identity thereto.

68. The fusion protein or conjugated protein according to any one of the preceding items, wherein the fusion protein or conjugated protein further comprises a PROTAC linker, preferably wherein the PROTAC linker is selected from the group consisting of: flexible aliphatic linkers, flexible PEGylated linkers, flexible heterochains, rigid linkers, triazole-based linkers, Bio-orthogonal clickable linkers and photo-switchable linkers.

69. One or more polynucleotides which upon expression encode the fusion protein or conjugated protein according to any one of the preceding items.

70. The one or more polynucleotides according to item 69, wherein the one or more polynucleotides have at least 70% sequence identity, such as at least 80% sequence identity, for example at least 90% sequence identity, such as at least 95% identity, for example at least 99% identity with a polynucleotide selected from the group consisting of: SEQ ID NO: 4, SEQ ID NO: 6, SEQ ID NO: 8, and SEQ ID NO: 10.

71. The one or more polynucleotides according to item 69, wherein one or more polynucleotides have at least 70% sequence identity, such as at least 80% sequence identity, for example at least 90% sequence identity, such as at least 95% identity, for example at least 99% identity with a combination of SEQ ID NO: 12 and at least one polynucleotide selected from the group consisting of: SEQ ID NO: 14, SEQ ID NO: 16, and SEQ ID NO: 18.

72. One or more constructs or vectors comprising the one or more polynucleotides according to any one of items 69 to 71 or encoding the fusion protein or conjugated protein according to any of the items 1 to 68.

73. One or more constructs or vectors encoding the fusion protein according to any of the preceding items.

74. A host cell comprising the one or more polynucleotides of any one of items 69 to 71 or the one or more constructs or vectors of any one of items 72 to 73.

75. The host cell according to item 74 wherein the host cell is a mammalian cell.

76. The host cell according to item 75 wherein the host cell is a human cell.

77. The host cell according to item 76 wherein the host cell is a Human embryonic kidney 293 cell (HEK293) cell.

78. The host cell according to item 75 wherein the host cell is a Chinese Hamster Ovary (CHO) cell.

79. A composition comprising the fusion protein or conjugated protein according to any one of items 1 to 68, the one or more polynucleotides according to any one of items 69 to 71, the one or more constructs or vectors according to any one of items 72 to 73, the host cell according to any one of items 74 to 78, or mixtures thereof.

80. The composition according to item 79, wherein the composition is a pharmaceutical composition.

81. The fusion protein or conjugated protein according to any one of items 1 to 68, the one or more polynucleotides according to any one of items 69 to 71, the one or more constructs or vectors according to any one of items 72 to 73, the host cell according to any one of items 74 to 78, or the composition according to any one of items 79 to 80, for use in medicine.

82. The fusion protein or conjugated protein according to any one of items 1 to 68, the one or more polynucleotides according to any one of items 69 to 71, the one or more constructs or vectors according to any one of items 72 to 73, the host cell according to any one of items 74 to 78, or the composition according to any one of items 79 to 80, for use in the treatment of an ophthalmologic condition, an oncogenic condition, or an inflammatory condition, such as a rheumatic condition.

83. The use according to item 82, wherein the ophthalmologic condition is neovascular age-related macular degeneration (AMD).

84. The use according to item 82, wherein the inflammatory condition is caused by organ transplantation.

85. The use according to any one of items 81 to 82, 84, in a subject donating or receiving an organ, and wherein the donated or transplanted organ is selected from the group consisting of: kidney, heart, lung, bone marrow, and liver.

86. A method of treatment of a disease or condition, comprising administering to a subject an effective amount of the fusion protein or conjugated protein according to any one of items 1 to 68, the one or more polynucleotides according to any one of items 69 to 71, the one or more constructs or vectors according to any one of items 72 to 73, the host cell according to any one of items 74 to 78, or the composition according to any one of items 79 to 80.

87. A method of treating an ophthalmologic condition, an inflammatory condition, or an oncogenic condition, comprising administering to a subject an effective amount of the fusion protein or conjugated protein according to any one of items 1 to 68, the one or more polynucleotides according to any one of items 69 to 71, the one or more constructs or vectors according to any one of items 72 to 73, the host cell according to any one of items 74 to 78, or the composition according to any one of items 79 to 80.

88. Use of the fusion protein or conjugated protein according to any one of items 1 to 68, the one or more polynucleotides according to any one of items 69 to 71, the one or more constructs or vectors according to any one of items 72 to 73, the host cell according to any one of items 74 to 78, or the composition according to any one of items 79 to 80 in the manufacture of a medicament for the treatment of a disease or condition, for example an ophthalmologic condition, an oncogenic condition, or an inflammatory condition.

89. The use according to any one of items 81 to 85, the method according to any one of items 86 to 87 or the use according to item 88, wherein the fusion protein or conjugated protein according to any one of items 1 to 68, the one or more polynucleotides according to any one of items 69 to 71, the one or more constructs or vectors according to any one of items 72 to 73, the host cell according to any one of items 74 to 78, or the composition according to any one of items 79 to 80 is administered systematically.

90. The use according to any one of items 81 to 85, the method according to any one of items 86 to 87 or the use according to item 88, wherein the fusion protein or conjugated protein according to any one of items 1 to 68, the one or more polynucleotides according to any one of items 69 to 71, the one or more constructs or vectors according to any one of items 72 to 73, the host cell according to any one of items 74 to 78, or the composition according to any one of items 79 to 80 is administered locally.

91. The use according to any one of items 81 to 85, the method according to any one of items 86 to 87 or the use according to item 88, wherein the fusion protein or conjugated protein according to any one of items 1 to 68, the one or more polynucleotides according to any one of items 69 to 71, the one or more constructs or vectors according to any one of items 72 to 73, the host cell according to any one of items 74 to 78, or the composition according to any one of items 79 to 80 is administered intraadiposally, intraarterially, intraarticularly, intracranially, intradermally, intralesionally, intramuscularly, intranasally, intraocularally, intraosseously, intrapericardially, intraperitoneally, intrapleurally, intraprostatically, intrarectally, intrathecally, intratracheally, intratumorally, intraumbilically, intravaginally, intravenously, intravesicularlly, intravitreally, or liposomally.

92. A method of increasing the local half-life of a therapeutic or diagnostic agent at the site of administration, comprising obtaining the fusion protein or conjugated protein according to any one of items 1 to 68, the one or more polynucleotides according to any one of items 69 to 71, the one or more constructs or vectors according to any one of items 72 to 73, the host cell according to any one of items 74 to 78, or the composition according to any one of items 79 to 80, wherein the therapeutic or diagnostic agent (ii) of any one of items 1 to 68, or the therapeutic or diagnostic moiety identical or different of items 1 to 68 comprises or consists of said therapeutic or diagnostic agent.

93. A method of increasing the local in-vivo half-life of a therapeutic or diagnostic agent at the site of administration in a subject comprising administering to said subject the fusion protein or conjugated protein according to any one of items 1 to 68, the one or more polynucleotides according to any one of items 69 to 71, the one or more constructs or vectors according to any one of items 72 to 73, the host cell according to any one of items 74 to 78, or the composition according to any one of items 79 to 80, wherein the therapeutic or diagnostic agent (ii) of any one of items 1 to 68, or the therapeutic or diagnostic moiety identical or different of items 1 to 68 comprises or consists of said therapeutic or diagnostic agent.

94. A method of increasing the local in-vivo half-life of a therapeutic or diagnostic agent comprising the steps of:
a) providing a therapeutic or diagnostic agent;
b) obtaining the fusion protein or conjugated protein according to any one of items 1 to 68, wherein the therapeutic or diagnostic agent of step a) is the therapeutic or diagnostic agent (ii) of any one of claims 1 to 68,
   thereby increasing the local in-vivo half-life of said therapeutic or diagnostic agent.

95. The method according to any one of items 93 to 94, wherein the fusion protein or conjugated protein, or the composition is administered to a subject suffering from an ophthalmologic condition and wherein the fusion protein or conjugated protein, or the composition is administered intravitreally, subretinally or suprachoroidally.

96. The method according to item 95 wherein the ophthalmologic condition is wet AMD.

97. The method according to item 93, wherein the fusion protein or conjugated protein, or the composition is administered to a subject donating or receiving an organ, preferably a kidney, heart, lung, bone marrow, or liver, even more preferably a kidney, or administered to said organ by ex-vivo perfusion, and wherein the therapeutic or diagnostic agent comprises or consists of a compound reducing inflammation.

98. The method according to any one of items 92 to 97, wherein the local half-life of the therapeutic or diagnostic agent is increased by at least 6h, such as at least 12h, for instance at least 24 h, such as at least 48h, for instance at least 72h, such as at least 96h, for instance at least 120h, such as at least one week, for instance at least 2 weeks, such as at least 4 weeks, for instance at least 8 weeks, such as at least 3 months, for instance at least 6 months, such as at least 12 months at the site of administration.

99. A method of increasing the binding of a therapeutic or diagnostic agent to the extracellular matrix comprising administering to a subject the fusion protein or conjugated protein according to any one of items 1 to 68, the one or more polynucleotides according to any one of items 69 to 71, the one or more constructs or vectors according to any one of items 72 to 73, the host cell according to any one of items 74 to 78, or the composition according to any one of items 79 to 80, wherein the therapeutic or diagnostic agent (ii) of any one of items 1 to 68, or the therapeutic or diagnostic moiety identical or different of any one of items 1 to 68 comprises or consists of said therapeutic or diagnostic agent.

100. A method of increasing the binding of a therapeutic or diagnostic agent to a predefined organ comprising administering to a subject the fusion protein or conjugated protein according to any one of items 1 to 68, the one or more polynucleotides according to any one of items 69 to 71, the one or more constructs or vectors according to any one of items 72 to 73, the host cell according to any one of items 74 to 78, or the composition according to any one of items 79 to 80, wherein the therapeutic or diagnostic agent (ii) of any of any one items 1 to 68, or the therapeutic or diagnostic moiety identical or different of any one of items 1 to 68 comprises or consists of said therapeutic or diagnostic agent.

101. A method of increasing the intracellular uptake of a therapeutic or diagnostic agent comprising administering to a subject the fusion protein or conjugated protein according to any one of items 1 to 68, the one or more polynucleotides according to any one of items 69 to 71, the one or more constructs or vectors according to any one of items 72 to 73, the host cell according to any one of items 74 to 78, or the composition according to any one of items 79 to 80, wherein the therapeutic or diagnostic agent (ii) of any of any one items 1 to 68, or the therapeutic or diagnostic moiety identical or different of any one of items 1 to 68 comprises or consists of said therapeutic or diagnostic agent.

102. A method of degrading an intracellular protein, said method comprising the step of coupling the fusion protein or conjugated protein according to any one of the preceding items to a ligand of E3 ubiquitin ligase, wherein the therapeutic or diagnostic agent (ii) of said fusion protein or conjugated protein binds to said intracellular protein.

103. The method according to item 102, wherein the ligand of E3 ubiquitin ligase is (S, R, S)-AHPC-PEG8-NHS.

104. The method according to any one of items 102 to 103, wherein the E3 ubiquitin ligase is the Von-Hippel Lindau (VHL) tumor suppressor protein.

105. The method according to any one of items 102 to 104, wherein the therapeutic or diagnostic agent (ii) is an anti-vimentin nanobody.

106. The method according to any one of items 102 to 105, wherein the fusion protein or conjugated protein is a Type 1 protein as described herein, preferably wherein the fusion protein or conjugated protein is anti-vimentin-nanobody-FSD1.

107. The method according to any one of items 102 to 106, wherein the method is in vitro, in vivo or ex vivo.

108. The use according to any one of items 81 to 85, 88 to 91 or the method according to any one of items 92 to 107, wherein the subject is a human or a non-human animal.

109. The use according to any one of items 81 to 85, 88 to 91 or the method according to any one of items 92 to 107, wherein the fusion protein or conjugated protein according to any one of items 1 to 68, the one or more polynucleotides according to any one of item 69 to 71, the one or more constructs or vectors according to any one of items 72 to 73, the host cell according to any one of items 74 to 78, or the composition according to any one of items 79 to 80 is administered systematically.

110. The use according to any one of items 81 to 85, 88 to 91 or the method according to any one of items 92 to 107, wherein the fusion protein or conjugated protein according to any one of items 1 to 68, the one or more polynucleotides according to any one of item 69 to 71, the one or more constructs or vectors according to any one of items 72 to 73, the host cell according to any one of items 74 to 78, or the composition according to any one of items 79 to 80 is administered locally.

111. The use according to any one of items 81 to 85, 88 to 91 or the method according to any one of items 92 to 107, wherein the fusion protein or conjugated protein according to any one of items 1 to 68, the one or more polynucleotides according to any one of item 69 to 71, the one or more constructs or vectors according to any one of items 72 to 73, the host cell according to any one of items 74 to 78, or the composition according to any one of items 79 to 80 is administered intraadiposally, intraarterially, intraarticularly, intracranially, intradermally, intralesionally, intramuscularly, intranasally, intraocularally, intraosseously, intrapericardially, intraperitoneally, intrapleurally, intraprostatically, intrarectally, intrathecally, intratracheally, intratumorally, intraumbilically, intravaginally, intravenously, intravesicularlly, intravitreally, or liposomally.

112. The method according to any one of items 101 to 111, wherein said therapeutic or diagnostic agent is transported to the cytosol, such as to the cytoskeleton or to the nucleus.

113. One or more constructs or vectors encoding the one or more polypeptide(s) (P1) (i) of the fusion protein or conjugated protein according to any one of the preceding items.

114. A composition comprising one or more polypeptides selected from the group consisting of: FSD1 (Q124A) of sequence ID NO: 47, FSD1 (E126A) of sequence ID NO: 50, and FSD1 (Q124 E126A) of sequence ID NO: 53, or variants thereof, having at least 70% such as at least 80%, for instance at least 90%, such as at least 95%, for instance at least 96%, such as at least 97%, for instance 98%, such as at least 99% sequence identity thereto.

1. A fusion protein or conjugated protein comprising:
(i) one or more polypeptide(s) (P1) consisting of at least one FSD1 domain comprising or consisting of a polypeptide having at least 70% such as at least 80%, for instance at least 90%, such as at least 95%, for instance at least 99% sequence identity to SEQ ID NO: 1,
and
(ii) a therapeutic or diagnostic agent.

2. The fusion protein or conjugated protein according to claim 1 comprising:
(i) one or more polypeptide(s) (P1) consisting of at least one FSD1 domain comprising or consisting of a polypeptide having at least 70%, such as at least 80%, for instance at least 90%, such as at least 95%, for instance at least 99% sequence identity to SEQ ID NO: 1,
and
(ii) a therapeutic or diagnostic agent comprising or consisting of an immunoglobulin domain;
and wherein the one or more polypeptide(s) (P1) (i) are linked C-terminally to said immunoglobulin Fc domain.

3. The fusion protein or conjugated protein according to claim 2, wherein said immunoglobulin is selected from the group consisting of: an IgG, an IgA, and IgM, and IgE, and an IgD.

4. The fusion protein or conjugated protein according to any one of the preceding claims comprising:
(i) one or more polypeptide(s) (P1) consisting of at least one FSD1 domain comprising or consisting of a polypeptide having at least 70% such as at least 80%, for instance at least 90%, such as at least 95%, for instance at least 99% sequence identity to SEQ ID NO: 1,
and
(ii) a therapeutic or diagnostic agent comprising or consisting of an immunoglobulin domain;
and wherein the one or more polypeptide(s) (P1) (i) are linked C-terminally to said immunoglobulin Fc domain, preferably wherein the immunoglobulin is an IgG.

5. The fusion protein or conjugated protein according to any one of claims 2 to 4, wherein said immunoglobulin is and IgG, and wherein the IgG Fc domain comprises or consists of an IgG Fc-homodimer or an IgG Fc-heterodimer.

6. The fusion protein or conjugated protein according to any one of the preceding claims, wherein the fusion protein or conjugated protein comprises or consists of an IgG Fc-homodimer, wherein each of the monomer of the IgG Fc-homodimer is C-terminally linked to at least one polypeptide (P1) (i) consisting of at least one FSD1 domain comprising or consisting of a polypeptide having at least 70% such as at least 80%, for instance at least 90%, such as at least 95%, for instance at least 99% sequence identity to SEQ ID NO: 1,
and wherein each of the monomer of the IgG Fc-homodimer is linked N-terminally to a therapeutic or diagnostic moiety identical or different.

7. The fusion protein or conjugated protein according to any one of the preceding claims, wherein the fusion protein or conjugated protein comprises or consists of an IgG Fc-heterodimer, wherein each of the monomer of the IgG Fc-heterodimer is linked C-terminally to at least one polypeptide (P1) (i) consisting of at least one FSD1 domain comprising or consisting of a polypeptide having at least 70% such as at least 80%, for instance at least 90%, such as at least 95%, for instance at least 99% sequence identity to SEQ ID NO: 1,
and wherein each of the monomer of the IgG Fc-heterodimer is N-terminally linked to a therapeutic or diagnostic moiety identical or different.

8. The fusion protein or conjugated protein according to any one of the preceding claims comprising:
(i) one or more polypeptide(s) (P1) (i) consisting of at least one FSD1 domain comprising or consisting of a polypeptide having at least 70%, such as at least 80%, for instance at least 90%, such as at least 95%, for instance at least 99% sequence identity to SEQ ID NO: 1,
and
(ii) a therapeutic or diagnostic agent; wherein the therapeutic or diagnostic agent comprises or consists of an IgG Fc-heterodimer, wherein at least one of the monomer of the IgG Fc-heterodimer is linked N-terminally to at least one of the one or more polypeptide(s) (P1) (i) and wherein at least one of the monomer of the IgG Fc-heterodimer is linked N-terminally to a therapeutic or diagnostic moiety identical or different.

9. The fusion protein or conjugated protein according to any one of the preceding claims, wherein at least one of the monomers of the IgG Fc-heterodimer is linked N-terminally to at least one of the one or more polypeptide(s) (P1) (i), for example at least two polypeptides (P1), such as at least three polypeptides (P1), for example at least four polypeptides, such as at least five polypeptides (P1) consisting of at least one FSD1 domain comprising or consisting of a polypeptide having at least 70% such as at least 80%, for instance at least 90%, such as at least 95%, for instance at least 99% sequence identity to SEQ ID NO: 1.

10. The fusion protein or conjugated protein according to any one of the preceding claims wherein the therapeutic or diagnostic agent of any of the preceding claims, or the at least one the therapeutic or diagnostic moiety identical or different of any one of the preceding claims is one or more cytokine receptor.

11. The fusion protein or conjugated protein according to any one of the preceding claims wherein the therapeutic or diagnostic agent of any of the preceding claims, or the at least one the therapeutic or diagnostic moiety identical or different of any of the preceding claims comprises or consists of the ligand binding domain of TNFR2.

12. The fusion protein or conjugated protein according to any one of claims 1 to 11 wherein the therapeutic or diagnostic agent, or the at least one the therapeutic or diagnostic moiety identical or different of any one of the preceding claims comprises or consists of the ligand binding domain of one or more growth factor receptor.

13. The fusion protein or conjugated protein according to any one of claims 1 to 12 wherein the therapeutic or diagnostic agent, or the at least one the therapeutic or diagnostic moiety identical or different of any one of the preceding claims comprises or consists of the ligand binding domain of VGFR1.

14. The fusion protein or conjugated protein according to any one of claims 1 to 13 wherein the therapeutic or diagnostic agent, or the at least one the therapeutic or diagnostic moiety identical or different of any one of the preceding claims comprises or consists of the ligand binding domain of VGFR2.

15. The fusion protein or conjugated protein according to any one of the preceding claims, wherein the one or more polypeptide(s) (P1) consisting of at least one FSD1 domain comprising or consisting of a polypeptide having at least 70% sequence identity, such as at least 80% identity, for instance at least 90% identity, such as at least 95% identity, for instance 99% identity to SEQ. ID. NO: 26.

16. The fusion protein or conjugated protein according to any one of the preceding claims comprising:
(i) one or more polypeptide(s) (P1) consisting of at least one FSD1 domain comprising or consisting of a polypeptide having at least 70% such as at least 80%, for instance at least 90%, such as at least 95%, for instance at least 99% sequence identity to SEQ ID NO: 1,
and
(ii) a therapeutic or diagnostic agent;
wherein the one or more polypeptide(s) (P1) (i) and the therapeutic or diagnostic agent (ii) are linked covalently or by non-covalent interaction.

17. The fusion protein or conjugated protein according to claim 16 wherein the one or more polypeptide(s) (P1) (i) and the therapeutic or diagnostic agent (ii) are linked covalently using click chemistry,

18. The fusion protein or conjugated protein according to claim 16 wherein the one or more polypeptide(s) (P1) (i) and the therapeutic or diagnostic agent (ii) are linked by a non-covalent streptavidin-biotin interaction.

19. The fusion protein or conjugated protein according to claim 16 wherein the one or more polypeptide(s) (P1) (i) and the therapeutic or diagnostic agent (ii) are linked by a nanobody with affinity for the therapeutic or diagnostic agent, such as an anti-Fc nanobody.

20. The fusion protein or conjugated protein according to any one of the preceding claims comprising:
(i) one or more polypeptide(s) (P1) consisting of at least one FSD1 domain consisting of a polypeptide having at least 70% such as at least 80%, for instance at least 90%, such as at least 95%, for instance at least 99% sequence identity to SEQ ID NO: 1,
and
(ii) a therapeutic or diagnostic agent;
wherein the one or more polypeptide(s) (P1) (i) and the therapeutic or diagnostic agent (ii) are linked covalently, such as by click chemistry or by non-covalent interaction, such as streptavidin-biotin interaction or by a nanobody with affinity for the therapeutic or diagnostic agent (ii).

21. The fusion protein or conjugated protein according to any one of claims 16 to 20, wherein the therapeutic or diagnostic agent (ii) comprises or consists of a full-length antibody.

22. The fusion protein or conjugated protein according to claim 21, wherein the full-length antibody a bi-specific therapeutic antibody.

23. The fusion protein or conjugated protein according to any one of the preceding claims, wherein the one or more polypeptide(s) (P1) (i) consist of a sequence having at least 70% similarity, such as at least 80% similarity, for instance at least 90% similarity, such as at least 95% similarity, for instance at least 99% similarity with a sequence selected from the group consisting of: SEQ ID NO: 23, SEQ ID NO: 24, SEQ ID NO: 64, SEQ ID NO: 65, and SEQ ID NO: 66.

24. The fusion protein or conjugated protein according to any one of the preceding claims, wherein the one or more polypeptide(s) (P1) (i) consist of a sequence having at least 70% similarity, such as at least 80% similarity, for instance at least 90% similarity, such as at least 95% similarity, for instance at least 99% similarity with a sequence selected from the group consisting of: SEQ ID NO: 23 and SEQ ID NO: 24.

25. The fusion protein or conjugated protein according to any one of the preceding claims, wherein the one or more polypeptide(s) (P1) are encoded by a sequence comprising or consisting of a sequence having at least 70% similarity, such as at least 80% similarity, for instance at least 90% similarity, such as at least 95% similarity, for instance at least 99% similarity with SEQ ID NO: 25.

26. The fusion protein or conjugated protein according to any one of the preceding claims, wherein the one or more polypeptide(s) (P1) (i), do not neutralize the activity of myostatin (SEQ ID NO: 45), GDF11 (SEQ ID NO: 46) and/or activin A (SEQ ID NO: 44).

27. The fusion protein or conjugated protein according to any one of the preceding claims, wherein said fusion protein or conjugated protein do not neutralize the activity of myostatin (SEQ ID NO: 45), GDF11 (SEQ ID NO: 46) and/or activin A (SEQ ID NO: 44) in a concentration range up to at least 5 nM, such as at least 7.5 nM, for example at least 8 nM, such as at least 8.2 nM, for example at least 8.4 nM, such as at least 8.6 nM, for example at least 8.8 nM, such as at least 9 nM, for example at least 10 nM, such as at least 12 nM, for example at least 15 nM, such as at least 20 nM, for example at least 30 nM, such as at least 40 nM, for example at least 50 nM, such as at least 60 nM, for example at least 61 nM, such as at least 62 nM, for example at least 63 nM, such as at least 64 nM, for example at least 64 nM, such as at least 65 nM, for example at least 70 nM, such as at least 80 nM, for example at least 100 nM, such as at least 150 nM, for example at least 200 nM, such as at least 500 nM, for example at least 600 nM, such as at least 700 nM, for example at least 750 nM, such as at least 800 nM, for example at least 820 nM, such as at least 821 nM, for example at least 822 nM, such as at least 830 nM, for example at least 1 μM, such as at least 2 μM, wherein the neutralization, or absence thereof, is determined by reporter gene bioassay of phosphorylated Smad2/3 signaling

28. The fusion protein or conjugated protein according to any one of the preceding claims where said fusion protein or conjugated protein do not neutralize the activity of myostatin, GDF11 and/or activin A in a concentration range up to at least 5 nM, such as at least 7.5 nM, for example at least 8 nM, such as at least 8.2 nM, for example at least 8.4 nM, such as at least 8.6 nM, for example at least 8.8 nM, such as at least 9 nM, for example at least 10 nM, such as at least 12 nM, for example at least 15 nM, such as at least 20 nM, for example at least 30 nM, such as at least 40 nM, for example at least 50 nM, such as at least 80 nM, for example at least 100 nM, such as at least 150 nM, for example at least 200 nM.

29. The fusion protein or conjugated protein according to any one of the preceding claims, wherein the one or more polypeptide(s) (P1) (i) do not neutralize the activity of myostatin (SEQ ID NO: 45), GDF11 (SEQ ID NO: 46) and/or activin A (SEQ ID NO: 44) in a concentration range up to at least 5 nM, such as at least 7.5 nM, for example at least 8 nM, such as at least 8.2 nM, for example at least 8.4 nM, such as at least 8.6 nM, for example at least 8.8 nM, such as at least 9 nM, for example at least 10 nM, such as at least 12 nM, for example at least 15 nM, such as at least 20 nM, for example at least 30 nM, such as at least 40 nM, for example at least 50 nM, such as at least 60 nM, for example at least 61 nM, such as at least 62 nM, for example at least 63 nM, such as at least 64 nM, for example at least 64 nM, such as at least 65 nM, for example at least 70 nM, such as at least 80 nM, for example at least 100 nM, such as at least 150 nM, for example at least 200 nM, such as at least 500 nM, for example at least 600 nM, such as at least 700 nM, for example at least 750 nM, such as at least 800 nM, for example at least 820 nM, such as at least 821 nM, for example at least 822 nM, such as at least 830 nM, for example at least 1 μM, such as at least 2 μM, wherein the neutralization, or absence thereof, is determined by reporter gene bioassay of phosphorylated Smad2/3 signaling

30. The fusion protein or conjugated protein according to any one of the preceding claims where in the one or more polypeptide(s) (P1) (i) do not neutralize the activity of myostatin, GDF11 and/or activin A in a concentration range up to at least 5 nM, such as at least 7.5 nM, for example at least 8 nM, such as at least 8.2 nM, for example at least 8.4 nM, such as at least 8.6 nM, for example at least 8.8 nM, such as at least 9 nM, for example at least 10 nM, such as at least 12 nM, for example at least 15 nM, such as at least 20 nM, for example at least 30 nM, such as at least 40 nM, for example at least 50 nM, such as at least 80 nM, for example at least 100 nM, such as at least 150 nM, for example at least 200 nM.

31. The fusion protein or conjugated protein according to any one of the preceding claims, wherein the one or more polypeptide(s) (P1) (i) bind heparan sulfate.

32. The fusion protein or conjugated protein according to any one of the preceding claims, wherein the one or more polypeptide(s) (P1) (i) comprising at least one FSD1 domain are encoded by a sequence comprising or consisting of a sequence having at least 70% identity, such as at least 80% identity, for instance at least 90% identity, such as at least 95% identity, for example at least 99% identity with SEQ. ID. NO: 33.

33. The fusion protein or conjugated protein according to any one of the preceding claims, wherein the one or more polypeptide(s) (P1) (i) comprising at least one FSD1 domain comprise or consist of a sequence having 100% identity with SEQ. ID. NO: 1.

34. The fusion protein or conjugated protein according to any one of the preceding claims, further comprising a linker between the one or more polypeptide(s) (P1) (i) consisting of at least one FSD1 domain and the therapeutic or diagnostic agent (ii).

35. The fusion protein or conjugated protein according to claim 34, wherein the linker is a chemical linker.

36. The fusion protein or conjugated protein according to any one of the preceding claims, wherein the therapeutic or diagnostic agent (ii), or the therapeutic or diagnostic moiety identical or different of any one of the preceding claims, is selected from the group consisting of: a peptide, a protein, such as an antibody or fragment thereof, a nanobody, a glycoprotein, a streptavidin or an interleukin, a nucleic acid, and a small molecule.

37. The fusion protein or conjugated protein according to any one of the preceding claims, wherein the fusion protein or conjugated protein further comprises a detectable moiety.

38. The fusion protein or conjugated protein according to claim 37 wherein the detectable moiety is selected from the group consisting of: a fluorescent protein, a gold nanoparticle, a radioactive isotope, biotin or a derivative thereof, and an enzyme.

39. The fusion protein or conjugated protein according to any one of the preceding claims, wherein the therapeutic or diagnostic agent (ii), or the therapeutic or diagnostic moiety identical or different of any one of the preceding claims binds a target selected from the group consisting of: cluster of differentiation (CD) proteins, cytokines, such as interleukins, growth factors, such as colony-stimulating factors, immune checkpoint proteins, angiogenic factors, hemostatic factors, chemotaxic factors, neurotrophic factors, inflammatory proteins, tumor antigens, bacterial proteins, and viral proteins.

40. The fusion protein or conjugated protein according to claim 39, wherein the therapeutic or diagnostic agent (ii), or the therapeutic or diagnostic moiety identical or different of any one of the preceding claims comprises or consists of the ligand binding domain of a protein selected from the group consisting of: TNFR2, VGFR1, VGFR2, and CBG.

41. The fusion protein or conjugated protein according to any one of the preceding claims, wherein the therapeutic or diagnostic agent (ii), or the therapeutic or diagnostic moiety identical or different of any one of the preceding claims comprises or consists of the ligand binding domain of a protein having at least 70% similarity, such as at least 80% similarity, for instance at least 90% similarity, such as at least 95% similarity, for example at least 99% similarity with a sequence selected form the group consisting of: SEQ ID NO: 19, SEQ ID NO: 20, SEQ ID NO: 21, and SEQ ID NO: 22.

42. The fusion protein or conjugated protein according to claim 36, wherein the therapeutic or diagnostic agent (ii) of any the preceding claims or the therapeutic or diagnostic moiety identical or different of any one of claims 5 to 32 comprises or consists of an anti-vimentin nanobody.

43. The fusion protein according to claim 42, wherein the anti-vimentin nanobody has at least 70% similarity, such as at least 80% similarity, for instance at least 90% similarity, such as at least 95% similarity, for example at least 99% similarity with SEQ ID NO: 34.

44. The fusion protein according to any one of claims 42 to 43, wherein the anti-vimentin nanobody is encoded by a sequence having at least 70% similarity, such as at least 80% similarity, for instance at least 90% similarity, such as at least 95% similarity, for example at least 99% similarity with SEQ ID NO: 35.

45. The fusion protein or conjugated protein according to any one of the preceding claims, wherein the therapeutic or diagnostic agent (ii) of any one of the preceding claims, or the therapeutic or diagnostic moiety identical or different of any one of the preceding claims comprises or consists of:
(a) the ligand binding domain of a protein selected from the group consisting of: TNFR2 (SEQ ID NO: 21), VGFR1 (SEQ ID NO: 19), VGFR2 (SEQ ID NO: 20, and CBG (SEQ ID NO: 22); and/or
(b) VGFR(½) (SEQ ID NO:40); and/or
(c) an anti-vimentin nanobody having at least 70% similarity, such as at least 80% similarity, for instance at least 90% similarity, such as at least 95% similarity, for example at least 99% similarity with SEQ ID NO: 34; and/or
(d) a full-length antibody, such as an anti-TNFα full-length antibody.

46. The fusion protein or conjugated protein according to any one of the preceding claims wherein the fusion protein or conjugated protein is ubiquitinylated.

47. The fusion protein or conjugated protein according to any one of the preceding claims, wherein the therapeutic or diagnostic agent (ii), or the therapeutic or diagnostic moiety identical or different of any one of the preceding claims, bind to a target and wherein the fusion protein or conjugated protein according to any one of the preceding claims is able to bind heparan sulfate and said target at the same time.

48. The fusion protein or conjugated protein according to any one of the preceding claims, wherein the fusion protein or conjugated protein has a longer local half-life at the site of administration than the therapeutic or diagnostic agent (ii), or the therapeutic or diagnostic moiety identical or different of any one of the preceding, alone.

49. The fusion protein or conjugated protein according to any one of the preceding claims, wherein the fusion protein or conjugated protein increases the binding of the therapeutic or diagnostic agent (ii), or of the therapeutic or diagnostic moiety identical or different of any one of the preceding claims, to the extracellular matrix compartment compared to the therapeutic or diagnostic agent (ii), or the therapeutic or diagnostic moiety identical or different of any one of the preceding claims alone.

50. The fusion protein or conjugated protein according to any one of the preceding claims, wherein the fusion protein or conjugated protein increases the binding of the therapeutic or diagnostic agent (ii), or of the therapeutic or diagnostic moiety identical or different of any one of the preceding claims, to a predefined organ or a tumor compared to the therapeutic or diagnostic agent (ii), or the therapeutic or diagnostic moiety identical or different of any one of the preceding claims alone.

51. The fusion protein or conjugated protein according to any one of the preceding claims, wherein the EC50 of the therapeutic or diagnostic agent (ii) or the therapeutic or diagnostic moiety identical or different, to a predefined organ or a tumor, compared to the therapeutic or diagnostic agent (ii) or the therapeutic or diagnostic moiety identical or different alone is decreased by at least a factor 2, such at least a factor 5, for example at least a factor 10, such as at least a factor 20, for example at least a factor 50, such as at least a factor 80, for example at least a factor 100, such as at least a factor 250, for example at least a factor 500, such at least a factor 1000, for example at least a factor 5000, such as at least a factor 10000, for example at least a factor 100000.

52. The fusion protein or conjugated protein according to claim 51 wherein the predefined organ is selected from the group consisting of: a musculoskeletal system organ, a digestive system organ, a respiratory system organ, a urinary system organ, a reproductive organ, and endocrine system organ, a circulatory system organ, a nervous system organ, an hematopoietic organ and an integumentary system organ.

53. The fusion protein or conjugated protein according to claim 52, wherein the organ is selected from the group consisting of: kidney, eye, liver, heart, lungs, bladder, pancreas, gallbladder, intestine, prostate, brain, skin, muscle, bone, hematopoietic tissue and subcutaneous tissue, such as a joint or synovial tissue.

54. The fusion protein or conjugated protein according to any of the preceding claims, wherein the fusion protein or the conjugated protein increases the intracellular uptake of the therapeutic or diagnostic agent (ii), or of the therapeutic or diagnostic moiety identical or different of any one of the preceding claims compared to the therapeutic or diagnostic agent (ii) of any one of the preceding claims, or the therapeutic or diagnostic moiety identical or different of any one of the preceding claims alone.

55. The fusion protein or conjugated protein according to any one of the preceding claims, wherein the therapeutic or diagnostic agent (ii) of any of the preceding claims, or the therapeutic or diagnostic moiety identical or different of any one of the preceding claims is linked to at least one polypeptide (P1) (i), for example at least two polypeptides (P1) (i), such as at least three polypeptides (P1) (i), for example at least four polypeptides, such as at least five polypeptides (P1) (i) consisting of at least one FSD1 domain comprising or consisting of a polypeptide having at least 70% such as at least 80%, for instance at least 90%, such as at least 95%, for instance at least 99% sequence identity to SEQ ID NO: 1.

56. The fusion protein or conjugated protein according to any one of claims 5 to 55 wherein each monomer of the Fc-homodimer or Fc-heterodimer is linked to at least one polypeptide (P1) (i), for example at least two polypeptides (P1) (i), such as at least three polypeptides (P1) (i), for example at least four polypeptides, such as at least five polypeptides (P1) (i) consisting of at least one FSD1 domain comprising or consisting of a polypeptide having at least 70% such as at least 80%, for instance at least 90%, such as at least 95%, for instance at least 99% sequence identity to SEQ ID NO: 1.

57. The fusion protein or conjugated protein according to any one of the preceding claims, wherein at least one of the at least one polypeptide (P1) (i) comprises or consists of a multimer of FSD1 domains, such as at least two FSD1 domains, for example at least three FSD1 domains, for example at least four FSD1 domains, such as at least five FSD1 domains consisting of a polypeptide having at least 70% such as at least 80%, for instance at least 90%, such as at least 95%, for instance at least 99% sequence identity to SEQ ID NO: 1.

58. The fusion protein or conjugated protein according to claim 57, wherein at least 2 monomers of the multimers of FSD1 domain are joined by a linker.

59. The fusion protein or conjugated protein according to any one of the preceding claims, wherein the fusion protein or conjugated protein comprises or consists of a polypeptide having at least 70% sequence identity, such as at least 80% sequence identity, for example at least 90% sequence identity, such as at least 95% identity, for example at least 99% identity with a sequence selected from the group consisting of: SEQ ID NO: 2, SEQ ID NO: 3, SEQ ID NO: 5, SEQ ID NO: 7, and SEQ ID NO: 9.

60. The fusion protein or conjugated protein according to any one of the preceding claims, wherein the fusion protein or conjugated protein comprises or consists of a polypeptide having at least 70% sequence identity, such as at least 80% sequence identity, for example at least 90% sequence identity, such as at least 95% identity, for example at least 99% identity with SEQ ID NO: 11 and a polypeptide having at least 70% sequence identity, such as at least 80% sequence identity, for example at least 90% sequence identity, such as at least 95% identity, for example at least 99% identity with a sequence selected from the group consisting of: SEQ ID NO: 13, SEQ ID NO: 15, and SEQ ID NO: 17.

61. The fusion protein or conjugated protein according to any one of the preceding claims, wherein the fusion protein or conjugated protein comprises or consists of a polypeptide having at least 70% sequence identity, such as at least 80% sequence identity, for example at least 90% sequence identity, such as at least 95% identity, for example at least 99% identity with:
(a) a sequence selected from the group consisting of: SEQ ID NO: 2, SEQ ID NO: 3, SEQ ID NO: 5, SEQ ID NO: 7, SEQ ID NO: 9, SEQ ID NO: 56, SEQ ID NO: 58, SEQ ID NO: 60, and SEQ ID NO: 62; and/or
(b) SEQ ID NO: 11 and a polypeptide having at least 70% sequence identity, such as at least 80% sequence identity, for example at least 90% sequence identity, such as at least 95% identity, for example at least 99% identity with a sequence selected from the group consisting of: SEQ ID NO: 13, SEQ ID NO: 15, and SEQ ID NO: 17.

62. The fusion protein or conjugated protein according to any one of the preceding claims, wherein the fusion protein or conjugated protein does not comprise any other of the follistatin domains FSD2, FSD3, and/or a N-terminal follistatin domain, preferably wherein the fusion protein or conjugated protein does not comprise any of the human FSD2 of SEQ ID NO: 41, human FSD3 of SEQ ID NO: 42 and human follistatin N-terminal domain of SEQ ID NO: 43.

63. The fusion protein or conjugated protein according to any one of the preceding claims, wherein the one or more polypeptides (P1) (i) do not comprise any other of the follistatin domains FSD2, FSD3, and/or a N-terminal follistatin domain, preferably wherein the fusion protein or conjugated protein does not comprise any of the human FSD2 of SEQ ID NO: 41, human FSD3 of SEQ ID NO: 42 and human follistatin N-terminal domain of SEQ ID NO: 43.

64. The fusion protein or conjugated protein according to any one of the preceding claims, wherein the at least one FSD1 domain of the (i) one or more polypeptides (P1) comprises or consists of a variant FSD1 domain encoded by a sequence selected from the group consisting of: SEQ ID NO: 47, SEQ ID NO: 50, and SEQ ID NO: 53, or a polypeptide having at least 70% such as at least 80%, for instance at least 90%, such as at least 95%, for instance at least 96%, such as at least 97%, for instance 98%, such as at least 99% sequence identity thereto.

65. The fusion protein or conjugated protein according to any one of the preceding claims, wherein the fusion protein or conjugated protein further comprises a PROTAC linker, preferably wherein the PROTAC linker is selected from the group consisting of: flexible aliphatic linkers, flexible PEGylated linkers, flexible heterochains, rigid linkers, triazole-based linkers, Bio-orthogonal clickable linkers and photo-switchable linkers.

66. One or more polynucleotides which upon expression encode the fusion protein or conjugated protein according to any one of the preceding claims.

67. The one or more polynucleotides according to claim 66, wherein the one or more polynucleotides have at least 70% sequence identity, such as at least 80% sequence identity, for example at least 90% sequence identity, such as at least 95% identity, for example at least 99% identity with a polynucleotide selected from the group consisting of: SEQ ID NO: 4, SEQ ID NO: 6, SEQ ID NO: 8, and SEQ ID NO: 10.

68. The one or more polynucleotides according to claim 66, wherein one or more polynucleotides have at least 70% sequence identity, such as at least 80% sequence identity, for example at least 90% sequence identity, such as at least 95% identity, for example at least 99% identity with a combination of SEQ ID NO: 12 and at least one polynucleotide selected from the group consisting of: SEQ ID NO: 14, SEQ ID NO: 16, and SEQ ID NO: 18.

69. One or more constructs or vectors comprising the one or more polynucleotides according to any one of claims 66 to 68 or encoding the fusion protein or conjugated protein according to any of the claims 1 to 65.

70. One or more constructs or vectors encoding the fusion protein according to any of the preceding claims.

71. A host cell comprising the one or more polynucleotides of any one of claims 66 to 68 or the one or more constructs or vectors of any one of claims 69 to 70.

72. The host cell according to claim 71 wherein the host cell is a mammalian cell.

73. The host cell according to claim 72 wherein the host cell is a human cell.

74. The host cell according to claim 73 wherein the host cell is a Human embryonic kidney 293 cell (HEK293) cell.

75. The host cell according to claim 72 wherein the host cell is a Chinese Hamster Ovary (CHO) cell.

76. A composition comprising the fusion protein or conjugated protein according to any one of claims 1 to 65, the one or more polynucleotides according to any one of claims 66 to 68, the one or more constructs or vectors according to any one of claims 69 to 70, the host cell according to any one of claims 71 to 75, or mixtures thereof.

77. The composition according to claim 76, wherein the composition is a pharmaceutical composition.

78. The fusion protein or conjugated protein according to any one of claims 1 to 65, the one or more polynucleotides according to any one of claims 66 to 68, the one or more constructs or vectors according to any one of claims 69 to 70, the host cell according to any one of claims 71 to 75, or the composition according to any one of claims 76 to 77, for use in medicine.

79. The fusion protein or conjugated protein according to any one of claims 1 to 65, the one or more polynucleotides according to any one of claims 66 to 68, the one or more constructs or vectors according to any one of claims 69 to 70, the host cell according to any one of claims 71 to 75, or the composition according to any one of claims 76 to 77, for use in the treatment of an ophthalmologic condition, an oncogenic condition, or an inflammatory condition, such as a rheumatic condition.

80. The use according to claim 79, wherein the ophthalmologic condition is neovascular age-related macular degeneration (AMD).

81. The use according to claim 79, wherein the inflammatory condition is caused by organ transplantation.

82. The use according to any one of claims 78 to 79, 81, in a subject donating or receiving an organ, and wherein the donated or transplanted organ is selected from the group consisting of: kidney, heart, lung, bone marrow, and liver.

83. A method of treatment of a disease or condition, comprising administering to a subject an effective amount of the fusion protein or conjugated protein according to any one of claims 1 to 65, the one or more polynucleotides according to any one of claims 66 to 68, the one or more constructs or vectors according to any one of claims 69 to 70, the host cell according to any one of claims 71 to 75, or the composition according to any one of claims 76 to 77.

84. A method of treating an ophthalmologic condition, an inflammatory condition, or an oncogenic condition, comprising administering to a subject an effective amount of the fusion protein or conjugated protein according to any one of claims 1 to 65, the one or more polynucleotides according to any one of claims 66 to 68, the one or more constructs or vectors according to any one of claims 69 to 70, the host cell according to any one of claims 71 to 75, or the composition according to any one of claims 76 to 77.

85. Use of the fusion protein or conjugated protein according to any one of claims 1 to 65, the one or more polynucleotides according to any one of claims 66 to 68, the one or more constructs or vectors according to any one of claims 69 to 70, the host cell according to any one of claims 71 to 75, or the composition according to any one of claims 76 to 77 in the manufacture of a medicament for the treatment of a disease or condition, for example an ophthalmologic condition, an oncogenic condition, or an inflammatory condition.

86. The use according to any one of claims 78 to 82, the method according to any one of claims 83 to 84 or the use according to claim 85, wherein the fusion protein or conjugated protein according to any one of claims 1 to 65, the one or more polynucleotides according to any one of claims 66 to 68, the one or more constructs or vectors according to any one of claims 69 to 70, the host cell according to any one of claims 71 to 75, or the composition according to any one of claims 76 to 77 is administered systematically.

87. The use according to any one of claims 78 to 82, the method according to any one of claims 83 to 84 or the use according to claim 85, wherein the fusion protein or conjugated protein according to any one of claims 1 to 65, the one or more polynucleotides according to any one of claims 66 to 68, the one or more constructs or vectors according to any one of claims 69 to 70, the host cell according to any one of claims 71 to 75, or the composition according to any one of claims 76 to 77 is administered locally.

88. The use according to any one of claims 78 to 82, the method according to any one of claims 83 to 84 or the use according to claim 85, wherein the fusion protein or conjugated protein according to any one of claims 1 to 65, the one or more polynucleotides according to any one of claims 66 to 68, the one or more constructs or vectors according to any one of claims 69 to 70, the host cell according to any one of claims 71 to 75, or the composition according to any one of claims 76 to 77 is administered intraadiposally, intraarterially, intraarticularly, intracranially, intradermally, intralesionally, intramuscularly, intranasally, intraocularally, intraosseously, intrapericardially, intraperitoneally, intrapleurally, intraprostatically, intrarectally, intrathecally, intratracheally, intratumorally, intraumbilically, intravaginally, intravenously, intravesicularlly, intravitreally, or liposomally.

89. A method of increasing the local half-life of a therapeutic or diagnostic agent at the site of administration, comprising obtaining the fusion protein or conjugated protein according to any one of claims 1 to 65, the one or more polynucleotides according to any one of claims 66 to 68, the one or more constructs or vectors according to any one of claims 69 to 70, the host cell according to any one of claims 71 to 75, or the composition according to any one of claims 76 to 77, wherein the therapeutic or diagnostic agent (ii) of any one of claims 1 to 65, or the therapeutic or diagnostic moiety identical or different of claims 6 to 65 comprises or consists of said therapeutic or diagnostic agent.

90. A method of increasing the local in-vivo half-life of a therapeutic or diagnostic agent comprising the steps of:
a) providing a therapeutic or diagnostic agent;
b) obtaining the fusion protein or conjugated protein according to any one of claims 1 to 65, wherein the therapeutic or diagnostic agent of step a) is the therapeutic or diagnostic agent (ii) of any one of claims 1 to 65,
thereby increasing the local in-vivo half-life of said therapeutic or diagnostic agent.

91. The method according to any one of the preceding claims, further comprising a step of administering the fusion protein or conjugated protein to a subject suffering from an ophthalmologic condition, and wherein the fusion protein or conjugated protein, or the composition is administered intravitreally, subretinally or suprachoroidally.

92. The method according to claim 91 wherein the ophthalmologic condition is wet AMD.

93. The method according to any one of claims 89 to 90, wherein the fusion protein or conjugated protein, or the composition is administered to a subject donating or receiving an organ, preferably a kidney, heart, lung, bone marrow, or liver, even more preferably a kidney, or administered to said organ by ex-vivo perfusion, and wherein the therapeutic or diagnostic agent comprises or consists of a compound reducing inflammation.

94. The method according to any one of claims 89 to 93, wherein the local half-life of the therapeutic or diagnostic agent is increased by at least 6h, such as at least 12 h, for instance at least 24 h, such as at least 48h, for instance at least 72h, such as at least 96h, for instance at least 120h, such as at least one week, for instance at least 2 weeks, such as at least 4 weeks, for instance at least 8 weeks, such as at least 3 months, for instance at least 6 months, such as at least 12 months at the site of administration.

95. A method of increasing the binding of a therapeutic or diagnostic agent to the extracellular matrix comprising administering to a subject the fusion protein or conjugated protein according to any one of claims 1 to 65, the one or more polynucleotides according to any one of claims 66 to 68, the one or more constructs or vectors according to any one of claims 69 to 70, the host cell according to any one of claims 71 to 75, or the composition according to any one of claims 76 to 77, wherein the therapeutic or diagnostic agent (ii) of any one of claims 1 to 65, or the therapeutic or diagnostic moiety identical or different of any one of claims 6 to 65 comprises or consists of said therapeutic or diagnostic agent.

96. A method of increasing the binding of a therapeutic or diagnostic agent to a predefined organ comprising administering to a subject the fusion protein or conjugated protein according to any one of claims 1 to 65, the one or more polynucleotides according to any one of claims 66 to 68, the one or more constructs or vectors according to any one of claims 69 to 70, the host cell according to any one of claims 71 to 75, or the composition according to any one of claims 76 to 77, wherein the therapeutic or diagnostic agent (ii) of any of any one claims 1 to 65, or the therapeutic or diagnostic moiety identical or different of any one of claims 6 to 65 comprises or consists of said therapeutic or diagnostic agent.

97. A method of increasing the intracellular uptake of a therapeutic or diagnostic agent comprising administering to a subject the fusion protein or conjugated protein according to any one of claims 1 to 65, the one or more polynucleotides according to any one of claims 66 to 68, the one or more constructs or vectors according to any one of claims 69 to 70, the host cell according to any one of claims 71 to 75, or the composition according to any one of claims 76 to 77, wherein the therapeutic or diagnostic agent (ii) of any one of claims 1 to 65, or the therapeutic or diagnostic moiety identical or different of any one of claims 6 to 65 comprises or consists of said therapeutic or diagnostic agent.

98. A method of degrading an intracellular protein, said method comprising the step of coupling the fusion protein or conjugated protein according to any one of the preceding claims to a ligand of E3 ubiquitin ligase, wherein the therapeutic or diagnostic agent (ii) of said fusion protein or conjugated protein binds to said intracellular protein.

99. The method according to claim 98, wherein the ligand of E3 ubiquitin ligase is (S, R, S)-AHPC-PEG8-NHS.

100. The method according to any one of claims 98 to 99, wherein the E3 ubiquitin ligase is the Von-Hippel Lindau (VHL) tumor suppressor protein.

101. The method according to any one of claims 98 to 100, wherein the therapeutic or diagnostic agent (ii) is an anti-vimentin nanobody.

102. The method according to any one of claims 98 to 101, wherein the fusion protein or conjugated protein is a Type 1 protein as described herein, preferably wherein the fusion protein or conjugated protein is anti-vimentin-nanobody-FSD1.

103. The method according to any one of claims 98 to 102, wherein the method is in vitro, in vivo or ex vivo.

104. The use according to any one of claims 78 to 82, 85 to 88 or the method according to any one of claims 83 to 103, wherein the subject is a human or a non-human animal.

105. The use according to any one of claims 78 to 82, 85 to 88 or the method according to any one of claims 83 to 103, wherein the fusion protein or conjugated protein according to any one of claims 1 to 65, the one or more polynucleotides according to any one of claims 66 to 68, the one or more constructs or vectors according to any one of claims 69 to 70, the host cell according to any one of claims 71 to 75, or the composition according to any one of claims 76 to 77 is administered systematically.

106. The use according to any one of claims 78 to 82, 85 to 88 or the method according to any one of claims 83 to 103, wherein the fusion protein or conjugated protein according to any one of claims 1 to 65, the one or more polynucleotides according to any one of claims 66 to 68, the one or more constructs or vectors according to any one of claims 69 to 70, the host cell according to any one of claims 71 to 75, or the composition according to any one of claims 76 to 77 is administered locally.

107. The use according to any one of claims 78 to 82, 85 to 88 or the method according to any one of claims 83 to 103, wherein the fusion protein or conjugated protein according to any one of claims 1 to 65, the one or more polynucleotides according to any one of claims 66 to 68, the one or more constructs or vectors according to any one of claims 69 to 70, the host cell according to any one of claims 71 to 75, or the composition according to any one of claims 76 to 77 is administered intraadiposally, intraarterially, intraarticularly, intracranially, intradermally, intralesionally, intramuscularly, intranasally, intraocularally, intraosseously, intrapericardially, intraperitoneally, intrapleurally, intraprostatically, intrarectally, intrathecally, intratracheally, intratumorally, intraumbilically, intravaginally, intravenously, intravesicularlly, intravitreally, or liposomally.

108. The method according to any one of claims 97 to 107, wherein said therapeutic or diagnostic agent is transported to the cytosol, such as to the cytoskeleton or to the nucleus.

109. One or more constructs or vectors encoding the one or more polypeptide(s) (P1) (i) of the fusion protein or conjugated protein according to any one of the preceding claims.

110. A composition comprising one or more polypeptides selected from the group consisting of: FSD1 (Q124A) of sequence ID NO: 47, FSD1 (E126A) of sequence ID NO: 50, and FSD1 (Q124 E126A) of sequence ID NO: 53, or variants thereof, having at least 70% such as at least 80%, for instance at least 90%, such as at least 95%, for instance at least 96%, such as at least 97%, for instance 98%, such as at least 99% sequence identity thereto.
